SEQUENCE LISTING (90 sequences)

SEQ ID NO: 1 - DNA, 214759 bp, Homo sapiens
gtaacacattcatattcacttgttcttggcaatgaaagatttaccctgctgtgcattttataaaacttgtcattatgaacacattgtaaacatacagtgtatgaagagaagaaaacagtgaactagcacatacaaggaactggaactggttccatcattatcagtattttgccatatttactttgtgtttattttaagtattttcaaataagctagagaaatcttaacgcttcttattctcccataaacatttttacaattttatttttgaatgtgtatgtatacacattcgtatgcttttatattgttctataactttcttttatatcatgtaatatataaatattttaatatttctatctgattattacatatccatagatgtgaagcttccctatttttaataacttatatttttacataagtaaaatttacatagttaaatgcattgatatatagtaattgtataattcaataagtttcttaaaagtaattactatcccattcaggagatagaatatttttattacttgagaacctaccctattgcctctttccaaccaagcaccttctcaaaaaaacaatgttgtgatttttatgatcatagataagttttgcctgtttggaaattaatataaatggaatcacacagtgtgtgtctggcttcttgcactcatcataatcatgttgagatttatctgtgctattccctatatcagcagtttaaaaaagtttctatagttgtaatatttcactaaatggttatattgcagtttatctattctcttgttgacggacacctgagtagtttctagtttttagctgttttgaatgttagcagatctcttatgtaaatacctaaaaattgaattgtttgatcataagacagattttgcttaacttataggaaactgctagagagtcttctaaagagatggacacttttcgcactcccaaccagtgatatgagatttttagttgctgtccatttttgccaacattggaaattgtcagtcttatctattaagtgggtgtgaggtgatattttgttgtgacttcaatttgaaatttgaccacttaagaaggcaaccagtttttcatgaagttatccaattgtaaatcttctttagtaaaatttgtatatttttcccaacttttaaattacaattttgaagtaattttggacttacgggaaatttgcaaaaatagtagagttcctgtgtattctgtacccagctttctggatgttagcattttatataatcattattataacattattacatacaatgataattgaaatcaggaaattaatataatactgtcaaccaatctgcagactatattcaggtttttcaagttatgctactaatgtcctttttatctagtccaggatccaattcaagatctcttattttatctggcatgtttcctttgccctctctcctgactacaaatctgggagagctccctcagtctttctttgtatttcatttaattgacacatttgagaaatactagaccataatttggtacactagtcctcaatttggatttgtccgatatttttattattagattaaggttatgcacttttgacaaaagataacacagtagtaacagtgtgcccttttcagtggatcatctggagtacatgaagttaatgtcttatttctgctgatactaaatatgacgcctttattaaggtagtgtctgccaggtttatccactgcaaagttactgttttcctttctttgtaattaatattttatgagaaaataatttgagactatgcaaatattttatttcatctaatttcagtattaattgaagattcttgcctgcagcaattatgattatggtgtttacctagtaatggtttctatttccatgatactttctctacttactagaatttgactgtaaggaagatctgtccttcttccccatttatttgtactcagttgttattttacatcagtatggatttgtggatatcttattttatggattgttatatattgcaatcattgtttgtgtttgattttcaattttttttctggatttgatcattgggagctccttaaagttggcccctgtgttcttttaatacaacctcataattttttgaccacttcttattttcgggtatcacaagatattctaggtttatcttgtattttccttccccacgcctgtaatcaaccatttcctcaaggggttctagttcttcatattggaaaatagtatttagaaaccaagatcttggtattaagtgtattaattgctaatggggttttattgcttctaggcctagagctgggagctgtgtgtccgtttacacacatacacatacacacacacccctatattgtctctattcatttatctatcttaaatatatatactaatagtatgtagaatactatagatagtatatatctatctttattaaaaatcacaagttcatactgttgtggatcataatccatcatcatatggttcattttattcttccctcttggcttatttgtgatttgcttcttcaacagtgagaaacctacctttcattatctacaatatgttttgttatttcttcagtgcagtatacacttaaataatttcagaattgctaaattatacccctttaaaaatgaaatcactaactacagcacagtatttaaatatagttcttttgtctttatccttaacagcgttcagtcaacatactattatttctaaacgtatttaggttagttatcttttttttcctatcccctgccatatggttacattaatcatataatcaagcaggtttttttattgtttatattccattttgagttccctcaccttttggattattttaactatattttgtatatgtgaaatattactatgtttctaaaaattaggactgtgagaaataccactctcccatcatccctgctacactgattctattcctcccttctttccaccctaatctcacccaacccttagagttaactagtctaatagtttctggtttatttttttctcttgttattcttgttcacaagtgggcagatacatgtatattttatcatatatcacatgtatattttatatccccttccttcttacatgattagtggcatactatagatttttttttgttgcagttttctttttccgcttaatatgtcctggaaatcacttcatgtgtattcaaagagatattcctcattctgtcatgcttagtgcttaactccatggatgcaccacaatttattcaacccctctgctctatgagaattgtttgcaatatgtgtaaagtatcacactacaaatagttttacattggattgtttatttgctcttcgtttggaagtgttctttgtattactgggacatacatttcttgttcaatatatgtgttgcaaaggtttctttcattaagtatcttagcctgcctgccttcctttccatttcctttctttttcttttctttcttctccttttaatgctgttatgctctaaattttaaaatggcccgaatttgccagtggctcatcccttattgctatttattcttcctctccttttctcccccatctcttttcctttaatttacgtgaaattaacataacgtagccatttagtgcattcaaaatgttttgcaactgccatccttaactagttccaaaacattttcagcaccccaaagacaacccatacccattaagcagtcagtcccaactctctttccccatcttttcatttttgttttttggagtttttttgagacaaagtctcactttgcacccaggctggagtgcaatggcacaatcatagctcactgcagcctcaaattcctgggcttatgcaatacttccaccttagcctcccgagcagctaggactacaggcatatgtcaccacaccaggctaatttataaaaaaaattttttagacacagagtcttgcagtgttgcccaggcttcttgtgtctttcgataaacagacattgaaaatttttaagtccagtatatcagtttgagtctcttatttttagtgcttattacttactattaaatctttgcttactccaggtttgcaaataatagttttctaagttttcttctagaagctttgggacataagtataacatctatctcaaatcattttcaaatgtagttgaagttacttgttttcatcaatctccagttgttgcagaaccatttgatcaaaagatttttctttttctactgaattatctaatgccattgttgaaaataagttggccatttacgtatggatcttttttgaactctctgtttttccttctttatgccattctttttcactgaagtttaatagtaaatcttgagattaagtagtgtgagtccaaaattttgttatttcaaggctttatagaggaaggagatggctacactgggtcctttgtatgttcacttaaattttaaaattatcttgtcaatttcctaaaaagcccaaccgagaaattgattttgattacatcaaaactatagataaatttaggggggattgacatcttaacaatgttgagtctgtagttctccatttattaggtcgtctataatttctcatagatgtcttttatggtgtttagtatagaagcataccttgtagttttcaatatagaaacattattaaaatttaacatttaataaactagattaaattgttaaatttatttgttttgttgttttgatgctattgtaaatgaatatgtttaaatttcgttttccagatttgttactattatatgaaaacagtaatattttcacattgcaactgcttttcagttgggttgtcttcttttcagtcttaggagaaaatatgtaatttcttaccattaagaaagatactagctgttatttttttgtagatgcccattaccaaattgagcaagttttattctagtgccagtttgctgttaatttttataataataatgtgataaatttgctgaatgtttttctacatttattaatataatcatgttttcccccttattctcttattatgggagactgcattggtcagcttttgaatgttagcccaacattgcattcttgatgtaatccaattggtcactgttatccttcttgtatatatattactgtattcaatttttctgctagttatttaagagttattcacctatgtccgtgatggccattatcctgtggtttcctttttaaaaattatttttgtcagatgttggcattaggcttcataaaatgtatttagtatagtgctctatttgctagtacacttgacccttgaacaacagacatggagattaggggttcagactcccccatagttgacaatctgtggacaattgtttgctctccagaaacttaactacgaatagcctactgttgactgaaagccttacagataacataactagtcaattaacacatattttatgttatatgtattacatactattttattaacaataaagtgaatgagagataaaatgttattacgagacataaggaagagaaaatatatttactattcgttacgtgaaagtagatgatcataaaggtcttcattctcatttttatgttgaataggctgaggaggaggaagaagaggaggggttgttcttactgtatcggggtggcagaagcagtacacttggtgtaactgtatggaaatatagtttctagcttttttgatttttcatttctctaaaaatgtttctatatggtacagtccttcttccactgtttacttatttgtagtacccacatcatagaaggagccatattgtaaagaagtcaaatgcagacttaaataattagaaaccttctgccagcgtgtttttctggcactccttctgtgtcgtcttccttatcgtctggtactggttcagaagcacttgtctccatcacgtcatcttctgttaattcctctagtgtgttatctattagctcttgaatttctccaaggtgtatatcttgaaagccttcactcaccacccttttttccgtgttcacgatctctttcgtgatttccttggttggctgtattgtaaatcctgtcaagtcatgcacaacatctggacacagtttcctccagcaggggagtgtgttgtgttgggcttgatgctttcacagctttttgtgtaacgatggcatcttcagtgctgtgatccttccagactttcatgatgttctatcagggttctcttgcatagtgttagataatcctttccataaagtactgtgtgtaatgagccttaatggtccttatgactccctgatctagaggatgatttagagatgttatgtctgggggcaaatagaccactttgacaccttcagtattgaactcatggggtcagggacattgtccaatcaaaaaaaaaaactttaaaaggcactcacttactgcaaggtacttcttgacatcagggtcaaaacatcaatgaaaccaatacagaaaaaaagacttcttattgtccaggcctttttgttttacaatcaaaagaccggcagctggtgttttaacttttcccttcgaggctcaggagttagctgctttatagataaggtcagtcctgatcataaacccaattacatttgcacaaaacagtagaattagcccatcccttcctgccttaaatcctggtgctcacttctcttccttactaataaatgtcctttctggcattttttttttttcagagtagggcactttcgtcagtttaaaaacctgttcaggcagatatcctttctcctcaatgattttcttaatggcatctgggaactgcttctcctgcttgactgtttttaagccagacttctttctaaaattatcaaaccatcctttgctggcattaaattctccagatttagaaccttcaccttccttttgcttaggatcatcttgcaccctagaggtatgccttacagcaatcctgtacccgcagaaaagatgcattttcaatagatagatactttgcaaaagtgcaaggttttcacagtgttggtacagctgcaacaatggcttcatgaatttccttttttttttttttttttttaacaattgtcctcacactggatttacttatcttgaaatggcaagtagcccctgctggagacctcaatctgtggtaccttttaagcaattaaacgttttcttgtaatgtcatgactttcctctgcttcttaggagtacttccagtatcaccagtggcacttcatatgggtcccatggtgttaaggtttatggtattacacaagacactatgaaaagtaatatgagaactgtgaaagcttttgttttgctgccacatgcaatttactggagggagaaactgtttcctggcatgatttcgggtcacctggcactttaagcagatgcaattgagctccccactgtagcaaaaggaggtggctacaaaattattacaataatgctgggtgtactacagctaattttatgcagttaggatttaataccacatatttacatttctctcaactgcaaatggcaccatgtaggatctgtgagtatgtgtgagtaggttttgataaattttaactttttataatttgtgtatattttatggtaataaatgataaaaataggctagtatctacatctattttatgcattcatgacatacctttttcttaatttttttggtatttctggactacgcagttgagttttttcaaattgtcacaaatttccaaaaatgtatccaatgtatttattgaaaaaagtctgcatataaatagatcatatgcagttcaaacttgtgctgttcaaaggtcaaatttctttgtggaatgtttgatagaattcattagtgaaatgatctgggcctggagtttccttcgtgatattttaatgaacaatttcagtttatttaataatacaggattgagagtttctgtttcatttaatggcagctttagtactgtttttcaaagagtatagtccattttatcagatttgtcaaattcagtagtataaagttgctcatgatagttccttattacccttatactgtctgtaagatctcacctttccaattctgatataggtaatattattttttctcaggttggtctttttttttttttttttttttaactatcttgtcatttaaaagaatcaatctttgatttgctaattttctctttatctgttttttatattattgatttctttccttcatcttaccttggttttaatatgctcttttttttggtagcttcttaatcttgagactattaagtcttctattatgtaccttacttattttctaatataaccattgaaggctataagcactacattgatagattccacaaactttgatatattgttttaattaccatttaattcaattattttcttcctttgtgttttaatttgatctgttatttttaagtggagtatttaatttccaaatatttggggcttttctgtgtagtttgttacaacttctgttttagttttgttttggttctaaaaagttactctgatatttcagctttccaaatttattgagacttatggtctagcatatgatctgtcttgacaaaagtttcacatgcacaggaatatatatttaaactcaaggtgtgcagattttgttcagatagatagtgtctagattttctagattgtcagtgaatttttacaattgttttataagttacaaagagaatagtgtaaaaaatttctgctgtgattatgattttctttatctttagttctctcagttcttcgtgtatttggaaatatcttattgtttatacttttatgaacatcatgactgtaataaattttatcttttcctaatttggcctgtcttaccttatgtaatatttctggtcttgatgtctgcttcgtctgatatctgaataatattaccagttttcttgggtttgcagtatgtctggtgtatctttttctgttctttaaatctgtcttcatagtgcttccattatagagagcatagagatggatcttttttgtccaatttaaccatcagtgacttttaagtctttggggtcttaagtccctttgcattaaacatactaacgtgactagacttatgcctactatttagctattgtttttccatttgtcccctctagtttgttcctctctgcctgctttgcttcgttcttttgggttaattgaatatttttagtatcctacattaacttctctgtttacatttcggctccacctctgggtattttgtatgggttataaaatgtacccttaatttatgacaatctgcttagagttagtattgtacacttctcatgaagtaaaattttatatatatatatatatatatatatatatatatatatatatatatatatatatatgagggagtctcaatctgtcacccagactggagtgcgggggcatgatctcagctcactgcaacattaggtggaactgtattaactctaaaaggactgtgataatttaaagatgtatattataaccatagaataatcactaaaaagtacagagaaatatagctaaaaaagcaacagcagaattaaaatgtaatagtaaaaatacttgattaaaagaatacaggaaagaatgaagaaaagaacaaagtaaactgtatagaaaacagatggcaaaatggtagacctaaacacagtcatattataaattactttaacggtacacttgtttttcattgctttaattgatcaaatgataggtaaagtattttaaagtctctttgtagttttctttaaatcttaacattttgtgcaatgtttgctttgtgaagattgatgctatgatatttaccaatcatttagaagagatattgctgggtgttttggtaagtttaggttacttacatatcttatatgatgaaatcaacattttttatttgatcaacttaatagaggcatcctgtttgaaagaagaaaataacagatctacaaaattctgattcatgtaataagtaaccattatgataataaatgtttttatttattgagtgcatactgaaaagtggtaggaagattttgtgctgaaatatttgtaaacctagtaattaaaaaatgttttggctgggcttgttggctcacgcctgtaatcccagctctttgggaggccgaggggggtggatcacttgaggtcaggagttcgagttcgagaccagcctggccaatatggtgaaaccctgtctctactaaaaatataaaaattagccaggcatggtggtgcacgcctgtagtcccagctactcgggaggctgagatgggagagtcgcttgaacccaggaagcagaggttgccgtgagtcaagattacaccattgcactccagcctgggtgtcgcagtgagactctgcctcaaaaaaaaaaaaaaaaaaaaagctgctaattaccccatgctaaatgtgttgtaatggtattaatttgatatataacatatatgtaacattttatatatgtgctcatatgatgatactgataatacctccctcaaattgctcttttgagtattaagaaaagtttatatagaaaaaccagtttggaaaggtttggaacataaggctacagtgattagatgttgagaacagcagcaataatagttgtcattataatatctgattaaggcaatatttgcacccaggtataccttgcttcagaactgtgtgattaaccattataccttgaggctattcataccctaccttcactttgttggcataatctgaagttttaaaccataatcacatattgcatttgcacaattaaattttaaaatatatacaaattacagtcaaattattgcattcatttcatgactatattctgtttccatccataagtaccgtatggtcaacaataacatgtcaaagagaattttgttgtcacctcgtttcattgtaccatgttcacactgttctttatctcttttttccattggcagtattgtgttctcaagaagaattattccagaagcacccctgaaataatatatttccttaattttggtattattagcccttctttgcttttctctataaattgtcctgatcagtcacctctctcaattatatacaacacaaagtagcttaaaggagaaaaaaaaggagataaataaccatgttgggtctggcgtcaggcttaatggagtccagtagtccagaatttaaagacacatatgaccgtttgctcccttatctcttagttctccatttcttggtttggcttaaatcttcagtcactttttctttatgtgaatttgcaaagggatcatttgaagccctagatgcaattattacctgccttgcaatcttggtaggatgtgtcctccttccctcctccttgtccatgtaaaagttggtgggtgaagagatggacaaggcttgagtcactaagacattcactgacctgggaataactcaggataggtctggtcatatccccaatggaggcaatgtaccctaatgaaaaggtcagggtcactggtctcattgtccaaggtgttatctgagctcatggtctcacaaccaagaaaattaaggagtatggacacagggtgaggttggttggagcaaaagtttagtaagcgaaagaagaaagctctctgcagcagagaggggagtccaagtggattgccgtttttacagtcattcaaaagcttttttaagaaactcctctcatctctgtagcactttgagtaacttctcttatcagcaaagctgtctgtacaactccccttatcttatgcagctgtgggtatgtctctaggcaagcacaaagcgccgcttatttttgtttgtgtaactgagggtttgttttaggtaagccccctcctttctgtgcaggcttccagggagcccaccatgtacatatctgaaaagggaaggaaactttttgctgggagcttgttaatcatacaaagaataaagggcttccatgccagactttgcctgcctgcttatctatggcttatctgtgcagctatagcctgagtttcccctggctgctctgtttttgcctgtagctgtgattttttcaggcagacagcttctctgaggactagtctcttagctgtttactcaagtaatttttcctgtccttttccctcactatgattaccagctcccctaggacttcatggtctggaatgaaatattccgtgggagtcagggaaagggagtttgttgcaaacatgtccaggccagacactttatataaagtgtcaaagtaaagtacacttacataaagtgtaagtgctgccaccaccactagggtgccaggtactctaacacaagtctttattcccatagatgtgagttaacatttataccaccagctcacactatcaaacaatcccacgtaccccaaataaacactggtgcctggtgggcagcaacaagccctccttcaaggaaggagttcatttgttggtttatccagctgctcccgctttgcttatggaagcgctccgttttctaggggtcttagaagctgtagcagtggtagaaagccccttttttgtggactattatgacttaggcttttatcagagcctcagagaattgctgttgttttgttggttaggctttaggtttccaatctgcatacacaattcctttgaagcaccaggcttccaggcaatttggttgagggaacttattgggatatgaaatattatcaggcaccatttctaatcaatgtacctaagttatgacttcaggtttagagaaaaaaaaaatttttttcccttggcttcaggcttccttctggaggtgataacctcctaaatggactgtctttaagggcctgtggcatgtgccctcctttcaggaaactagtgtatgcctggaggcattggtaatggcttctctcatcttactgtggcttgggaagaatatggctattgcagattttctagtttcattaccacctgaccttcaggctccttacatgttataggcagaaaaggtgcctccacaaagttgtgggctattctctgctttcagaaggacagctggaaatggtacttaattctttgtagcagcaccatcatgaatagctgcccacacccatatacatcattatcttttttggtgaagactctaagacttcaggccatgatatccacaaggctatgttcatgcactacttcggtataatctcaaaataattgccagctccccagtggaacgcctgccttggtccatgtggaatgtgggcatatggtaggatttccttggaggaactagaagaggaacataaagaatgttaggcatataaattattgtttctttttcttcttgtttctttccactgagttatgtgtgagcccccacacccaccctaccttcctgtcctctaccttactgatgctattttcaggctgttgtagaattgggtgtgtaaaattaaattagcctttctcctcttaatatctgtaaaatgaacaaatttgacaaagaaactacctatctgatatataagaaggttattagcctgtaccacaagtgaaaaagtttcatcctgatatatcaaaatagtaccacttgaaggaaaacgttggggccatctgtttctctcattgtagttagagtacagatttttaaaatatgagcctcaggcataaaaatgcagtattagcaactcaacttgaagaagcagttactagtgtcctgggtgggccccagggaaaatagtaccaactgcctgttatatttaactgcaattaaaaatggcagtgctgggtatcttctgtctctgggtcatctcctaaagatcctgcagagggagccatagctagtgtagaggccagccataactagccatggcaaagaccacagggaacgctcttcaacatgctctgcactgatcctctaccctggtgtggagaaatgtcagattcatcactaacaaggttaaaggtgtaaattgagggaaaaaaatgagaaaagttatctttcacataagacttcagaaagaaattgcgtcgtcaggtttggaactggtttgtaatttcatccagttattttcattttaattactgtcactgttttcttcatccttgataaactatgagctctttgagattgtctttttcatatcctgatgtcagagcttcctgtattttctgatcatggtatgtgctaagtaaatgcaatttttagcattttgaatggtttatcaaatgattgaaggagtgggtaataatatagattacatatacagtcagccacatatgtgtatgtatgtgtatttgtgtgtgtgtgtgtatatgtatatactgttgaccctccgtatcaatgggtttcacatttgtggattcatccaatcatggatcaaaaatattctttaaaaatatggttgcatctgtactgaacaggcacaaactttttttcttatcattattccctaaacaatgcagtatgacaactatttatcatagcattgacactgcattagatatgataagtaatctagagattaaagtatataggaggatgcatgtggatcatatgcaaataatacactacatcttatataagggacttgaacatccatggagtttggtatgtggtgggtgatacctcctctttgaggaggtgagtatcagagacgcaggtaaatatgatccagagggcaaagaggcattgccagcctcacactggctgtcaacaccatgctgagttccacgaaagcagcgaggagagggtggtgttcttgtttctggtacaatacctggcatagagaagatgtcacagcaatgttcgtctattgaataagtcaccaaaattgtgaatgactgaataaatgggcaaagggatttgttccctacttgctgtgggaagtttcttgtagttatgacctccatgaaagagagactcgtatctggagaaagttcatagctgaaaccatgagatttaacttttgaagttataatggatctatttttttaacatcataaatgaagattatgcaatactgtatgtgcttcaattgacacaaccccttcccattcagggctgatttatgttctgtactgaatgacaggcacagaagtaaatgtgataatcctgtcccgtcgttaccctttattgtggtcttgctgtgtcccagccacacacttcatactgtatctgtgtcatcattctcatatacatctatataggtacacttccatagacttaggcccatttaatggaggagcaactccaacatttagagatcatgaaaggtacctcatgacatatattgacctagtggcagagctgcttagttctacagcctctgagtggaatcctcacgacagtaaaaacacaagcttggtttctcacacactctacctgctttttctcatcccctttagtctctattccttgctgagatgtgttaggcattaaatggttacagtctacttctgtgtgtagtctccaaacactcagaaacctcaaaatttaaaaaaatacagaacaatatgttattaaataatcagggacaagttaaaatggtaaagagaataggccactttgtgaaatcatatgcagggaagaaaaaaatactggagtgaaaacaatataaaaataatactagatgctgtttcgtagcaccaagtgtgttctagcttctgtggttgttgctttatatcattttatataatctttacacaaaacctacaagcagatattatttcccctatttcaaaaatgagaaaaccaaggcaaatagttggtaagtagtacatatatattatcctaactctgaacttctggattcaaagaccatgttctaggataccattttgtgtaccagcatggaatatgtttttagttcatctatttactctaaaaatgtggagcatgtagtgtgagacagaactgttctaggctgtgaggaagtagctgtgaggatggtagtatgggtccctgtaataatgaaaatggaatatattggtatgtacaagtacatgctcattaaaggaaattgatgttggcatagattgcctgggttcaaattcccatttaccatgttttcctagttgtctctgtggtgagaggaggtggaatgtactagccattaccaaaattaagagagattgttggaaaaagtctcatttagcacaataacacattaaatgtattcaataaatgttacttatgactctttgagaaaacatacgtttaaaaaacatggcttatgttacacatactatatgctggctcccaaaaataaacatatttcccaagaagtgtctgttgataaagccaaatatattcttactacactaaggggaagcccatcttagcagtgtctcagcagcatattaggggcgtgggggacaaaacaggatatctgggatcttgaagtttcattttagatgggtctttcagtgtgggagcttgattaggattgggtgagggtcatgacacagcattttaagattgaacgttaggattttgagatgagggattctacagtgtggaggcataagctgtggtctgatgctttctgttgatgagttgatggtctttgggaagttcccaggatgaacaaaaatatttgcaacttttatcttcctgggcaagagtctcagaacatctacacttatatcaatgaaggtaaaatgataataatgtccccagcgaagacaggaagccatgtgggtagataatgtaagttgtgtcatttcatggttccatggataagcagctgtacttaagctgcttatcctggtggattctggagatccacacttgaaggtatcaacacagaaatatactaagaggattgtggtcagagtttgttgtcagctactgagtgaggagccaggaacatacagcttttaccaagaaaatctataccatccacactgtgatgagcccacaagaccagatatgggactttagccaaattacctaattttattatgtctttgattgctttctggtccttatctgggatatcagagatcccagagagatggcagggatgcaaattaaacattcttcccccaaaaacaacacatttttcccttataaaattactatattctgaaaaatttcatgaacctccaagtcaagatggggcattgtctaacttgcctgctgtttgctatacaatagcatggagtcagctccaggcttcccaaccctttcgaaaagcatgaattgaagaggcttaaggagagaagaaagcaacccagaattccctgtgtggttacagtttgggttcttgcctcaaggcctggtttttctctttttggagtaaagttatctgaagacctctttttctaagagaagaaactaatgggtcaaaatggaccctgggggatgtcagggactacagtgaaggcagacgttagttcacccctgaaataaatttctgtccagtgtgcagacagtagccagcagacctttgtctccatcatccaataccagtcttttcagatctagtcaatgatctatttaaagccaaaatatccttatgagactgtcaaatagtattgtatttttttgtatcactcgtcaagaatagcttagtacccggcaataaattttatagtactgatattaatagatctcaggtgaaagaaaagtcatgtgtcttataagatgggtgaaagccacctccaaatagatactctgttctctagttgtttttcatgttctattcctctgtaagttcagttacaaaagctgacttccacaaaaagttctccagtattgttttggaaccttattttctaatatcttgtcatatacgagttgagtcccaattaatgattcattatgaggaactggaaacgagaaactgagtggctcaccaaggaaaatatgtgcaaacctaacggacaatttggttatatgatttaaccactgcatgcataaattataactaggtattggactcatactgaggagtgattaaaatgtaactatgccttataagcaacataattaaatgtagctttaagccagataggcaagaaaataacaaaaggacgcagattaaaacatgacaatatatggtattgttctatgagtcttgagtggaaactctacatttcatgcagtcatctacttgggctaaaaatcatgtgtagtggccatctccttccaaagaccatcttacttctaatgagttctcattgaggtctcacgttctcagtctgaggtctccaggtctcaagatttctcagcttgaggtctccagtaatgatttaagatggtttatgcctctttgattggaagacatcactctcagtatcaactccctggaattttacttctggatgagtcgttaacagtatccgatagggtcttttccatcagggttcgagggctttctttctcaggtatctctcctggagatacctgagaccaagcctctgaatttagaccatgtgagggtcatttagtagaggaaaagctgcttgtttacctcttaataagactggataaattgcctgagttacctgcaatatttggctgtgatcacttccaataatgtagagtctagaagcagaggagatattcctagactcgtgggttggcctgttgctaactcaaaagggaagatttgatgaaccccagaagttgagctcattgtcattaaagcaaacattaggccatgtgagtttgagagtctttgatagttttgctaatttgagcttaaactttttgtttattctttctatatttcctgaagattagagtaatatggacagcagcgtttctgtgtggactggcagagctgtacaaagctctttatcatctcagtaaaatggagatagtttatacttgtactagagagagagtttgggaacataaaatcatgtcaaattttgctctggcagagctggaaatctccaaacaaggcctcaatctatcctccaaataagcgaacaattaccatacatactcaaaacccattgcagaaggtacttacaaaatctgtaagtgttctaaggggccctgagattcttgttcatgccccatctttgcatttttaccaagattatactggtaacaaataagacaccgcatgcaacctcaataatcttagtgaaatttctccatcaatgttgattgagtgcagtagccaatttttctttgcttaggtagttaattgcatattaaatgtttgaaacatcccgtttaaggtcctctggcgctactaagcatctatcctagagtgtgagagattttcctcttgcaatttacctgcagatttatattagtaacggtttgccaactgtggaaccaattgttgacattctataagaacctgtttgaacccgtcagaaggtttgattatgaggaccaggatcttagtgagggctccatgctggggcctcctggtatccccatcttgtattagtgtttgtttttttaatagccacatctttagcaagctttacaatactcaaaagttcttttatttgctgaccatttttagtagaggctcctgctgagattgagaactctgtttgttcacaaacaagaccaagaactacacacaaagcatacctgccaccagcactctcctatctcactcaaactgaccagatctgtagagcgaaatgagtcctgtcatctggcagattttgcctctggtagtgattataagggtgaagtatgtcacatcatgtgccatctgatattcttttgtcccagtcttaaagtacagcacatcaaagaacaagatgaaacatggatttttaaaaagagattactaatacatctatttagggcatagtttgtaaacaaagacagtacacagcttcttcttaaggtaatggaaaaagtagcaattttgaaagtattgccataagtaataaagatgtgaggtgctgatgtgaagtaatatttcataggaatttaagcctctatgctgaaaagtatgtattttcaattttgtgtattttcagtaaaggttatccagcaagagggactatgaaacttaaaggagaccctaacctaaaccaagccagcagaggcttccagtaatttagctgttgtggctattgccactgggtgaagatacagacttcaatgagctggcccctgaggattctcctgaaatgcatttagaacaccaagagcttatccccagcactcatatacaaactggtagagagttctgtgataattaggtaggcctaaagtgggaggtcattggaaggctaattttaactcacaaaaatctctttgtgattaggttcttagggctcagttttggctactcataaagtgggtaagtcagagagatgggaaattttggaaaattttggaatgttctgcaataagcatccaaggaaacccaggaatcctcttaatgatctcttaactactagcctgaaaagttttggatagcttataatctatcaggcataaaggattcatcttcttgggataaatcacatcataggtaacgaacttaaacaaaattctaacttctctttggaaattgtatgacctttctgagctaaaacaaagtagataggtagaataagtcttagaacttgctttgtctttagaacacagtcactggtcattcatattatctattgataagagtggaatcacagaaatttagggtccttgaaatcgttgtgaaaagtaggaaggtatctaagtaaatccaggggatatagtacaagtatgctgttgattttgcctagggtgtaccaacaagtatagagtattttaatacaaaggcactataacaaaaaagtagaacacaaatctgccactataagatatgcagcctcaaggactcagtggaaataagatggtatttgggtggggtatgataaacagcgattattctatttatggttctgaagtcctgaaaaaatctgctatctctgccactgggtttttgagcatggagtgtaaaagaattataagggctgttacaaagtttttagctattattttagtcctttagtcctttttaatctcttggttttagaagagattgtgcaagttgggtagcagttaaggtggataaatctgagtcttaaccgggtttatccataagaatgtcaagcacttccttgagatcttcaattttggcttgatttagattcaatagtagtcgtgaatcaatgtccagattatctatggtttctttatgagtgagggagtcagagactcaaggtctagaccatccagatagcatttaatttacagttccacccacgtagtaacttcctacctatcatgtgagtgaatatggtattacagagaaagaaagagtgtaatttagttaaagtccaagggtggctgtaatgggctgggatgtgaggaatatttgataattattgcagaaacctaccatgtgagtagtttattttcaatgaagaggttgtgtaatagtgataaggtttaaggttgatgatatttagcacccatatcagttaaaaaaatttgcagagttgtcatttaatatttagagaaagttttctttttgacttgaaagatgaaatgttttgtgcttgatacccaccggaagcaccttgatttgtcacttatttcatgttttgccctttgattctgagctaatgcaaaacaagtgttttccaatttccttactgcttacaggcgtctttatcaagagacttccttattccgggtttaatgtctgggataggaggatgtaattttatctctactttgattttttttaatgaaactctttcaaaatgtttagtgaggtgctagagatctgataatgggaataatattgctcattgtagtttttttttacatattaaatcctgtttttttggcttcagttcctttctgcaaaatggggaaagatgtgtgtgggctcatatctgcttcaaggtctactgagtccttcctaagggtattaaagagcctatcttcaatttctccaatggacacatcctttctttgcttgcaggactggattataaaacaatctattttcacagggaagactttaggggtagtctccaaaagaccttgtccaatatcttcagtttttctaaaattctttgtttcctgtaaaatttgaaactctttggtttcctgtggtcttttatgtcaccactgagactgacccattttgcctttttcatgtacttttttgcatctagggttatcactaaaatatgaactccttggtatagatttgggagccctggattctaacttcctaaagatgttttataatcctcgcaagttttcctctattctgcctgtgctttgaagagcctttgacggggtctctcatctcagagttagatggcttaaaatcaaccacagatgtttccctgttgtgagggtagtttaacatgaagaggtcattgagctttggggttttctggctgtctgaaagagaagaaaaattgttcagagggagagatgggggagagaataataataggataaagtctatttggtttgagttatgatttgttggtatctaaaacagagagagagttttttttttcttttgccctggctaacaaatttcataagaaagcagttttgaaatctgaattgcctttggatgtttctcagattaattaaaaatgtagactactggatgtttgaaattttgatctatttttattttaatgtactcttaaaaaattttgttcatagaaaaacttcccccaaagtggctgctataattgtaactcatctctgttgaaattatgccatttaaataagtatatgaatgtggattatcttataaacatatggaagaagaagtccagagggcgtttggaattagaatgagactggaggcataccctgaaagtgtggtaaaactctgaaaaaaatgttgcttatgaccccttgggtctctcaacctgatttctacattgtctaactgtagacctgaaggaatttgcagtttcttgggggtacataagatgttaataggggatttctcttagaaccagactgatgatcaagacaagattgaggaaaattgtcataggttttgataaggacctagggaaaaatttctccaaaggacagcaggctgagaagaaatcttacaaacttctccatccctggggctgtcttgagggatccacttacctgggtctctctcactctcttccctatgaacattccttatatatacacatgaagtggcaaagacaaaggcgaagagtagcatcactagtaataaggcagtttcaccaaagataaatttttttcaagtctgatcaaagtgttcctgaaaagagttccttacccttaggagacaaaataatctcagtatactgggagttcaatgaaaatatactggggctcagtccaatttgatttacttaatacactgttgcactaagagatagtcctgaccaggaagctaaaaggatatctgaggaggacaatgaattaaggttctgaattaaggttcttgtgcctaggctgcaaagaggtctcaatctttatggatcttggtgagatctccaagattgttcaatactgaattcctagaaatacaacctgaagaatattttgataaggcaaagctgagtttattcttaatgtggtaagagaaagcaatcccctaatagatattaataacattttggacaaggaaggacaaagttagaatatttatgagactttaaaggctcatttaagatgggtctttaaatgccagggcttgattaggatttggcgaggattatgataaaagagtttactgttggtggacacagcaaagtaaagatttggaggtgagggattcaaaaactcttagaatataagctattgtctgatgttttctactgatgagttaataagtttctaaggaagttcttgtaataaataataaacattattcacaacttttatttttctgagcaagaatttactggaatagtaaagtgaaaatagtgccagtgaaaatagtggaaaatttaaattggaggcaattagccatgagagtgtacatgattttgtttctcacagtgtcagttaacatttcccctctgtgcaaatccttcaatagtttacaatttgtttataataaatgtccgtagaatggcccacaaaactggacctggtcaagccactttctgtcacctcttacctcttagctactacttgctgttctggaaccactgggccagcttctgcaccagattctctcttggccccaagcatttttccaccgtctctctctctctgcttactcctctgccatcatcagttgtcaactaggctaacctggtacacttctctaagtatcaatatcactacaatgttgctctttacccctttccactattttcaacactgatcatgtaatataaaatattatttaatattttaaatgattatccatctccttgcactagaatgtaaacatgctgactgtagagatttttgtctttgtcttttactgctccaacaccaattctctgactttgaaggcaataaaaactgactttaaacctgaaaagaaagagctaaattgccttccagttctttgaagtaaaagtttatacttcaaaagaattgctctggaaagggagaaactgcaagagaaatttcaacttcttgcttctcaaagagaaaggcaaaattatataaaattttgaggaagggtggcatttaggttaaatttataataaacagtatagctgggttagatggaagcaaatggagttggtcagatggggttgatttgatcaaagccaagtggaaatgattggaaggattagagagcagaccagtacaagcagtgcttgctgatgtggacatttaagtgaaaactcattttgaccaattttgcagcttcctgagtatggggtgaggatatcctgttctttaggggataataagattttcagcttcttttgtacatgtgatgatgcctttaggcaagggtgatatttttcattctcatgaccaaagcacagaccataagtctgaacacagaacactatacatggtaatctaggtactctatttgcctttgtaggtgattgcaattttttccttatttaaaattttaagaccataaagaatacttgaaaagtaactttttaccaatcatttaaaaaatactgattaaatgataaaatcccttaaaaacacagttaatgcaaaagccacaaaaagaaatataaaatatagctttacatctgttaaagacactgaacacataattaaaatattcccaaacaacaatgctccatgctgaaatgttccactagtaaatttttacaaatatttaagggaaaaatgtataattttcattttctcaggatagctgacttctagtaactaaacatgacaaagaactataaaaattgaatttacagaacatggctactaggaatatagatccaagtagcctgaacacaataatagtaaatcaaattatgtgaatttttttttttttgagatggagtcttgctctcttgcccatgttggagtgcagtggcacgatctctgctcactacaaccttacaacctccacatcccaggttcaagtgatcctcctgcctcagccccactagtagctgggattacaggcacttgccaccatgccagtttaatttttgtatttttagtagagacagggttttgccatgctggccaggctggtcttgaactcccgacctcaggtgatccacctgcctcggcctcccaaagtgctgggattataggcatgagccaccgcacccagccaaaatatttttttaaaaggtcactgcattataatcatttggggtttacttgagaaatgaagattttttttttaaacattcaaaaaatcattcaacacttctggtttcagctctgatatgtgaggaacttgtaagttataattactgtccttataacaagataaagcagaacaaattgaaaaaaaaatcaattatttcctttaacccatcagagaactgaggcttcagggaaagttgccaccccaaagtctggggacaccgattgatacacagctgaaatttgcttagctggaacagaagccactggagccacaaattgataggaacatttaaatagcaatttagatgaatagctgggctgaatgtgggctagagtgaaaggaagaaactcctgggaacccccacatttttgtgggttctacctccagaacctcaacaatttctcatggtaaagatttaagaaagatcccctcctcctttggaaagggagagaagagtaatcattgtgaaatacacccagagcattcttcataacaaagacctaacctccaggggaaaaattttatctgagccatatctcagtaagggataaggggaagggcattcctttcatttcagccttctctagctttcctgttttaccaatcaggaaataaaaaacccacaattgtcaatagagatcagcacttcaaggaaatagattggaagcaaaagaaggagggaaaagagtagagaggtcaggtggagttgagggagaggttctgccactgaaggaatgtttgtgatggttgtaacaccaagacacaggcacacagagacttattcaaaagatcatagaacactctcccctatgtcttacgaccccaccagcagtgttctgtgtaatagtagattacagttgaaatagctgtaagacaccaattctctcagaggagtacttagggaagtccaaagtcaagagcagaaacaaaagacactagaggaatttggcatttctggtgcctctagtaacagcaaacattaaacaaagcccaattcctcatcagatttctgtaaatagcattctaaagctccatttacctcagtacttactaccagatacagttcaacaaaaaattacaatgcacatcctatgacaagaaaaaacagtctgaagagagaaagcaattagcagaaccagactcagaacaaagatgttggaattaataatagagatttaaaataactatgattaatatgttaaggactcttagacatgcaaaaatagattatataagcagagagatggaaactcaaaagaagcaagaggaaatgctagaaataaaaaacactataacataaatgaagaatgccttcaacaggctcgtaagtagattcagcacagccgaggaaagattcagtgatcttgaagttaggtcaatagaaattttgcaaactaaaatgcaaatacagaaaaagaaaagaatgaagaaaatgggacagaacatccaagaactgtggaacaatttttaaaggtgcaacatagatgtaatggggatactaaaagaatggagtatagagcataatatatgtttgaagtaaaaatggccaagaatttttcaaaattaataacagacaccaaaccacaggcccagaaatctcagagaacacaaaacagtaaaaatgcaaaaaaagcaaaaacaaaaacaataaaacctacaaatatcatattcaacctgcggaaagacaaacacagaaaaagaaatctttaaagaaggtagataaaacatcctcgtcttctcatcaggaaaaaaaatccgaaagtagtaatgtggtagtcaattatggcatatggatgtgtgaaaacaatggcagcaatgccaccagagataggaaaaaaaatggaaatcctctgttataaggcacctgcactaagcgtgaagtggtatggtgtaatttgaagatgggtttagattagttaaaaatgtatattgtagccagggtgacatagcaaaacccagtcttaactaacaatacaaaaattagccaggtgtggtggcacacatctgtagttccagctacttgggaggctgaggtgggaaagttgcttgagcccgggaggcagaggttgcagtgagccgagattgcacccctgtactccagcctgggtgatacagcaagaccctgtctcaaaacaaacaaacaaacagaaatgtatattgtaagttctagctcaaccagttaaacaaataagaaaagataattgatatgctaagaaaggagataaaatggaatcatgtaacatgatcaagtaaaagtagagaacacagtaaaatagaggtcttaaagaaatagtaatgcaatgactagaaagcagttgcaaacatggaatatattgatccaactgtataaataatcacttcagatgtgaattatctacattgaaaaacagaaactctcagaatggactaagacaagaccaaactaaatggctacaagaagcctactttaaaattaaagatttggatactctgaaagtaaagggatggagaaagatataacatgcttacagtaatcaaaagacagctggagtagctatattgattttcagacacagcagaattcagaacaaggaaggttaccaggcatgaaaagtggcaatacatagtgagaaagggccaattctacaagaatatctaaacgtgtatgcacctattaacagtgtcaagatatacatgaggtaaaagttgataaaactgaaaaaagaaatagataaaccctatttatagtcagaggttccaacatccctctggcatagacataaataagaatattgataacctgaacagcattatcagttaagttgtttgatgtttatagagtactccaacaacagaagaatttacatgaatatacattattccaaagtttacatgaaatattcatgaaaacatcatattatgtgccataaaacaaatttaaaagattataaatcatactaagtactcaggtcacaatgtaataaaactagaaaccaataaagaaagacatagctgggaaatctccaaatatttggaaactaaacaacaaacttgtatataatagacacatcaaagaacaagtcattagagaaattaaaaattattttgaactaattccagctggacaatactggttacaatttgtgggatgcagctaaagtagtgcttgcaaggaaatttttagcattaaatacattaaaaagaaaaagaccaataatcaatctgaccttccaccttagaaaaatacagaacagtttaagcaaacagaagaaccttaagcaaacagaagacaagaaataataaaagaataaaaatcaatgaaatttaaaacagaaaaacagtatggaaaaccaagaaaaccaaaagttgatttgtttaaaagattggtaacattgctaaacttctagtcaggctaagcaaagaaaagaaaaaagacaaatgaccaatatcaggagtaaaggaggggacatcagtattgatcccatggacgtgaaaaggataacagaatactaagaacaactctatgcctccagatttgataaatgaaatggaccaataccttgaagtaaactaccacaacctccaaaaggaaaaataggtaacctgaatagcaatatatctattaatgaaattgaaataataattaaccttcccccaaaatagcaccatgcccagagagttttactagtgatttttactaaacatttaaggaagatgcaataccagttctccaccattacttccagaaaatagaaacagagggaagacttcccaactcattacatcacaccatcattaccctaacataataaccagataaaaacattaaaagaaagaaaaactacagaccaatatctctcatgaagattgacgtaaaactccttgacaaaatattatcaaatcgaattcaacaatgtatagaaaaaattagtaccattatgtacattatgggatttattccaggtatgcaaggctctttgaacattcaaaattcagccaatgtagtagaccgtatcaatagcctacagaagaaaaattatattattatatcaatagatgcagtaaaagcatttcacaaaattgaatattcatgatttttaaaatctcagcaatctaggaatacaaggaaatttcctcaacttgatgaaaaaattctacaaaaaaccccatagctaatatcatactgaaatgtaagagactgtatgttaatttcaggagcaaggcgaggatgtcctccaattctatgcaacatcatactggaagtcttagctagtgcaacaagacaagaaaagggaatgaggcatacagattgtgatggaagaaataaaacagtctttactcaaggatgacatggagaaaatcccaaagaatccacaaaagaaaaacttggagctaataagtgagtataacaaagttaaaggatataaggttgatatacaaaaaccaattgcttacctataaaccagtaatagtctattgaaattagaaaaaaactataatagcaccataacaaaagcaaataaaacccttacttataaacctaaagaaaaacaggttcaggatctatatgtctatatgtagaagactataaaactttgataagagaaatcaaagaagatcaaaataaatgaacagatatatcctgtatccatagatagaagactcaatattataaaaatgtcagttcttcctaactttatagactcaattcaattctattcaaaatcccagcaggctattttgtagatgttgacaaagaataaccaacataacactgaagaagcacaaagacagaggagaaatactacctgattttaagacttttactagaagctgcagtaatcaagactttgtggtactggcaaaagaacagatacataagttaatagtacagaatagagagcccagaaatagacaccacacaaatataattaactgacctgtgtcagggaacaaagacaatccaatgcataaaagaatagtcttttcagttaatggtgctgcaacaaggagatgtctgtatgtgaaaaagtgaacctagacataggttttagatatttcacaaaaattaactcaaaatagaccatagaactaaatatgaaatgcaaagtgttaataattctaaaagaatacaagagaaaatgtatttgatcttgggttttgtgatggggaacaccaaagacacagaccatgaagggaaaaaaatggtaagcttttaaaatataagttctgtttggaaaaaggcacttaattaaaagataagccacagattgggggaaaatattttcaaagtacatatctaataaaggtattctagtcaactgtatagagaactcttaaaactcagcaataagaaaacaaatgaccaattaaaaaataggcaaaagatctgaacagacaccataccaaaaaacaaaaacaaatacaaatgataaatacacttatgtaaagatgcttaacattttctgtcactggagataaaaaataataaaataccactacacatgtactcatatacatgtacacagtacacatatacactacacacagtacacatgtattcccatgtaatagaatggctaaaatccatccaaagcctgatcatgccaacttctgttgaaaaggcaagcaacattaattggtgatggaaatgcaaaatagcacagccacttttgaagacagtttggcagtttcttagaaagctaaatgttgtcctgccatatatctagccactacgctccttggtatttacccaactaatctgaaaacttacgtccacataaatgccagcacatgaatattacagctttattcataatgaccaaaacctgaaattaactaaaatatcattcaataggtggatggataaactgtgatacatttatacaatagaatactatcaagcaataaaaaggattaacccatcaagccacacaaaaacatggataaatcatagatgcatattgctgagtaaaagaagccaatctgaaaagaatacatacaatattcttcatttttgtaacattctgggcaaggcaaaactatagatatcaatggttggcagggttcaggcgaaggggcagggatttaatagataaatcacagcaaatctattctgtatggtactgtaatagtgtatacattatactgtgcgtttgtcaaaacacagtgaactttaaagcacaaatgaaccttaatgtacacaaattttaaaaaatcatttaggagtttgggggatccaagatggaacacagtgtctgacaaaagaatccaggtgttttacaaatgtattagacaagcttacgaataatgttggggagaaaagataatgacttaagtaactttgaaaatgagtggaatctgttaagactagaggcaaagtgactgtacaggagcactgcacactatttgataaagttgttttctgtaggtgtatgagttagcaattctaaatcatgatacatatgtatattggaattgaacaattaagtaaatggttggcagatggtgtacattttcttattgttggaatgaaaagttacagacaagaagaaaagaatgatttgtgttgtaatggattagagttggaaacgtcagtgtaaacttatgtttagtttaatatgcagacagatggttacacatagaaatatagatgcatgtatttacatagttattgtacacaccctgtgtcaactgagaatgcttagaagcaatgacactctagtaacaatgggcacacctagaacccagcccttagtttctaataccattctccaatagaacaaatcagggatccttgtagaaatggctgattctaagactggttaggaaatgtacaagatgagcctggagcatcatatagtgccagagagtaaggaagtactaaaacaagaaccccacattgatgaaagtatatcaaagggactcaggaaccaaccaaaagagcttccagtggtcaaatctggaacaaatttttaaaaataaatagcataatattagattacaacccaaagtataaaatatccatgaatccatattgatgtaaataatcaaatgaataaataaactggggagaacatagattttttgtgcagaataatttcatatagtttttatagatacttttgcccttaaggaggttgaactccttactcgtcaagtgtgggaagaaagtcattaggcacattggtgactttctttcaaagagtacagggtggaaatgggggatggaagggaagagtaactgtgcaggggagaaacctgacaagcactatctctacatggtaatcaaggttaacatcagtagtgataaatcatgctgatagtatatacctttgatatgacgtgataaaaattgcactttacctctacagtcttcctcccaaaaacccataagatcagtatcattatgagaaaaatcatcaaacaaatcccacatgagatatactctacaaaatacttgactagttttgctcaaaactgtgaaggtcctcaaaaaggaaagcttgagaaagtgtgacagtcaagaagagcctaaatagacaagacaactaaatggaatatggcatcctatatgggatcctagaagagtaatagtacattatgtaaaactatggaaatctgaataaagtgggctttagttaataataatctatcaatattggttcgttgagtgtgacaaatgtgccatattaatacaagatgttaacaggaggggaaactgggtatagggtatttgggaactctgtttcagcttcactttttaaaaaatcttcaaatgtttacttcaaaaatgaattcaaaatcactcaatgtaaatcactatggtaacataagagagaagaaaagttatatgatcatctcaaaagagaaaaagcatttgataaaatataaaaactattctttataaatctctttattggaactacgaatttatgggaactaaaaattgaaaaaaatttaatgagataaagggtatttacaaaataaattaattgtaaaattattgacagtggagatagagaaagcttttttatgagattgggaataaaagaggattttttttcatcatttctattcattactatactggatgtcctaactaggggagtagagtcatagaataaggattggaaatggaaaggtataagtttcattatttactgacaagaaacaagtctgaacatgatcaattaaaattaggaagttaattgagcaagattgctgggttaaaaagcagataactaaaatgtatttctatgtaaaatgacaaatgaaaaatgaagtttttaaaagataacatttttgggaacatcagaaacatcaattccataggaaaaaattcacacaaaatattatgaaacattatggaaaggaagcaagtaataccttacaaatggagggatataatatattcacagtggcaaattcaattacatatactatacatgttaattctcccccaaattcacttaaagatcccaacgtaagagcattctaaatctgaatgccatttattaaatgttcaatctaatttcaagaattatatatgacacaaaaagtcaagaagagccaagatagtcttgatgaagatgacaattttgcatgagttacatggccaaaatttaaggcctactattgcaaggttacagttactgacaaaagacagaaaactagcctaatggagcaaaatagagactccactccacaagcagatcaatagatatatgattacttgctttatgataaaagtgccactcctgcatgatgaggaaagaatggtttgttcaatagttagtgctggatatcaatacataaaaaagtgaattccttttcaaaccatacagatccatcatttctaggtgtaatgttggtctattcatgaaaggcaaaacagtaaagcttcttgagcatagcataacataggagggtgtcatcataatttggcagaaagtgaaaaataccaataacactaagcataaaggaaaatattgatgaattagtctgcatccaaattaaaatttttatttctcaaagtatataatcagtgaagaagcatcgtataaaagatacaacttgtacatgtcaaaaagttagaaaacacttaaatgggtacttcacaaaagaggatatctgaatggccaataagcctatgaaaaggtattcaaggtcattagccatgagaaaaataattcagacaacaatcagataaacatcttccagtatatctagacagtccccaatttataatggtttgatttatgatttttccactttatgatgttgtgaaagtggtatgcattcagtagaaaccataccttggattttgaattttcatttcccgggctagccactggcagtatgatactctagtgaggctaggcagcagcagtgagccatagctcctagcagtcatgcagttatgaaggtaaacaaccaatacttcatcgcgtactgtgctgccaccattttgacccactgtaggctaatgtaagtgttctgagcacatttaaggtaggctaggcaaagctatgatgttcagtacattaggtgtactaaaagcattttttatttacagtgtttttaacttatagtaggtttattaggaggtaactccttcataaatcaaggatcatctgtttaaaatttgaaagtgtgtgttttttagaattctcttagtattcactgaagttgaatggaggcaacatactctcaggtatatggttaacaaaaattcatgcatgattgacccaaaatatgtatacatgaatgcttatcatggtgccatatctaatagcaaaaattgaaaacaaagtgcccaggaggagttgaaggataagtaaaccttaaaatatgcacatattctctcaaggaacgtggatagtattgtgactgattcttctccatgtgtgatccacatagatgataagatcatcatgagaagaacttaattttatacttgagctttgctttgactgaaagatgagaaattttgtgattattggtatcatctctatgatgtcatattacctagattcacatattcttaactccattaaaatagaaattatatttgtgggcggggaacttattttcatccatctctgtttccttccaattgagcaattccttattttatgttaacctgattttagtggttcattgccataattcttagaaattatatctcattttttcctgtgattttactgtattgaagtaaatctggttggcaggatatacaatattacatgaatgaaatttgcctttggtaactgagaaagttcaaatagaccttggttctagcgaagacttgtttctaaggaatttagattcattctttcagacagtatgaatatcattatggggtcttcttcatgagttatgtatgtgatgggattcttgtaagattttcaccttttacctgagcttgccttgactgaaagttgagaaaattttgtgattcattggtataatttctacagtgcaatatttatttggatctaagcatgcaagtaaaaaaattcatccctgttatagtatttcaggtttgcagtacgttatgacatttgtagatcattgatggactatgaatattcaagggacctggcaattgtacccaggggattgagatccagatacttgtttgtttgcccatagatataaaattaggagcttcctgatatctaatttacttttttaggaataacacccatgagatacagttctcatctactgccctactcctgtccttagataaataacaccggaagaagaagactctctggcctcttacatcagggacacatgaaaagccacactgaatatgtatgaactccaatatactttcacagaagggtttaaggatgccatccctggttataatttttaggaccaaatatatatatatatacacacatacacacacatttaggatagatatatatttaggatatataggataaatatatatatttagtgccatggtttgggtcatgaggaggtttgatgacataaaaattatgctcataagtattatgctgagacccattcttgggaggagatgtgggggtgtcctaggctgtgcaaaacatagctatggaaacagaagttattaagagaaaagaacaagcagtcagtgagaatttagataagagactgtaatagaataaaagtatcagattagtgaatggttgtcctagtaatttggaagaaaaggaataaaaaatggagtcagactactaaatggcaaaaataaagacttgaagaacttaattttatacctgagctttgctttgattgaaagttgagaaatcttgtgatttgtcggtatcatcatctcaaatcaacttatttttcatctcaacatgaagaatatcagctctagccttttggaatttcagcaactcagatgcttgttagaacttatccaggactgcttgcaggacccaaaatactaataatccatatgcctttttttccagctgaaattagctgtcttaagagttgtttttcagtgctctgaggatatccacaaatatatcccaaggcactctgaagtgtgttttcaatctttggccctgcacactggggatgcccactggatgttgcatggagtgttgcctccagttgactttgatagacacaataaacaccacgtgagagccccctcatgaaggagggctgaggacttggagtccaattctccacctcccctgacagataggctattcaatgccttgggaatccgacatgaaggactgcttatagcagtccttgaaaactttctacagaatgtgaactgggagaagctaaatgctgtactcacaagctgggtgacttctccattgatttttaccatagctcactgaaacattttctaaattaacaggtataaattcacagacagatcagaatagatccttggtcctttccatttttctggcacttttgttgccaggaattcaaatacaactaagagaaaaagtgggtagctagcaagaacagatagtttctcaaatattagtggcagtaacccaactttgagaaaatatggagaaagagaagaaaaaacccttaagactgcagtgttagctgcccagttacaattattcacaaagtgagcaaatacttatttgggccaccaataaataagagggcacaagctaacgggaaattaatacgtcactcatgtaaaaagtcaacacattagaaaaaggaatcttggatgagattgagaaagatgcaagggataaaacctgacaagctttacaacctccttgaggaggtcttccctactctgtaaaatcagacagaaaatcattggcaaatggagcaaagcatgggcaaaactgatataatgaggaaacagagctgggaaaggataaatttatgtgtctgtttctgtttcctatcttttaatccttttttaccctttcttctggatactggagcaacgtattctgcaatttctacaggccttttctgttttatcctaaactaggaaaatatccagatataagtatttctggactatctgctacctcttctttctcctctgaggttttcatgcaaataggcgaggtcctatttgcataaaataggaagaatatgccttgtctcctaatatgcccaataatcttttaagaaagggcctgctgtgtaaaccaagggctatgatcttctgtactctgaagagagtgcttttggagcttctgggatgcaaggtatctacattgctaaatatgtgctttctgaaacaaaagatggctgtgggagagtaggagtctcactcaatgctggagaagcactcccaagttacagaaaataaacccatatctatgagtcctaggaacaaatgataaagatttaacaaaagagatcaataccattgttgtaacttacaacagagaaaaaccttggccatgtatagaacattttctctatttttacctcaaatatgatgtaattagaaaaattttaaaaaacgtggcatagttaaaaaaaaatggcacattcacttcataatgcttccatattgcctgtgggaaagttaggaaaacttgacagggaaggtcaaactgtttatagatttatgcatgatcttagagaaacaagttgtgattcctctgacacttgtaattcccagccttgccactgttttaatatctgttcctgagttatctaagtatttttctgtcatggatttatgttcaatgttttgttctccatgtttagctggggagtcgaggttcctgtttgagtttacctgtggagcgggaggctttcaatatttgtggcaacggatcttcctggaattcagagagtcacctactccattctctgggcatttataagaaaatataaagaatattcctcacattaggattccatactaattgttattgatagctgctaataacttacatgctagtaaagaagacattttagctttactaaatttcttagactcccaggacgtaaaacctcgctagctaagttgcaatattaccaactgaagtaaaatatttgggatctgtgttattggcagatggaaggtggttgcatcttgaagtcagcctaatatacaaatgaaacaacatgccaaaaaaaaacttcaccgattttgaggattaattgtagatctttgcgttttatgaaaaggctaagcctctcactgagatgttacatgaaacatcactagaactctttattaaagtcaaaataaggctgaagaagtcttttatgcccttaaaatagctgctatttcttcacttgtattacttttttaaccttgttaaaaaaggagttctaccttttctgtcttgagattaagggttactccaaaattaggccttgattataggcacattgcttactttcccaggttcttgtattttgtctctctggaaatgcgatgatatttataaggtacagtggcaaccactcccctgattaaaaaggctcattcagtccttgactctgagaaatttaacttacttacaggtacttcatgttgttactgctattttacaagttcataagacacaacacttgtcagtatgctgtcaaacgagctaagaacaagtcttattgtgtgatcctaatgctattctaggaaggtgtaaccttttgacagctattctgtggccagatcttgacagaataagttgaacttgactgtctcagagtgattgaggaagacactggagcatggtctaatttttgtaatattcctattcccaatgttgactgaatattcatgaatatattctgtgtgaaagacccacaaaggaatataaaagcatgttctacggtaatcaaccctcatgaagcttagaggcttctgtttttcgaaacataaaatcagcccaagtggcagaattgtagttattataccagctgttaaattagccaccagttataaagcagatatctatactggtaataactatatgtttgtggtctgttatgtgactagccaactttggaagaatagatgctaacgtcaataggaactaaaatattacatgagaagctaatttctgatttagtagagtcattcaaacttctctaacagatttatgtaatttattgtagagctcacagtggaaaacaagattaactctttaaaaagaaattaatttgcagacctagctgtgaagggcttctgacgtttagactggggaaatcaaacctctatcacttctgtccaaaacctcctattcataatttcagaaatagccaactctcttaagaattagaaaattaggaatgaaggagaactaagatgaacaccataggaaaatgagaattacataatgaaatctttcctttattccgattgttatatgattgagtggctttttcatgccaccaacaagttcgttcaagcaaaaggaatatgctgacaacttgaaatctttgtaatagcatcatataattcaggaaataatatgaagatatgctctttgtcagaaaaattctttccttacctaaaataagtcaagaaagcctactaaggcccatgatacctaggatctgctggcacaagaactttatagaacttccacctttagaagatagtaaatatttttaccagtgttgtgtattggtgtctggataggctaaagcagatgctcaaacagtgatcaagctcttactgaatcatatcatctcaaccactggaattcctgaatatctggaattcagtaggggaatcattctgtatttgtgggaattcgggaattttgtaatactctacaaataccaataatttacctacctgttatccccagtcttctgaacaagtggaatgagtgaaccacataccctaaagaattatttagctgaattatttagctaagttatgtaaaaccttaccatccgtttcatttaaaataagattaaatccctctagcaagcccaacattactccctttgagtttggaaggtctactaatcttgggcttaagcctaaccctttctctgatacaatggaaaatcctcatgaccatatatgatacttaaagagtttgtgggtttccctaggctcaccttgccaacatgtggccagatgctgaaaaagccgcctggggaagtgtgtcattgatattgaacaggagattcagtctatatcaaagtgttctgatgacagactgtgaccacactggacagaaccctatcaagtgtggttgagaattcatagtgctatcaagatgaaggagaaacccaagtggatacacgcttcacatgtgagaccaacatccaccatcgagtggggtataattcccatttaggaccttaaggataaattctctagatagtaaccttgaataatatgcaagctaaagagaaaaacttatttgatagacagcaggagagatagactgttttgttattaatacgttcttcttaattattttgaccattataattttgatacttagtttgtattaattttgatacttagtttgtatcaattgaacataaagcttattattagtctttcttttaaaagttagtatctcaacctcaaggaattttgtggtaattaaaaattgtcctgaatatgagatcaggacttaagaaacccctttctttgtcataatacttcaaactcaccaactggatgatttaaattcagaagacatgaaaaatttcaaatcttcccactgattagtcaatattacaaaaatcttaaatatcatcaccctttgtgttattgaagatactgatgagaagaaatttttaactaattaagttgaacgtaattatattcaaattgaaagtccctgaagggtatcacaaaactccaacaactctgtatcttttttttttttgagcagagtttcaccttattgcccaggctagagtgcaatggcacgatcttggctcaccacaacctctgcctcccaggttcaagcgattctcctgcctcagcctcctgagtagctgggattatagacatgcaccaccatgcctggctaattttgtattttttgtagagatggggtttctccatgttggtcaggatggtctcaaacttccaacctcaggtgatccacccgcctttgactcccaaagtgctgggattacaggtgtaagccaccacgcccggcccaactctagatcttaaatagccatgggggtccaatttaataaagcttaaatagacacacagatgagaaaatatcccagcccaaatggtgagggttagtttcttcaaaacccccaaaacgtatcatcacctaaaaatgagcttaccctgtttagaacctggcagtctccagtccccatacttacaaactcgaggataattaaatggtctgtatcatttaatattacaatagctaatgtgagtaaactgaattctacttgtacatgataggctttaccactatgaaatgtttttacacagggactaaaatggcactatgatcaaactgaaagagatttctttttttaatagatggggtctggctttgttgcctacactggagtgcagtagcacaatcatagctgtctatcgcctgtaactcctgggctcaagggcttcctccgtctcagcttcctgagtagctgggactacagggacatgccaccatgcctgcctatttttttttgtagagtcagggtcttgctcaggctggtttcgaacttctggcctcaagtgatcctgctgcctcggccttccgaagcactgggttcgcagacataagccaccatacctggccagagattattaaatacagtattagggggaataaaactaggactacaattgtaaactcttctgatatagaaacaattactgataaacagtgcattggacaatttgcagaaagatgcaaacatgatagaatcaaaatctaaacaagatatgacaaagaaaaacaaaaatggaattgggaaagcatttggaattggacatctggtaataaaattctattgcaaaaaaatatatagtaaagaattttcagaagccttcacttgcattagtgcacaaccactggttattcaacaactacaatgtatacttgctggtactgaagtacaagattatacctactcagacaaacaattaggtattggacatactactgtctatgtagagattaaatgatagtgtaaaaaggttgatcatattataggtaaaagagctgaaatctggcattgcagattgaaatttctgaaaactgaaataataaaactaatttcactaaagtatgtacattttcttatttacatagtttataatgtgttatataattcatgaatcttcaaaagaaatatactgagctattcaataaaaatttacattacagctttaggccaccaagccccatctattgattatgtacttggtgtaaattggcggggggaacaaaacaagagtgaaaactgagcctcaaaatacaaagcatgtagcaaaacagactactaaaattattctcaatttctaagcccattatatgatgcaggagaattcttatgtgtaggtaataaagaatctgttcaaatatgcatcagtggtatgacagtttcaaataacccactgaactaggaaatattcaccaccaaggaggtcaggtatactgatttggtcaacctggatattgacatgggatgttcttacggattgacagctccacttgatgctatcctatcacaaaatattattacctgatgttgatataattaacaagtcaatacaaaaagtaaggtctctctacaagacaagactaacatctctgtaatataaatgggactctgagtgtaatagatttagggtttctgcaagaattttgaaagttaaggatccaacgcctgtaatcccagcactttgggaggccgagacgggtggatcacgaggtcaagagattgagaccatcctggccaacatggtgaaaccccgtctctaataaaaatacaaaaaaaaaaaaaaaatagctggacatggtggcacacacctgtaatccccgctactcgggaggctgaggcaggagaatcgcgtgaaccagggaggcagaggttgcagtgagccgagatcgtaccactgcactctggcctggcaacagagcgagactctgtctcaaaaaaacaggaaagcaaaagaaaaagttaaggatcctaccaacaaaattggaatatgagactgaggaaattcctattccagctaaacccagaattgattatgttgatgaacacatgaatttcatgtgtgtgaaaacatatgaactcctcattgtcagtctaatgagagtgaaaacaacacatcgaagatgcggggagacgacaatacagagtgggctcataactgcattctagatactgggattactttccattaaggtatataattttagaatatctcacaaatagcccatgcaaaatcctgcggaaaggcaaccataaatatagagagaatatgtaaaaaagtggcatggtctagtatatgccaaacaaggcaagggtatacaaaaacctcaaggtcccagtatgacaaatcatgcttgtattgcacacccattaaaaagtcttctaaaaagtttttttaatggtgtgcaggcaggtttattcagaaaaagctcggattcatacttgtaaaagtctgagaaaagcattaaaatgctattatgatacagttgacccaaacaacacaggtttgagctgtgcaggtccacttatctgtggattttcttctacctctgccactgagacagaaagacaaactcctcctgctcctcagactactcagcatgaagatgaggatgaagacctttaggatgattcacttcttcctaatgaatagtgaatgtattacttccttaataacgtttttttttctctagcttcttgtaagaatacagtatataatatatgcaacacaaaaaatacgtgttaatcaactgctttatgttatcagtaaggcttccagtcaatgggagactaactactaattaagtttttggggagtcaagttatatgcagattctccactgagcaaatgttcagcactcttaactcctgtggtgttcaagtgtcaactgtatatatgtttaagagttattgtttacccagtagatcaagccaagaccaagttaggtttatgtgtttttggcttaacctaagaaaatgttcagactaaagcagtccatcaatagaacagactgccttttaaaccagggaacaacttgtctttgtaggtattttaggtatatgaatgaccatatgtgatctgctttgggtcatctatggtcccttccaacttaaaacttctctattcatgaacattgcataactgaattgtggatttcatataaataatatcttgttgcctactttaggtttgaaatgttttccttctcagttgcatagatatgtatagcaccatccccactatgaactctcatgtagaaaataccagattctaaagtcatctgtgaggctaaatgtcagacttaggcacttccacataatgctgcaaaaagcaacagcttaaaggtattacttgctcaacatgtcattccaaacacatacactatgattagcagctgaatttaacagtatctttccactttgaaaaacactttatatacctttacaaagaagccatagtatctcagggtagaaaaactcaacaggtagaaaacatggccttctcttagaaatgagctacgtaattttgagtacttatctcaaaatcattgcagaaacatatctagtgcttatggaaaaagaatcaaaagatctttttttttagctaaagattttgagtttttcttgtgacatttaagtcttttaatgtatgctgatttttatgattatctaactgtttcataatggttattcttgcctccatgaaagggaagactagaacaaaaacctgacaagaacaagttctagctccatcaccgacttactgagtgaccctaggcaaatcatgtgggctctttcttgtgttttaagttttacctccaatgaagtaactgcctcagactagctgtgaagctctcataaagtgatggatttgaaaatgccttgcaaagaatgagcagttaatacatagaaagtttcatgtacttccgtatattgcatgatgcccacccagggttaggttgataacagttgttgatccaataatgaaagaaataaaaagaactctttaaaataacgaagtaaaatagctaattttcctgttgtagttttaaaaattatataaactatataactaaacatatttatgtttattatatataatatgatatgcaattacataagacattattatataatgtttttatataatgcttaagtaattaaacaattatatttaaatataagttatactttccttccccagataagacaaagaacctgtttacttctctgagggatttattggttcctgtgaaatctgtggaggccgctaacactccatagccagagaatgacaacatatgattttcttttcagtcttgtagtatccacagtagtgatgtctgtccatgtacaagtgtctgtccaggacatccattaaattccatgcctgctctgtgtgttatgagggcagggacagaagacagattcactggtggtactttcaaacaatgtggtaggccttccttttggggtctgccatgacaatgataccccaggcggcaatgctaaggcccatgaccatcttcataatggtccatggacccggggggtatcccatcttcatgggcttctgtgccacccagtacacatacccgcctgcccccatcagccgcaacccagaaagcacgtgacagctccagcaggtctccaacaggtggtgttctgctggtgaggtcggcgatccgggtgtagctggggttgcaggcttggcaggcacggcggcggtatcgggaggcacagcgatcttgagggactcaaaaggctgggacaactgagaccccgtgttctggaactcggcctccctaaaaattatgttttaaaaaatacatagaatcggtaagatgaggaaaaagcataaaacggtcaaaaacactgggataattcaaggttcattttgacaatggtaatacaaatagaaaaactgacaaaaaagaaaatacatcttacattagaaaacatacaagagtagttgtatacaaacgaagacacgtaccaaaaataagttacaaaatatgaatgcatatacataaaaaatgtagtttcaaacaattcaaaagtaaaatgatgcaagacattaaaatattcacaaacacgctcgaaaaataagagctgacgcccaaattagaccagttcaacggaagtgtggggtgtggttggggatccgccgcagaaatcacgtggcgcccaggccaaggctgcaatttcccatgctgcttggagaagacgcctgggagggctcccaccatgcccaggggcaggctatgtgactgcccggtctgcagctgtaagtggtttctgggccagtctctttgtctggtcatgacctggttggtggccctagcttgtgccgagtggtcccggagtggcccagggggttgtgtgtggggagccagcgtgggcagcggaggtgctccctggggcctgcagtagggcacagcctctgcatcttgtgtgagcccagtgacccgcctgtcctgtccttccagggttgggtggcagggagggccaagggtgccctgcgtccgtgacgggtcagcgtttccagcaaatgggtccatggagggagagcctagctgagccggcgggctctaccaccttcatgaggttctgtttggacacctgtcctactcctgttcccccagatggtgaccatggaggaagacttgtgttgggcccaatggccctcggccagtgtccgtctgccaggtgccagcccccggtgcgctgaagctcaggccattcctgatgccctggcctcctgcactgagctgtggtgagcccatccgggtcctgctggatacatgggcggggaggggggtgccctgggttgtgttgatgatgagaaccttatattatcctgaagagaggtgatgacttaaaaatcatgctcaataggattacgctgaggcccagcctaggtgagaattttggaagaggatgctgggatccagaggcccccgacggggccactgtatttcgggctgcagacctagaggccctgaagggcatctgaaggtggcccaagcctatctctgagcaaccccgtgaggcagtcaaggatccctgtgtgggcataggctggggctctggttccctaggggcagggcatgtgggtggctcctccctgacccccattctcccctgtgtctttcagtgagctcttctgcccaggcaggccccctggcattgaccggcataggtgagtggatcctgctggggtcatgggccatgagccaggctgctggggtcgccaagggtcagcctttgggacaggagggtttccttagggagccaacctgaatccccactggggagggctaggttctccaggagggcagcttccttgggagtctgacccaaggaggatatcttctctaaagccctgcagtggtgagggtggtccaggcagcaaagggttccttcaaccccacccaggacagcgcccagctgagactcagtgtgcaggtctggccatgaggactgcccaggtcacataagggggtccataggagctccggggcaccacaggggtcacacaggagctctccttccccagagaggggtgagcgattgggcctatggggtcaggcctttggagatcaaggattgggccccacccatgaacctgaatgggactgagcctttgagcaatggaccatcccagtggtgcctcggagcctagtcgggggtgatgtggcttcaagggggttgggtcaaggctgagctctggccggtgcctggtagtgctgcaggctggccctgggactcgctggctttgggcaaaggggccagcccgtgtcccactggaggtcaccccttccctgtgggcaactagcaggaagggtggcatccttagctgggaaggcccactgtcgagggtcctgtccagggaatttgcctcccaggcttcttctgatccacggactcacctccagggtggtggggtcttgggcccgaggaaaggagaagataccctgtgttccccagttggctgagtgtggcttggctctgtgtttgggaggtgtgtagggtatgaatggggacccaccagagaacacacatggcccagggccccaggttatgatttcccatgcacacttggagagaactctgggtgagtgccctccactcccaggcacaggctatgcaactgccaggtctgcagtggcatgtggcttccaggtcagcctccttggttggccatgagcaggttggttgccctgtcctgtgcccagtggtcttggagtggcccaggggtcctgcaggggagctggtgtgggtagtggaggtgcttgtggggcctgcaatagagcgcagcctaggtgccttgcacgagctcagtgacccacctgccctgtcctagtcctagctcagtgacccacccccgctttgcagtgatggggagtaccaagcgtctcctgcgtctgtgatgggtcagtgttcccatcacatgggtccatggagggagagcatgcatgagctggcaccacccaccaccttcccactatggaggcgtgaggccatgtttggacacatgtcttcttgtccccccagacagtgagcctggaggaagacttgccctggtcccactgtccctgggccagtgtccatcagccaggtgcccagaccccagtgcgctgaagctcagccctccctggcactctggtctcctgcactgagctgtggtgagcacatccaggtcctgctggatgtttgcatggggaggggctttccccgggttgggtcgatgatgagaagcttctgttttcttgaagagaggtgatgacttaaaaatcatgctcaataggattatgctgaggcccagcctaggtgagaattttggaagaggatgctgggatccccggcaggagggattttgggctggagacctggaggccctgaagggtatctgcaggggccccatccctgtctctgcacttgtctgtgaggcagcccagtttccctgtgtgggcttcgtgtcagtgctctggttccctggagcagggcacgtgggtggctcatccctgaagtcacgttctccctttgtgtctttcagtgagctcttccgcccatgagggccccctggccttgagcagcataggtgagcggatcctgctgggctcatggaccaggccacatggggtcactgagggtcagccttcaggacaggagggattcctcagggagacaacctgaatccccactggtgagggattggttctccaggagaaccacctccttgggaatctgacccaaggaggacaccttccctgaagccctgcaggagtgacagtggtccaggcaggaaagggttccattagtcccatgcagggcagctcccagctgagacttggtgctagggtctggcctcgaggactgcccaggccacatgaggcagggtctcaggctgctatttccagtgcatgcttggagagggagcctgggtaggccccctccctgtgcaggggcaggctgtgcatctgctggggctgcagcggtgcctgccttcaggaccagcctccttggttggcttgagctggatagtggccttggccccacccaggggtcatggtgcagcccaggaattgcaggggagctggcatggggagtggaggtgctccaggggcttgcagtagggcacagcctgggcatcttgcttgagcccagtgaccagcctgccctgttttccagggttcagtggcagggaggaccaagggtcccccacgtccatgactatcactgtttcacatggatcccatcaggtgggtccatggagggagagcatgtgtgagtcagcaccccctgcctcctttcccgcatggaggcatgaggttctgttcggacacttttccctgtctctccagatggtgaccctgaaggaggacttgtgttgggcccaatggcctggggccagtgtctgtcagccaggtgcccagcccctggcgtgctgaagtttgggcccttcctggtgccctgttctatcgggggaacccgcccccaataattcaacgtaggtccttttctattttccctaagtgtcggccggtctgagaaataaagggaaagagtacaaaagaaagaaattttaaagctgggtgtccgggggagacatcacatgtcggcaggttccatgatgccacccaagctgcaaaaccagcaagtttttattagtgattttcaaaggggagggagtgtacaaatagggtgtgggtcacagaaatcacatgcttcacaaggcaataaaatatcagaaggcaaatgagggcagagcaccagggccaaattgaaattgctaatgaagtttcgggcacgcattgtcattgataacatcttatcaggagacagggtttgagagcagacaaccggtctgaccaaaatttattaggcaggaatttcctcgtcctaataggcctaggagcgcttcgggagaccagggcttatttcatcccttatctacaactgtataagacagacattcccagagcggccattttagagacctccccctaggaaggcattctctttctcagggttgttccttgctgagaaaaataattcagcaatatttctcctattcgcttttgtaagaagagaaatatggctctgttccgcccggctctcaggcagtcaggcctgatggttgtctcccttgttccctgaacatcgctgttatcctgttcttttttcaaggtgcccagatttcatattgtttaaacacacatgttttacgaacaatttgtgcagtgaacgcaatcatcacagggtcctgaggcgaaatacatcctcagcttacgaagatgacgggattaagagattaaagtaaagacaggcataggaaataataagagtattgatttgggaagtgataaatgtccatgaaatcttcacaatttgtgttcttctgccatggcttcagcaggtccctccgtttggggtccctgacttccctcaacactggtctcttccactgagccttggtgagcccatctaggtcctactggatgcatgtgtggggaggggggtgccctgggttgggtcaatgatgagaaccttatattgtcctgaagagaggtgatgacttaaaaatcatgcttagtaggattacgctgaggcctagcctaggtgagaatgttggaagaggatactggatgctgaggtccctggctgagctactgtattttgggatggagatctggtgactctaagggcacctgaagggccagaccctgtctctatgaggcagcccaggctccctgtgcaggcttgctgttggcactctggttccctggggtgggacatattggtggctcctccctgagccctgttctccccttgtgtctttcagtgagctcttccaccaaggagggccccctggcattgactggcataggtgagtggatcctgctggtgtcatgggccatgggccaggccacgtggggtcaccaagggtcagccttcaggaaagaagggtttcctcagggagccaaactgaatccccactgggaagagatgggttctccaggagggcggcttcctgggaaacaggaaccaaggaggacgccttccctgaagccctgcaggggtgacggtggtccaggcaggaaagggttccttcaattccacccagggcagctcccagctgagtctcggtgctcgcgtctggactgccctgactatgtggggggccacaggggctctggggtgccttgggggtcacacaggtgctctttgtccctggagaaaggtgggtgatcaggcccatggagttgagcagtggagattcaaggattgcacctgacacatggtcctgaatggaacccaccctgttgagcaatgggccatcccatgggcctcttcaagcctgaagaggggcggtgctgtttcactgggtttaggtcaaggctgagctctggccggtgcctggtggtgctgcagtctagctgttggactcgtgggctttgggccagtggggcagcctgcatccctctggaagtcaccccttccctgtggctggaagttaccccttcccagtgggcgaatgtggggaagggtcacctcctgaagtgagaaggcccactattgagggtcctgtcctggcaatttgctgcccatgctgcttctgagccatggagacacctccagtgtggtgggatcttgggcccaaggaagggagaagacactgtgccccccgattagctgagtgtggccaggctgtgtgtttatgaggtgtgaggggcatggatggaaactcaccagagaaagcatgtggccctgggaccccaggtcgtggtttccagtatgtgcccagatacagcacatgggtgggtgcccaccatgcacagggactggctatgtgactgccagctgtgcagcagcatcaggcatatgggccagcctccttggttgacaatgaactggttgctggccctggcctgtgcccagtagtcctggggtgaccctggtgtcatacaggggagctggcatgtgtagtggaggtgcttgtggggcttgcagtagggcacagccctggcttctagcctgagcccagtgacctgcctgtcctatcctttcggtgttcagtggcggagagagcattgggtgccctgcatccatgatgggtaagtattccacatggatctcatcacatgggtccatggaaggtgagcatccctgagctggcacaccacaccttctgtccacatggaggggtgaggttgtgtttgggcacttgtcctcttcctgttccttgagatggtgaccccagaggaggacatgcgttgggcccgctgtccctggccagtggccatcagccaagtgcctagcccctattgcactgaaactcgggcccttcctgtcaccttggtctcctgcgttgagcttgggtgagcccatccgggtgccactggatgcctaggcagggaggggggtgccctgggttgggtcgatgatgagaactttatattgttctgaagagaggtgatgacttaaaaatcatgctcaataggattacgctgaggcccagcctaggtgagaattttggaaggggacactagaatcccgaggtccccagcagggctgctgtattttaggctggatacctcgaggccctgaagggcacctcatggaggggcccaatcctgtctgtgctcctctattaggcacaccagactttctgtgtgggcttggtgtccacgctcagcttccctggggtgggacacgttggcggctcctccgtgagccccattatcctctgtgtctttcagtgagctcttccccccagcagacccctgggattgacgagcataggtgagtggagactgctggtgtcatgggccatgagccaagctgcgtggggtccccaagggtcagccctcgggccacctgggtttcctcaggagctgatctgaatcccaactggggatgtgttctccaggagggcagcttccttgggaatcagacataaggaggaggccttctctgaagccccgcaggggtggcagttgtccaggcaggaaagggtttcctcagccccacgcagggcagtgctcagctcagacttggtgcttgggtcaggcctctaacatggccaggccacatgaggggggcaacaagggctccggggagccaagggcatctcacgggggctctccaaccctggagaagggtgggtgattcggtccatggggtcaggccatggagattcatggattgcagcaaacccaggaccctcaatgggactgaccctataagcagtgggccctcccagtggcacctctttgagccatttgtggggcggcgctgtttctctgaggttgggtcaagactagctctggccagtgcctgatggtggtgcaatccactcctgggactcgtgggctctgggccaaggctgcagcccatgtcccgctgggggtcaccccttcattgtgggcgagtggcagtaatggcctcctgagttgggaaggcccgcagtggaaggacctttcctggaaatttgcagtccaggctgcttctcagccattaggacaactccagcgggttggggggcaggggcgggtcttaggcccaaagaaaggagaagataccatatgtctcccggttggctgagtgtggccctgctgtgtgtgtgggaggtgttgtgtggggcatggattgggacacaccagagaaagcttgtggccctggagccccaggctgcgatttccagtgcaccttgaggaaaggagggtgggtgccctccatgcacagggcagactgtgcaactgccaggtaggcaacagcacgtggtgtccgggccagggtccttggtttgctgtgagctctttggtgtccgtggcccctacccaaggatccagataccacccggggtcatgcaggggagctagcatgggcaatggaggtgctcaaggggcctgcagtagggcacagcccaggtgtcttgcctgagcccaggaacccacctgccctgtccttccatggttcagtggctgggagagcaaagggttccgctgatctatgacacatcagtgtttcccatggatcccatcacatgggtccacggagagagagcatgtgtgagctggcacccccaaccccattccctgtatggaggcgtgagcccatggttggacacatgtcctcctcctgtccccccagatggtgactgtggaagaagacttgctttgggaccgctggccccaggccagtggttgtcatccaggtgcccagcccttggtgcacttgagctcaagcccttcctggtgccctggcctcctgccctaagctgtggtgagcccatccaggtcccactggatgcatgcatggggaggagggtgccctgggttggatcgatgatgagaaccttatattgtcctgaagagaggtgatgacttaaaaatcatgctcaataggattacgctgaggcccagccttggtgagaattttggaagaggatgctgagatcccaaggtctctggcagggccactgtattttgggctggagacctggaggccctgaagggcatcttaggggtgcccgaccccatctctgcgctcctccttgaggcagcccaggctcccttgcgggcttggtgtcagccctccagttccctggggttgaggcacattggaggctccttcctgagcccccattctcccctgtgtctttcagcgagctcttctgcccagggggacccctggccttaagtgggataggtaagtggatcctgctggggtcgtggaccatgagccaggtcgctttgggtcactgagagtcacccttcaggccacgagggattcctcagggagcccatctgaaacccaccagggacggatggtttctccaggaggacagcttccttggaagtcggtcccaagaaggatgccttccctgaagccgtgcagagatgatggacgtccaagcaggaagggcttccttcaactcaatgcagtgcacatcctagctgagactcagtgcacaagtcaggcctggaggacttcccaggccacatgagggggtccacagggtctccaggggccacgggtgttatgagggtgctctctgtccccgaggcttgcagaggggcagggtgtgggtgatcggacttatggagtcaggccgtgaagattcaaggattgggcccaacccatggcccagcatgggccgaacctgtagagcaatgggccatcccggtggtgcatccttgagccttgtgggggtggtgtgaccttagtggggttgggtcaagcctgagctctggccttcacctggtggtgctgcaggctggccctgggaatttgggctttgggccaagggtgcagtctgtgtcccgctaaaggtcacccctccccaaagtggagtggccagaaggatggcatcctgtggtagaagtcccactgtcaagggtcctgtcctggcaatttgttgcccaggctgcttctaagccatgggttcacctccaaagtggcggggtcttgagccgaaggaaaggggaagacatcatgtgttccctgattgactgagcatggcctggctctgcatttggaggtgtgcggtgcacgaatggggacccatcagagaaggcatgtggccctggggctccaggatttgatttctggtgtgagcttttacagggggtctgtagtggcacgtagcattcaggtcagcctacttggtttgctctgagcttgttggtggtcctggccctgcccagtggtcccattgtggcatagagtcatgtgagggagctgtggcaaggagggccaagggttccccacattcatgatcgttactgtttacatggatcccatctcttgagtccatggagggacagcatgtgtgagccagcaccccctgcccactttcctgcatggaggcgtgaggccgtatttctatacgtgtcccccacccctccagatggtgaccccagaggaagacgtgcattgggtccgctggccccaggccagtgtctctcagccatgtgctcaacacctggtgagctgaagctcaggcccttcctggcatcctggtctcctgcactgagctgtggtgagcacatctgggttcctctggatacgtgtgcagggagggggatgcactgggtcgggtcaatgatgagaaccttatattgttctgaagagaggtgatgacttaaaaatcatgctcaataggattacgctgaggcccagcctaggtgagaattttggaagaggatgctgggatcccaagttccccagcatggccagtatattttaggcaggagaccctgaggctctgaacggcatctccggggggcacaaccctggctctgggctcctctgggaggcagcccaggttccctgtgcaggcttgggctctggttccctgagcccccattctccctcgtgtgtttcagtgagctcgtcctcctaggtggggcccccaggcattgaccggcataggtgagtggtcctgctggggtcatgggtcatcagcaggttggagtcaccaagggtcagcctttgagacaggagggtttccacaggcagccgacctgaatccacaccatggagggatagattggtcctccaagagggctgttttttttcggaatcagacccaaggaggataccttccctgcagccctgcggggatgacggtggtccaggcagggaagggttccttcagtctcacgcaggtcagctcccagctgagtctcagtgcattggtctggcctccaggactgaccaggccacgtgaggggggcaacaggggcttcgggatgcagtggggtcacacaggatctctccatccccagagaaaggtgggcatagaacccatggattcgagctgcagagattcaagaatcgcacacgaattattgccctgaatgggacccaccctgttgagcaatgtcccatcccagggcacctctttgagcctgaagggggtggtgctgtttccctaggtttgggtcaaggctgagctctggccagtgcctggtggtgctgtagtcctgtcatggactcgttagctttacgccaaggggtcagcccacgtcctactggggatctccccttccctgtggctggaagtcaccccttccccattggcaagtgtcaggaagggtgacctcctgaggtggaagtcctgttgagggtcttgtccctggaatatgccacgcatgctgcttctgagccatggggtcacctccagggtagcgggtcttgggcccaaggaaaggagaagtcaccatgtgtcccccgattagccaagtgcggccctgctctgtgtttaggaggtgtgtggggcatggatggggacccaccagagaaagcatgtggcccagggaccccaggttgcaatttctaggacatgctgggatagggcacctgtgtgggtgtccacagtgcacagggataggctatgtgacttgcaggtctgcagcggcatctggcatccaggccagcctccttggtgggccgtgaactgaatggtggcgctggcctgtgcccagtatatctggtatgaccctggggtcgtgtgggggagccggcatgggcagtggaggtgcttatgggacctgcagtaggacacagtcggggcttcttgcccaagcccagtgacccgcttgccctgtccttccagggttcggtggctgagagagcttttgttgccctgtgtccgtgacgggccagtgttctgcatgcatcccatcacatggatccatggagggtgagcatccctgggctgacacaccccaccttctgcccccatggaggggtgaggttgtgttttcacacctgtcttactcctgtttccccagatgttgagcccagaggaagacttgcactgggcccaatggccccaggtcagcgtccatcagccaggtgtccagcccctggtgcactgaagatcgggcccttcctggtgccctggtctcctgcattgagctgtggtgagcacatccggatcctgctggatgcgtgtgcggggaagggggtgccctgggttgggtcaatgagaaccttatattgtcctgaagagaggtgataacttaaaaatcatgctcaataataggattacgctgaggcccagtctaggtgagaggtttggaagaggatgctgggattccgaggtccccaggagggcccctgtatttttggctggagacctggaggccctgaagggcatcttgtgggtgcccaaccctgtctctgcgctcctccttgaggcagcccaggctccctgtgcaggcttggtgtcagccctccagttccctgggtttgaggcacatgggaagctcctttctgagccccgttctcctcttgtgtctttcagtgagctcttccacatggaagacccctgtcattgactggcataggtgagtggctggcatcatgggccatgagccaggccgcgtgaggtcgccaagggtcagccttcgggaaagaagggtttcctcagggagccaacctgaatccccactgggaagatatgggttctccaggagggcgacttcctgggaaacaagaaccaaggaggtccccttccctgaagccctgcaggggtgacggtggtccaggcaggaaagggttccttcagttccacccagggcagctcccagctgaggctcggtgcttgcgtctggacttaaggactgccctggccatatgaggggtgccacagtggctctggggtgccatggggtcacacaggcactctctgtccctggagaaaggtgggtgattgggcccatggagttgagctgttgagatgcaagttttgcacctgacacatggccctgaatgggacccaccctgttgagcaatgggccatcccatgggcctcttcgagcctgaaggggggcggtgctgtttcactgggtttaggtcaaagctgacctctggccgccacctggtggtgctgcagtccagcccttggactcgtgggctttgggccaagggggcagcctgcatcccactgaaagtcaccccttccctgtggctggaagttatcccttcctcatgggcgagtgttgggaagggtggcctcctgaagtgggaaggcccgctattgagggtctgctcctggcaatttgctgcccatgctgcttctgagccatggagtcaccaccagtgtggtggggtcttggacccgaagaaaggagaagacactgtgccccccgattagctgagtgtggccaggctgtgtgtttgcgaggtgtgaagggcatggatggagactcaccagagaaagcatgtggccctggggccccaggttgtggtttccagtatgtgcttggatatggcatatgggtgggtgcctaccatgcacagggactggctatgtgactgccagctgtgcagcagcatcaggcatacgggccagcctccttggttgaccatgaactggttgctggccctggcctgtgcccagtagtcctggggtgaccctggtgtcatgtaggggagctggcatgtgtagtggaggtgctcgtgtggtctgcagtagggagcagccctggcttctagcctgagcccagtgacctgcctgtcctgtcctttcagggttcagtggcggagagagcatcaggtggcccacgtctgtgatgggccagtattccacatggatctcatcacatgggtccatggaaggcgagcatccctgagctggcacaccccaccttctgccagcatggaagggtgaggttgcatttggcacctgtcctcttcctgttcctcgagatggtgaccccagaggaggacttgcattgggcccactgtccccgggccagtggccatcagccgggtgcctagcccctgttgcactgaagctcaggcccttcctgttgccctggtctcctgcattgagcttgggtgagcccatccggttgccactggatgcctaagcagggaggggggttccctgggttgggtcaatgatgagaaccttacattgttctgaagagagatgatgacttaaaaatcatgctcaataggattacgctgaggcccagcctaggtgagaattttggaagaggacgctgggatccctaggtccctgacagggccactgtattttgggctggagacctggagtccttgaagggcatctggagggggcccaaacctgtctctacgttcctccatgaggcagtccaggctccctgtgcgagcttcgtgtcggtgctcagattccccttggtggtgcatgtgggtggctcatccctgagcccctgttctccccttgtgtctttcagtgaactcgtttgcccaggaggacccctggccttgagcagcataggtcagtggatcctactggggtcatgggccatggaccaggccacgtggggtcactgagggtcagctatcaggacaggagggattcctcagggagacaacctgaatccccactggtgagggattggttctccagaagggccgcctccttgggaatctgacccaaggaggacaccttccctgaagtgctacaggagtgacattggtccaggcaggaaagggttccattagtcccatgcaggtcagctcccagctgagacttggtgctagggtctggccttgaggactgcccaagccacatgaaggtggccccaggctgtgatttttggtgcgtgcttggagagggagtctgggcaccctccctgcacaggggcaggctgtgcgtctgccaggtctacagtggcgcctagcttcagagcaagcttccttggttggccttagctggttggtggccctggctcctacccaggggtcacaatgcagcccaggaagtgcagggaagccggcttggacagtggaggtgctccaggggcctgcggtagggcacagcctcggcatcttgcctgagcctagttactcgcctgtcctgtccttccagggtttggtggtggggagatacaagggttccccaggtccataactgtcctcgtttcacatggatcccatcacatgagtccacggatgtagagcatgtgtagccagcagcccctccccgctttcctgcatggaatcttaaggccctatttggacacctttccccgtccctccagatggtgagcacagaggaagaattgcgttaggccctttggccccggaccagtgtctgccagccacgtgctcagcctcccgtgcgctaaagctcaggtccttcctgtcatcctggtatcctgcactgaggtgtggtgagtccatccaggtccctctggatgtgtgagtggggatgggggtgtcctgggttgggtcgatgatgagaaccttatattgtcctgaagagaggtgatgacttaaaaatcatgctcaataggattacgctgaggcccaacttaggtgagaattttgaaagaggatgctgggaatcacaaggtccccggcaggaccactgtattttaggcgggagacctcgaggccctgaagggcatatcatggaggggtacccaacccagtctgtgctcctccattaggcacaccaggcttcctgtgtgggcttggtgtctgcactcagcttccccgggggtgggacacattggtggctccttcctgagtcgcattctcctctgtgtctttcagtgagctcttccccccagcaggccccctgggattgactggcaaaggtgagtggatactgctcgtgtcatgggctgtgagccaagctgcgtggggtccccaagggtcagccctcgggtcacctgggtttcctcaggagctgatctgaatcctgactggggatgtgttctccaggagggcagcttccttgggaatctgacacaaggaggaggccttctctggagccctgcaggggtggtggtggtccaggcaggaaagggtttcctcagctccacacaggacagtggagctgggcagtgctcagctcagacttggtgctcaggtcaggcctctaggatggccaggccacatgacgggggccacaagggctccagggcaccacagacatctcatgggggctctccaaccctggagaagggtgggcaatttggcccattgggtcgggtcatggagcttcaaggattgcaccaaacccatatccctcaatgggaccgaccctattaagcaatgggccatcccagtggcacctctttgagccatgtggtgggtggcgctgtttctctgaggttgggtcaagactagctccggccagtgcctgatggtggtgcagtccagtcctgggactcgtgggctttgggccaagggtgcagcccgtgtcctactggggatcaccccttccttatgagggagtggtgggaagggtatcctcctgaggtaggaaggcccgctgtggaaggaactttcctggaaatttgcagtccagactgcttctgagccatgaggtcatctccggggtggcggggtcttgggcccaaggaaaggagaagacaccatatgtctcccgattggttgagtatggctccactctgtgtgtgggaggtgttgtgtggggcatggattgggacccaccagagaaagctcgtggcactggaaccccaagctgcaatttccagtgcaccttgggaaggggactgggtgctcaccctccatgcacagggcaggctgtgcagctgccaagttggcagtggtgcgtggcatccgggccagcgtccttggttcgctgtgagctggttggtgtccttgacgcctgcccaggggtcccagtgctgcttggggtcatgaggggagccagcgtgggcaagggaggcctgcagtagggcacagcccgggggtcttgcctgagcccaggatcctgccggccctgtccttccatggttcggtggctgggagggcaaagggtcccccacgtctgagacacatcagtgtttcacatggatctcatcacatgggtccatagagagagagcaagcgtgagctgacaccccccacccccttccctgtatagaggtctgagcccctggttggacacatgtcttcctcctgtccccccagatggtgtctgtggaggaagacttgccattgggttgctggccccaggccagtggttgtcatccaggtgcccagccttggtgcactgaagctcaggcccttcctggtgctctggtctcctgcactgagctgtggtgagcacatccgggtcccactggatgggtgtgtgtggtggggggtgccctgggttgggtcgatgatgagaaccttatattgtctgaagagaggtgatgacttaaaaatcatgctcaataggattacgctgaggcccagcctaggtgagaattttggaagaggatgctgggaattacaagttccctgtgaagtccactgtattttgagctagagacctggaggctctgagggcatctggagggtgcccaaccctgtctctgcactcctccatgaggcagcccaggctcactctgtgggcttggtgtcggcgctcctgttacccaagggtggggtactttggcatctcctccctgagcccccattctcccctgtgtctttcagtgacctcttttgcccggcagaccccctggcattgaccagcataggtgagtggatcctgctggggtcatgggtcatgagccaggctgtgtggggttgccgagcatcagcctttggctgcaagggtttcctcaggaactgacccgaatgcccacctgggatgggttctccaggagggcagcttccttaggaatcggacccaaggaggataccttccatgaagccctgcaggggttgtggtggtccaggcagtaaagggtttcctcagccccatgcagggcagcacccagctgaaactcggtgcttaagtcaggcctctaggatggcccgtccacatgaggggaaccacaagggcactggggcgccaagggggtcttatgggggctctccgaccttggagaggtatgtgtgattcggcccatgaggtcaggttttggattttcaaggatctctccaaacccatggccctgaatgggaccgaccctattaagctatgggccatcccagtggtgcctctttgagccaagtgtggggtggcactgtttccatgaggttgggtcaagactagctctggtgaccccatgatggtgctgcagtctggccctgggactcgtgggctttgggccaagggtatagcccatgtcccaccaggggccacaccttccttgtgggcaagtggcggacagggtgtcctcctgaggtgagaaggcctgctgtggaaggacctgtccaggctgcttccaagctatggggtcacctacagggtggtggggtcaaggaaaggagaagacaccatgtgtctcccgattggctgagtgtggccccactctgtgtgtgggaggtgttgtgtgaggcatggatggggacatatcagagaacacacgaggccctgggtccccaggctgcaatttccggtgtggtgtggagcagggcctgggtgcttgccctccacacacaggggcaggctgtgctactgccagattggcagcagtacgtggcatccaggccagcctccttggttggccgtgagacggtaggtggccctggctgacacccaggggccagtacagccctggggtcatgtgaggcaactggagtgggtagtggaggtgctcaaggtgcctgcagtagggcatagccagggcgtcttgcctgagcccagtgacctacctgccctgtccttccagggttcagtggtgcagagggccaaagttcccctgtgtccatgacacatcagtgtttcacatggatcccatcacatgggtccatggtgggagagcaatcgtgagctagcatcccccacccccttcctgcatggaggcgtgaggttccatttggacacatgtccacctcctgtcccccagatagtgagactggaagaaaactttcattgggcccgctgtcccggggtgagtggtcatcagtcaggtgcccagcccttggtgtggtgaagctcaggctcttcctggcgccctggtctcctgcacggagctgcggtgagcacatccgggtcccgatggatgcatgcgtggggatggagggcgtgccctgagttgggtcaatgatgagaaccttatattgtcctgaagagaggtgatgacttaaaaatcatgctcaataggattacgctgaggcccagcttaggtgagaattctggaagaggatgctgagatccgaagtcccctggaagggccactgtattttgggctggagtccttgaggccctgaaggacaccttcggggtgcccaacccagtctctgcactcctctgtgtggcagccaaggctccttgtgcaggcttggtgttggcattctggttccctgggggtggagcacatcgacggctcctccctgagcccccgttcttctctgtgtctttcagtgagctcttctacccaggcgggcccctgcccttgagcggcataggtgagtggatcttgctggggtcacgggccaagagccaggccacggggggtcgctgagggtcacctttcaggccacgagggtttccttagggagctgacctgaatccccaccagagatggatggtttctccggaagggcagcttccttgttaattgatcccaagaaggacgccttccctgaaaccctgcaggggtaatggaggcccaggcagcagagagttccttcaacccaatacagtgcagcagcttgttgccttgatgctccccctccctggagaaaggtgggcatagggcccgtggagttgagttgcagagatttaaggatcgcacaggacttattgacctaaatgggactgaccctgttgagcaacgtgcacttggtgcatgggtcaggtgtggagcactgcccaggccacatgaggggtccactgggtctctgggggccacgggtgttatgcaggagctctccgtccccaaggcttgcaggggggtaggatgtcggtgatcgagcttatgcagtcaggccgtgaagattcaaggattgggcccaacccatggccctgatgggaccgaccctgttgagcaatgggctatcccactggtgcctccttgagcctggtgggggcagtgcggcctcagtgggcttggatcaaggctgagctctggccagcacctggtgtttggccctgggaatcgtgggctttgggccaagggttcagcctgtgtcccactggagttcatccctaccggaagtggagtggatggaagggtggcatcctgtggtggaagacccaccgttaagggtcctgtcctggcaatttgatgcccaggctacttctgagccatggagtcacctccagggtagcagggtcttgagccaaaggaaagttgaggacatcatgtgtcctctgattggccgagcgtggcccagctctgtgtttaggaggtgtgcggtgcatgaatggggacccaccagagaaggcatgtggccctggggccccaggatgtgatttctggtgtgtgcttataaagggtgcctggatgggttcatttaatgcccaggggcaggccatgtgactgccaggtctgcagtggtacgtggcatccaggccagcctccttgatttgctgtgagctatttggtgcccatggccctgcccagtggtcctgatgcagcccagattcgagcaggggatttggcgtgggcagtggaggtgctccagggtcctgcactagggtgcagcctgggcgttttgctcgaaccctgtgaccctcctgccccgtccttccagggttcagtggccaagggttcctcgcgtaaatgaccatcactgttttacatggatctcatctcatgagtccatggagggagagcatgtgtgagccagcaccccctgcccactttcctgcatggaggcatgaggccttgtttggacacctgtcccccacccttccagatggtgaccccagaggaagacttgcgttgggcccactggctctgggctcagcccctggtgagctgaagctcaggcccttcctggcaccctggtctgctgcactgagctgcggtgagcctatctgggtcccactggatgcatgggttgcggggcagtggagggtgatgccctgggttgggtcgatgatgagaaccttatattgtcctgaagagaggtgatgacttaaaaatcatgctcaataggattacgctgaggcccagcctaggtgagaattttggaagaaggtgctgggatccggagttccctggaatgaccaatgtattttgggctggagaccttgaggccctgaacagcagcatctcgtgggggcccaaccccttctccgtgctcctctgtgaggcagcccaggctccctgtgtgggcttggtgttagtgctccggttctgtgaacccccattctccccttgtgtctttcagtgagctcttccacccaggtgcaaccccctggcattgaccagcataggtgagtggtcccgctggggtcatgggtcatcagtcatgcagtgtggggtcaccaagagtcagcctttgagacaggagggtttccacaggcagctgacctgaatccacaccatggaagggtaggttggtcctccaagagggctgtttttttgggaatcggacccaaggatgatatctttcctgcagccctgcagtgatgacggtggtccaggcaggaaaagtttccttcaatctcatgcagggcagctcccagctgagtctcagtgcactggtctggcctcgaggactgaccaggccacgtgaggggggccacaggggctttgggatgccatggggtcacacaagagctccccatccctggagaaaggtgggcatagagcccatggagttgagctgcagatatttaaggatcacacaggatttatagccctgaatgggaccgaccctgttgagcaatgtgccatccatcctatggtacctctttgggcctggatggggtgttgctgtttccctaggttttggtcaaggctgagctctggccagtgcctggtggtgctacagtccagccacggacttgttagctttaggccaagggatcagtccacgtcctgctggggatctccccttccctgtggctggaagtcaccccttccccattggcaagtgtgaggaagggtggcctcctgaggtggaaggcctgctgttgagggtcttgtccctggaatatgccacccacactgcttctgagccatagggtcacctccaggatagtgggtattgggccaaaggaaagaagtcaccatgtgtcccctgattagccaagtgtggccctgctctgtgtttaggaggtgtgtggggcatggatggggacccaccagagaaagcatgtggccctgggactccaggttgtgatttccagttcatgctgggatagggcacctggatgggtgcccacaatgcaccggggcaggctatgtgactgccagctttgcagcggcatctggcatccgggccagcctccttggttggccatgaactggttggtggccctggcctgtgcccagtagctctgtcgtaatcctggggtcatgcgagggagccggcatgggcagtggaggtgctcgtgggacctgcggtaggacacagccaggtcttcttgcccgagcccagtgacccacctgccctgtccttccagggttcagtggttgagagtacagggtgacttgtgtctgtgacaagccagtgttctgcatggatcccatcacgtggatccatggacggcaagcatccctgagccgccacaccccaccttctgcctccatggaggggtgaggttgtgtgtggacgcctgtcctactcctgtttccccaggtattgagcctggaggaagacttgaattggtcccaatggtcccaggccagcatccatcagctaggtgctgagcccctggtgcactgaagattgggcacttcctggcaccctggtgtgctgcaccgagctgtgatgagcacatccgggtcctgctggatgcatgcgccgggaaggacgtgccctgagttgggtcgatgatgagaaccttatattatcctgaagagaggtgatgacttaaaaatcatgctcaataggattacgctgaggcccagtctaggtgagagttttggaagaggatgctgggatcctgaggtcccctggtagagccaattgtattttgggctggagacctggaagccctgaagggcatctgggaggcccaaccctgtctctgtgctcctccatgaggcagccctggctccccatgtaggcttggtgatggccttcctgttccctgggggtggggcacatgggtagctcctccctgacccccattctctcctgtgtctttcagtgagctcttccccacaggtgggccccctggcattgactagcatcggtgagtggatcctgctggggtcgtgggccgtgcaccaggctgcatggggtcaccaagtattagccttcaggacaggagggtttcctcagggagtcgaccttaatcctcacctgtggagagatgggtttgtcctctaggagggcagcttcctgggaaatcagacccaagacgatgccttccctgaagccctgcaggggtgacggtggtccagtctggaaagggttccttcagtctcatgcagggcagctcccagctgacacttggtgtgagagtctgtccttgaggactgcccaggacaaatgagtgggacaacagaggctcccggataccacagggatcactcaggggctctccgtccctggaaatgggtgggcgatcgggcccatggggtggggctgcggagattcaaggatcgtgccctgctcatggccctgaatgcaaatgaccctgttgagcaatgggtcacaccagtagctcctttttgagcctggtgggtggcggctcagcttcaactggttgggtcaaggctgagccatgactggtgctttgtggtgccgcaggccaaccctgggactcatgggctttgggtcaagggtgcagcccgcctgctgctgggggtcaccccttcttcgaggtcagtggcgggaagggtcacgtcccgaggttggaaagccccctcatgagggacctgtcctgggaatttgttgcccaggctgcttctgagccatgtggtcacttccgggtggcggggtcttgggcccaaggaaaggagagggcaccatgtggctcctgattggctcagtgtggcccagctctgtgtttgacaggggtctggggtgtggatgaggacccaccagacaaagcacgtggccctgggcccaggctgtgatttctggtgcgtgcttggagaaggatcctgggcaggcattctctccaaacaggggcaggttgtgggactgccagattggcagcagtgcctggcatccaggccagcatccttggttggctgtgagctggttggtgaccttggaccctgcccagggtttccgttgtggtccagagtcatgcagtggggccagtgtgagcagtggaggtgctccaggggcctgctgtatggcacagccagggcatcttacccaagccctcctgcactggtctcctgcactgagctgtggtgagcccatctgggttcctgtgatgcctgtgcggggagggggttgccctgggttgggtcgatgatgagaaacttatattgtctgaagagaggtgatgacttaaaaatcatgctcaataggattacgctgaggcccagcctaggtgagaattttggaagaggatgctgggaattatgagttccctggcagaaccactgtattttgggctggagacctggaggctctgagggcatctggagggtgcccaaccctgtctctgcgctcctccgtgaggcagcccaggctcactatgtgggcttggtgttggcactcctgttacccgggggtggggcattttggcatctcctttctgagcccccaatctcccctgtgtctttcagtgagctcttctgcccagcagcctccctggcagtgaccagcataggtgagtggatcctgctggggtaatggcccatgagccaggctgtgtggggtcaccaagcatcagccttcagccataagagtttcctcaggagctgacccgaatgcccacctgggatgggttctccagaagagtggcttccttggaaatcatacccaaggaagataccttccctgaagccctgcaggggttgcagtggtccaggcagtaaagggttttctcaacctcatgcagggcagtgccccactaagactcagtgctctggtcaggcctttaggatggccaggccacatgagaggggccacaagggctctggggcaccaaggggttcttgcaggggctcttcaaccttggagaggtgtgtgtaattcagcccatgaggtcaggctgtggattttcaaggattgcaccaaacccatggccctgaatgggacaaaccctaataagttatgggccatcccagtggtgcctctttgagccaagtgtggggtggcactgtttcctaagggtgagtcaagactagctctggctggcacctgatggtggtgcaatcctgccctgggattggtgggctttgggacatgggtacagcccatgtcccattggggtcaccccttcattgtggacaggtggcgggaagcatgtcctcctgaggtgggaaggcctgctgtggaagaccctgtctcagaaatttgctgtcttggctgcttctgagctatggcgtcacctctgtggtggcgggagtctcgggtgaatggatcctgctggggtcatgggccatgagccaggccggggtggggtccctgagcgtcacctttcgggtcatgagggtttcctaagggagccgacctgaatccccaccagggatgaatggtttctctgggaagcaggtttccttggaaatcagtcccaagaaggacaccttccctgaaaccttgcaggggtaacagaggtccaggcaagagagggttctttcaacccaacgcagtgcagatcctagctgagacttggtgcacaggtgagtcctggaggactgcccaggccacatgagggcatccacagggtctccgtggccaggagtcttatacgggagccctctgtccccgaggcttgcagaggggttgggtgtgggtgatctggcttacggagtcaggccgtgaacattcaagggtcgggaaccacccatggccctgaatgggaccgaccctgttgagcaatggccatcctggtggtggcaccttgagcctggtgggggtggcgctgcctcagtggggttgggtcaaggctgagctctgcctgtcaccttgtggttggccctgggaatcatgggctttgggccaagggtgcagcctgcgtcccgcttgaggtcactcctacctgaagtggagtggccggaagggtggcatcctgtggtgggagacccacggtcaaggatcctgtgctggcaatttgctgcgcagcctggttctgagtcatggggtcacctccagggtgacagggtctttgtcaaagggaaggagaggacattatgtggcccctgattggccaagcatggcccagctctgtgtttgggaggtatgcggtgcctgaatggggacccaccagagaaggcacatggccctggggccccaggatggtatttctggtgtgagcttataaagggtgcatggatgggtgcattccatgcccagtggcgggttatgtgactgccaggtctgcagtggcacatggggtccgggccagcctccttggtttgccatgagctggttggtggcccaggccctgcctagtggtctcattgcagcccagagtcatgtgggggagctggcgtgggcagtcgaggtgctccagggtcctacagtagggtgcagcctgggcgtcttgctggaatcccatgacccacctgccctatccttccagggttgagtggcagggagggccaagggttcctcgtgtgcatgactgtcactgttttacatggatctcatctcatgagtccatggtgggagagcatgtgtgagccagcacaccctacccactttgccctatggaggcacaaggccctgtttggacacctgtcccccacccctccagatgctgaccctggaggaagaattgcactgggcccactggccccgggaaaatgtccctcagccaggtgcttagcccctggtaatctgaagctcaggcccttcctgatgccctggtctcctgcactgagctgtggtgagcacatctgggtcccaatggatgcatgcatgcgttgtggggtggtggtgtcctgggttgggtcgatgatgagaaccttatatgttctgaagagaggtgatgacttaaaaatcatgctcaataggattacgctgaggcccagcctaggtgagaattttggaagaggatgctgggatttctaggtccctgtcatgggcactgtattttggactggagacctccgggccctgaacagcatctcgggggggactctaccctgtctccacattacatgtgaggcagcccaggctccctttgtgggcttggtgtctgcactctggttccctgtggtagggcagcttgctgggtccttcctgagcccctgttctccctttgtgtcttctgtctttcagtgagctcttcagcccaggtgggctccctgacactgatgcgcataggtgagtggattctgctggcatcatgggccatgagccaagctgcgtagggtcaccaaaggtcactctttaggccacgtgggtttcctcagggagccgacctgaatccccaccaggttgggacagtttctccaggaggctggcttccttgggaatcagacccaagacaatgccttccctgaagccttacaggggtaacagttgtccagacaggaaagggtcccctcagtcccattgaaggcagctcccagctgagactcagtgtgcgagtctggcctcaaggatggcccaggccacatgaaggaggccacaggggctctggggtgccacaggggtcacacgagagctctctttccctggagtggggtgctcgaatgagcccatggggtcaggctgcagagattcaaggatcgcatcccaaccgtggccctaaatgcgaccgaccatgttgagtaatgggccatctcagtggggcctctttgagcctggtggggggcggggctgtttccctgggtttgggtcaaggctgagctctggccagcgcctggtgatgctgcaggccagcccaggttcttgtgggcgttggtccattcggcagccctgtcctcctggaggtcaccccttgcctattggtgattggctggatcctgaggtggaaagacctgctgttgatggtcctgtcccaggaatttaccacccaggctgcttctgagccatggggtcacctccagggtggcggagtcttggggctaagaaaaaagaggacactatgtagcccacggttggccaagggtggcctgactctgtgtttgggacatgtgcggggcatggatagggacccgccagagaaagcacgtggcccggggactccaggcttcgatttcaggtgtggcttgcaaatagcacctaggcaggtgcccaccacgccctggggcaggctatgcaacttccatctccacagtggcgcatggcttctgggccattctccttggttggccatgagctgattggttcccctggcctgtgccctgtggtcccggagtggctctgggatcgtgtggggcagccagcatggacagtggagatgctatcaaggccttcagtagggtgcagcccaggtttcttgcctgaacccagtgacccacctaccctgtgttttcagggtttgctgacagggaaggccaaacgtgccccacgtccatgctaggtcagtgttcctcatggaccccatcacatgggtatcccttaccccttccccacatggaagtatgaggctctgtttggacacatgtcctcttcctgtccccccagatggtgagcctggaggaagacttgcattgggcccaatggacccaggccagtggccatcagccaggtgcccaccctggtgtgctgaagcgcgggacctacctgttgtcctggtttcctacactgagctgaggtgagcacatctgggtccctctggatgcatgcatggggcggggggtgccctggtttgggtcaatgatgagaaccttatattatcctgaagagaggtgatgacttaaaaatcatgctcaataggattacgctgaggcccagcctaggtgagaatttggaagaggatgctgggatcccaaagtcccagtaggggactttatttatttattttgggctggagccctggaggccctgaagggcatctggggggcccagccctgtctctgtgctcctccgtgaggcagcccagcctccctgtgtgggcttgctgttggtggtccagttccttggggcggggcacgtggacagctcctccttgagcccccctgctccacttgtgtctttcagtgagctcttcaacccaggcggggcaccctggcattcaccagcataggtgagtggatcctgctgccatcgtgggccatgggccaggctgggtagagtcactgagggtcagccttcgggacaggagagtttcctcagggagcgaacctgaatccccaccagggagggataggttggtcctccagaagggtggcttcctgagaaatcggacccaagggggacgccttccctggagccctgcagtgatgacggtggtccaggcaggagagggttccttcagtctcacacatagtagctcccagctgagtctaggtgttcgagtctggccttgaggactgcccaggccacctgaggggccaacagggactctgggacaccatgggggtcacataggagctctccattcatggagaaagatggatgatagggcccatggagtcgagctgtggagatttaaggattgcgcccaatgtatggccctgaatgggacagaccctgttgagtaatgagccattctatgggcctcatcgagccttgagcggggcggtgctgtttccctatgtttgagtcaaggctggactctggccggtgcctggtggtgctgaggtctggccatggactcgttggctttgggccaaggggtcagcctgctccccctggaagtccccccttccccgtggctggaagtcatcccttcccaaggggcaagtgtcgggaatggttgcctcctgaggttgcaaggcctgctatctagggtcttgtctttggaatatgctgcccatgctgcttctgagtcatggggtcacctccagggtagcggatcttgggcccagggacagaagatgccgtgtgtcccctgattagctgattgtagccctgctctgtgttcaggaggtgtgtggggcatggatgcagacccaccagagaaaactgtggcccttagaccccaggttgcaatttccattatgtgctgggatagggcatctgggtgggcacccactatgcacagagataggctatgtgactgccagggctgtagcggcatctgacatctaggccagcttccttggtttcctgcgaatgagttggtggccctggcctgtgcctagtagttctggggtgaccccgggtcgtgtgggggagccagcaggggcccagtgaccagcctgccctgtccttccagagttctgtggctaagagagcactgggtaccctgtgtccgtgatgggccagtgttccgcatgcaaacgatcacatgggtccatggagggcaagcatacttgaactgccacaccccaccttctgctcacatacaggagtgaggttgtgtttggacagctgtcctactcctgttcccctagactgtgaaatgggaggaagaattgtgttgggcccaatggccctgggccagtatccgtcagcctggtgtccagcccctggtgtgctgaagctcatgcccttcctggtgccctagtttcctgcactgagcttggtgagcccatttgggtactgctggatgcatgggtggggagggaggtgccctgggttgggtcgatgatgagaaccttatattgtcctgaagagaggtgatgacttaaaaatcattctcaaaaggattatgctgaggcccaccctaggtgacaattttggaagaggacactggaaatcaatgaggtccctggcagggccactataatttggggtggagacctggaagccctgcagggcacctcgtgggggcccaaccctgtctcttcactcccctgtgaggcagcccaagctccctctgtaggcttggtgtcggcactctgctttcctggaggcggggcattttggctgctccttcctgagcccctgttctcccctgtgtctttcagtgagctcttttgcccagaagactccctggcattgatcggcataggtgagtgtctcctgcttgggtcatgggccatgagccagaccatagggttgccgagggtcagcatttgagccatgagggtttcctcaggaggcgacctgaatccccacctgggatgggttctccaggagggcagcttccttgtgaatctgagccaagtaagatgccttctctgaagccctgcaggggtgccagttgtgcaggcaagaaagggttccctcagccccaggcagggcagcgcccagcagagactcggtggttgggttaggcctctaggatgaccaggccacatgaggggggccacaagggctctggggcacaacaggggcttcgcagggtctctgcgaccccaaagagtggtgggcgattcggcccatgggatcgggccccagattcaaagatcacaccacacccatggccctgaatggaaccgaccctgttaagcattggggcatcccaggggtgccactttgaacctggtgtggggtgccgctgtttgcctgaggttgggtcgagactagctccggccagcacctgatggtggtgcagtccggccctgagattagtgggctgtgggcccagggtgcagcctgtgtcccgctgggggtcacctcttccttgtgggcgagtggtggaagggcatcctcctgaagtgggaaggcccgctgtggaaggaactgtcctggaaatttgccatccaggctgcttctgagccatggggtcgcctccagagtggcagggtcttgggcccaaggaaaggagaggacactgtgtttccctcaattagctgagtgtggccctgctctctgtgtgggaggtgttgtgtggggcatggatagggacccaccagagacaacacatggccctcgggccccaggctgcaatttctcagtgcatgcttggagacggtcctgggcctcctccatgcccagggtcaggctgtgcaactgtcaggttagcggcggcatgtggtgtctgggcctgtctacttcgttgtccgtgagctgctggtggccctagccctttcccaggggttcaggtgcagccctggggttgtgtgggacagccagtgtgggcagtggagctgctctggaggcctgctttagggtgcagcctgggtgtcttgcccgagtgtagtgacccgcctgccgtgtcctcacggggttccctagcaggaagggccaagggtcccctgtgtctgtgacacgtgagtgtttcccatgggtcccatcaaatgggtccacggagggagagcatgagtgaactggtaccccccacccccaccctcttccccacctggaggcatgagcccctggttggacatatgtcctcctcctgtacccccagatggcagcgctggaggaggacttgtactggggccgctggccccacatcagtgtccgtcatcccggcgtccagcccctggtgtgctgaagctcaggcccttccttgtgccctagtctcctgcactgagcttggtgagcccattcggatactgctggatgcatgggtcgggagggaggtgccctgggttgggtcgatgatgagaaccttatattgtcctgaagagaggtgatgacttaaaaatcattctcaaaaggattatgctgaggcccggcctaggttagaattttggaagaggatgctgggatcctgaggtccccaggagagcccctttatttttggctggagacctggaggccctgaagggtatctggggggcccagccctgtccctatactcctctgtgaggcagcccaggctctctgtgtgggcttggtgttggcactctggttttctgggggcggttcaggttgctggttccttctgaaccattgttctccccttgattctttcagtgagctcttctgctcaggcgggtcccctggcattgaccagcataggtaagtggatcctgctgggttcatgggccatgagccagaccacatggagtgactgagggtcagccttcaggacaggaagatttcctcggggagccaacctgaatccccaccgtggagggatgtgttagtcctccaggagggtggcttcctagggaatctgaccccaggaggacacctttgctgaagccctgcaggggtgatggtggtccaggctggaaagttttccttcagtctcatgtggggcagctccctgctgacattccgtgcctgcgtctgaccttgaggactgcccgggccacatgagtggggcaacaagggctccagggtgccacggggattatgagtggggctctccatccccggagagaggtgggcaatcaggcccatgtggtcaggcttgtagggttcggtggtggggagagccaaggtttccctgggtccattaccgtcactgtttcacctggatcccatcacatgggtccatggagggagagcatgtgtgaacaggcaccccttgctccctttcccacatgagcccctggttggacatatgtcctcctcctgtccccccagatggtggccctggaggaggacttgtattggggccactggccccacgtcagtgttcgtcagcctggcatccagcccctggtgtgctgaagctcaggcccttcctgttgccctagtctcctgcactgagcttggtgagtccattcggggactgctggatgcatgggcgggtagggaggtgccctgggttgggtcgatgatgagaaccttatattgtcctgaagagaggtgatgacttaaaaatcattctcaaaaggattatgctgaggcccggcctaggttagaattttggaagaggattctcggatcctgagatccccagccaggccactgaatttttggctggagtccttgaggccctgaagggtgtctgagggctgctgaaacctgtctctgtgcttgtccgtgaggcaacccacattcgctgtgtggacttggtgttggccttcccgttctccggggatgggacatactgtcagcttgtccctgagtacccgttctcccctgtgtctttcagtgagctcttctgcccaggtggtccccctggtcttgatgggcataggtgagtacatcctgctggtgtcctggtccatgagccagggtgcgtacggttgccggaggtcacctttaggccatgtgggtttcctcagggagctgacctgaatccccactgggagggatggtttctccaggaggatgtcttccatagaaactggttccaagaaggatgccttccctgaagctgtgcaggggtgacgatggtccaggcaggaaagggtccattcaacccaatgcagtacagcttctagctgagactcggtgcacgggtgagggcttgaggactcccaggcaacatgaggggttccacaggatctccggaggccacaggtgttaagcaggaactctccatccttgaggcttgctgggggtggcgggatggcagcagggtgtgggtgatcgagtttatggattcaggccaagattcaagggtcaggccccacccattgccctgatgggaccaaccctgttgtgcaatgggctgtcccactggtgcctccttgagcctggtgtgggcagcgtggcttcaatggggttgggtcaaggctgagctctggcccgcacctggtggttggccctgggaattgtggactttgggccaagggtgcagcccgcctgcctctggaggtcatcctacccagaggtggagtggccggaagggtggcaggcatcctgtggtgaaaggcccactgttgagggtcctgtcccaccaatttgctgttcaggctgcttctaagccatgggttcacctccagagaggcgggatcttgagcccagggaaaggagagggcatcatgtgtcccctgattggctgagaatggggagagtgtttgggtggtatgtggtgcatgaatggggacccaccagagaaggcgcgtggccctgggccccaggatgcaatttccggcgtgcttatagagggcgtctgagtggacactctccatgcccaggcgcaggctatgtgactgccaggtctgcagcagtttgtggcatctgggctagcctccttgattgcttatgagctggttgatagccctggcctgtgccgaggggtcctggtgcggccctggggttgttcaggggaagccggcatgggcagtggaggtgctcttggggcctgcagtagggcacagcccaggtgttcttgcttgagcgcagtgaaccacctgccctttccttccagggttcggtagcagggagggccaagggtcccccacgtccatgacaggtcagcgttcctcctggatcccatcacatgggtacatgcagggagagcatgtgtgagccggcactccccactctcttccccgcattgcagggtgaggccctgtttggacccatcttccagctatctcaccagatggtgagcccagaggaagaccccgtggctctgggccagtgtgcgtcagccaggtccccagcccccagtgtgctgaagctcaggcccttcccaacgccctggtctcctgcactgaggtgtggtgagcacaaccaggtcccactggatgcatgcacagggagggatgtgccctaggttgggtcgatgatgagaaccttatattgtcctgaagagaggtgatgacttaaaaatcatgctcaataggattatgctgaggcccagtctaggttaaaattctggaagaggatgctgggatcccaagttcacaggcagggccactgtattttggactagagaactcgaggccctaaagggcatcttgagggggtccaacctgtctctgtgctcctccatgaggcagccctggctccctgtgtggccttggtgttggtgctctggttcccaggcgtggcatgtgggcacctccttccagagcccccattctcccctgtctctttcagtgagctcttccgcccatttgggccctctgacattgaccggcataggtgagtggatcctctttgggtcatgggtcatgagcgaggcaaagtggggtcaccatgcatctgccttcgggccatgagagtttcctaaaagtgtggacctgaattcccaccggggatgagttatccagaagggccacttccttgggaatcggacccaagaaggacgcctgtcctgaagcccagcaggggtggtggtggtccaggcaggaaagggttcattcagcaccaagcaggcagctcccatctgagacttggtgcatcggtcaggccttgaggactgcccaggacacatgaggtggccacaggggctccggggcactacagggggtctgcagagaggggtaggcgattgggtccgtgggggtcatgccgcggagtttcaaggacacccatggccctgaatgggactgaccctgttgagtaatgggccatcccaatggcacctccttgagtctggcggtgggtggtgctgtttccctggggctaggtcaaggctgagctctggctagtgcctgtggtgctgcaggctggccctagaactcatgggctttgggccatggggacagcttgtgtcatgcttggggtcaccccttcccggaggttgaatggcgggagtggtggcttcctaaggtgggaagtcctgctattgagcaacctgtcctgggaatttgccacccaggctgcttctgagacatgaggtcacctccagagtggcagcatcttgggtccaaggaaagaggagcataacatgtgtccccagattggttgagtgtggcctagctctgtgtttgggaggtgtgcagggtgtggatggggatgtatcaaagaaagtacatagccctggagccccaggctgtgatttccagtgtgtgcttggagaggggcaggagcaggctctcgactgccaggtctgcagcagcgcgtggcatccgggcaagccttctggttggccatgtgctggttggtggccctggcctgtgcccagtggtctcagggtagacctggcattgtgcagggcagccagcagggcagtggaggtgctctcaggacctgcagtagggcgcagcctggactttttgtgtgagaccagtgacccacctgcactgtccttccagggttgggtggctgggagggccaagggtggtccatgtttgtctccgtgacacatcagtgctctgcatggatcctatcacatggatatcccctacccaccttccccacatggaagcatgaggccttacttggacatgtgtcctcctgtcctagatggtgagctcggaggaagacttgcattgggcccaatggagctgggccagtttctgtcagccaggtccccagctcctgatgcactgaagctcaggcccttcctggagccctggtctcctgccctgagctgtggtgagcccatctgggtcccactggatgcatgcatgggagggggttgccctcggttgggtcgatgatgagaaccttatattttctgaagagaggtgatgacttaaaaatcatgctcaataggattacgctgaggcccaacctaggggagaattttggaagaggacgctgggatcctgaggtccgtggcagggccaccgtattttgggctggagacctcaaggccctgaagggcatcttggcagcccccagccctgtctctgcactcctccgtgaggcagcccaggctccctgtgtgggcttggtgtcagagtcccattccccaggggcggggcaggttggcggctcctccctgagtcccctttctcccttgtgtctttgagtgacttcttctgcccaggtgggccccctggcgttgaccagcataggtgagtggaacctgctggtgtcatgggccatgagccaggccatgtggggactccaggtcagccttcaggacaggagagtttcctcagggagcaggggaatccccactggggagggatgggttctcccagagggccactttcttttgaatcggacccatggaggacatcttctctgaagacctgcagaagtgatgatgtttcaagcagcaaagggttttttcagcccctgcagggcaggcagctcccagatgagattctgtgcatgggtcaggcctcaagaactgcccaggccacatgcggggtgccacacaggctgtgaggcaccacaggggtcatgcaggagctctcggtcttcaggcttgttgtgggggtggtgacagagtgatcaggcccacagggtcaggctgcagtgtttcaaggatcgcgcccacctgtggccctgaatgggaccgatgctgttgagcaatgagccatcccagtggctccacattgagcctggtgggggcgccactgtttcagtggggatgggtgaagcttgagctctggccagcacctgatggtgttgcaggctggcccagggacttgtgggctttgggccaggtcggcagcctgagttctgctgggggtcacatctttcctgagtttcagtgtcaggatgcatggcctccttaggtgggaaggcctgctgtggagggtcctgtcctgggaatttgctgcccaggctgcttctgagccatggcgtcacctccaggtggtggggtcttgggcccaaggagcagagaagacaccatgtggcccctgattggtcgagtatggcctggctctgagtttgggaggtgtgcagggcatggatggggacccaccagagaaagcacgggccaatgaagccccaggctgcgatttctgatgcgcacttggagagggcgcctggttgggcgcccaccatgcccaagggtaggctatgcaactaccaggtctgcggcagtgcctggcatcaggaccagcctcctttgttggctgtgagctggttggtggccctagcccgtgtccagtgatctctgtgcagccctggggtcatgtggagcagccagtgtaggcaggggaggttatcttgaggtgtgcagtaggcacatcccaggcatcttgcccgagcccagtgacctccctgccctgtccttccagggttcggtggcggggaggcccaagggtaccctgtgtccatgacaggtcagtatttcatgtgaatcccatcacatgggccctcctcctgtccccccagatggtgagcctggaggaagacttgcactgggccttctgtgcgccggccagaggccatcaaccaggtgcccaggtccttttgtactgaagcttgggcccttcctggcaccctggtctccttcatggagctggtgagcccatccgggttcttctggatgtatgcatggggagcggggtgcactgggttgggtcaatgatgagaaccttatattgtcctgaagagaggtgatgacttaaaaatcatgctcaataggattacgctgagtcccagcctaggtgacaattttggaagtggacactgagaatcacaaggaccctggcagggccactgtattttgggctggagacctggaggccctcaagggcctctcacagggacccaaccctgtctctgcactcctccgtgaagcacagcaggctccctgtgcggacttggtgttggcactgtttcccaggggcagggcacattggtggcttttccctgagtccccattcttgccttgtgtctttcagtgagctcttcagctcagaagggccccttcaccttgattagcctaggtgagtgcatcctgctggtgtcatgggccatgagtcaggccgcgtggggtcaccaagggtcagtctttaggacaggagggtttcctcagggagccaacctgtatttccaccggggagggatggtttctacaggagggcggcttccttgggaactggacccaagatgttgccttcctgaagccctgcagggatgacagtggtccaggcagcaaagacttccttcagccccgtgcaggagagctcccagctgagactcagtgcatgtgtcaggcctcgaggattgccgaggccacatgatgggggccacagggcctccggggcaccacgggggtcatgcaagagttctctttccctggagaggggtgcttgaatgagcctatgggattgggccgtggagattcaaggattgtacccaactatgtcccaaaatgggactgaccctgttgagctatggaccatcccatggtgcctctttgagcttggtggggggcagtgctgtttccctgggtttgggtcaaggctgagctctggccagtgcctggtgaggctgtgggccggacctgggactcgtgagctttaggccaagtcgacagcttgtgtcctgctggaggtcaccccttccccatgggtgagtggctggaagagtggaatgtgcggtgggaaggcccgctgttgatggtcctatcctgagaatttgctgcccagactctctgtgccatggggtcatgtccagggtggcagggtcttgggcctagggaaaggaaaggacaccatgtggcccaggattggccaagggtggcccggatctgtgtttgggacatgtgtggggcatggatggggacccaccagagaaagcacatggccctggtactccaggctgtgatttcaggtgtggcttggagagagtgcctgggcaggtgcccaccatgcccagcggcaggctatgcaacttccatgtccacggtggcacatggcttctgggccaggctcctcggttggccatgagcatgtcggtgaccctgtcccatgctcagtggtcctggggcagccctgggatcgtgtgaaggagaagtcagagggggcactggatgtgccctcaaggtctgcaatagcatgaagaccaggggtcttgcccaagcccagtgatccacctgccctgttttttcagggtttggtgatggggagggccgagagtgccctgtatccgtgacaggtcagtgttccacatggatcccatcacatgggtatcccttaccccattccccacctggaggtatgaggccctgtttggacacatgtcctcctcctgtccatccagatggtgagcctgaaggaagacttgcatcaggaccaatggaccctggcaagtgtccgtcagccaggtgctcagcccctggtgcactgaagctcaggcccttcctggccccttgatctcctgcactgagctgtggtgagcacatccgggtcccgctggatgtatgtgtgggcagggggggtgccctgggttgggtcaatgatgagaaccctatattgtgttgaagagaggtgatgacttaaaattaccatgctcaatgattacgctgaggcccaacctaggtgagaaatttggaggaggatgctgggatcccgagatttccggcagggccactgtattttgggctggagccctggaggccctgaaaggtatctggaggaggcccaactctgtctctgcactcctctgtgaggcagcccaggctccctgtgcgggcttggtgttggcctttctgttccccagtgaagtggcacgtgggtggctcctccctgagccccagttctccccttgtgtctttcagtgagctcttctgccaagggggggcctcctggcattgaccaacataggtgagtggatcctgctggcatcatgggcatgggttcactgagggtcagacttcaggccatgagggtttcctcagggagctgaccccaattcccatcagggagggatggtttctccaggagggcggcttccttgggaatcttacctgaggaggatgccttccctgaagatctgcaggaatgacaatggtccagatagcaaagggttccttcagccccatgcaggacatcgcccagttgagacttggtgtgcaggtcaggccttgaggactgcccaggccacatgaggcgggccagaagggctccagggtgccacgggtttcacttgggagctctcggtccccgggcatgttcctggggggggtggtgatggggtcgggccatggaggttcaaggatctcatcccacctgtgaccctgaatggggctgatcctgttgaacaatgggccatcccagtggcacgtattgagcctggtcaggggtggtgcagtttcaatggggttgggtcaaggttgagatctggccagctcctggtggtgcttcaggccagtcctgtgacccatgggctttgggctaagggggcatcccatgtcccatgggggtcacctgttctccatgtttgagtggcagtaacagtggcattctgaggtgggaaggtccactgtctagggtgttgtaccgggaatttgccaccccgccttcttagccaaggggtcacctccagggtctcgggatcttgggcccaaggaaacgagaagataccatgtggcccttgattggccgagtgtggcccagctctgtgtttgagaggtgtgggatatgaatggggtcccaccagtgaaagctcatggcccaggtccccaggctgtgatttttagtgcacacttggggagggctcctcagtgggtgcccaccacgcccagaggcaggctatgtgactgccaggtctgcaacggtgcatagcttccaggctaccctccttggttggctgggagcgggttggtggccatggcccatgcccagtggtcacagggcaaccctgggatcatgcaggggagctggtgagggcagtggaggtgctctccggaacggaagtatggctcagcccaggtatctggcccaaacccaatgaccagcctgccttgtttctcagggtttggtgatggggagggccaagggtgccctgcatctgtgatgggtctgttccacatagaacccattacaggggatcccttctccacatggagatgtgaggccctatttggacacatatcctcctcctgtccccccagatggtgagcctggaggaagatttgcattgggcccaatggccctgggccagtgtctgtcagccagtttcccagcccctggtgcactgaagctcaggcctttcctggcatcctggcctcctgcactgagctgtggtgagtatatcatggtcctgctggatgcatgtgcagggagggaaggtgccttgggttcagtcagtgtcgagaaccttatattgttctgaagagaggtggtgacttaaaaatcatgctcaataggattacgctgaggcccagcctagttgagaattttggaaggggacagtaggatcccgaagtccctggcagggcccttatattttaggctggagacttcaaggccctgaagggcacctggaggtggctcaacctgtctctgtgctcctccatgaggcagtccaggctccctctgtgggctttctgttggcactctggttccctgggccagggaacgttggtggctcctccctgagcccctgttctccccttgtgtctttcagtgagtgcttctgcccaggcaggccacttggcattgaccggtataggtgagtggatcctgctggggtcatgggtcatgagcaaggccacatgggtcgccgagggtcagccttacagccataagggttttctcagggagcccacctgaattcccaccggggaggcataggttctccaggagggcggcgttttggggaatcagacccaagggggacaccttccctgaagccctgcaagggtgatggtggtccaggcaggaaagggtttttttcagccccacacaaggcagctcctagctgagaatcggtgcttgggtcagacctcaaggattgcccaggccacatgagggcgactgagagcctccggggcaccacaggggtcacgtgggagctctcggtgcctggcgtgtgatgcacaaatggacctcttgggtttgttcatggagattcaaggatcgcaccccaccatgcccctgaatgggatggtccctcttgagaaatgggccatcccagtgggtctcctggagcctggttggggggtggcattgtttccctggggttgggtgaaggcagagttctggccggcacctggtggtgctgcaggtcagcactgggacccgtgggcgttgggccaaaggagagcccgaatcccactgggggtcaccctttttccaacatcaaatgtctggaagggtggcctcctgaggtgggaagtcctgctgttgagggacatgtcctgggaatttgtcactcaagcttcttctgagctttggtgtcacatccagggtggcagggtcttgggtccaagcaaagcagagggcaccatgtgtctcctgattggccaagtgttgctggctctgtgtgggaggtgtgcagagtatggatggggacccacgagagaaagcacgtagcactgcaactccaggctatgatttctgttgcatgcttggagagggtgcctggacaagcgtgccatgcccaggggcaggctgggcaactaccaggtcttcagcggagtgtgacatctgggccagcctccttggttggccgtgacctggttggtggcccggctctgtgcctactggccccagggcagccctgggatcatgcaggggagccattttgaacagtggaggtgctcttggggcctgtagtatggcacagcctaggtgttttgctcaagcccagtgacccacctgccctgtcctctccgggatcggtggaagggagggccaaaggtgctccgtgtacttgatgagtcagtatttcacattgaactcatcacatgtgtccatggagggtgagcaagggtgagatggcactgctgacccccttccccacgtagaggtcagtttggacacttgtcttcctcctgtctccccacatggcgaccccagaggaaaacttgtgttgggcccgctgtcactgggccaatgtcagtcagccaggtgcccagcccctggtgcgctgaagcttgggcccttctggcacccttggctcctgcactgagctgtggtgagcccatctgggtcctgctggatgcatgtgcagggagggggatacctgggttaggtcgattatgagaaccttatattgtcctgaagagaggtgatgacttaaaaatcatgcccaataggattacgctgaggcccagcctaggtgagagttttggaagaggatgctgggattctgaggtccctgacagagtaaatgtattttgggctggagaccttgaggccctgaagggcatctggtgggcccacccctatctctgtgctcctccgtgaggcagcccaggctccctgtgctggcttggtatctgggctccggatccctggttatggggcatgttggagtctccttcctgagtccccgttctcccctgtgtcttttggtgagctcttctgcccaagtggtccccgcggccttgagcggcataggtgagtgcatcctgtttcggtcatgggccatgagtgatgcagcatggggtcctcgacattcagccttcgggccactagggttttctcaggctgccaacctactccccaccagggagcaatggtttctccaggagggccacttccttgggaatctgacccaaggagcacatctttcctgaagccctgcaggggtgacagtggtccaggcagcaaagggttccttcagcccaatgtagggcagctcccagctgaaactcggtggacagctcaggcctggagtactgtccagggcacatggggaggaggggcacagggaatgtggcgcaccctagaggttacactggagttctctctccctggagaggggtgagtgattgggcccacacagtcaggctgtgaagattcaaggaatgctccacacccatggccttgaatgggaccaaccctgttgagcagtgggccatcttatggtacctccttgagtctggtgagggatggcactgtttcagtgaggttgggtcaagactgagctctggctggcacctggtgctgctgcaggccctgggacttgggggccttcagtcaagggggcagctctgatcctgctggggacaaaccttccttgaggtccagtggtgaagagtgtcctcctaaggtgggaaggcctgctgtcaagggaatttgccacccaggctgcttctgagccttggggtcacctcagggtggcagggtcttgtgcccaaggaaaggagaagacaccatctggttcctgattggctgagtgtagccgggctctgtgtgtcggagttgggcagggcatggatggggacccaccagagaaagtgtggcccaggggccccccaggcttggagagggcaccagggccggtgcccacaatgctcaggtgctggctctgggagggccaggtctgcagcggcttttggcatctggccaaccaccttggttggccaggagctggttgatggccctgtcctgtgtcctgtgccctgtgccctgtgtccctggtatggccctggggtcatgtgtgggagctggcctgagcagtggaggtgctcttggggcctgcagtagggtacagcctaggcatcttgcctgagccagcactgtcttttgctttccctgtctttccagggtttggtggcagagagggcaaaggatgccccacatctgtgttgggtcagtgttcagcatggatcccatcacatgggtatcccttagccccttccctgcattgatatgtgaggccctggttggacacatgtcctccttctgtccctctagatggtgagcctggaggaagatgtgtgggacccgcaggccgagggccagtgtctctcagccaggtgctcagcccccagtgggctgaaactctgacccttcctggcaccctgctctcctgcactgtgctgtggtgagcacatccgggttccactggatgtttgtgcagggagggggttccctgggttgggtcaatgatgagaacagggccacatgaggggggccagaggggctacagagtgccatggggtaacaagggagctcttggtccctggagtgtggtgtgtgaatgggcccatggggttgggtagcagagattcaaggatcctgctctacccatggccctgaatgtgaccatcgctgttgagcaatgggccatcccagtgggtctcctggggcctggttggggggcggagttgtttccctgggattgggtcaaggttgactctggctggcacctggtggtgttgcaggtcagccctgggactcatgggctttgggccagggacagcccgcatcccgctgggggtcacccaagtgttgtgaagggtggcctcctgagttgggaaggcccgctattgaggcatctgtactgggaatttgttgctcacgctgcatgggagccatggtatcacctccagggtggcagtcttgggaccaagcaaaggagggggcagcatgagactccggattggctatgtgtgactggctatgtgtcttggaggtatgtggagtatggatggggacccaccagagaaaacaagtgaccctgaggccccatgctgtcttttctggtgcctgcatggagagggtgcctgtacgcatgcccaccatgcccaggggcaggctgtgccactgtcaggtctgcagtagcatgtggcatctgggccagcctccttggttggctgtgacctggctggtggcccggacctctgcccagtggtcccagggcagccctagggtcatgcggaggagcagttttggccagtggaggtgcccttgggtcctgcagtcgtgcataccctgggcgttttgcctgagcccagtgacctacctgccttgtccttcccgggttcggtggcagggagagacacttgcataggtgatgagtcagtatttcgcatggatctcatcacatgtgtccatggagggggagcaagcgtgagctggcacccaacacccctttctccacatggagggtggtcacatgttctccacctgttcccctagatggtgagacttctgttgggcttgcttttcctgggccagtgtccatcagccaggtgctcagccatcggtgcgctgaagctccagcccttcctggtgccctgatctcctgcactgagctgtgatgagtacatccgggttccactggatgcatgtgcgggaaggggggtgccctgggttgggtcagtgatgagaaccttatattgtcctgaagagaggtgatgacttaaaaatcatgctcaataggattacgctgaggcccagcctgggggagaattttggaaaaggatgctgggatcccagggtccccaacagggccactgtattttggacctgggacctggtggctctgaagggcatctggaggtggcccaacctgtctctgcactctagtttccctgtgtgggcttggtgtcggtgctccagttccctgggggcagggaatgttgatggctccttcctgagtccctgttctcgcctttgtctttcagtgagctctttcacctaggagggccccttggcattgactggcataggtgagtggatcctgctggtgttatggatcatgagccagaccatatggggtccccaaggatcagcctttgggccacaagggttttctcagggagctgacctgaatttccacctgagacggaaaggttctcctggaaggcaggctttttggaatcggacccaaggaggacaccttctctgaagccctgaaggggtgatggtggtccaggcaggaaagggttccttctgcctaacataggacagtgcctagttgagactcggtgctgcagtaaggccttgaggactgcccaggctacatgaggggggccagaggggctacggggcaccacagggttaatggtgacctcttggtccccagagtgtggtgcgtgaatggacccatggtgttgggttgtggagattcaaggatagtgccccacctatggccctgaatgcaactgtccctgtttagcaatgggccatctcagtgggtctcctggagcctggtggggggcagagttgtttccttgtgactgggttaaggtagagctctggccagcacctggtggtgtggcaggtcagccctgggactcttgggcttttgaccaaggggcagcctgtatcccgctgggggtcatcctatctctgaggtcaagtgtcgtgaagggtggcctcctgaagtgggaaggctcgctgtcgagggacctgtcctaggattttattgctcaggctgcttctgagccatggtgtcacctctagggtgatggggtcttgggaacaagcaaaggggagggcaccatgttgctccggattgggcaagtgtggcccagctctgtgtgtgtgaggtgtgcagaacatggatggtgacccaccagagaaagcacatggccctgtagctctaggttgtgatttccgtgcacacttggagacagcatcttgatgtgtgcccaacacgcccaggggcagactatgcgatagccaggtctgcagcggtgtgtggcatctgggccagccacctttgttggccatgagctggctcatggccctgacccatgcccactggtgtcagggggccctggcgtcgtgtgaggagccatctgtggcagtggaggtagactcagggcctgcagtatggcgcaacccatgcattttgcctgagctcagtgacctacctgccctgttcctccagggtttggtggcagggggagccaagggtgccccacattggtgacgagttagtacttcacatggatctcataacatgtgtccgtggagggcaagcaaatgtgagcccgcacccactactgccttccccacatggaggtcagtttggacacatatcatcctgctgtcccccaaatggtgagaccgaagaagacttgccctgggccctctgttcttgggccagtgtcagtcagccaggtgcccagctctaagtgcgctgaagcttgggcctttcctggaatgctcggctcccgcactgagccagggtgagcacatccgggtactgctggatgcatgtgtggggcaggggtgcgccctgggttgggctgatgatgagaaccttatattgtcctgaaaagaggtgatgacttaacaatcatgctcaataggattacattgaagcccaggataggtaagaattagggaagatgatgcagggatcccaagatctccagctagaccacacttattttgcactggagaacttgagaacttgaagggcatctcaggggagcccaaccctgtctctgagctccttcgtgaggcagcccaggctccccttgcgggcttggtgtctgtgctccgcttctccagggacagggcaatttggtgttccttcctgaatgctccttcttccctgtgtccttcagtgagctcttctgcccaggtggggtccctagctttgagcggcataggtcagtgcatcctgttggggtcatgggccatgagtgaggcagcatggggttggtgagagtcagccttcaggccactagggttttctcagggagccaaccgattactcaccgggagggatggttcctccaggagggtcacttcttgggaatctgaccctaggagcacacatttcctgaagctcggcagggagatggtggtccaggcaggaaagggttccttcaacctaacgtagggcagttcccagctgaaactaggtgggcaggtgaggcttgaggactgcccagtccacatgaaaggggccacaggggctttggggtgccctgggagtcatgctggagttcttcccccaagagggatgggtgatagggtccacgtggtcaggctgcggagattcaaggaatgccacccacccatggccctgaatgggaccaaccctgttgagcaatgggccattttcggcatgtttcaatgcagttgggtcaaggctgagcgctggctggtgcctggtggtgctgcaggccggccctggggctcatgggctttatgccaagggggcaactcccatcccactggggtaatcccttccttgaggtccagtggtgggaagggtggcctcctgaggtgggcagacctgctgtcaagggacctgtcctgggaatttgccacctaggctgcttctgagccatggggtcacctccaggatggcggggtcttcagcccaaggaaaggagaggacaccatgtggtttctgattggctgagtgtggcctggctgtatgtgtgggaggtgtgcagggcatggatggggactcatcagagaaagaatgtggccctggaggcccccaggctttgatttctggtgtgtgcttggagtgggcaccagggtggccctaccatgcccaggggcaaggtctttgcctgccaggtctggagtggctcctggcatcaggaccaccctccttggctcaaccaaccttcagcaggttggctgccttggcccgtgccccatggtcccggtacgaccctggggttgttcgggggatctggcccaggcagtggaggtgctcttggggcccacagtagggcacagcccaggcctcttgccccagcccagtaacccacttgccctgtctttccagggttttgtggcagggaaggccaagggtgctctgcgtccatgttgggtcagtgttctgtaaggattccattacaagggtatcccctacccaccttccctacattgatacatgaggccctggttggacacatgtcctcctcctgtccctacagatggtgaccctgaagaaagacttcagttgggcctgaaggccccgggccagtgtccatcatccaggtgctcagctcccagtgcactgaagctcagacccttcctggttccctggtctcttgcactgagctctggtgagcacaactgggtgctgctggatgcatgcatggggaggggggtgccctgggttggggtggtgatgagaaccttgtattcttctgaagagaggtgatgacttaaaaaccatgctcaataggattacacttaggccgaacctaggtgagaatgttggaagaggacgttgggatcctattatccctggcagagccactgtattttgggctggagacctggaggccctgaaagggcatctggagggggcccaaccctgtctctgtgctcctgcatgagggtccccaggctccctgtccagccttggtgttggtgctctggtaccctggggatggaacaagttggtggctctttcctgagcccccattctccctttgtgtctttcagtgagcttttctgcccaggtgggcaccctggcattgattagcctccttggttgtccatgacctggctggtggcccgatcctgtgcccagtggtcccagggcagccctggcatcatacggggcagctgttttgggcagtggaggtgctctccaggcctgaagtatggcacagcctgggcattttacctgagaccagtgacctgcctgccttgtccttcctgggttcggtggcagggagggccaagggtgccctgcgtcagtgatgagtcagtattttgtgtggatctcatcacctgtccatggagggaaagcaagcatgagctggcacccaacacccccttctccgcatggagggtggtcacatgtcctccacctgtgcccccagatggtgagacttgaggaagacttccactgggcctgcttttcctgagccagtgcccatcacccaggtgctcagtccctggtgcactgaagctccagcccttcctggcgccctggtctcctgaactgagctgtggtgagcacatccgggtttcactggatgtttgtgcggggagggggttctctgggttgggtcaatgatgagaaccttatattgtcctgaagagaggtgatgacttaaaaatcatgctcaataggattacgctgaggcccagcctaggggagaattttggcagaggatgccgggatcccaagatttccgggagggccacttgtattttgggttggagacctggaggccctgaagggcgtctggaggtggcccagtgctttctttgcgctcctctgtgaggcagcccaggctccctttgtgggcttggtgtttgcgctcctcttctccagtgatggggcacgttgggggctcctccctgagtcctggttctccccttgtgtctttcagtgagctcttctgccaagcggttcccctggcattgaccaacataggtgagtggatcctgctggcatcatgggctatgagctaggccgcatgggtttgctgagggtcagacttcaggccccaagggtttcctcagggaaccgacctcaattcccaccatgtgactgccaggtttgcaacagtgcatagcttccgagctaccctccttggttggccgggagctggttggaggctgtggcccatgcctagtagtcacagggcaggcagactgccctgttttttcagggtgtggtgacggggagggccaagggtgccctgcatccatgatgggtcagggatttcaggagggatggtttctccaggagggcagcttccttgggaatcttacccaaggaggatgtcttccctgaagccctgcaggggtgacgatggtccagatagcaaaggtttccttcagccccatgcaggacatcacctagttgagatatggtatgtgggtcaggccttgaagactgcccaggccacctgaggggcccagaagggctctggggtgccacaggtgttatgtaggagctctcagtccccagacttgtttcgtggggttggtgacggggttgggttgtggaggttcaaggatttcatcccacctgtgaccctgaatggggccaaccctgttgagcaacaggccatcccaatggcacgtattgagcctggtggggtgtggtgcagtttcaatagggttgagtcaaggctgagatccatctggtgcttggtgctgctgctggccagccctgggacccgtgggctttgggctaagggggtagcccacatcctattgagggtaacccattctccgagtttgagtaacggttaacagtggcattctgaggtgggaaggcccactgtctagggtcctatactgggaatttgccaccccgggttcttctgagacaagtggtcacctccagggtcatggggtcttgggcccaaggaaaggagaagataccatgtggcccttgattggctacatgtggcccaggtctgtttgggaggtgtgtgggatgtggatggggtcccaccaatgaaagcacatggcctgggtccccaggctgtgattttcagtgcacacttggagagggtgcctcggcgggtacctaccacatccatgggcaggctatgtgactaccaggtttgcaacagtgcatagcttccaagctaccctccttggttggtcgggagctggttggaggctgtggcccatgcctagtggtcacagggcaggcagacggccctgttttttcagggtatggtgatggggagggccaagcatccatgatgagtcggtgttccacatggaacccattacgtgggtatctcttaccccttcctcatatggagatgtgaggccctatttggacacatatcctcctcctgtacccccagatggtgagcctggaggaagacttgcgttgggcccaaaggccctagaccagtgtctgtcagccaggttcccaacccccgatgcgctgaagctcaagcctttcctggcaccctggtctcctgcactgagctgtggtgagtatatcctggtcctgctggatgcatgtgcagggaggtgggtgccttgggttgggtcaatgatgagaaccttatattgttctgaagagaggtgattatttaaaaatcatgctcaataggattacgctgaggcccagcctagttgagaattttggaggaggacactgggataccgaagtcccccgcagggactctgtattttgggctggagaccttgaagccctgaagggcatctggaggtggcccaacctgtctctgtgctcctccctgaggcagcccaggctccttttgtgggcttggtgttggggctccagttccccaggtcagggaatattggtagctctttactgagcccctgttctccctttgtgtctttcagtgagctcttctgcccaggcgggccccctggaattgatgggtataggtgagtgcatcctgctggggtcatgggccatgagccaggccacgtggggtcaccaggggtcccccttctggccatgagggttttctcagggaaccgacctgaattaccactggggaggcataggttctccaggagggcggctattttgggaatcggatccaagggggacaccttccctgaagccctgtgggtgtgatggtggtccaggcaggaaagggtcccttccaccccacacagggcagctcatagctgagagttgatgctcaggtcaggcctcaaggatttcccaggccacatgagggggacagaggagctctgggtgccacgggggtcacataggagctctcagtattcggtatgtcatgcacaaatgggcctcttgggttggattgtggagattaaaggatcgcgcaccaccgtggccctgaataggaccatccttattgagcaatgggccatcccagtgagtctcctggagcctggtggaggggagggggtggcattgtttccctggggatgggtcaaggcagagctctggccagcaccttgtggcttctgcaggtcagccttgggactcatgggcattgggccaagggggagcccgcgtcccactgggggtcaccctttttccgacgtcaagtatctggaagagtggcctcctgatgtgggaaggcctgctgtcgagggacgtgtcccaggagtttgtcgctcaagtttcttctgagctttggggtcacatccagggtggcggggtcttgggcccaagcaaagcagagggcaccaagtgtctcctgattggccatgtggctgtctctgtgtgtgggaggtttacagagtatggatggggacccaccagaggaagcacgtggccctgcagcaccaggctgtgatttctgttgcacacttggagaggttgcctggacacgtgcccttcatgcccagtgacagactatgcaactaccaggtctgcagcagggtgtgacatctgggctagcctccttggttggccgccacctggctggtggcccggatctgtgcccaacagtcccagtgcagcactggggtcatgcaggggagctgttttggacagtggaggtgctctcggggcctgcaatatgatgcagcctgggcgttttgctcaagcccagtgacccacctgccctttcctttctgggttcagtggaagggagggccaacagtgtagtgcgtacttaacaagtcagtatttcacattgaactcatcacatgtgtccatggagggcaagcaaaggtgagctggcacccccgacccccttccccaaacggaggtcagtttgggcacacgtctccctcctgtctccccagatggtgaccccggaggaaaatttgtgttgggcccactatccctgggccaatgtcagtcagctaggcacccagcccccagtgcgctgaagctcaggaccttcctagtgccctcggctcctgcactgagctctggtgagtccatctgggtcctgctggatgcatgagctgggggcgggagggtgccctgggttgggtcagtgatgagaaccttatattgtcctgaagaaaggtgatgacttaaaaatcatgctcaataggattacactgaggcccagcctaggtgataattttggaagaggatgctggaattctgaggtccctgacagagcagctgtattttgggctggagaccttgaggccctgaagggcatcttgtgggcccacccctatctctgtgctcctctgtgaggcagcccaggctccctgtgctggcttggtatccgggctctgggtccctggtgacggggcatgttggaggctccttcctgagcccctgttctcccttgtcattcagtgaactcttctgcacaagtgacccccccagccttgagcggcataggtgagtgcatcctgttttggtcatgggccatgagtgatgcagcatggggtccctgacattctgcctttgggccaccagggttttctcaggctgccaacctactccccaccagggagcgatggtttctcaggagggctgcttccttgggaatctgacccaaggagcaagtctctcctgaggccctgcaggggtgacagtggtccaggctggaaagggttccttcagcccaatgtagggcagctcccagctgaaactcagtgggcaggtcaggcctcgagtattgcccaaggcacatgggggagtgaggcacgggggatacagtgcgccgtggaggtcacactggagttctcccctccctggagagggatgagtgactgggtccacacggtcaggctgtggagattcaaggcatgctccacacccatggccttgaatgggaccaaccctgttgagcaataggccatcttagtggcacctccttgagcctggtgggggacggcactgtttcaatgaggttgggtcaagagtgagctctggctggcacctggtggtgctgcaggccctgggacttgtggactttgggccaagggagcagctctgatcctgctggggacaagccttccttgaggtccagtggtgaaaagagtgtcctcctgagatggcaaggcctgctgtcgagggacctgttctaggaatttgctgcccaggctgcttctgagccttggggtcacctcagggggtggcagggtcttgagcccaagaaaggaaaagacacgatgtggttcctgattggctgagtgtgacccggctctgtgtgtcagagttgggcaggacatggattgagacccaccatagaaagcatgtgatcctggaagcccctaggcttggagagggcaccagggccagcgcccaccacgcctaggtacgatctctcggacttccaggtctgcagcggctcttggcgtctgggccagcatgcttggttggccaggggctggttggtggccctgtcttgtgccctgtgtccccggtatggccctggggttgtgtgtggtagcaggcctgggcagtggaggtgctcttggggcctgcagtagggtgctgcccaggcatcttgcccgagccaggtgacacgcttgccctgtctttccagggtttggcggcagggagggccaagggtgcccagtgtccgtgagggatcagtattcagcatggatcccatcacatgagtatcccttagccccttctctacattgatgtgtgaggccctggttagacacatttcctcctcctgtccctccagatggtaagcctggaggaagacttgtgttgggcccacaggccccaggccaatgtctgtcagccaggtgctcagcctctggtgcgctgaaactctgacccttcctggcacactggtctcctgcactgagctggggtaagcacatctgggttgcactggatgtttgtgtggggagggggttccctaggttgggtcaatgatgagaacctgatattgccctgaagagagatgatgacttaaaaatcatgttcaataggattacgctgaggcctagcctaggtgagaattttggaagcagatgctgggatcccaaggtcccttgcagggccatcgtatgttgaactggagacctggaggctctgaagggcatctggaggtcgtgcaacctgtctctgcactcctctgtgaggcagcccaggctccctgtgtggggttggtgttggtgctatggttccctgggggtggggaacattggtggctccctccagagcccctgttctcaccttgtgcctttcagattttccacctaggaggtccacctggcattgactgacataggtgagtggatcctgctggggtcatgggtcatgagccagaccatggggggatcccagagattcagtctttgggcaacagttttctcagggagcagacctgaatttccacctgggagggaagggttctcctggagggcgacttttttgggaatcggacccaaggaggatgccttccctgaagccctgccggggtgatggtggtccaggcaggaaagttccttctgctcaatgcagggcagcacccagctgagactcggtgctccggtaaggcctccagaactgcccaggtcacataaggggggccagaggggctacgaggtgccacggggtaacaggggagctcttggtccccagagtgtggtgcatgaatgggcccattccgttgggtctcagagatttaaggattgcaccacaccatggccctgaatgcaaccgtccctgttgatcaatgggccatcccatgggtctcctggagcctggtgggaggcagtgttgttttcctggggttgggtcaaggcggagctctggctggtacctggtggtgctgcaggtcagccctgggacttgtgggctttgggccaaggggcatcccgcatcccgctgggggccaccctttctccatggtcgagtgtcgtgaagggtggcctcctgagttgggaaggcttgctattgaggcacttgtactgtgaatttgttgctcaggctgcttgggagccatggtgtcacctccagggtggcagggtcttgggaccaagcaaaggagtgggcaccatgtcactgcagataggccatgtgtgactggctctgtgtctgggagatatgtggagtatgaatggggacccaccattcatagagaaagcaagtggccctgcagcccgaggctatgatttccggtgcacacttggagagggtgcctttacacccaagtaccaggctcagaggtaggctatgccactatcaggtctgcagtggcacgtggcatctgggccagcctccttggttggccatgacctggctggtggcctgggcctgtgcccagtgttcccagggtagttctagggtgctgcgggggagctgttttggctagtggaggtgctcttgggtcctgcagtacagcacagcctgggcgttttgcctgagcccagtgacctgcctgccttgcccttcccaggatcggtggtagggaagaccaggggtgcccggcataggtgatgagtcagtatttcacatggatatcatcacatgtgtccatggagggggagcaaacatgagctggcacccgccacccccttctccacatgaagggtggtcacacgttctccatctgttcctccagatggtgagacttctgtcggcctcgcttttcctgggccagtgtccatcagccaggtgctcagccccagtgcactgaagctccagcccttcctggcgccctggactgagctgtggtgagcacatccgggttccactggatgcgtatgtgggaaggggggtgccctgggttgggtcaatgatgagaaccgtatattgtcctgaagagcggtgatgacttaaaaataatgctcaataggattacgctgaggcccaccctaagtgagaaatttggtaggggatgttgggatcccgagatttctggcagggccactgtagtttgggttggaggcctggaggccctgaagggcatctggaggtggctcaactctttctctgtgctcctccgtgtagcagcccaggctccctgtgcgggcttggtgttgttgcttctgttccttgggggcagggcatgtgggtggctcctccctgagccccagttctccccttgtgtctttcagtgagctcttctgccaagggggtcctcctggcattgaccaacataggtgagtggatcctgctgctgtcatggtccgtgagccaggctgcttggggttgccaagggtcagacttcgggccatgaaggtttcctcagggagctgaccctaattcccatgagggaggcatggtttctccaggaggtcagcttccttgggaatctgtgacttgacagggttgagtcgcagaggttcaaggatctcatcccacctgtaaccctgaatggggcccaccctgttgagcaactggccatcccagtggcacatattgagcctggtggggggtggtgcagtttcaatggggttgggtcaaggctgagatatggctggtgcctggtggtgctgcagtccagccctgggatccgtgggctttgggctgagggggaagcctgcatcctattgggggtaacctgttctccaaatttgagtgattggtgggaaggcctactgtctgaggtcctgtactgggaatttgccacctcagcttcttctgagccatagggtcacctccagggtcatggggtcttgggaccaaggaaaagagaagataccatgtggcccttgattggccgagtgtagcccagctctgtgtttgggaggtgtgtgggatgtggatagggtcccacctgtgaaagcatgtggcccgggtccccaggctgtgattttcagtgcacacttggagagggcaccttggtgggtgcccaccatgtccaggggcaggctatgtgactgccaggtttgcaatggtgtgtagcttccttctaccctccttggttggctgggagtgggttggttgctgtggcccatgcccagtggtcacagggcaggcagactgccttgttttttcagggtatgctgacagggagggccatgggtgccctgcattcatgatgagtcagtgttccacatggaacccattatgtgggtatctcttattccttctacatatggagatgtgaggccctatttggacacatgtcctcctcctgtacccccaggtggtgagcctggaggaagacttgcattgggcccaatggccctggaccagtgtctgtcagccaggttcccagcccccgatgcgctgaagctcaagcctttcctggcaccctggtctcctgcactgagctgtggtgagcatatcctggtcctgctggatgcatgcgtggggaggggttgtcctaggttgggtcaatgatgagaaccttataatgttctgaagagaggtgatgacttaaaaatcatgctcaataggattacgctgaggcccagcctaggtgataattttggaagaggacactgggtcccgaagccccctgcagggcccctgtgttttgggctggagaccttgaggccctgaaaggcatctggtggacccaactctatctctgccctcctcaatgaggcagcccagcctccctgtactggcttggtatccgggctccgggtctctggggacggggcacattggaggctccttcctgagcccccattctctcctgtgtctttcagtgagctcttcggcccaagtggtccccccggccttaagcggcataggtgagtgcatcctgttttgatcatgggccatgagtgttgcagcatggggtccccgacattcagccttcaggccactaggcttttctcaggctgcaaacctactctccaccagggagcgatggtttctcaggagggccacttccttcggaatctgacccatggagcacgcctttcctgaagccctgcagaggtgacagtggtccaggcaggaaggggttccttcagccccaatgtagggcagctcccagctgaaacttggtggacagatcaggcctggagtactgcccagggcatatggggaggagggacacagggaatgtggcgcaccctagaggtcacactggagttctgtctccctggagaggggtgagtgattgggcccacatggtcaggctgtggagattcaaggaatgctccacaccgatggccttgaatgggaccaaccctattgagcaatgggccatcttagtggcacctccttgaacctggtgggggatggcactctttcaatgaggttgggtcaaggctgagctctggctggcacccagtggtgctgcaggctggccctgggacttgtgggctttgggccaagggggaagcttccatcctgctgggaacaacccttccttgaggtccagtggcaggaaggggggcttcctgaggtggaaagccctactgttgagggacctgtcctgggaatttgctgtccaagctgcttctgagccatggggtcacctctagggtggtggggtcttgagcccaaggaaaggagaggacaccatgtggttcctgattggctgagtgtggcccggctctgtgtgtcggagttgtgcagggtatggattgggacccaccagtgaaagcacatggccctggaggtccgcaggcttggagagggcaccagatctggcacccaccatgcccaggtacaggctctgggactgccaggttgcagcagctcttggcatctgggccaccctccttggttggccaggagctagttggtggccctgtcccatgccccatgtccctggtatggccatggggtcatgcagaggaaccggccctggcatggaggtgctcttgcaacatgcagtagggtgcatcccaggcatcttgcctgagccaggtgacccgcttgccctgtctctccagggtttggtggcagggagggccaagggtgccccgtgtccatgttgggtcagtgttcagcatggatcccattgcatgagtatcacttagctccttcccagcattgatatgtgaggccctggttggacacttgtcctcctgtccctccagatggtgagcctggaggaagactcgtgttgggcccacaggccccaggccagtgtacgtcagccaggtgctcagcccatggtgtgctgaagctctgacccttcctgactccgtggtctcctgcactgagctgtgatgaccatatccaggtcctgctagatgcatgcgtggggaggcagggtgccctgggttgggtcaatgatgagaaccttgtattatcttgaagagaggtgatgacttaaaaatcatgctcaataggattacactgaggcccagactaggtgagaattttggaagaggatgctgggatcctgaggtccccaacagggccaccatattttgggctggagacctggaggtcctgaagggcatctgtagggggcccaaccctgtctctgcactcctccctgaggcagcccatgctccctattcgggatcggtattggtgctccacttccctgggggcaggacacgtgggtggctactccctgagcccccgttctccccttgtgtctttcagtgagctcttccaaacaggtgggctccctagcattgaccgacagaggtgagtggatccttctgggattatgggccatgagccaggccatgtggggtccccaaggttcagcctttggatcccgagagttgtctcagacagccaacctgatttgccacctgggagagatggcttctccaggagggtgggtttttgggaattggacccaaggaggacgccttccctgaagccctactgggatgatggtggtccaggcaggaaagggttccttcacccagctgagactcggtgttccagtaaggcctcaaggacctccctgccacatgaaggggcccagaggggctacgaggcaccacaggagtgacaggggagctcttggtccccagagtgtggtacatgaatgggcccatggggttgagtcgcagagattcaaggatgctgcaccacccatggccctgaatgtgactctcctcctggagcctggtggggggtggcgttttttccctggggttcggtcaaggtggaactctggtcagtacctggtggtaatgtaggtcagccctgggactcgtgggctttgggccaacggacagcccacatccctctgggggtcaccctttctctgaggtcgagtgtcgtgaagggtggcctcctgaggtgggaaagcccaatgtcgaaggacctgtactgagaatttgttgcttaggctgcttaggatccatgttgtcacctccagggtggtgggatcttgggaccaagcaaaggagagggcaccatgtggctgccagtggctatgtgtgactggctctgtgtctgagaggtgtgtggatggggacccaccagagaaagcaagtggccctgcagccccaagatgtgatttctggtgcaggcttggagagggtgcctatacctgctcccaccacgcccaggggcaggctttgccactgcaggtctgcagtgccacgtggcatctgggccagccaccttggttggccgtgacctggctggtagcccgtgcctgtgcccagtgttcccagggctgccctggggtcttgctggggagctgttttgggcagtggaaatgctcttggggcctgcagtatggcacagcctgggggcattttgcctgagcccagtgacctgcctgccttgtccttcctgggttcggtggcagggagggccaagggtgccctacattggtgacaagtcaatattttgcatggatctcatcacgtgtccatggaggtgggggcaagcgtgagctggcacgcaccccattctccccatggagggtggtcacatgtactccacctgtgtccctagatggtgagactggaggaagacttccattgggcccacttttcatgggacagtgtccatcagccaggtgctcagcccccagtgcattgaagacccagcccatcctggcaccctagtctcctgcactgagctgtgctgagcacatctggcttccactggaagttttttttgcgaggaggggcttccctgggttgggtcaatgatgagaaccttatattgtcctgaagagaggtgatgacttaaaaatcatgctcaataggattacgctgaggcccaacctaggggagaattttggaagaggattatgggatcccaaggtccccaacaaggccactgcattttgggctagagacctggaggctcttaagagcatctggaggtggcccaacctgtctctgctccttcgtgatgcagcccaggctccctgtgtgggattggtgttggtgctccatttccctggcatggggaatgttggtggctccttcctgagctcctgttctcgccttgtgtctttcagtgagctctgccacccaggacatccccctggcattgactggcataggtgagtggatcctgctggggtcattggtcatgagccagaccatgtggagtccccaaggttcagcctttgggccacaaaggttttctcagggagcagacctgaatttcacccgggagggaagggttgtcctggagggtggcttttttgggaacctaacccaaggaggacccattccctgaagccctgcaggggtgatggtggtccaggcaggaaagggttccttctgctcaatgcagggcaacacccagctaagatgcggtgatctggtaaggcctccagaactgcccaggccatatgaggggggccagaggggctacagggtgccacaggggtaacaggggagctcttggtccttggagtgtggtgcctgaatggacccatgggattgggttgtgtagattcaaggatcacaccctacccacagccctgaatgtgactcttcctcttgagcaatgggccatctcagtgggtctcctggagcctggtcggggggtggtgttgtttccctggggttgggtctaggtggagctctggctggtacctggtggtgctgcagatcatccctgggacttttgggctttgggccaagggacagcccgcatcccgcttggggtcagcctatctccatggtcaagtgtcatgaagggtggccccctgaggtgagaaggcctactgtcaagggatctgtcccaggattttgttgctcaggctgcttctgagccatggcgtcacatccacagtggcagggtcttgggcctgagcatagcagagggcaccatgtcgttctggattggccaagtatggcctagctctgtgtgcgggaggtgtgcagagcatggacggtgacccaccagagaaagcacgtggccctgcatcccaggctgtgatttccaatgcacacttggagagggtgtcttgatttgtgcccaccatgcccaggggcagactacgtgatagccaggtctgcagcctgagccacgtgtggcatctgggccagccaccttggttggccgtgagctggctggtggccttggcctgtggccagtggtgccagggcggccctggggtcgtgtgggggagccatgttgggcagtggaggtgctgtcagggcctgcagtacagtgcagcctgggcatttggcctgagcccagtcacccgcctgctctgttcctccagggtttggtggcggggagggccaagggtgccccgcatccgtgacgactcagtaattcacatggatcacatcacatttttccttggagggcaagcaaatgtgagcccccaccccccactgccttccccacatggaggtcattttggacacatatcctcctctagtccccctagatggtgagactggaggaagacttgcattatgcccgctgttcctgggccagtgtcagtcagccaggttcccagctccaagtgtgctgaagctcaggcccttcctggcaccctcagctcctgcactgagctggggttacatccgggttccactgtttgcatgcaaagggaggggtgtaccctgggttgggctgatgatgagaaccttatattgtcctgaaaaaaggtgatgacttaaacatcatgcttaatagtattatgctgaagcccaagctaggtgagaattttggaagatgaggctgggatcctgagatccccggcagggccacacttattttgggctggaaaatttcaagacctgaagcgcatcttgtgggggggcaatcctgtctctgtgcttctccgtgaggcagcccaggctcccgttgcgggcttggtgcccatgcttcgcttccccaggggtggggcaatttggcatcctcttcctgagccctccttcttccctgtgtctttcagtaagttcttcttcccaggtggggcccctggctttgagcggcatgggtcagagcatcctgtcagggtcatgggccatgagtgaggcagcgtcggttcgtcgagagtcagcctttgggccactggggttttctcagggagccaacaggttacccatcaggagggatggttcctccaggagggtcgcttccttgggaatctgactctaggagcatgcatttcctgaagctctgcagggtgatggtggtccaggcgggaaagggttccttcagcccaacataatgcagttcccagctgaaactcggtggcagatcaggctcaaggactgctcgggccacatgatgggggccacaggggatttggggtgccctgggggtcatgctggagttcttgccccaagaggggtgggtgatagggcccatgcgatcaggctgcggagattcaaggaatgctacccacccatggccctgaatgggactgaccctgttgaacaatgggccattttaatggtgcctccttgagcctggtaggtagtggcattgtttcaatgcggttgggtcaaggctgaactctggctggtgcccggtggtgcccacatgggctttgggccaagggggcagctcatatccctctggggtcaccccttccttgagattcagtggcaggaagggtgttctcctgaggtggaagacctgctgtcaagggacttgtcctgggaatttgccacctaggctgcttctgagccatggagtcacctccagggtggcagggtcttgagcccagggaaaggagacaccatatggtttctgactgggagtgtggcctggatctgtgtgtgggaggtgtgcagggcatggatggggacccatcagagaaagcatgtggccctgtaggcccccaggctttgatttctggtgtgtgcttgcagtgggcaccagggcagccctaccatgcccaggggcaggctctgtgactgccaggtctggagtggctcctggcatgaagaccacgctccttggttggccatgagcaggttggttgccctagcctgtgccctgtggtcctggtgcagccctggggttgtttggggagacagcaccggcagtggaggtgctctcaaggcctgcagtagggcgcagcccaggtgtcttgcccgagcccagtaacccacttgccctgtctttccagggtttggtggcagggagggccaactgtgccccgcatctgaaatggatcagtgttctgcatggatcccatcacaagggtatcccctacccaacttctgtgcattgttatgtgaggccctggttggacacatgtcctcctcctgtccctgcagatgctgaccctggaggaagacttgtgtcgggcccccaggccccaggccagtgtccatcagccaggtgctcagctcccagtgcgctgaagctcagacccttcctggcaccctggtgtccagcactgagctgtggtgagcccatccgggtcctgctggatgcatgaatggggaggggcctgcccagagttgggtcaatgatgagaaccttacattgtcctgaagagagatgatgacttaaaaatcatgctcaataggattacgctgaggcccagcctaggtgagaattttggaagaggacggctgggatcctgagatcccagtcagggtcactgtattttgggctggagacctggaggccctaaagggcatctggaagggggcccagccctgtctctgtgctcctccgtgaggcaacccacactccctgtgtgggcttggtgttggtgctctggttccccagggacagggcgggttggtggctccttcctgagcccctattctccctttgtgtctttcagtgagcttttccacccaggtgggccccctggcattgaccgacataggtgagtgcatcctactgggataatgggccactagatagtctgtgtggggttgctgaggtttagcctttgggccacaagggttttctcaggcagccaacctgaattcccacctgggagagatgggttctccaggaggcagcttttttgggaatcagacccaaggaggacaccttccctgaagccctgcaggggtgatggtggtccaggcaggaaagggttccttctgcccaactcgaggcccaatgcagggcagtgcccccctgagaattggtgctctggtaagggctcgaggactgcccaggccacatgaaggaggtcagaggggctatggggaaccatgggggcaacaggggagctcttggcccccggagtgtggtacatgaatgggcccatggggttgggtcacggagattcaaggattgcatctcacccacggccctgaatgcgaccatccctgttgagcaatgggccatcccagtgggtctcctggggcctggtgtggggtggcattgttttcctggggttgggtcaaggcggagctctgactggtacctgatggtgctgcaggtcagccctgggactcgtgggctttgggccaagaggcagcccgcatcctgctgggggtcgccttttctccgaggttgagtgtcgtgaaggatggcctcctgaggtgggaaggcccactgtcaaggcacctagactaggaattgttcctcaggctgcttgggagccatggtgtcacctccagggtggcgggatcttgggactaagcaaaggaggggacactacgtggctcccaattggccatgtgtggctggctctgtgcctgggaagtgtgtggagtatggatggggacccaccagagaaagcaagtggccctgcagtcccagaatgtaatttctggtgtaggcttggagagggtgcctatatgcatgcccaccatgcccaggagcaggctatgccacttcagggtctgcaggggcacctggcatatgggccagcctccttcgttggctgtgacctgggtggtggcctgtgcttgtgcccagtggtcccagggtagccctggggtcgtgcaggggagccgttttgggcagtggatgtgctctcgggtcctacagtacagcacagcctgggtgttttgctcaagcccagtgacctgcctgccttgtccttcccgggttcagtggcagggagagccaagggtgccccacattggtgacgagtcagtattttgcatggatctcatcacacgtgaccatgaaggacgagcaagcatgagctggcaccagccacccccttctccgcgtggagggtggtcacatgtcctccacctgtgcccccagatgatgagactggaggaagacttccattgggcccacttttcctgggccagtgtccatcagccaggtgctcagcccctggtgtactgaagctccagcctttcctggtgccccagtcttctgcactaagctgtggtgagcacatccgggttccactggatttttgtgcggggagggggttttctacgttgggtcaatgatgagaatcttatattgtcctgaagagaggtgatgacttaaaaatcatgctcaataggattacgctgaggcccagcctaggtgagaattttggaaggggattatgggatgctgaggtcctcagcaaggccactgtattttgggctagagacctggaggctctgaagggcatctggaggtggcccaacctgtctctgcgctcctccatgaggcagcccaggctccctgtgcagggttggtgttggtgctccggttctctggtgtggggaatgttggtggctccttcctgagccctcattctcaccttgtgtctttcagtgagctcttccacccaggagggccccttggcattgactggcataggtgagtggatcctgctggggtcattggtcatgagctagaccgtgtggggtccccgaggttcagccttcagtccacaagggttttctcggggagcagacctgaatttccacctgggagggaagggttctcctggagggtagtttttttgggaatctagcccaaggaggatgccttccctgaagccctgcaagggtgatggtggtccaggcaggagagggttccttctgcccaatgcaggacagctcccagttgagactgagtgctccagtaaggtctccaggactgcccaggccacatgaaggaggccagaggggctatggagcaccactggggtaataggggagctcttggtccctggagtgtggtgtgtgaatggggtttggtcacggagattcaaggatcacaccccacccatggccctgaaagtgaccatccctattgagcaatgggccatcccaccgggtctcctggagtctggtgcagggcgacgttttttccctggggttgggtcaaggtggagctctggtacctggtcatgctgcaggtcagccttgggactcgtaagctttgggccaagggacagctggcatcctgctggggggtcaccctatctctggggtcgaatgtcacgaagggtggcctcctgaggtgggaaggcccattgtcgagggacctgtcccaggattttgttgctcaggcttcttctgagccatggtatcacctccagggtgacagggtcttgggcccaagcaaaggagagggcaccatgtcgctctgagttggccaagtgtagcctagctctgtggtgggaggtgtgcagagcatggctggtgacccaccagagaaagcacgtggccctgcagccccaagctgtgatttccagtacatgcttagagagggcatcttgatgtgagcccaccacacccaggggcagactacgtgactgccaggactgcagtggcacatggcatctgggccagccttcttggttggccatgacctagctaatggccctggcccgtgccttggggtcatgcaggggagccctcttggacagtggaggtcctctcagggcctgcaatatggcgcaggctgggcatttggcccgagcccagtgacctacctgccctgtccttccagggttgggtggcagggagggacaagggtgccccatgtcagtgacaagtcagtatttcacatggatctcatcacatgtgcccatggaggacgagcaagcgggaactggcacccgctacttccttccctacatagaggccattttggacacatgttctccacctctctcccagatggtgagacgggaggaagacttgtgttgggcctgctgttcctgggccagtggccatcagcctgttgcccagcccctgaggcgctgaagctcaggcccttcctcacaccctggtctcctgcactagctgtggtgagcacatccgggtcccgctggatgcatgcaagaggtggttggtctcgtgggttgggtcgatgatgagaaccttatattttcctgaagagaggtgatgacttaaaaatcatgctcaataggattacgctgaggcccagcctaggtgagaactttggaagaggatgctgggatcctgagatcccaggcagggccactatattttggggtggagaactcaaggccctgaagggcctctcagggggtgccaccctgtctctgtgcccctctgtaaggcagcccaggctccctgtgtgggcttggtgtctggtctctggttccccgggggcggggcatgttggtggctccttcctgagcccgctttctcccccatgtctttcagtgagctcttctgcccaggtggggcccatggccttacacgacataggtgagtgtatcctgttggggtcatgggccatgaatgaggcagcatggggccatttagggtcagccttcgggccactagagttttctcagggtgcgaccggggagggaacatttctccagtagggtcacttccttggaaatctgatcctaggagcacgcatttcttgaagtcctgcaaaggtgatggtggtccagacaggagagggttccttcagcccaacatagcgcagctcccagctgaaactcggaaggctggtcaggcctcgaggactgcccaggccacatgaggtgggcaacaggggctctggggcaacctgggggtcacgctggagttctccctctctggacagtggtgggtgattgggcccacgcagacaggccccggagattcaggactgcaccccatccatggccctgaatgggaccgaccctgttgagtaatgggccatctcagtggtacctccttgagcctggtagggggagacactgtttcaatgcagttgaatcaaggctgagctctggctggtgcctggtggtgctgcaggcagccctgagggttgtggtctttgggacaaggggtagctcacatcccactggggtcaccccttccttgagattcagtggccaaaagggtgtcctcctgagatgggaagtcctgctgtcgagggacctgttctgggaatttgccacacaggctgcttctgagccacggggtcacctccagggtggtagggtcttgatcccaaggaaaggagaggacaccatgtggtttcttattgaccaagtgtggcctggctttgtgtgtgggaggtgtaacgcaagatgaggacccaccagagaaagcacgtggctctggaggcccccagggtttgatttccggtgtgtgcttggagagggcaccaggttgggcacccaccacgcccagggcaggctctgcgactgccaggtctgcattggctcctggtgtcagggccagactcattggttggccatgagcaggttggttgccctgacccatccccatggtcccagtgtggccctggggtttttcaggggagccggctcaagcagtggaggtgcgcttagggcctgcagtagggaacagcccaggcatcttgcctgagcccagtaacctgcttgccttgtctttccagggtttggtggccgggagggccaatgatgctctgcgtctgtgttggatcagtgttcttcatggattccatcacgtgggtatcccctacccaccttccccttgctcatacgtgaggccctggttggacacatgtccttctcctgtcaccccagatggtgagccttgaggaagacttgtattgggcccccacaggccccaggccagtgtccgtcagcctggtgctcacccccagtgtgctgaagctcagaccctccgtggcaccctggtctcctgcactgagctgtggtgagcacattcaggttccgctggatgcatgcatggagaggggcttgcccttggttgggtcagtgatgagaaccttctattgtcctgaagagaggtgatgacttaaaaatcatgctcaataggattacgctgaggcccaacctaggtgagtatgttggaagaagacactgggatcccgagatctccaacagggcactgtattttgggttggagacctggaggccctgaagggcatctggaaggaggcccaaccctgtctctgtgctcttccttggggcagcccaggctacctctgcgggcttggtgttggtgctccagttccctggggccagggaacattgctggctccttcctgagcccccattcctcccttgtgtctttcagtgaacttttcctcccagatgggccccctggcattgactggcataggtgagtgcatcctgctggggtcatgggccatcagccaggccatgtggggtcgctgaggttcagcctttgggccacaagggttttctcaggcagccgacctgaattcccacctgggagagacgggttctccaggagggcagcttttttgggaattagacccaaggaggatacctgaagccctgcaggggtgacggtggtccaggcaggaaagggttccatcagcccgggcaaggcagctcctagctgacactcggtgttcgggtcagtccttgaggactgcctgaacacgagggggtccagaggggctctgggtgccacgggggtcacatgggagctctcagtccctagagtgtggtgtgtgaatggacccttagggttaggtcttggagataaaaggattgtgctccacccatggcctggaatgtgaccgtccctgttgagtaatgggccattccagtgggtctcctggagcctgatgggaggtggcgttgtcttcctggggttgagtcaaggcagagctctggcccatacctggttgtgctgcaggtcagccctgggactcgtgggctttgggccgaggggcagcccgcatcccgctggagattaccctatctctgagatcaagcgtcatagagggtgacctcctgagaagggaaggcccactgtcgagggacctttcctaggattttgttgctcaggctgcttggaatccattgtgtcacctccagggtgatggaggtttgggcccaagcaatggagagggcaccatgtgtctcctgattgttcaagtgtgtcggagatatgcggggtatggatggtggcccaccaaagaaagcatgtgactcaagctgtgatttccagtgcatgcttagagagggcgcctgtatgcgtgccccacatgcccggggtaggctatgcaactgccaggtctgcagtggcgcatggcatcttggccagccttcttggttggccatgacctggctaatgaccctggcctgtgcccagtgatcccagggaagccctggtgtcatgcagcggaggcctcttgggcagtggaggtgctcttaaagcctgcagcatggcacggcctgggcatttatcccaagcccaatgaactacctgccctgtccttccagggttcggtggcaggcagggccaagggtgccccgtgttggtgacaagtcagtatttcacatggctctcatcacatgtgtccgtggaagataagcaagtgtgaactggcacgtcctacctccttccctacatggaggtaactttggacacatgttctccacctctctcccagatggtgagaccggaggaagacttgcattgggcctgttgttcctgggccagtggccatcagcctgttgcccagcccctgatgcgctgaagctcgggcccttcctcgcgccctggtctcctgcactgagctgtggtgagcacatccgggtcctgctggatgcatgcacgaggtgattgatcccctcagttgggtcgatgatgagaaccttatattgttctgaagagaggtgatgacttaaaaatcatgctcaataggattacgctgaggcccagcctaggtgagaattttggaagaggatgctgggatcccgagatccctggcagggccactatattttgggctggagaacttgaggccctgaagggcatctcaggggggtccaaccctatctctgtgcccctccatgaggcagcccaggctccctgtgtgggcatggtgtccgggctctggttccctgggggtggggcatgttggtggctccttcctgagcctgctttctcccccatgtctttcagtgagctcttctgcccaggtgaggcccatggccttaagcggcataggtgagtgtatcctgttggggtcatgggccatgaatgaggcagcatggggccatttatggtcagccttcgggccactagggttttctcagggtgcgaccggggagggaatgtttctccagtagggtcacttccttgggaatctgaccctaggagcatgcatttcgtgaagccctgcaaaggtgatggtggtcccggcaggaaagggttccttcagcccaacatagagcagctcccagctgaaactcagcgggctggtcaggcctcgaggactgcccaggccacatgaggtgggcaacaggggctctggggcaacctgggggtcacgctggagttctccctctctggacagtggtgggtgattgggcccacgcagacaggccccggagattcaggactgcaccccatccatggccctgaatgggaccgaccctgttgagtaatgggccatctcagtggtacctccttgagcctggtagggggaggcactgtttcaatgcagttgaatcaaggctgagctctggctggtgcctggtggtgctgcaggcagccctgggggttgtggtctttgggacaaggggccagctcacgtccctctggggtctccccttccttgagattcagtggccgaaaggatgtcctcctgaggtgggaagtcctgctgtcgagggacctgttctgggaatttgccacacaggctgcttctgagccacggggtcacctccagggtggtagggtcttgatcccaaggaaaggagaggacaccatgtggtttcttattgaccaagtgggacctggctctgtgtgtgggaggtgtagggcaagatgaggacccaccagagaaagtacgtggccctggaggcccccaggctttgatttctggtgtgtgcttggagagggcaccagggtgggcacccaccatgcccagggtaggctctgcaatggccaggtctgcattggctcctggtgttagggccagactcgctggttggccatgagcaggttggttgccctcacccgtccccatagtcccagtgcggccctggggttgttcgggggagccggcccgagcagtggaggtgctgttggggcctgcagtagggcatcttgccccagcccagtgacccaccttccctgtctttccaaggtttggtggccagcagggccaatggtgccctgtgtctgtgatgagtcagtgttcttcatggattccatcacatgggtatcccctacccacattcccctcgctcatatgtgaggccctggttggacacatgtccttctcctgtcaccaccccagatggtgagcctggaggaagacttgtattgggaccctacaggccccaggccagtgtctgtcagcctggtgctcatcccccagtgcgctgaagctcagaccctccctggcaccctggtctcctgcactgagctgtggtgagcacatccaggttctgctggatgcatgcatggggagggccttgattgggtcaatgatgagaaccttatattgtcctgaagagaggtgatgacttaaaaatcatgctcaataggattacgctgaggcccaggctaggtgagaattttggaagggagcgctgggatgccaagatccccggcagggccactagccactgtattttatactgaagaacttgaggccccaaagggcatctcaggggtcctagccctgtctctgtgcccctccgtgaggcagcccaggctccctgtgttgacttggtgtccaggctctgcttcctggggatagggcatgttggcagctccttcctgagcccacgttctcccctgtgtcttctattgagctcttctgcccaggtggggcccatgcccttaagcagtataggtgagtgtatcctgttgcggtcatgggccataaatgaggcagtgtggggtcatcgagggtcagccttcaggccactagcgttttctccaggtgcccccggggagggaacattgctccagtagggtcacttccttgggaatctgaccctaggagcacgcatttcctgaagccctgcaaaggtgatggtggtccaggcaggaaagcgttccttcagcccaacatacagcagctcccagctgaaatttggtgggctcgtcaggccttgaggactgcccaggcttcatgatgggggacacagaagctctggggctccctggggctcacactggagttctccctccctgaagagtggtgggtgattgggcccacagtcaggtggcggagattcaaggaatgcccccacccctggccctgaatgggaccaactctgttgagtaatgggccatcttaatggcgcctccttgagcctgttgggggtgggttgctgtttcaataaggttgagtcaaagctgagctctgactgggcgcctggtggtactgcaggccggcccttccttgggccaagggggcagcttgtgtcccgctggtgtcatcccttccttgaggtccagtggtggaaagggtagcctcctatggtgggaaggcctgctgctcagggacctgtcctggaaatttgccatccaggctgcttctgagccatggggtcacctccagggtagtggggtcttgagtccaagaaaaggagaggacaccatgtggttcctgattggccaaggctccttggttggccaggagcaggttggttgccctggcccatgccccatggtcctggtgtggccctggggttgttcaggggagctggcccaggccgtgaaggtgctctcagggcctgcagtagagtgcagcccaggcatcttgccccagcccagtgacccacttaccctgtctttctaaggtttggtggccaggagggccaatggtgccttgtgcccatgttgggtcagtgttctgcatggatccccatcatatgggtattccctacccaccctccctgcactgatatgtgaggccctggttggacacatgtcctcctcctgtcccttcagatggtgagtctggaggaagacttgtgttggacccccacaggccccaggccagtgtccatcagcctggtgctaatcccccagtgctctggagtttggaccctccctggcaccctggtctcctgcactgagctgtggtgagcacatggatgcatgcttggggtgggggttgcccttggttgggtcaatgatgagaaccttatattgtcctgaagagcggtgatgacttaaaaatcatgctcaataggattacgctgaggcccagcctaggtgagaattttggaagaggacactgggctccccggaatggccactgtattttgcgctggacacctgcaggccctgagggccatctggaaggtccaaccctgtctctctgctcctccaggaggcagcccaggctccctgagggggcttggtgtcgggctcctgttccccggggttggggccggttggtggctccttcctgagcccccattctccccttgtgtctttcagtgagctcttccacccgggcgggcccccggcattgactggcataggtgagtggatcctgcttgtgtcatgggccatgaaccggtccatgtggggtcaccaagggtcagcctttgggataagagggtttcctcagggaaccaacctgaattcccatcagagaggtatgggttctccaggagggccgcttttttgggaattggacccaaggaggataacttccctgatctctgcaggagtgacggtggtccaggcaggaaagagttccttcagccccacccagcgcagcgttctgttgagactcagtgctcaggtcaggcctggaggatttctcaggccatctgagggggttagaggggagctctttgtccatggagtgtggtgcttgaatctgcccttggggttgggtcatggagattcaaggatggtgcctcacccatggccctgattggaattgtccattgagaaatacgccattccagtgggcctcctggagcctgatggggggtggtgtttttttccctggggttgagtcgttgatggggactcaccatagaaagcatgtggctctggaggcccccaggctttgatttccagtgcctgcttggagtgggcaccagggccggcacccaccatgcccaagggcaggctctgcgactgccaggtctgcattggctcctggcatcaggaccagcctccttggttggccatcagctgcttggttgccctggcccatgccccgtggtcctggtgtggccctggggtggttgagtggagctggcccaggcagtggaggtgctcttggggcctgcagtagggtgcagcccaggcatcttgcctgatcccagtgacctgcttgccctgtctttccaggattttgtggcagggagggccaagggtgccctgcatccgtgatgggtcagtgttccacgtggatcccattacgtgggtccatagaggatgagcatgcgtgagccagcgcccccttcccacttcttctcatggagctgtgaggccctgtgtggacagctgtcttcctcttgtcccaccaggtggtgagcccggaggaggacttgcgttcggcccgctggtcccagtgtgtccggaattggtgggttcttggtctcactgacttcaagaatgaagctgcggaccctcgctgtgagtgttacagctcttaaggtggcatgtctggagtttgttccttctgatgttcggatgtgttcggagtttcttccttctggtgggtttgtggtctcgctggcttcaggagtgaagctgcagaccttcgcggtgagtgttacagctcataaaagcagtgtggacccaaagagtgagcagtagcaagatttattgcaaagagtgaaagaacaaagcttccacagtttggaaggggacccaagtgggttgccactgctggctggggcagcctgcttttattctcttatctggccccacacacatcctgctgattggtagagctgagtgatctgttttgacagggcgctgattggtgcgtttacaatccctgagctagacacaaaggttctccatgtccccactagattagctagatacagagtgttgacacaaaggttctccaagtccccaccggagtagctagatacagagtgtctattggtgcattcacaaaccctgagcttgacacagagtgctgattggtgtgtttgcaaaccttgagccagatacagagcgcggattggtgtatttacagtccctgagctagacataaaggttctccaaggccccaccagagaagctagatacagagtgtcgattggtgcattcacaaaccctgagctagacacagggtgctgattggtgtgtttacaaaccttgagctagatacagagtgccgattggtgtatttacaatccctgagctagacataaaggttctccaaggccccaccagactcaggagcccagctgtcttcacccagtggatcccgcactggggctgcaggtggagctgcctgccagtcctgcgccatgcgcccgcactcctcagcccttgtgtggttgatgggactgggcaccgtggagcagggggcggcactcgtcggggaggctctggctgcacaggagcccatggagggggtgggaggctcaggcatggcaggctgcaggtcccgagccctgccccgagggaaggcagctaaggcccggtgagaaatcaagtgcagcgccggtgggctggcactgctgggggacccagtataccctctgcagctgctggcccgggtgctaagcccctcattgcctggggccggcagggccggcccgctgctccgagtgtggggcccgccaagcccacacccacccggaactccagctggcccacaagcgccccgcgcagccccggttcccacttgcgcctctccctccacacctccctgcaagctgagggagcaggctccggccttggccagcccagaaaggggctcccacagtgcagcagtgggctgaagggctcctcaagtgccaccaaagtgggagcccaggcagaggaggcgccaagagcaagtgagggctgtgaggactgccagcacgctgtcacctttcatcaggacagtgtctgtcagccaggtgcacagcccttggtgtgctggagcttgggcttttcttggcagcctggtgtcctgcattgagcggtggtgagcttgtccaggtcccgctggttacctgcacggtgacgggggtgccctcggttaggttgatgatgagaaccttattttgtctcgaggagaggttatgtgatgggtgttgtggcagctctttgtatttcattttcttcagtgctttttgtgtgttctctttgtattttttaccgctttgtgtattttactttcacaaaggcagcagcaaatgttttattggtggtggtatttttcatttttatttacttcctaatttgatagtgttttagttggaatacacagtctcattgtagttgttcagaaatatgctttttttctgtacattatatgattgtgcataatttttgccagttttttttttaattggggactacagttgaatgcactgcctatctcctactttactgaaacagattgtatggaaatctaaagacgagaaagtagacaagcacagcagaggacatttattttgatataaccacagaaattcattgatatgaccttatcatattgatggtttttcaaagaaatataaaatctttcgataatggattgtctcagttgcgtttctccttatatcatgttttgccttcaatacaaattgcaatgcttagaaatttaaagaacaccttctgaagtggaagatctagctaaagaattagattggtattggctgaaatgttgcagtaatcacatgatagggttaatacaacatgagttacatgctgagtgctctaaggtagaaagtaggtaacaaggaataacaggtaggtaatgtaagtcgggagaaggaaactctaggacagaatcattagaaaaggcttgacgtcaacagcagtgtatagaatttacagtgtatagaatttacagtgcatagaatttacaataagtgcctttttttgtgctcatcaatgctcttgcttatattgtaggaggagttagttcccttgaagaaatatcaatacaaatttccaaattgcaatgcagagggaaaaaagaatgagaaagtaaaaaatatccaagaactctggaagcatttcaaaaagtgtaaaaatgcatagtgacaatgtcaggagaataaggagataaaggaaaagaaatatttgacacaacaatgacttgagcactttctaaaattaatgatggacagaaacccacatgttcaggaagctcagagagcactctgcagaataaaaatattttacaaatcgacacctttgcatttcatatttaaagtccagaaaatcatagactaagggaaaagcttgataggagcaataggaagaaaaccatcttacctatggagcaacaagaataaaaattgctttgaaattatcttcagaatccatgaaagcaagaagtgagtcagggtgaaatatttagtgttaaaagtaagaaacaaaaaaaagtaccaactttgaattttctgctcatcaaaattattcttcaaaagtgaaggagaaatactctctgagaccaacaaaaattgctagaacttcttgccactacaccaaccttttaataaacgtaacaagaaattcttcagaaaggataaaaatgacataggtcggaaaatctaatttacataaagaaatgaaaagcattggagaagaaatatatgaagaaaaattaaatatatatgtattttttgattcataattgatctttcaacaatttgttcaaaataataacaagattatatttggtgattataggttatggaaaagtgaaatgaatgacagcaatattataagtcatccggttccaagatggccgaataggaacagctccagtttacagctcccagtgtgagtgacgcagaagacagttgatttctgcatttccaactgacgtaccaggttcatctcactggggcttgttggacagtgggtgcagcccatggagtgtaagccgaagcaggacgaggcatcacctcacctgggaagtgcaagaggtcagggaattccctttcctagccaagggaagcgtgacagatggtacctggaaaattgggacactcccaccctaatactgtgcttttccaactgtcttagcaaacggcacaccaggagattatatcccgtgcctggcttggagggtcccacatccacggagccttgctcactgctagcacagtagtctgagatcaaactgcaaggcagcagtgaggctgggggaggggcatccaccattgctgaggcttgagtaggtaaacaaagcggctgggaagctcgaactgggtggagcccaccacagctcaaggaggcctgcctgcttccgtagactccacctctaggggcagggcatagcagaacaaaaggcaacagaaacttctgcagacttaaacatccctgacagctttgaagagagtagtggttctcccagtacagagtttcagatctgagaacagacaggctgcctccttaaatgggtccctgacccccaagtagcctaactgagagacacctcccagtaggggctgactgatacctcatacagctgggtgcccctctgagacgaagcttccagaggaaggatcaggcagcaacatttgccgttctgcaatatttgctgttttgcagcctccactggtgatacccaagcaaacagggtctggagtggacttccagcaaactccaacagacctgcagctgaggatcctgactgttagaaggaaaactaacaaagagaaaggacatccacaccaaaaccccatctgtacgtcaccatcatcaaagaccaaaggtagatgaaaccacaaagatggggagaaaccagagcagaaaaactgaaaattctaaaaatcagagcacctcttctcctccaaaggaatgcagctccctgccagcaatggaacaaagctggacggagaatgactttgacgagctgacagaagtaggcttcagatgatcgctaataacaaacttctctgagctaaaggatgaggttcaaacccatcgcaaagaagctaaaaaccttgaaaaaagattagatgagtggctaactagaataaacagcatagagaagaccttaaatgacctgatggagctgaaaccatggcacaagaactacgtcacacatgcacaagcttcagtagccgatttgatcaagtggaagaaagggtatcagtgattcaagatcaaatgaatgaaatgaagcaagaagagaagtttagagaaaaaagagtaaaaagaaacaaagcctaaaaaagtaaaaaagtaaaacaaagtaaaaaagaaacatgggactatgtgaaaacaccaaatctacgtctgattggtgtacttgaaagtgatggggagaatggaaccaagttggaaaacactctgcaggatattatccaggagaactcccccaacctaggaaggcaagccaacattcaagttcagaaaatacagagaatgccacaaagatactccttgagaagcgtaactccaagacacataattgacagatacaccaaagttgaaatgaaggaaaaaatattaagggaagccagagagaaaggtcaagttacccacaaagggaagcccatcaaactaacagcaaatctctcagcagaaactctacaagccagaagagagtgggggccaatattcaacattcttaaatgaaagaattttcaacccagaatttcatatccagccaaactaagcttcataattgaaggagaaataaaatactttacagacaagtaaatgctgagagattttgtcaccaccaggcctgccctacaagagctcctgaaggaagcactaagcatggaaaggaacaaccagtaccagccactgcaaaaacatgccaaattgtaaagatcactgatgctaggaagaaactgcgtcaactaacgagcaaaataaccagctaacatcacaatgacaggatcacattcacacataacaatattaaccttaaatgtaaatgggctaaattctccaattaaaagacacagactggcaaattggataaagagtcaagacccatcagtgtgctgtattcaggagacccatctcatgtgcagagacacacataggctccaaataaagggatggaggaagatctaccaagcaaatggaaaacaaaaaaaagcaggggttgcaatcctagtctctgatgaaacagactttaaaccaacaaagatcaaaagagacaaagaaggccattatgtaatagtaaaggggtcaattcaacaacaagagctaactatcctaaatatacatgcacccaatacaggagcacccagattcataaagtaagtccttacagacctacaaagagacttagactcccacacaataataatgggagactttaacaccccactgtcaacattagacagatcaacaagacagaaagttaacaaggatatccaggaattgaactcagctctacaccaagcagacctaatagacatctacagaactctccaccccaaatcaacagaatgtacattcttctcagcaccacatcgcacttattccaaaattgaccacatagttggaagtaaagcactcctcagcgaatgtaaaagaatagaaattataacaaactgtctctcagaccacagtgcaatcaaactagaactcaggattaagaaactcactcaaaaccactcaactacatggaaactgaaaaatctgctcctgaatgactactgggtacataatgaaatgaaggcagaaataaagatgttctttgaaaccaatgagaacaaagacacaacataccagaatctctgggacatatttaaagcagtgtgtagagagaaatttatagcactaaatgcccacaagagaaagcaggaaagatctaaaattgacaccctaacatcacaattaaaagaactagggaagcaagagccaacattttcaaaagctagcagaaggcaggaaataactaagattggagcagaactgaaggagatagagacacaaaaaacccttcaaaaaatcaatgaatccaggaactggttttttgaaaagataaacaaaattgatagaccactagcaagactaataaagaagaaaagagagaagaatccaatagatgcaataaaaaatgataaaggggatatcaccaccaatcccacagaaatacaaactaccatcagagaatactataaacatctctatgcaaataaactagaaaatctagaagaaatggataaattcctggacacatacaccctcccaagactaaaccaggaagaaggtgaatctctgaatagaccaataacaggctctgaaattgaggcaataattaatagcctaccaaccaaagaaagtccaggaccagagggattcacagctgaattctgccagaggtacaaagaggagctggtaccatgccttccgaaactattccagtcagtagaaaaatagggaatcctctctaactcattttataaggccagcatcatcctgataccaaagcctggcagagacacaacaaaaaagagaattttagaccaatatccctgatgaacatcgatgcaaaaatcttcaataaaatactggcaaaccgaatccagcaacacatcaaaagctaatccaccacgatcaagttggcttcatccctgggatgcaaggctggttcaacatacacaaatcaataaacataatccatcatataaacagaaccaaagacaaaaaccacatgattatctcaacagatgcagaaaaggcctcgacaaaattcaacagcgcttcatgctaaaaactctcactaaactaggtgttgatgggacgtatctcaaaataataagagctgtttatgacaaacccacagccaatgtcatactgaatgggcaaaaactggaagcattccctttgaaaactggcacaagacaggaatgccctctctcaccgctcctattcaacttagtgttggaagttctggccagggcaatcaggcaggagaaagaaataaagggtattcaattaggaaaagaggaagtcaaattgtccctgtttgcagatgacatgattgtatatttagaaaaccccatcgtctcagcccaaaatctccttaagctgataagcaacttcagcaaagtctcagaatacaaaatcaatgtgcaaaaatcacaagcattcctatacaccaataacagacaaacagagagtgaaatcatgagtgaactcccattcacaattgcttcaaagaataaaatacctaggaatccaacttacaagggatgtgaaggacctcttcaaggagaactacaaaccactgctcaacgaaataaaagaggacacaaacaaatggaagaacattccatgctcatggataggaagaatcagtatcgtgaaaatggccatactgcccaaggtaatttatagattcaatgccatccccatcaagctaccaatgactttcttcacagaattggaaaaaactactttaaagttcatatggaaccgaaaaagagcctgcattgccaagtcaatcctaagccaaaaggacaaagctggaggcatcacactacctgacttcaaactatactacgaggctacagtaaccaaaacagcatggtactggtaccaaaacagagatatagaccagtggaacagaacagacccctcagaaataataccacatgtctacaaccatctgatctttgacaaacctgacaaaaataagaaatggggaaaggattccctatttaataaatggtgctgagaaaactggctagccatatgtaaaaagctgaaactggatcccttccttacaccttatacaaaaattaattcaagatggattaaagacctaaatgttagacctaaaaccataaaaaccctagaagaaaacctaggcaataccattcaggacataggcatgggcaaggacttcatgactaaaacaccaaaagcaatggcaacaaaagccaaaataaacaaatgggatctaattaaactaaagagtttctacacagcaaaagaaactaccatcagagtgaacaggcaacctacagaatgggagaaaatttttacaatctactcatctgacaaagggctgatatccagaatctagaaagaagttaaacaaatttacaagaaaaaaatcaaccccatcaacaagtgggcaaaggatatgaacagacacttctcaaaagaagacatttatgcagccaaagacacatgaaaaaatgctcatcatcactggccatcagagaaatgcgaatcaaagccacaatgagatcccatctcacaccagttagaatggcaatcattaaaaagtcaggaaacaacaggtgctggacaggatgtggagaaataggaatacttttacactgttggtgggatggtaaactagttccaccattgtagaagacagtgtggcaattcctcaaggatctagaactagaaataccatttgtcccagccatcccattactgggtacatacccaaaggattatatatcatgctactataaagacacatgcacatgtatgttcattgtggcactattcacagtagcaaagacttggaaccaactcaaatgtccatcaacgatagactggattaagaaaatgtggcacatatacaccatggaatactttgcagccataaaaatggatgagttcaagtcttttgtagggacatggatgaagctggaaaccatcattctcagcaaactatcgcaaggacaaaaaaccaaacactgcatgttctcactcataggtgggaattgaacggtgagaacacttggacacagggtggggaacatcacacacgagtggctgccgtggggtggggggaggggggagggatagcattaggagatatacctaatgtaaatgacgagttaacgggtgcagcacaccaacatggcacatgtatacatatgtaacaaacctgcacgttttgcacatgtaccctagaacttaaagtatgaaatatatatatatatatatatatatagtcatgagggagagaaaatactcttagttacttggactaacccacgaagtaggacagtataatttgaaaaaggacttggattagttgtaaacgtataatgtaatatccagcacagccactaaaaatgattttgaaaaaagcagtataatcaatatactaacagaagaaaaagcagaatcaggccagacatggtgactcatgtctgtaatcccagcactttgggatgctgaggcaggaagatcacttgagccccggagttcaagtccagcctggcaacgaataagagcccatctctaccctccaccccccccaaaaaaaaaaagaaaaagaaaaagaggccgggcacggtggcttactcctgtaatcccagcactttgggaggccgaggtgggtggatcgcgaggtcaggagatcaagaccatcctgcctaacacggtgaaaccccgtctctactaaaaatacaaaaaaaaaaaaaaaatagctgggcatggtggcgggcgcctgtagtcccagctactcaggaggctgaggcaggagaatggtgtgaacctgggaggcagagcttgcagtgagccgagattgcgccactgcactccagcctgggagacagccgcaagattctgtctcaaaaaaacaacaacaacaacaaaaaaaaacaaaaacaaaaacaaagaaaacgaaaagaaaaagcagaatctttttttttttttttttttttttttttttgagatggagttttggtcttgttgcccgggctggagtgcaatggcacgatttcagctcaccacaacctccacctcccaggttcaagcaattctccgggctcagcctcccgaatagctgggattacaggtgtgcaccaccatgcctggctaattttttgtatgaaaaagcagaatcttatacaatgctcaattaaaatcagggagaggagaaaaagagtagaataccaaaaaagtaacaaattacaaggttgatataaatagaaaacaatcacaaatactgagtatactaatctaagtatatcaataatcactttaaacatcgcttaaaagacagagattgtcagtggattaagaacaagacccaaatatatgttgtctatgagaaatgcactttaaatagctgggcatggtggtgtgcacctgcagtccttaggaggccaaggcggagtgtcacctgagcccaggattttgggactacagtgagctatgatcatgccactgcactctagcctggttgacagaataagacctcatctctatagaaaaaagtaagtaaatctactttataaaggcacaaatatattcacagtaaaggaatagagaaagatatcccatgctaacattaatcaaaataaagtgaaatagccatattaatttcagacacaaccgactttagaacaaggaatattatcagagatcaaaagggacattccttaatgataaagggatcagttttctaagaagacataacaatccttaatgtgtacgcactgaacagcagatcatcaaagtacatgaagctaaatctgatagaactgcatagagaaatagataaattcactcttagagttggaggcattggcatccctctatcagaaatggacaggtttagcaagcagaaaatcagtaagggcataacatgatcaatcatgatcaaaaacttgattcagtcaactatatgtaactgacatctatggaccactttacccaacaacagcagatgacacattttctcacacacacatgaaacattcagcagaatagaccacattctgggctagaaaatgcacctcaacaaatttaaaagaatagaaatcatacaaagtatgttctcagactacaatggaatgaaagtaaacaaaaatcactaaaaaagatagctgaaaaacatgaaaatacttggaggttaaacaacagactgaaataactgaaggtcacagaagtctcaagataaatttttaaaatatttcaaactaaatggaattgaaagcacaacttatcaaaaatttaggaacgcaacaaaggcagtgcctactgggaaatttataccactgaatgtatgttaaaaagaacaaagatctaaataaataatccttcccattggaaagctagggggaaaattcaaattaaattcaaagtaagcaagtaaaagaaataataaaatatgaatacaattctgagtggcctgatatctgatttctggaagctgagttttatgtggctttagaagtttcatgttatgagtgtgaaatggcaagtgaccatggcgattgcttctgatatggccacctcaattacatagagaagggatgatcagtggcatcatcccagttctgcatgataccacaccagtgattctccaaagtcaagaaacctgtatatgttgtccttgtgtttggtgaatctgtggtctatagactacataatgggctatttctggggtactgaccattttgctccattaaggaaaaccatgcatgagccatataactatgggaaacaatgttttcctttttgacacctggcctactctcttcttaggtgagagcaaaggaagagcaccacttggcctgctgcacccagggcaatttataccatccagatatccagcactgaatatgtagcaactccaatccaccatagaacattaatctatctctgataagcatgctaatatctatgattctgcctttgtggggagatgactccatagattaaccccctggcttggggcaatatggagatgttatattgtcttcgacagggaagatgacataaaaattatgttcaataggattatgtggagacttaacataagtgacaaacctaagacaagaagttagaaatccttgtttcttttaggtgaataattttggagcttgagaaaaagacaccattgtggcgaacttgctttgccaaaccctgtgtatgcatttttgggtaaacaacacattttcatcctttaagcattgtttccattatttataattcctaatagtatgagacatttaggactcattccttggttcttcttctctcattgtatctttcattgtatctttcagtgctgcaagagatgcaatcatcccattaatgaaagcagagtgactgccttcgtcacacatacattcaaaatagatcactaggctagtgatgattctctcttaaggctattatgtttactcatttaaaaaatctggatctcagtatagattttcttctcagtgagtgactgacctatctgatatgatgctatgacaaaatctttactttttacctgaatttggtgggattgaaagttgagaacatattaatttattttctgtttcacttgattaataatgcccaatctaacatgcagtgttcacttgggtacatatatacaactacatcaaattttttcttgttttaccttaaaagtgttaagatattgtaatggtatggccagtttactcactggtaaacttttcataacagaaatatcatgccagtggacacaaatatgtaacatcagtaatttgctttcctcatgaaacagagaactgaataagttgatgggtaaactagtatctgaattataaaccccaaatatcatgtgacttcatcattttcagtataatgaaaatacaggctgtgaatgcttccagttggtcctgaaagggcctttgaggtgagtaggttcaccaaagcggcaacatatgtgtcccagtgggcattctactcctcagtaagtgacttgacattctttatggcttcctcatgattaaaacaagtctttttattgaaattttactgaaataatgatgaaacaatgtaatattcttttcatctacatataagagtagacatagattaacctctaccttttttttttttttttttgagacggagtctcgctctgttgcccaggctagagtacagtggtgcgatctcagctcattgcaacctctgcctcccaggttcaagcgattctcctgcctcagcctcctgagtagctgggattacaggcacgtgctaccattcctggctaatttttttgtaattttagtagagacatggtttcaccatgttggtcaggctgttcttgaactcctgaccttgtgatctgcccacctctgcctcccaaagtgctgggattacaggcttgagcccccgtgcctggccttcacctctacctattataatattttcatgttattccattttgtcatgtttgtgggtcatttgatgagggttcacaatatgaaaactgggcattttggccaagatgcatgtaaatgaatacaactccatgtgccctgataactgatttctagatcctgagatgcaagtggcttccagagtgtctgtattttatgaaagttcatatacagtaaaccatgctaattgttactgatacagcttccttgaatatataaagcgatgattaatgggatgttttgtttctgcagaaaaccactgaattgcttctccaaagccaagtgacctgtatgatttgtccttattatattttgtgaaccagtggactaaaacctttataatgggtcatttttttagtattgtcaatattgctcaattaacaaacatgactcatatacacaggtgagatgtagttctccctttgacatttgctctacatctgtcccttagatgatgatatggaagaaaagcactctttggcctgttgtgactgggacagttgacagcacccaggtgtcctttaatgaaaatgctcttgacaccaatgcatcctagcatcacagcttcaggaagccttctcaagtgtgcatggggagtactatgtctttcatcaataatgaaatcttctgatttggtgagaaataatgccttaaaattacactcaataggattatgctgaggctcagcctacctagatgaaaatgctgaaacagataatagtgacttcttatttcccttcaaggggaattttattttgaacttgaaataacaaaatctttatgggtaacttgtttgcccaactgtatcagtgcacctataaatgaacaaaaaattctcagcctataagctttgcttcagtgacctttaatttctaatagtaaggtatattcaggtctcattctttggctttttctcttattgtgtatttcagtaagacatgctgccaagagatgtgccattctattataaaagatcagtagcttcctttaccgacgtgtatattctatctagaacattgagctatggaagactcccacctaagggaattagttttacaccttcaggtaatctgaacttcaatatgagttttgctttatgatataatccttatgacaaaatctttactttttatctgaacttcatacaatcaatattgaagattttttaaattcactgttacctagtccaatatttaatatacatttggctgtagatacatgactagacaaaaattttttatattattattgtgggggctctaacagtgtttcactgttttgagttactgatgaactttctgtaactgtgagaccatggtaatggaagagaccgtggtaatggggaggagaatgtgaggatcagtatgtactttgcctataaaacttaaatatttagtaatttgatcaccaaattgatactcaaatttcagttcctatcatgtgtaaaccaaaaagtatctgagatttagaagtttatttagaagtggattttgccaaggttaagcacatgctcagaagaaataaacccaaaatcacagaaaccgtctggggtctatgtctttctccaaagatgattttgagggcttcagtgtttaaaggggaaaagtgggctggagggggaaaagggagggtatgataatccgtatgttccaagacaaaaggagcaggtaggggtatagtcaataatgtattcatctcatgctcagtaaatcagcactttacataagataaggtgaacatagagaagctacctgtggagatctttaacctttaatctgtagctatctgcttaagatcaaaaggaaattcttgcatgactcagctttcagcttattttttttttctcttggcgtagtaaattggggtcccaagtttattttcctttcacacatgtgaccccactattttcagaataatgaaagcacatgttagaaatgatgacacttattcctaatatggactttgaaatgactgggttcacaaatttagaatcatggatctcattataggtttttattttcaatgagtgactggcctacttgatggaattctttcaaaaaatgttcactttttactgagattatattaaaataatgttggaatgtgttcagtcatgtttaccatctctatagtatgatatatgataatatcgcagattactcagtgtcaaaaacctggcataccttgtccttatgatgttgatgagtcaatggtctagagccttagtaatatgtctttttgtggtagtacggaccgtatctgtccactaagaaatagcctgtgtaagccatatacacatgaacaatttatgtctgattttgaactatggtcccattcctatgcctgtagataaagactgctgagaagagcaccctctggtgttgtcacagaggcaagtgctaccgcacaggcatgctgcagtgaatttaactgatcctctgtccctgcaaccgttgtttaaggatgctattctggtaagggtttacaagacaacgtaaatatatgtataaagagtatcttcaggagacgtaataatgtaaaaatcatgctcaatagaattaagctgaggctcaaaatagtgccaatccttgcaagaaggtgtgagaagtcattagatttggggggtatgggttattccattttgaacttggtaggaagaaacccttgggggaatcttacatcatttgcctctacctacttctaaaggaacttttaaaattcttttatcttgtctttattcttaatactcatacattattagagagaatatcttggtttattttttcctcatggtgtgcttcagaatgttcatccagcaagaggtgtgtcattacacggattatatatatgagttctgtctttcgtaattaaaaaatttcaagtggaccatttgaatattaaacattggccttcaaaatgattacatttacctgttcatacaaaattagtgagcaatgatctattctttccaatgagtgactgatctaatgaggtcctcattaaaactccttttttttttttttttttttttgcctgaacgtactttgatttaaagatgtgacatttagtaattcattgcaatcaactctttagtgattgcacaaatagacaatccttttctattattttattttttggttgctgtgtatggtgttatgatgtttgtggatcattgaggcactactcacaactgaaagactttggcaattttagggaatggcttgagattcagctataggttacccttgattgaaattgaatacaatacaggtagtctgaaatctcatttccttatcctgagtctcactcaggtgtcattagattcattataaagaaatgcatacactatgtatgatgccagttgttactgatatgaagacgataagaagcttttatttctgcagggaaataatcccttaccaggtcatcaaaagacatgatacctatgtgcttggcccttactgtacacggggaatcagtggtctaccacagcttaagtaacgggtcatatttggagtatcacacatctcagtcttgtagaaattaggaacagcaattaggagtcatgcacatataagagatgtaatcccaccctttgactatagcctactcttgtcttttacagaaaagactgtggaggaagaaaaccctttaccctgttgttcagggagaaactgacaccactcaactgcctggcactgaaaatgtggcatccagtccactttaccatcagtgtttaaggaaaccatctctggtaagcatatttgatccaagtgtgcatatgcacatgagtgccatgggctgggtcaatgatgagatgttaccttgaagagaaatgatgacgtaaaaattaagttcagttggattacgctgaggcccagcctaggtaacaattgtgagacaagaagtttggatttcttagtttccttccattggggactttattttgggattctggcaatggaatcttattgcgaaacttgttttgcacgcttctatctgtgtgcttctaagtaaactataaattctgattctttaagtgctatttaagtttcctacaattcctaattttcttggatatttggcactaagacccttgctctttttctctcattggatgatttagtaagtcctactggcactattctattgagtggaggtgagaggctgcctttgctgactgaaaaatttcaaatagataattgaactagtgaagactgagctctataataattagatttattcattcagacaacatgaatatcacgatagggttttcttctcaatgattgactgacacatgtgatgcaatcctcatgagaaaatccttacttttgccctacgattgattgttttcagggttgagaaatatgatacattggaattttgctcaatgcgctacagttatcgtggtctaaatatacagacaaaattttctgtaatgttagtataatattttaacagtgctaggatatgttttgatcattaatgaatctttatgacttaagggtccttgcaattgggataaaaatgtgagtttcaataatatgtgtttgcccatgaaacttgaaattagataaattgatgagcaatctggtgcctgacttttctttctcaagtacctttggcttcgttttcattatgatgaaagtgtgtgctgtgaataatgccaatggtaaataagacctttatggacacttatatgaatgacagtcagggccgttccttcaggccacatgattatcgttatgcattcttcttctcaatgagtgactagcatacttgccaggatctacaagagggaaactttattttttactaaaattgcatcgatataatgttgacaagttttgtgattattggtatcatctctaagatgtgatgctcatttggtctaaatatagaagttaacatattaacttctaatttatatatgtcaggttgctatgtttttacaatgtttgtggtttattgatgatgactcacaacataaagactatggcattatggctacatgttctaattctgatatatacatatcccatcattcattaaattaaatacaattatgagtagtatgatgtctgttttctaaaatctgagtttcacctggctccagtatgatcattatatgatggagtgcaagcaacatgttaatgccaattgttattgatatggcttccttgaatatgtaaacaagactgatcaattgaaggtcttatttctgcattaaactacctgagatattctccaaattcaagagacctgtatagttagtctttattctgtttgatgaataaatggcctatagcctatctaatggatcagtttttccagtactaatcatatctcttcattaagaaatagcatgcataagccatacatacaggaggtaatatttgtcatcttgatctactgtcttatcctcctccccttagataaggatgactgaggaagagtactctttggcttgttgacaccagcacagctgacacacccagatatctgtttggtctcctgtgaactttcaaccaggatttaaggatgccactctggtaggtattttcagacaaagtgaatatgtgattgtagagttccatggcttgtttcaacaataagaccttatgttgtcttgaggacatgagatgacatgaaaactagactggctggtccttatcttttctggggtattctagtttgagcttaagataaagaaatatatttgggatatagtttctctcacctgtttctattagcttctagaagaccacttatcaattctttcctcctgatttagtggaaaacattttggcagttcctcaaaaagttaaagataaaattaccatgtaacccaacaattctgctcctaagtacacactcaaaagaattgaatacaaatgttcaaatgaatacatacacatgaatgttcatagcagcactattcactggctggagttccagaccagcctggccaacgtggcaagaccctgtctctactaaaaatagaaaaattagctgggcatggttgctcacgcttgtagtctcagctactcaggaggctgaggcacaagaatcacttgaacccaggaggcagaggctgcagtgagctgagatcacaccattgcactccagcatgggtgacaagtgagattctgtctcaaaaaaaaaaaagtgaaggtatgatgacaatgtcacacaaaatagtgaataccaataaaaagatagaaattattaaaaagaaccaaatgaaaattctggagttgaaaagtacaataactaaaatttaaaaatttattagaaaggctcaatagtagatttgaactgggagaagaaacaagaagtaaacttgaagatagaacaatagagattatgttcaaaagaacagaaagcataaagaataaagaaacaaagagagtctcagagaatgtggaacatcattaagcataccagcatacacatatgggagtaccagacaggcaggggagatacaaaggatcagaaaaaaatatttgaaggaataatggctgaaacctcccatatttgtggaaaacaataactgacatatccaggaagctcaatgaattccaatgaataaatgcaaagattgacaaatggaaacatcacagtaagatatctgaaagtcaaaaacaagaagaaagtcttgaaagcagtgtgagaaaaatgatttgttacttacaagggaaccccaggaagattaacagctgacttcagcagaagcaatggaggccagaagcaaaggcataacatattaaaaatgctcaaagaaaaaagaacctgtcacctaagaattctgtacctggcaaagctgtctttcaaaaatgaaggcaaaataaagacttcctcaattaaaaaaaaaaaagttggagaatctactgctctcaaaacccccttaaaagagttctctaaatacttaaagaagttattcaagctgaaagcaagtgagcctaggtagttattcaaatcaacaactacaaagcaaatgcttgtaaaggtaattatataaatataaaagacagtagaaatgtaatttttctctgaagtgattttaaaataaattctataaaataatatgtatataatgtattttggggtctataacatataaaagtgtaatatatttgccaaagaagataggtggatgcaaagcaatacaagactaaggaaatgactacagatggtagattaatatttattataatgtctttcttggcttatagcatcaataaatgtaatatgtataataataccacaaaagatggcaaaaagggaatagagctatataggtgtaacatttctgtataagactgaaattaagctagtgtaaattagaaacttatttgggtgagttaagatatatgcagtaaaccctagagcaaccactgaaaaaattcaaaacatatagtgaaaaaaattattaaggaagtttaaatgctacattataaaataattacttaatgaaaaagaagtaaagaggaatagaggaacaaaaacacatgagacacagaggagaagaagaatgacataataaactcaactaaataataaaattacatgtgaatacagtaaatatgtaaattaaaaggcagagattcccaggctggataaaatctccaggtttaatatccctttgccccaaataaactccagctttttttcctatgatgtatttgatcattgtgctggtttacaaaagatttctaacctttctaaaatctttaaaatcagcggcgaagtgttctatctcatttcacccatatatatcagagactcaaatcctgaaaaataccaagttgacctgcccattcttgagttctgctgaatggtgtgttcccagatcactatggatattgctgtttacttgaccaattacttttagtcaacaagatttgaacaatgcttttgattcacacagcgtgagacctagacttccctcatattgaaataggagcattagcatcctggattcctgcaccggcttttgaaccttatgtttcctgcaccccaagaagccatgttatctggacattgtcctctctgtgatgggaaagaagagttttttcaacaaatgttgctaggacaactggatatctagatacaaaagatactgcatttatgttaccagttttctgtgtcagtccattttgctctgatgtaacagaatactgcagactgggtatttgtaatgagtggaaacttattggctcacatttctggaggctggaaagatcaaggggctggcatatgataagggccttctcactctgtcatcctatggtagaagggcaaagagagggtatggatgggacaaatatcttcttttataggaacccacccctgaaataacagcattaatccattgatgagggcagagctctcacagcctaatcaccttttttttttttttttgagacggagtctcgctctttcgcccaggcgagagtgcagtggtgctatctcggctcactgcaggctccgcctcccgggttcacgccattctcctgcctcagcctcccgagtagctgggactataggcgcccgccacggcggccggctaattttttgtatttttagtagagacggggtttcaccgtgttagccaggatggtctcattctcctgacctcgcgatccgcccgccttggcctcccaaagccctgggatcacaggcgtgagccaccgcgcccggccgcctaatcaccttttaaatgttgctttattaaacactgttgcattgagggttacgtttccaagacaggcttttgaagtgaaatgaaaaatttgaccattcccaattatagtttgaaatttcaatgctcctttcttagtatgtgattgaacatggataagataatcagtaacggtggatgtaagaacactattaaccaacttgactccactgtcatatgtagaacacaacacccaacaatagcagaatgcacattatattcaagtgctcataaagcattattttgtatatatgtattttctcccttttttgataaagttagctagtagagtgcctatgtagttaatattttttaaaaaccacaggcttctcaattattaattacatctatggtttttaaaatattatcttcattaatttctgctttgatccttattcctttctttcttgtactttcttttatttttgttcttttttagtgttttaagatgaaaattgaattgtttcattcatctttttcattttttaaaataaaaacatttagtgcaatgaattttcctctagaactgcctttaaatgcatattacagattctcatatgttgtatattttaaaataatatgtaaatatatatttgcatagaaaaacatatttacatacaaaatatgtatttacatatttaaacatatgtattttctgtttatatttttccttttactcaggagttaataggatatttttaaaatttcttggtgaaagcgcctttatcttattgttagtaaagtctatgtttttgcattgtgatcagatagtgcagtttttaatatttcttgagttactaatttttctttccaacctaatatataatcagttttttgtgactatgccatgggcacttaagaacaacacagatcttctattatctgtcaagtttaatatgtagccataaaatctaccttatttttatgttgctatttttgcccacttgatctgtctttttctgataatggtgtattatagttttctattatgggatcatctatgtcttgtctccttacatctcatacatctcatgcagttttttattcataaaggtggttattatgttatttggtatataagtctccataagggtcaaatcttcatgtgtattatatcttttagcataaaaaaagtgtccttctttgccttgattaacatggtgggacttgaatacattgtgtgatataagaattactacgcttgcatgcgattggcatgaaaaaaaaaaagattactcctatctctttttgttttcatgtgcctgatatactttcgcccatccctttattttcagtcttttagaatccatttgttttagattcatctctcatagaaaacatattatgttttattttgtgagcccactgaaaatatttttcttctagtaggtgtgtggagtccactcacgtgtgtgtgtgtgtgtgtgtgtgtgtgtgtgtagctatgactgatatttattgtgtttgtattttctttatatttgctacgctcttttgtcacggtgtagtctttgcttgcctatttattcatacttaggagagtttttgtttttgttggagtgtttacttttgtacctctcttaattctgttagttctgtgttttcctagttaatcttttacttctgatttatcagtttgggggatgttcttcagcactcccctattctcatgcaaagtgctttcggttttcctctttttttttttttcctcctcctgtgtttagttgtcttatttctactttgtcacaatattagtgacatttgtatgctagtcttctaccctggatagggaatatacacctgtcagatggaaggaatatcatgttgtatgctgctgtcaaatagatcctaacatactttaataatcttttaaatgatataatccaagttcatagttaactcctgactttttatttctatcatattcagtagatatatcagcattgacagccatcccactgcaatatcacttcacattttgcttagaccaaagggttctgagaagcttctggagctgttgtgaaagccagtttcattaaagtcaagtcagacatccacctgtgctgttttctgctataagctgaatgcaggggatacaatgttaggagcatcagtgtggtatgaggaatgtttacttattggtagtagacttgtctggatcaaggataagtacaagtatctgagatgctgaggtgtggctgaatgaatggccatctgttctattgcaacaggataaataacacattcggagcaggcctaactggtgaactcctcaattatctcacaatagcactatcacgtgtctttcatttttcacagcacagtgtattttcataaaatctttattggtagaccacaactttagcatttcccattcaagatacaactactgtgtcacttggatagcttggtagagaggaaattggctattttcagagtaaagaattcaaggaggaaacagattataaatgaaatgacaaaggtgatgagatcaagcaatgctgacgtttgtagcaaggagtagagggctgagatgcctgtatgataagaaattataatggaaccaatcttgtgctttattttttattaatttggagcattctctgatctgatttattttctatgtgatattttgctgcaggttaaaagctgaaacaacttcaaggaaacttcagggaaaagaggtgagcagccactttggtacttttgaaattctttgttctgtgggcaatgatgtcagaatgtcaaagactgtgagttcattatttcagaggcttaaaggagattattcttttggtagatatatttccccatgggtgtccctaccttgttcaaggattccttattattgtttcttctctttgtttaaatctctttttgagatgacctaaacaactgtggagaatcattgatatatttccttttttcactgttcatgttgggtgaaaataatcttgtagtgaaattcacatgttctaaatattgtttttttacatctttatctggcacattcataacatagatgtttctatacatattagtactgtaatcataccatatattattctgttaccccacttactccttaaacttttagttaattaaagagtttttataaagtcccccaatagattttttttttttgagacatagtctcactctgttgcccaggctggagtgcagtggcgtgattttggctcactgcaacctccccatcctgggttcaagcaattctcctgcctcagcctgccaagtagctgggattacaggtgcctgccaccacgcccgactaatttttgtattttcagtagtgacagggtttcaccatcttggccaggctggtcttgaactcctgacctcgtgatccacccgccttggcctctcagagtgctaggattacaggcttgagccactgcacccggccagattttaatctaattttattagaacaattcagtcatatgttttttcatgctatgtatatgagagttccattattcagatactaaacaaatgtctactgtacatttactgttctcactgatgatgcattagataaccatgcacaaaataagcctggctgtggaaacgcttatttgttgggagggtgcttgtttggatcgatgatgagaataattgtctgaggatgctgagggactcattccagatgtcaatctgaggtccagatgtgcggccctccaataggacaaataagactctcagagcctggctctatttggggatccctcagtgacaacatagtacccctgtgagcgtgccttttctatctcttcgaagagggcagtggcatcctgtcttatgagtcagtgtgcactttagtgtgcctagtgacccaagacttgctttaattgtagatagatacttacatataggaaatatttcttaagtaacaaatgaaaaactttagaagattgaattaagggtcaagcaactgtgatatgtctgaaaatctcattagtgttgtgctgaaagaaggaaatatggcatgcctctattaaataatgacagtggaaccaagtttattgctttgttatttttactgtggagtattttctaagattatttttgctttttttttctttcatgttttgctgagatagaaggcctggaatctgatcctccacttcagagaacaggggtgagtagctaagccattatcttttgaaattcatatgtcatgtgctctttgctaggtctttaggtcgttttgtacatcttttcagaagcttattggaggacattttcatgatatgtccttttcctcattgagaccctcaccatgtcacctacactattgaatccttatcatttctcttttaattttaactctcttttgcttttatggaaaaatgtagaatttaagagaatttttggcaatttcatattggatcaaaatgtattgtagtgaaatccaggtgtgccaaaatattaacagattttccccatctgtttaattattggggtttcagaatagagactccatggttcataatatctttgtggtcatactacattatatttctgcttctaatttaattattaaatattgacttgaattagtcttttcctcattgttgcaacaaggtaagttatataggaaattttcttctcttgatggcatgtctgagataatcatagatataagacacctggctggtttctagaatgcatgtaatttttatttcttgatctgtgtgttgagtacttgctgtgattgcatcatgcaaatacattgagctttacaattttagtgtatgcacttttctacatgtatattattcttcgatagaaagtaaaaaaaacttatcgaactagtcaaaatattggttaatacataaaaaaagtctcaagtagattgtgtattacatggtgcttgttgattgatgccctcataatagatcaagtgggttctctctttagcacagggctttttagcaaatcatgtcatgagtagttactcaagtatttttattttaacacatttatattttttctatgtatattcttaaattctcttatacttttttctctgttataaaaacatgctgaacaatctcaagtcttaaggattgcagtattgtccccacatattcatgtattttggtactcaattctttatactttctttgacagatcacttgaactggcacatgtctcttgttttgcagagagggaattaatgtgataccttcatgcttttctattctatgtgctacataattgaatatacaagcaaatatagttgttaagatttagtgtgattatttctacaccacatgcaaagaagtttctcatagatcttaatagaggcccacatgcattgtacagtttagaatttggggaaatattgatgaagttgggtaaagtataaagccaaaagtcagaacagtgaactccttgcttaaggatttccttggagattacttagtcaatacacaactgataaatttaagtgcttttcaccttttgagttctcgacatactaaagctaaaatgtgtttcaacttttaatcctgcttccctgattttcccttttttagtctgagatcaaagagtttcagccataaattactgccaagagtaatcacttcattttaagaaagcttaacaatatagaagaatataaaattatttatgacagatgtatttttaaccttttccccatgctttccagaggaaatatgtttaatcatctgccctatattagggaaaaactttctatgctaatacaagtatctatcaatccatttatctttctatataagatgtattgatcataaccaattaactttactgtaaatgagctttagatttgacattttggtagtaatatatgttgtacaatctcctgaggtcctataggtcttgaggtctctatgtcaaaaactatagatgtgccagtgtcctcagtgaatgtgaaggacaccacattttccttagccatttcttgttttcagaataatggttatcaacattttgctactgcaagataccatacatttataatcggaatatgccagtttttatgcactcatgcctctgtttctgtagagcattcccagaatgagtaatgcttgaaaattaggtccatgtgatttctttaatgagttatagtcaaatcatgaaatattcaggttaccattatttcagtaatgtattagaatgtcaaggtaaagttatctacattgtatatatacacacaacatagatataatttatacaacatctatatttatacaacagatatatatttatgtatatatttatacaacataaaatatattttattatttaaacataaaatatattttattatttaatatagattcttaagtgataaatatgtttaatattattaaaataggttaaaataggttatagttagtacagtgaaaattggcagcccttttacaaaacatatgccataactatagcatttatcactgacagtcataccaggatagtcttttatttccaatcacttaaatattcctaattgcaaaagaaatttgaagactaaaattcagaagttttgaaagagccattgcctgggtaaactatacaggtttcagttttatttataataattatgaggccaggcgcagtggctcacacctgtaatcccaacactttgggaggccgaggcgggtggatcacgaggtcaggagatcgagaccatcctggctaacacggtgaaaccccatctctactaaaaatacaaaaaattagccgggcgtggtggcgggcgcctgtagtcccagctactcgggaggctgaggcaggagaatggcgtgaacctggtaggcggagcttgcagtgagccgagatcgcgccactgccctccagcctgggagacagtgcgagactccgtctcaaaaaaaaaaattatgtatatatttataaattaatacttaataaattaataacttgtgataggcaatgcaaagatgacagtaaaaggacaaaattgattagattgataaagtcctgttaacatgaagaaattgaccaggataccatccacactataaagttagagaaatttatcaggacaattccctaaaatactcttctcaattttaacattgtaacaggaatttttaaaattttggtattatgtgtgtttccttccagataatttgaacagattcatatttggtatttttaaaagccatatctttgtccttagtgctggcaatgtattcttgagaatgaacaaataagagatacgtaaaagcataagagaaggtatcaggttgaagtagtcaatcagttatacagaacacaaagaattttatcttgtataatgtttatatagctttatagaagtgtgctgaaagggctataaaacatggacattattatctcattgaaaggtccaatacgtactgaaatacatgcttttattttgaaccaaccaccctataaacgttgtatggcttatttagatgagagcccaggttgtgtgtgtctgtgtgtgtgtgtgtgtgtgtgtgtgtgtgtgtgtgtgtgtgtgtacctgacagggaagcaagaacatcgagttgccaatgcactctgtctatggttagaatcatgctgaaaacatggctcccccagttctggaatgagcccacagatcaagcattccccaaagacatagcaggctcaaatccctgtgtacacaatattttatgattatcttatgtcagtactttcaaagtatacagtttgtgtgaagacaaatccaatgtcatttttcttggctagcctatatgtgtggtaaatccattatttacttacttgcttcctgaaaattacaattagattaacaaactgcagcaaagtgggcatgatgagatagagattgaagtgtaagcttatgttaatgatgcccttggtttggataaacacatctaagagaaaaatggaaaaacacacatggcagggaagccttgatagagccaaaatataggattgtatgtagtaatgcaatccatagatgagcatttggcagtaatattatttttcagatatggataaaaattgcttaggagagtaaagagagacaaagttgaaagcaggtttatagtaggtgttgttttagtgttgatccctttttgctccaataatcaaagtgataaatattgaaaattgattcatgcagcattacttactccattctaatttttatatatgtcaaaagtgccatctcccaaactgtgctatccccttcaggagaagagactctgctgaagtttataaggttgacatattgccagcttcaataatgtaaagatgaagtgtatactgaattcttaatgcaaataacaactctattggaaagtaacccagttatagaagtgctaatttgtcaggagctgccttaccaagatcatgatgagtacagttatctcaggattctgaaagattgttttccgatttcaactagtctagctgaatgttccttgatagaaagagaggacttttagaattggttcaatatgatgacctcctgaattatctcacatagcccgtttgtacatgcctttcttttctctcagaaaatggcactatcataatagctttcttacacagacttcaccttagggttttacattaagggaggggtctggtgtttcatttattttgaagtatttgttgttgattgtgtacagtgcttgagtaaaaaattgaatatagaaacatctagaatatttttttaaaggatcagtgtttataaagtgaattattagtgtcaataatgttgggaaagttttaagagaatataggaaacttgaacattacacaactacaatgggaccaaattgtggggtctcattatagttaatatttatgtatttttttccaattgatttgtgtgctttttttctgcatgtttttggcagatagaatggctataacaagtaacagcatgtcaggtaataaaaataagcagagccctattcctttaaaaatcttcactgatgggagggccataaaataagtcttaatacatttaaagaattaaattcatgtaaaccatgttaatttaattccacaatgatattgaattagaaataagaggaatatctcttgaacatctcctaaatgtttggaaatttaaattagcatttctgacctatttattggttaaaaaagatacaaagaaaggaaaattgaaaagtcttttgaactgaataaaaataaaaatatagaatctaaaactttatgggatactgacaaaacaggatatagggaataatttatagcactgaaatgcctatattagaaaagaaaaaaggttttaaatcagtaaatttgtattttaccttaagaaacttagaaaagaacaaattaacccagacttaagtaaaataaaggcactaataaagataagagcagaaatcaatgaaatataaaacaacaaaacacagagaaaaattgagaaaatttaaaaatagcctagtgagaagatattgataaacttgtaaccagaccaatttaagaaaaaaagtcaaaacacaaataccaatatttgaaaatgtaggagggcaaatcattacagattctatgaatactaaaatgataataaggaaaaattatttaaaaggggcatgtcagccaggcatggtggcttacccctgtaatcccagcactttggcaggccgaggtgggaggattgctagagctcaggcattcgagaccagcctgggcaacatgttgaaaccttgtctacacaaaaagtacaaaaattagctgggtgtggtgttgcacacttgtagtcccagtcacttgggaggctgaggcgagaggatcacttgagcccaggaggttgaggctgcagtgagccatgtttgtaccactgccctccagcctgggtgacaaagtaagaccctatgtaaaaaaaaaaaaatgtatgccaacatttttcaataacttaaatgaaatggaaaaattccttgaaagacacgaactacaaaaactcagtgaacaagtaaataacctgaatagccctgtatcaagtaaattgaatttgtagttaaaagccttccaacagagaaaacttcaggtacctatagcttcatatgaaatgaaaaaaaaaataccaatcctctacaagattccagaacatttaaaagaagggaatatttcccaacttattccatttggacagcaatacccaggtaagaaaaagagacacagaaatttaaaaagaagaatatacattattccttaggaacataaatgcaaagaaatctaatcaaaattttggcaaatgaaatgtagaaatactttatgaccaagtgagagttacccaaagaatttaaggttggttttatatgtaaagatcaaccaatataggaaaatcacttctggaaagtcagagtaagaaactccaaaaatctactcctccataaaaccaataacagccttgatagaaatagttgaaattaattttccaaaactttggaaattaaccaaaggcttacaaaattccagagaacattaattcaagaaaaatggctgaatcagtaagaacagccagctttgtggcattttaatatgaccccttcccatgcttttctccctagtgctgagatagtcttaaaaattagcaggatagcaaccactggagaagaaaggtttggaaatttcccaaaaagttccatccccatagaattatcactatttgacctctaaagcccaatctataggatttatattcatttggactgactcagagctcactcagtaaggaaatctcaatttcaaggtattggtcaaaaagaatccatggcaattgttgactatcacaactgcctgaagtcttggtaacagttgggataaacaagaagctgatcaaaaactgaaaactaaaatcttgggaatgagatatctacaggatgcttcaaaaagctttgatacattcctgtttatctagaaagctacatgcaggctgggtgcagtggctcacacctgtaatcccagcactttgggaggccgaggcgtgcggatcatgaggtcaggagtttgaggccagcctgaccaacatggtctctactaaaaacacaaaaattagccaggcgtggtggcgtgcatctgtaatcctagctactcaggaggctgagacaggagaatcgcttgaacccgggaggcggaggttgcagtgagccaagattgtgccactgcactccagcctgggcgacagagcaaggctctgtctcaaaaaaaaaaaaaaaaaaagccacatgcatgtaattgtttacctctggctttccttttatgctctgggcaagctaaggaagagttgtgaactacctaagtgctgaatgggaaccataacacacacacacacacacacacacacacacacagcaccttagtaaagggtgagaggcatgttagttagaagcattaaaggaaatctctttctagtcattatctgtgcactaacctaactgagcagagacttcagtatccacatactacagggcatataaactttacagaattagtccaggaaaatcatatctaaaaaaaaaaaaaagcagtaacaaaaataaactctgggaaaggggagaatatgatttaaagagttgccacattatacataatatgtctagtgttcaacaaaaaattacgagacatgcaaagaaatagaaaaatatggcacaaagaggataagatgaagtcagtgaaactatcctcgaggaagaccagatgttggtcttactagacacagacattgaaccagctattaaaaatacgtacacagaactaagaaaaacatgtcaaaagggttaaagaaaggtataaaaatagtgtcttaccaaatatagactaccaataaagagatagaaattataagaaaagacaacatgaaaaaatataaagcaaaaaaattagacaattgaaacaagagggcctctattcgcagatttgagcaggcagaagaaagaatcagtgaacttgaagatatgtcaactgagattatccagtctgagcaacaaaggtgggaaaaaatgaagaaaactgagcaacaaagaactgtagaacagcatctctcataccaatggatacataaactggagccctagaaggatgaaaaaaggagaaggaaagaaaacttcccaaattttaagaaaaacattaatttatatacccgagatgaccaataaaatccaattaagataatctcaaagagaccaacacctatacacatcatagtcagcgtgtcaaaagacaaacataaggagagaattcttgaagatagtaagaaaaaaattattcataacatacacaccatcctcaataagtctgacaattgacttctcactgtaaaccatgcaggctaaaaaggcaatgtacataaccaaagtgatgaaataaaaaccttcaaccaatcattctagatccaacaaaactattgttcaaaaagaagaaatgaagacattcctaaacaaaatctcagagaaatgttctctataagacttgtcctaatagaaatgctaaaggaactccttcggtctgaaatagaaaggcactggagagtaaatcaaatccacgagaagaaataaagagaaccagtataagtaactacatgtgtaagtttaaaacaaagtataaatttattttgtttgtaacatttgtcttttcctatttgatttaaaatataatctcaattataaacttgtgttgatggtattatataaagatgtaattttgggttatagcaccaaaatggcagaataggaattttctgtaggtgtttcccacataagtatcaattttgacaaccatccatgggcaagagtaccttgtgggagttcaggagttgacagtaaaacttcagcacaccagaggagtaaagaaatctaagaatagattcattggaaagggtataaacagtttcactttacctgcatcaccaacccccaaaagtggcacagctcagtaacaagagcccattatttcttccacagaggaaaaggagagtataataagtaagtgtccagtttctcaagacatacaggcccctgcccaagagatccactttattttcatctcacccagaatattgaggtgatcagcaaggtggagtggttgggagagggtaaaagcaggaaagagagatggggactcaaacagcagcccatacttggaactgccatagatcctaccagttacttcatggactccatcaggaacccacctataagccacaggggatgcatccctcccatcttgccaacaaaaccccgaatgctccaaatgcctcacccactctttggctggctcccaagtgcgctcctgtgaaaagcgagtgagtatctctgcagatggcttgcaagcacatgttgacagctggctccactctgtagaactggaaaaaagctcacatacatgagaatttcaggacactaccctaaaaaaaaaaatgagacttcagcacctggcctggctttatgcaacctagagaaggtgatatgatttggctctgtgtccccacgtaaatctcatgtcaaattgtaatccccacgtgttggacaagggacttggtgggaggtgattgaatcatgcgggtggacttccctcttgctgttcttgtgatagagttgttatgagatccagttatttgaaagtatgtagcatgtcccccttcactctcttgcactcctgctccaccttggtaagacttgcttgctacccctttgccttctgccatgattgtaagtttcctgaggcctcccagctatgcttcctgtatggcctgcagaactgaactgtgagccaattaaacctcttttcttcataaattatccagtcttaggtagttctttatagcagtgtgagaatggactaatgcagaaggcatacaacctttagaatttgcccccttgagggaacaagatgtgtgaagcaggttcatccatagaaaatgtctgagagaacctcaaaatccctaacctgactaactgatgaaagtgtttctctcctaaggccagtcagtaaagaccagagggggtgactgtttctttaaatgcaaaggcagcagcacaataattcaagaaacatgaaaaatcaagaaaacatgacaccaccagaagaacacaatcattttccaataaccaactccccaaaaatggagatttacaaattggtttataatgaattcagaacaattatgttaaggaagctcagcaaactaaaaggaacaccaatagactactctgtgaagtcaggcaaacaattcatgaacaaaactagaaattcaaaaaagagaaaaattatcttaaaagaaaacccagaagttatggagctaaagaatacaatgcatgaaatgaaggagcgtatcaacagcaaagttgatcaagcataagaaaaaaaaaatctgtgaaactgaagactggctatttgaaattattcatcagaggattaaaaaaaaaagaatgaaaagaaataaagaaagcctacaggatgtataaaacaccatcaagagaactaatataaggattattggagtcataaaggagaagagagaaaagggtagaaaacttatttaagaaataatggctgaaaactctccaaatctaggaaaagatatgagcatccaggtatatgaagctcaaagatccccgtacaggatacattccaaaaagacttcaccaaaacacatgataatcaaactgtcaaaagcaaaatcaagacgatgaataaaccaccaatcactaagagggagacaggattcttatgttgtcattattatttacatataatttcagtaaatgttattggaaaatttataatgttttaaaaaaagaaatttgaaagcaccgaaagaaaagagactcatcacatacagggaacccttttaaggcattcaagagatttctcagtagaaaccttacaaaataggagagagtgggatgaactatacaagtgctgcaaggaaaaaaatgccaaccaacgctttacctggcaaatctgttcctcagaaatgaaggagagagaagaactttcctagacaaacaaaagctgaggcagttcatcaccactagacctgccttacaagacatactaaggggagttcttcaagctgaaatgatatggcaatagttagtaatatgaaatgataaacctcactggtaaaggaaagtacatagtcaaatttagaacactttgatactataatgatggtgtataaataattttactgtgctatgaaggttaaaagacaaaagtattaaaaaaaacccatagctgcaatagcttgtcaatgcatactacagtataaaaagatgtaaattagaacattaaaaacatagcatgcaagggtagggaagtaaaagtgtagttttcatatgtaatcaaatttaatttgttatcagcttaaaataaattgttatacctatgttttatgtaagtgtcatggtaactataaaggaaaaacctctagtagatacacaaaagaaaaagagaaaggaatcaaaacataacactacagaaaattatcaaattacaaaggaagacagcaagggaggaacaaagtaaaaagagcaagaaaaaatttaacataatgaaaacagtaagtccttacgtgtcaataattactttaaatgtaaatggattaaattatccaaacaaaaaaacagactggacaaatggattttagaaacaacaacaacaacaaacaccgcacacacacacacacacacacaaaccacccagccccaactatgtgctgcctacaagagatttacttccactttaaggacacatacaggctgaaattaaaagaacagaaaaagatattgcatgcagatagaaaccagaagagaggagaggcatctatacttacagcatacagaaaagattttaagttaaaaactatatcaaaaggctaagaaggtcaaaatggtgaagcagttaattgttcaagaagacataaaaattgtaaatatttatacacccaatattgaagcacctaaatatataaggcaaatattaatacatataaaaggagaaatatacagcaatacagtaatagtagtgaacttcagtgcctccctttcaaaaatggataatccagacataaaatcaataaggaaacatttaacttaaacttcactttagaccaaatggatctaacagacattatactgaacatttcatccaacagtggtagaattcacattcttctcaagcacacatggaacattctccaggatagattatatgttagctcacaaaataatattacaaaatttaataaagctgaaatatcaattattttgcaccacaatagtataacactagaaatcaataacaagatggaaactggaaatttacaaatatatagcattaacatattcctgaacaaccaatgggtcaaagaaaaaaatcaaaataattttgtgacagcaaagtagaaacacaacataccaaaacatacaggacacagcaaaagcagttctatgaggtaagcttatattgataaacacatttaaaaaaagattttaaataaacaacattacacctcaaggaactacaaggaagaaaaaaaaacaagccccatgttatcaaagggaaggaactaacaaagatcagacagaaataaatgaaacatagactagaaaaacaatagagactattaataaaacttagagttagttttttaaaaaataaaatcaacaaacctttagctagactaaaaaaagagaagactcaaataaaataaaaaatgaaagaggagacattacaactgataccacagacatacaaattaagagaaaactatatgccaacatattagttaacttgcaatgggtaaatccctagaaacatacaacctacaaaaactgaatcatgaagaaatggaagatctgaacagatcaataatgaataagggaattgaatcaatattcaaaaatctcacaaaaagaaaagctcaggatcagatggcttcactggtgaagactgccaaccatttaaaaaaattaataccactctttattaagctcttccaaaaaaattgaagaggagaaaacactttcaaattcattataagaggccagttttaccttgatatcaaagatttaaaaagaacactttgagaaaggaaaattacaggccaaaacccttgataaatatagatgcaaaaatgctcagcaaaatactagcaaacctaattcagcaacacattataatggcatacatcatgaccaagtgagattcatgcctcggatgcaggatagttcaatataatcaaatcaacaaatgttacactactttaacagaatgaaggataaaaatcatatgatcatctcgatggttgaactagtttacagtcccaccaacagtgtaaaaatgttcctatttctccacatcctctgcagcacctgttgtttcctaactttttacagatcaccattctaactggtgtgagatggtatcttattgtggttttgatttgcatttctctgatggccagtgatggtgagcatttttcaagtgtctgttggctgcataaatgtcttcttttgagacgtgtctgttcatatccttcacctactttttgatggggttgtttgtttttttcttgtaaatttgtttgagttctttgtagattctggatattagccctttgtcagatgagtagattgcaaaaattttctcccattctgtaggttgtctgttcactctgatggtagtttcttttgctgtgcagaagctctttagtttaattagaccccatttgtcaattttgtcttttgttgccattgcttttggtgttttagacatgaagacagtgtggtgattcctcaaggatctagaactagaaataccatttgacccagccatcctgttactgggtatatacccagaggattataaatcacgctgctataagccataaaaaatgatgagttcatgtcctttgtagggacatggatgaagctggaaaccatcattctcagcaaactatcacaaggacaaaaaaccaaacaccgcatgttctcactcataggtgggaattgaacaatgagaacacatggacccaggaaggggaacatcacacactggggatggttgtggggtggggggaggggagagggatagcattaggagatatacctaatgctaaatgacgagttaatgggtgcagcacaccaacacggcacatgtgcacatatgtaacaaacctgcacgttgtgcacatgtaccctaaaacttaaagtataattaaaaaaaataatgctgctataaagacacgtgcacacgtatgttcattgcggcactattcacaatagcaaagacttggaaccaacccaaatgtccatcaatgatagactggattaagaaaatgtggcacatatacaccatggaatactatgcagccataaaaaagatgagttcatgtcctttgtagggacatggatgaagctggaaaccatcattctcagcaaactatcacaaggacaaaaaaccaaacactgcatgttctcactcataggtgggaattgaacaatgagaacacttggacacaggaaggggaacatcacacactggggcctgtcgtggggtcagggtagggggagggatagcattaggagatatacctaatgtaaatgacgagttaatgggtgcagcacaccaacacagcacatgtgtgcacatgtaccctagaacttaaagtataataaaaaataaatcatatcatcatctcagtagatttagaaaagcatttaacaatattcaacatcctttcagaactaaaaactctcaataaatcaggtatagaaagaatgtgcctcaacactataaaagccacatatgacaaacctggaggtaatatactcaatggtgaaaagtaaaaagctttgactctaagatcagaaccaaaacaaggatgtccattctcaccacttatatttaacatagtagttgaaattctagctagagcaattaggcaagaaaaaaggcacccaagttggaaagaatgaagttaaattgtctctgtagatgacatgatcttatatatagaaaacactaaagactccaccaaaatgctgttttaattagagcttaaaaaaataattcactaaagttgcaggatacaaaatcagtatacaaaaatcagttgcatttctaaacaccaaaaacaagttatccaaaaaattaagaaaacaatcctatttgtgatatcatcaaaaaataaaatactaaagaataccaaagaaactgaaaataaatggaaataaatggaaagatagcccatgttcatggattagatgaattaatactgttaaaatgttcatactacccaaagcaacctacagattaagtgcaattcctactaaaattccaatgacatttttcacagaaatagaaaacacactcctaaagtttgcatggaaccgcaaaagactcaaatagatgaagcaattttgagcaagaacagtaaagctggagacatcacactacctaacttcaaaattttattaatcaaaacagcatgacataaaaacagacagaaaagaccaatggaacagaatagagagcccagaaataaactcacgtttatagagtcaactaatattcaacgaaggtgccaagagtacacaatggggaaagtatagtctctacaataaacagcactgggaagacaatatccaaatgcaaaagaatgaaattacacccttatcttataccatacacacaaatcaaatcaaaattgataaagacttaaatataagacctgaaaccatacaatctttaggcaaaaactcattgacattggtcttggcaatgatttttttgatatgacaccagaagcacaggcaacaaaagcaaacctaaacaagtgggaccctaacaaactaaaaagtttctgcacagcaaaggaaacaatcaatcatcagaattaaaaggcaatttatgaaatgggagaaaatgtttgcaaaccacatatctgataaagggttaatatccaaaaatatataaggaatgcataaaattcaatagaaataaacaaacaaataatccaattttaaaatgggtgaagaacctgaatagacattttttcaaagaagacttacagataggcaacaggcatatgaaaaaatgcttgacatcactaataatcagggaaatgcaaatcaaagctccagtgaaataccacctccaactattaggatggctattatcaaaaactcaaaagaaaacaagctgggggaatgtagagaaaagggaaaagaagatccttatacactgttggtgtgaatttaaactggaatagcccttatggaaaacagcatggaggttcctcaaaaaattaaaaatagaactactatatgatccagcaatttcactattgagtatatatccaaatgaattaaaatcactgtcttgaagaggtatttgcacactcatatttatttcagcattattcacaatagccaagacatggaatcaacctaagtgttcatcagtagatgattagataaagagaacgtggtatatagacacagtggaatctatttagtgttcaaaaagaaggaaatccaacttttaaaatcctttaaaaagttaaactcataaaaacagagaggagaatggcggtttccaggaactggagggtgggagaatggggagatgttggtcaaaaggtacaaactttcagttataaaatgaataagttctagagatctagtgtacaacagcattactatagttaataataatattttttatacttgaaatttgctaagagtaaatatcaatattctcaatacacacaaaacagaactatctgaaggcactgatatgttaattatcttcattatagtaatcatttcacaatgtataatgaatatcaaaacaatagtgtacatcttaaatatatacagttttgatttgttaatcatacatcaatgaagctagaaaaaatgttgtaatttttaaaacaatagtaatataaattaggggtgaaggaattgacctatgttggagaaaagtttttgtaaactatttaaattaattggtatccattcaagctagattatttttaattgttaatttaattgtaatactaaggcaaccactaaaaaagcctttaaaaaaatatagcacttgaggctgggcatggtggccctcaattatatatataatatatataaaatatatattatatatatagtagatgaacaacaatgggatttaaatggtacactagaaaatatctgtttaacaaaaagaaagcaatagtggagaaatataagaacaaaaccatgtaagatttatagaaaatgaatagcaaattggttgacctaaaccctatcttataattatattaaagataaatgaaataaatactacatcaaaaggcagagattatcagaatagagaagaaaaatccaaaccataattcaaccttatgttatctgtatttagaatatttagagtcaagacacaaatagatagagttccatttgggcgtgaaaatatttaccatgcaaaatgtaattaaaatagagctagagcagcaatactaatatctgacaaaatatactttaacaaaaattgttgctaaagacaaaaaagaacattttataataagacacaacaattataaacatatacaccaaacaatagagcccaaaatatacaaagcaaaaactgctagaattgaagaaagatagaaaaatcaatgattacagttggaggtgtcaataccaaactttcagtaatacacagaacaacaagtcaaaagcaaaaaggaaataaaaaactacacattatcatacaacaccttaatacgatatccaacaatagcagaatacacattattctgaagtgcacatggaatattcttcaggatgacacatattaggctgtaaaacatatcttaataatttaaaagaattgaaataatacagcacatattctctgactgcaaaatattaaaccaattacagaagaaaatgtgggaaattcacaatgatatggagatttaaaaatacttcaaagtaaccaatgaatcaaagaacaaatcacaagagaaattagaaaatattttagatgaataaaactgaagaaacaacataccaaaattttgtggttgtagctaaagcagtgcttcaagggaaatgtataacagtatatccctaaattttaaaaaaatctcaatccaataacctaatttttcaccttaagaaatgagagtgaaagagcagacttaacccaaagtaaacagaaggaagaaataataaagattagcatggagataagtagtgaaaataaaaacaatagagaaaattaataaacttgaaagttggttcttctgatatatcagtaaaattgacgtattattagtttgaccgagaaagaacaaaagagagaagattcaagttactagacataaatgaaagtatagatatcaccttacagaaattaaaagaattctaacagaatattgtgaaaaagtgaatgtcaaaaaattaaattatatgagatacacaaatccctctaaaggcacaaactaccacagctggtgtaaaaacatgaatacaccatttacagttaaaaagactgaatagataaaaattttaagaagaaattgagtaagtaatttaattttcaaactataatcccaagaccagatgttttcattggtgaattctaccaaactttaaaaggattaatatctatttttcatacactctttccagaaaatagaaaaggagggaacactctataactcactgtatgaggtccgtattacccttatatcaagaccaaacatcataagaaaagaagactaaagacttatggtttctgctctgatatgtttggaagtcatcactactattgtcacaaggaaaaatctgaacaaactaaagtcaacgatttcttaaactaaccatagaattgaggtaacgggcgaaaactggagatgtaggcaaatacaaaaaaaatcacagtttatcaggagcagaaactgctgaaaccagcaactcgtatgaacatgttaagtggtaattgacaaatttctggagattgaatgtggactggattgagagttaggaactcctaagtgcccagtttttgatgaccccacacacttttgtaaactagacttccaagagccccagcaagtttcttacagtgaagactgcagaaaaatcccctgatgcttcagataggaggaagggaaaagcaactattttgaaataagcccaggggacaagtagttatttctaaacactctcagagcattttctttcacacggcagggggctccctgcaagggaagctactttgcctgagccttgtctgatgtaggagaaaaggaattggatggctcaagctccatctagcctttctgatttatataagggaagcaaaaaataggttaagaaactcttctgaaagtcacaactcagatttattttatatttatttatttatttatattttttgagacggagtctcgctctgttgcccaggctggagtgcagtggcgcgatctcggctcactgcaagctccacctcccgggttcacgccattcttctgcctcagcctcctgagtagctgggactacaggcgcccagctaattttttgtatttttagtagagacggggtttcaccacgttagccaggatggtctcgatctcctgacctcgtgatccacctgcctctgcctcccaaagtgctgggattacaggcgtgagccaccgcacctggcctcacaactcagatttaaccataagattatagaacacttctcctcccaaaacagagatatagatcaatggaacagaacagagccctcagaaataacgccgcatatctacaactatctgatctttgacaaacccgagaaaaacaagcaatggggaaaggattccctatttaataaatggtgctgggaaaactggctagccatatgtaaaaagctgaaactggatcccttccttacaccttacacaaaaattaattcaagatggattaaagacttaaacgttaggcctaaaaccataaaaaccctagaagaaaacctaggcattaccattcaggacataggcatgggcaaggacttcatgtctaaaacaccaaaagcaatggcaacaaaagacaaaattgacaaacgggatctcattaaactaaagagcttctgcacagcaaaagaaactaccatcagagtgaacaggcaacctacaaaattttcacaacctactcatctgacaaagggctaatatccagaatctacaatgaactcagacaaatttacaagaaaaaaacaaacaacctcatcaaaaagtgggcaaaggatataagcagacacttctcaaaagaagacatttatgcagccaacagacacatgaaaaaatgctcatcatcactggccgtcagagaaatgcaaatcaaaaccacaatgagataccatctcacaccagttagaatggcgatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattgctatatatatatatatatacccaaaggactataaatcatgctgctataaagacatatgcacaggtatgtttattgcagcactattcacaatagcaaagacttggaaccaacccaaatgtccaacaatgatagactggattaagaaaatgtggcacatatacaccatggaatactatgcagccataaaaaagatgagttcatgtcttttgtagggacatggatgaaattggaaatcatcattctcagtaaactatcgcaaggacaaaaaaccaaacactgcatgttctcactcatagatgggaattgaacaatgagaacacatggacacaggaaggggaacatcacactctggggactgttgtggggtcgggggaggggggagggatagcattaggagatatacctaatggtaaatgacgagttaatgggtgtagcacaccagcatggcacatgtatacatatgtaacaaacctgcacattgtgcacatgtaccctaaaacttaaagtataataataataaaaaaaggctacctaaaaaaaaaaaaaagaacacttctcctcccaacaccatatcaccacatcaaccaggactccagtgtaatagcagtgaattctaactgaaagaggtgaaagacactgattgtatttaagaaagatcttctaaggaaatccaaaaatagtaggggagatcaaaacaaagatactagaggaaattgaatatgtgacacctatagctacaaaaaaattaaacataacatagccctaaccatataaacataaaacctcacacaaagacctattatctgagattctgttgcctgatacattgcgttttatttcaataaaaaaattagagggtatgttaaaaagcaggaaaagttagtctaaagagacaaattgagcctcagaagtaggctcagatatggcagagatttggcaattatacctagagtttaatataaatgattaatataataagtgttctaacagaaaaaggcaacatgcaagaacggatgggtaatgtgatcagcaagaaggaaactctaagaaagaagtcaaaaggaaatgctaggaataaaaacctacaagaaataaagaatgcctgtgatgggttcctcagtagactggacaaggtcaaagaatcagtggatttgaaaatatgtcaacaaaaactgccccactgaaatacaaaagaaaaatagaattttaaaaacgtaacacaatctccaaaacagtgggacaattacaaaagatgtaatgtgcctaatgcaaatgacagtaggagtataaagggagaaaggaatagaaaatctgaagtaataatggcagagagttttccaaaattaatgctaaaccacagatacagcaagcccagagaacaacaaggaggaaatttagtaaagcgtctgcaaccaagtatgtcatattcagactgacaaaaccaaaggtgaagagaaaatattgaaagaagacaaagaggaaaataaatatcaagaaaatacatacgaaatacatcatacatacataagaaatacatcagaccatacaagcaagaagagaatggagtgaaatgtttaaaatgttgaaaaaaaaactatcaatttgcaattctgtatccagtgagattatctttcaaaagtgaagagggaaatggcagagaagtcatctccaagacctatggtttcccttcacagaaacactgaaaaatatgaacaaaagtggtcagaattaactttctaagaattctataaaatggtaaaatgtttacaccagtaaagcaaatgctgaattgagaaggcaacttaaaaaggtgaagaaaacttcgtattatttttatgtgtccttgccccacgtccttccctaccttagtcttgaagatggcagcccacatttctactgtggggctctggtttctgtttcctggttcaagagggagaataacagaccttacttttagtcattattatttccttctttctgatttccttgggtttattttgctcttctttctactttattgaaatgagaactaagattatgatttgagacatttttctaatgtaagcatttagtgctataaatttccatctcaacactgctttagtcacatcccacaaatttttatatgttgtaatttcactttcatttagttctatttttaaattttttctttttatacttcctctgactcacagattacttagaattgtgttgttcagttttcaaggatattgtagattttcctgtttctctgttgtctaatagttctgttccattttgtacagatagctcacgctgtatgatttcaattttttaaaaaattgtgctttgttttatggcccagatatggtctgtgctgtgaatattccatgttattataaagtatgcctgttatattattatatatatataatatatataattataaagcatgcccgttttgtattgttagcagagtattctagaaatgtcaatgagatcttgttggttgatggtgcttttcagttctatatctttgctaattttttttttttttgcttagtagctatatgagattctgagagaggaaattgaagtctccaaccataattgtggatttgtctatttctcctatcagttctatcagtttgtgcatcacatatttgaggctctgttgtttggtgcatacacaagtggaatcattgtgccctcttggtggcttattttatgattatatagtgcctatctttgtggtatttttatttgctcttaaatctactttgtgttatattcatatacccattcttttttaaaaaaaattgtttgcgtgatacatcttttccattcttttaatctcagcctatctgtgccattgaatttgaagtgagttttcatatagagaacatattattgaatcatcattttaaaaattccttttgccaatcttttttatactgaggtaaaattgacataaaatttatcattttaaagtgtacaatacagtggcatttggtaatacacatgttatgcaacgttaactctacctggctcctaaatgttttcatcatccccaaaaggaaacttcatactcattaagcagttaattcccattccttctctcggccactggcatccgcaaacctacttttctgtctctatgaatttacctattatggatattttgtataaattgaattatacaataagtgacctttatgtttggcttcgttcgcttcgcatactatttttcgatattcaaccatgttgtagtatgtatcagttttatttgaataactcaattctttttgttgtatagctaaaagttgattcctaggtcataatgataattctatgtttagtttattgaatagctgccaaagtttttccacagtggctctgtcattttaaaatcccactagcaatggatgagagttccaatatctccacatccttaccaatattgttattttatatttttataattataattttcctagtgaatacgcaatggtatctcattgtgtttttggtttgcctttccctaatgactaatgatgttgagcatcttacaatgtacttgttaactatttgtgttctttagagaaatgtctattcaagtgccttgtccattttaaaaatcgagttgtcttgttgacttatgagttctttaatacagtaaacgcttatcagatatgatttataagtattttaacccattctgaaggtcaccttttcacttttgtggtagaccattatgcacaaaggttttaattttgataaatccaatttatccgcttttgttgttgtttttgttgttcgtgcttttgcaaaacctagtgtcatgaggttttctcattatctttggagaattttatagtttgggtctatacatgtagattattgatctaatttccattaatttgtgtgtatgctacgaggtaggggtccaaattcaatttttgcattgaattgaaaattcatattttcagtttcaaatttcaactgcatattcagttgttgcagcacaatttgttgaagagatcattctttaccacagggaatgatctgggacccttgtcaaaaatcaattgatcatagatgtatgggattatttcagactttagatcttgttccatgaatatgcctatttttatgccagcactgcagtattttcattactgtagctttatcataaattttgaaatcaggaagtatgtatcctccaagtgtgattttcatttgctagagtgttttgactatttggggtctttgcaattccatatgaatttcagaattggcttttcatttaaaaaaatggtagttgagattttcataggaattatattgaatctacagatcactttgggtagtattgccatcttaacaatattgtattccaatccataaacacggatgtattgccattcatatctttttttcttttctttcggcaacatttagtatatgacacttgtaactccttggttaaatttatacctaagcattttatcctttctgatgctgtttaaatggaattattttattaatttctcttttagatggcttgttgcaggtgtatagaaatagaactgattattgtgcttttattgaattatctgaaactttgctgcatttattaactctagtaggttttttttttctttaaagttgtctatatatcttgctctgtgaatagataattttacttcttgatctccaatatggatgcctttctttctttttcttacctaattgctgtagctagaattttcagtataatgtatgatagaagttgttcacaactatccttgtcttctttctgatcctaggggtatagctttcagtctttcaccattaagcacaatgttagctgtggggttttcttagatgccatttaatatattaaggaagtgctctgctatttctaattggttgagtatttttattataaaatggtgttagattttatgactttatgtactgcataattgagattatcatgtggtcttttcattcgattaatgtggtatattttatgattttcatatgttgatccacctttatattcttggggcaaatcccactgtgtacacgggttttttagggtctcctaattttcttcattctttttcttttctctccctgagactgaataacgttaactgacctatcttcaagtttactattttttcttctgctgttcaaatctgcttgaacctatagagtgaatttttcattttacttattgaacttttcagctccaaaatttctctttggctactttgtataatatctatctctctatagatattctctatttggaaagacatttttctcctggttttctttacttatttgtattttttaaagtctttaagcatatttgagacagttgatttaagtatttgtgtactaagtccattgcctaagcttttgcatagagattttatattaatttcttttttttctgtgaacaggtcttttttttttttttttttttttttttttttttagaaaactggacattttgagtagtataaacgtggctattttagaaataattttcctccctccttaggttttgtttccacttgttgcatgttgctgttgtttgctcattagtgacttttctaaagtattttgaaaagtctgtgttcttgatcatgtgggtccattgaattctgtattctgttaatttcatagtcagctagtgttctgaaagttcccttaagtgcatagagccaataaaagaaaaagaaaagaaacacagaaaaagaaaagaggaaaaaaggagggaaagagatttaaaaaaataatgtcggttgggcatggtggctcatgcctgtaatcccagcacttttgggaggccgaggcaggcaaatcacttaaggttgaccatcctggccaacacggagaaaccttgtctctactgaaaatataaaattagccgggcatggtggcacatgcctgtaatcccagctactcgggaggctgagaggtaggagaatcacttgaactcgggaggtggaggttgcagtgaactgagatcacaccactgcactccagcctgggcaacaagaatgaaactccatctcaaaaaaaaataataataataatgcctttgccgatttgctctgtgtttgggccctcattcaatgcctagtcaggccatttacaactctctcttaactttcactacctgcttgtgtactgactgaaggccacctataggtgaaagcttaacatcatctcagatctttttggaccatgaatcctaccctgggtatgcacatgatcttcttaatttcccagtagatgcaagagttttagtgttttaaaagtccttattccctcatctatcttcttttctgacctttttcagtctgcttattgttcatctgaactgacatcctttgccccaagcggctgtggcaaaaacttttacctttaaatgctttcaccacaagccacttgggaagctgccccagacctgggactgctctgaccctgatgaaacaaagacaagaccttgtacagccaggcagccataagacaagtccatacccaaaccacagttctttcagaataaggtctatattggattctctggccctagtaaccagcatgagtctgggcttgccatcttcatggccacttgtcttagtttgctagggctgccataacaaaatatgagagactgagtggcttaaacaagagaaatttattttttcacaattctgaagaccagaagtccattactctgaaatctctctccttggcttgcagatactgccttcttgccgtgtcctcacacagccttttctctgtgtacacatccctggtgttttttaagcctgtccaaattttctgttcttctgaggacaccaatcagattggattagcgaccacccatatgatcattttaccttaagtacctcttcaaaggtatcagagtagtatatttgctacagttgatgaacctgcatacaacatcatcacttgaggtctgcagtttacattagagttcatcgttagtgttatatattctagggtttgttttgttttgagacagaacagagtcttgctgtgttacccaggctggagcgcagtggtacaatctcattgctacctctgcttcccagattcaagcaattctcatacctcagcctcctaagtagctggaactacaggcgcacaccaccatgcccagctaattttcgtatttttagtggagatggggtttcaccatgttggccaggcaggtcttgaactccttgcctcaagtgatctgcccgcctcagcctcccccagtgttgggataacaggcatgagtcaccatgcctggccttattctatgggttttgaaatgtataatgacatgtatccatcgttgtagtattagatagaatagcttcattaccctaaaagtcttctttgcactgggttgagttttaaaagctctttgattattttatgacagttccttattagatatatcttttgcaagtatttttatcagtctgtggttatcttgtcttttacagagcagtaatttttaattttaataaaatccaatttgtcaattacttatctcatatgactctggtgttacatctaaaatgttaccaccatactcaaggtcacctaggttttctctaggaattttatggttttgcattttacatttggtgtatgacccatttgaagttaatttttgtgaggttgtaaggtctgtgcctagattcattttttttttttttggcatgttacttcagtattcataagaaacattgttctgtaatcttctttcttatagtatcttagtcttgctttagtttttgggcaatgctggcctcactgaataaattcaaagtgttccctcctcttcaattatttggaaaagtttgagaaagactattgttaactgttttctaaatttttggtggaatttaccagtgaaccaactggtcctaggcttttctccagtaggtggttttgattatgctttcaatctctttacaagttacacatctatacagacttttataattcagtcttggtaggttgtgcgtatttaggaatctgaccacttcatctaagttatccaattagttggcatgcaattattcgtagttctctgaataatcatttttatttccacaaaattggtaatatcccagtttccattttttatttcattgaatcttcttttttcttagctaatctagctaaatgtttgccaattttgttgatcttttggaagaaccaacttttgatttattaattttctctactctttttctgttctttatattatttatttccacactaatctttactattttcttccttctgttggcctttaattttttttttttaatttttaaggtgtaaatttaggttgagaaattttttaaatgaaagcatttagagctataaattttccttctggtgttcctttcactacctgccataaattttgatatgttgagtttttgtttgtcttggagtattttctaatttgtcttctaatttcttccttgacctattggttatttaaatgtatttaatttttgcatattgtggatttcccagttttccttctgttattgatttctagttttattccattgtgatcacagaagatattttgtataatcgcagtctattaacatttattaagtcttgtggcctaacagaggatctatgttggagaatgttccaagtgcaattgagaatactattctggtgctattaggtgaagtattctctatatgtctgttaagtccaattcatctatagtgttgacgtttcctgttccttactgattttctgacttattattctatccattattaaaagtggagtggtaaagtctctattattgtagaactctctgtttttcaagtctatcaatatctgtttcatatattttggagctctgtttgctgcatatgtgtttacaattgttatatcttcttggcaaattgaccagtttcatcaacataaaatataattcttattgtcttctaacagtttatttttctttttacataaagcctattttatctgatcttagattccctcacactccaccccagcacgcttttggttacatttacataatatatcttttccatcctttcattttcaacctgtttgtgtctttagatctaaagtgaatgtcttacagacagcataagctatgtcattaaaaaaatccattctgcttatctctgccttttgactggggagtttaatccatttgcatttaaagtaatcactgatcattaaatactttcagtattttgttgttttatgtatgtcttataactcttttgctcttcatttccttcaatattgcctttgtgtttagcttatctttttgtgtcacactttgatccccttctcatttcttttttatattttctttgtggttaccaggaggacaatgtatcaacttttaaagttattacaattttatttttttaaatctctcccattcggggattttaggaaggttaaataataatgtaaatgagatacctagaacaatataagcattcaggaattattaactcaattccaatccttcctccacctccacctctttctctgtgagattatagaaaagatgacaaaaaggatgttttctgagccctttaattgttgagaatgatctttgagaaaaagaaaaaaaatgaaagcactaggaatgtacaacagcctggaagtataattaagtgtaaattaaatagataaaagttataagcagaggaaagtatagtagaactcagtatttaaaagagaatcaatgtgaaaattatataaatttatgtaaaataaaactaccagacaaatctgatatccttaggatttttctttctttcatgtgatttctaattgctacatatgacactaaaccattgatctgagctgtaagagaaactggaaattgttctgttatcttttgtaagatttctagaacattttgccctcagacttaaatgccaacgtatttctcacttattgtttactgcttttggatttacatatgatttgattctttcttatctcttatccttacaatgtaattcaaactgatgccaatttaagttcaattgcgtacaaaaactctactcctatgcagctccgccccatctaatttacattattgatgtcgcaaattccatctttgtacatagtttacatattaacatggatttatacatttttatgtatttggtttttaaatcctgtagaaaataaaaagtcaacacaccaatattaaaataatactggtttttatatttgtccatgtgcttacctttatcagtgttctttacatctttatacgggtttgagttactgtcttgtgtcctttagttccaacctaaagaactccctttagcatttttatagggcaggtctagtggtaatgaactctctgagtttttatttagggatgtcttaatttctagctcctgtttgaagtaaatttttctggatatacaattctctgttgattgatttttgttcattttcccttcagctcttttaaatacattatcccactttcttctgccttccaaggtttctattaacaaaattcagcttataatcttattaaagatctcatgtacatgagtggcttctctcttgctgttttcaagattctgtgactttggtttctgatagtttaaatataatgtgtcttattgtgggtctctttggatttatcctagagtttcttggtcttcttacgttggtatatccatgtatttcaagacatttgagtagttttcagccattatttcttcaaacaatctctcctctttggggacttccattttacctatattggttcttttgatggtgtggcaccagtcccctagactttgttcacttttttccagtcttttatttctgcttctcagactcaacagcttcaagtgttctgtattcaagtctgctgactctttcttcttccagctcaaatctgctgttggatccccccttgtaaaatttttaattccattttagtgtttttcaagttaagtatttttattaggttcctttttataatttctttttgttgatattctcattttattacacataatttgtctgatttccattagtttttttctttgttttcctttagctctttgaaaatatttaagacattttaaaagtctttatccaagttcaatttctatggttctgtagagatattttctgccagtttatgttcttcttttccatgggccatgttttcctgtttctttgtatactttctaatttttggttgaaaactgagcatttgaaaatagagccaactttcccagtttctgcagagagtctttatgccacagtattcgttcactgattaactgggtatatctaagcttagggagcagctgagtcaaaagtttaaggtcttctcaggtcttttctgagtatacatgtttcctatgcctgtgtgaaatgttctcaatttccctatataaacagctacttcttcttttttttttttttttgagatggagtctcgctctgtcacccaggctggagtgcagtcatctcagctcactgcatcctccacctcccaggttcaaacaattctcctatacaggtgtgtgccaccacgtctggccaatttttgtatttttagtagagacagggtttcgccgtgttggccaggctggtctcaatctcctgacctcaggaggattacaggcttgagccacagtgtccagcctaaacagctacttttgaatgctttaatttcctgaatagtctcaacccagtttttccttgaggtcttaggtggtccattgtatgtctccacccatagttgcttgccccaggcatctgtgggtctgtggtaccactgcagctttcaccacctgtagctgccacctttccctatctgagatccaggttaggtgagagagatcattccttcacgcagtcccatgacaggttggaacatttcaaataaggtctgttctgctcctctggttgaagggagaaaattgggaaccggtttcccaccttctacaaaccaagatctcatgttgccacgggagtggcagggcaagtgcaagtgaaaatgccatacaattttctaccattttgaacgcgggtttttcttcaatggtcatttgcttggttgctgtaggcctttcactgttttccagagctcccataagattactttagccagttttttgttctttcctgatgcttccctggcagagtaagggttggaacttccaccattttgctgattcataactctgtagtcagttttaaatatattgatacttgagtttgttttatggcccagaatatggtcttggtaaatgtttcacatatactcagaaagaatgtgtattctgatgttgttacatgggctgttctataaatgttacttaaggtggttaataatgttgctcaagtcttctatattcttgctgattttcttttatttatttattttattttttttattatacttttaagttctagggtacatgtgcacaacatgcaggtttgttatatatgtatacatgtgccatgttggtgtgctgcatccattaactcatcatttacattaggtatatctcctaatgctgtccctccctgctccccccaccccatgacaggccccagtgtgtgatgttcccctttcctgtgtccaagcgttctcattgttcaattcccacctatgagtgagaacttgcggtgtttgtttttttgtccttgtgatagtttgctgagaatgatggtttccagcttcatccatgtccctacaaaggacgtgaactcatccttttttatgactgcatagtattccatggtgtatatgtgctacattttcttaatccagtctatcattgatggacatttgggttggttccaagtctttgctattgtgaacagtgctgcaatgaacatacgtgtgcatgtgtctttatagcagcatgatttataatcctttgagtatatactcagtaatgggatgggtgggtcaaatggtatttctagttctagatcttgaggaatcaccacactgtcttccacaatggttgaactagtttacagtcccaccaacagtgtaaaagtgttcctatttcttcacatcctctccagcacctgttgtttcctgactttttaatgatcgccattctaactggtgtgagatgggatctcattgtggttttgatctgcatttttctgatggccagtgatgatgagcattttttcatgtgtctttggctgcataaatgtcttcttttgagaagtgtctgttcatatcctttgcccactttttgatggggttgttttgttcttgtatatttgtttgagttctttgtagattctggatattagccctttgtcagatgaggagattgcaaaaattttctcccattctgtaggttggctgttcactctgatggtagtttcttttgctgtgcagaagctctttagtttaatgagaccccatttgtcaattttggcttttgttgccattgcttttggtgttttagacatgaagtcctcgcccatgcctatgtcctgaatggtattgcctaggttttcttctagggttttttatggttttaggtctaacatttaagtctttaatccatcttgaattaatttttgtataaggtgtaaggaagggatccagtttcagctttttacatatggctagccagttttcccagcaccatttattaaatagggaatcctttccccatttcttgtttttgtcaggtttgtcaaagatcagatggttgtagatgtgtggtattatttctgagggctctgttctgttccattggtttatatctgttttggtaccagtaccatgctgttttggttactgtagcctcgtagtatagtttgaagtcaggtagtatgatgcctccagatttgtccttttggcttaggattgtcttggcaatacaggctcttttttggttccatatgaattttaaagtagttttttccaattctgtgaaggaagtcattggtaacttaatggggatggcattgaatctataaattaccttgggcagtatggccattttcacgatactgattcttcctatccatgagcacggaatgttcttccatttgtttgtgtcctcttctatttcgttgagcagtggtttgtatttctgcttgaagaggtccttcacgtcccttgtaagttggattcctaggtattttgttctctttgacgcaactgtgaatgggagttcactcatgatttggctctctgttagtctgttactggtgtataagaatgcttgtgatttttgcacattgattttgtatcctgagactttgctgaagttgcttatcagcttaaggagattttgggctgagacgatggggttttctaaatatacaatcatgtcatctgcaaacagggacaatttgacttcctcttttcctaattgaataccctttatttctttctcctgcctgattgccctggccagaacttccaacactatgttgaataggagcggtgagagagggcattcctgtcttgtgccagttttcaaagggaatgcttccagtttttgcccattcagtatgacattggctgtgggtttgtcataaatagctcttattattttgagatatgtcccatcaatacctaatttattgagagtttttagcatgaagggctgctgaattttgtcgaaggccttttctgcatctattgagataaacatatggtttttgtctttggttctgtttatatgatggattatgtttattgatttgtgtatgttgaaccagccttgcaacccagggatgaagcccacttgatcatggtggataagctttttgatgtgctgctggattcagtttgccagtattttactgaggatttttgcatcaatgttcatcagggaaattggtctaaaattctctttttttgttgtgtctctgccaggctttggtatcaggatgatgctggcctcataaaatgagttagggaggattctctctttttcctatttactgtacatttattccaccagtgactaaaacaggtgtatcaataaaatctgttccctcaggttttgcttcaggtattttgagggtctgttatcaggtgcataaacaagattgttatgtcctattcttaaattaatctccttataattatgaagttaattttttttttcttgagatgcagttttgctctgtcgcccaggctggagtgcagtggcacaatctcggctcagtgcaacctctgcctcctgggttcaagcatttctttgcctcagcctcccgagtagctggggttacaggtacctgccaccacgcccggctaatttttttgtatttttagtagagatggggtttcaccatcttggccaagctggtcttgaactcctgaactcttgatccacccaccttggcctcccaaggtgctgggattacaggtgtgagccactgcgcctggacctggcccgaagtaaacttctttacccttgctaatgatctttgctctgaagcatgctttgctggtattaatatagtcattccttctttctttgattcatgtttgcagggtatatctgtttccattcttttacttttaacctattgtctttatatttaaagtgcatttcttgtaagtataattggtttcttaaaatccaattatctgccttttaaatgtcatttttatatgatttgcataaatatgattattattacagctaaattgaaatctgtcatcttgctatttggtttctatttatcccatttttttcccctctttttttgctttccttgagattgaacattgtattagttttctagggttgctgtaagaaagtgacataaagtggatggcttaaaacaacagaaatttattgtttcagtttggaggctagtcatctgaaaccaaggtgtcatcagggccgtattctctctgaaacctgtagggaagaattctttcctgcctgttctagcttctagcattttccagcaattcttggcattcctttgcttgtagatgtatccctccaatctctgcctctatcataacatagccatcttctccctttatctgtctattcttctcatcttataagaacattaattactgaattggggcccagcatagattaggcctaatctcatcttgaataggttacatctgccaaagattcttcttccaaataagatcacttttacagcttttacaggtactgagagttagaacctcaatatatcctttggtggggaccgacctcttacccataaaaagtattttatatgattccattttatctcctttttaggttattaactacaatttttttttcttttttttgagatggagttttgctcttgtagcccaggctggagcgcgattttggctcactgaaacctctgcctcccgggttcaagtgattgtcctgcctcagcctcctgagtagctgggattacaggcatgtgccaccgtgcctggccaatttttgtatttttagtagaaacagggtttcaccatgttggctagggtgggtctcaaattcctgaccttaggtgatccacccacctcggcctcccaaagtgctgggattacaggcgagagccaccacgcctggctttataatttttttttaattcagtggatgttttagggtttatagtatacatctttatcacagtctagctccaagtgatatatccctttatgtatagtacatgacccttacagtagtgcatttccatttttcctctctggcatttaggctattgcacacacatacactcaattcatccctttgtgtagatccgtatttccagctgctatcttttgcttctgtctgaaatatatgtatgatttcttttatgactattttgagaaatttggttatgtgcatttgtctatttttcttatgtacttttagctcatcaatcttaagtctgtgagtttatagtttttaaaaacaaatttgaaattatttggctattatttcctcaaatatttttttctgccgcccctgctttccctttccttagggatctctgatttccacctatattactctgattgaagttgttccacgtctcttttgaaaatctctgaaaaatcttttatcatttggataatctgtatttgtacatcttcaagtacattaatattttcttttgcaatgtttaatctgctgttaatcccatgtagtggattcttcatctcaggtattgctgttttaatctctagaaattccattgaagtcttacttgatgaaaaaggcagataaaaacaaatgttggcaaagatatggagaaatcagaatctgcacacactgatgatgggaatgtaaaatggtcaaggcaatttggaaagcagtctggcagtttctcaaaaggctaaacgtagttcccatatgacagcaatcattcatctaggtatatactccagagaaataaaaacatatccacaccagaacttgaacattaattttcatagcaacattattcctaggggtttaaaattttttacattatttttatttaaaatagagacagggtctcactacgttgcataggctggtctggaactcctggaattaagcattcctcctgcctttgtcctgttttctccactggaaaagaataaaactttgtacacttggactaacaactcccgattccctcctttaccaacatgccccacagcctctagtaacttactctctactttcatgaattcaacttttttaagattccacatataagtgagatcatacaatatttgtctttctgtgcctggcttatttctcttagcataatgtcctccagattcatacatgttgtcttaaatgacaggatttaccttcctttaaaggctgactagtacttcattgtgtatatgtatcacattttctttatccacttatctgttgatgggcacttaaattgtttcaatgtcttggctactgtaagtaatgcttcaataaacatgggaatgaagatatcccttcaacatattgatttctgttcttttggataatactcagaagtgagattactggatcatatggttgttctatttttttcagaaacctccatactgtttttcatagcggctgtactaatttacattcccactaacaatgcatgagttcacttttctggacatcctccccaacacttgttatctttcatctttttcataaaagccattatataataggtgtgaagtgacatctcactgtggttttgatttgcattactctaataattagtgtgagcattttttttttttcatgtaccgaatgtcttttgagaaaggtctcttcattcctttgcccattttaaaatcaggtggttttcttgctcttgagttgtttgagttccttatgtattttagatatttacccatttccagatatatcatttatattttttcctattctttgagttccctcttcactgtgttgtttccattgctgtgcaggtcttttattttgatgccaccccatttgtctatttttgccatgcttttgcagtcatatccaaaaaaatcattcccaagaccaatgctgtggagatttccccctatgttttcttcagtaggtgtacagttttaggtcttatatgttaagttttaaatctatttttttatatggtgtaaataagggtctaatttaattcttttgcatgtggatatccagttttcccaacaccatttattgaagaccctgtccttttatacttttcagtatgcagatcttttacctccttaaatttacacctcagtatttaatatttgttgctattatgagattttcataatttccttttcagatagctcattaatagtagatggaaacactactgatttctgtaagatgattttgtattacggaactttactgagtttgtgtatcagttctaccaggttttagttttgttctggtggagacattacagttttttgtatatggttatgtcatcagtaattacagatgatttaacctattcctttcctattaggatgccttttttttctttctcttgtccaactgctctggttaggacttctagtactatgtcaaaaagtgatgagggtcgtacatggcctccatacctaatctgttgagagtttttaccatgaaaccaggttgaattttgtcaaatgctttttctgcatccattgagatgatcatatgatttgatttacaccctccattttgttatgtggtatatcacactttttgatgtgcatatgttgaaccacccttgcatcctaaggataaatcccacttcatcatggtgaatcattctttgtattcgtgaatccagtttgctaatatattgttgaggatttttgcatccatgttcatcagggatattactttgtaagtttctgtccttaaagtgtctttctctggctttaataacagtgtaacactacccttgtaaaatgaatttagaagtattccctctgcttcattgttttggaaaagtttgagaatttttattagttctttaaatgtctggtaaaattcagtagtgaagctgcctaatcctgggctttcctttggtgggatactttttattactggctcaatctcttttcttgttattggcttattcagatttgtttcttcatgattcactctttgtaggttgtatatgtctaggaatttattcatttctttaggtcatccaatttgatggtgcataacttcatagtagtttcttataatcctttgtattttggtgatatcagtagtaaatgtctcctctttcatttctgatcttatttgagtactctttttttctcctagtctaggtaagaatttgttgattttatctttcaaaaaaaaaaaaaaccaactcttagcaactcttagttttgttcatttttttccagtctttatttcaactgtgatctttgttacttacttctttatgctaactttcgggcttagtctgttcttttcctagttcctttaggtgaaaagtgagattgtgatccttcttctttattggcgtaggtttgtatcgctataaatttccattaggactgattttgctgcatcacataagttttgtttccattttcatttgtctcaaggtaattttttatttactttttgacttcttctgtgaactattagttgtttgggagcatattgtttaatttccacatattgctgtattttccaccagaattgattcttgttcttgatttctagtttcacgccattgtaatcagaaaagggatttgatatgatttctgtccacttaaacttaagattagttttgtggactaacatatatcctggagaatgttccatgggcatttgagaacaaaatgtattttgctgcttctggatggaatgtttcatatatgcctgttaagtccgtttggtctaaagtgtaattgaaatccattgtttctttattgattttctgtctaggtgatcaatctgcccatggtgaaaagtagagtattgaggtcccgtattatagtattgcagcctatctccctcttcacatcatttaaaaattgctttatgtatttaggtgggtcaatgttgggtgcatatacttttacaattgttatgtcttcttggtgaattaatccctttatcattatataacaaacttctttctttttatagtattgacttaaagtctattttgtctgatagaagtatagctacccctgctctcaatttccatttatatagaatatctttttccatcccttcactttcagtctatgtgtatgtttagtagaaaagtgaatctcttgccgggcgcagtggctcactcctgtaatcccagcactttgggaggccaaggcggcgggatcaccagaggttgggagttagagaccagcctgaccaacatggagaaaacctgtctctattaaaaatacaaaattagccaggcgtggttgtgggcccttgtaatcccagctactcgagaggctgaggcaggagaattgcttaaacccgggaggtggaggttgcggtgagctgagatcatgccattgcactccagccttgacaatagcaaaactccgtctcaaaaaaagaaaaagaaaaagaaaaaaaagtgagtctcttataggcagtatgtggttgtgacttaaaaaaaaaaaaaaatccattctgtcattctatgtctttttgttggagaatctaatccgtttacattcaaggtaactattggtaagaaactgctagtgtcattttgtaatttgttttctgattgttttgtaggtcccttgtttcttttttccctattgctaccttcctttgtggtttggtggttttctgtggtggtgtgctttgaatcctttcttttatagtatatgcgattaccattgtatttgctgtaaggctaacttaaaacattttatccttaggctactttaagcgataaaaacttgcctttagttgcatacaaaaactctacgctttcacaacccccccgcctttcgtgattttgatgtcaaagtttacacttttttaaatttgtatctcttattgtagctacagttacaattacttcttgtagctacaacttattgtagctacagttgtttttaatagtttcatcttttaaacctcctaataggaataacattgctttacctgccacctttacaatactagagaattctgactatgaattacttataccatttagttttttacttttatgtttctcattaactagcagtcttttatttaagcctaaagaactcccttttagtaattccagtagagcaggcctagtagtgacaaactcccttaggcattgtttatctggaaaagtgtttatttctccctttgccaagtaaagtattcttaattaacagctttgtttccttcagcactttgaatataccatcccactctctactggcctgtaaggcttttgctgataaagccactgaatctgtattggggctaccttgaatgtgatgtttcttatctttgctgctttcagtattctttgtctttgatttttgataacttggttgtgatgtgtcttggtgaactctttaggttgaatctgatcggtgacctcagcttcctgttcctgaattttgtcatcttttctcagatttgggaatttttcagctattacttccttaaatatgctttctaggcctttttctttcttttctccttaaggacctcctattatgcaaaagttagctagcttgatgtgtcctgtaattcttataggcattcttttttgtttttgtttctcattggataatttcaaatgtcttacctttgagctcattgattcttctgcttgatcaagtctgctgttgaagcgttctacttagattttcagttcagttactgtattctttatctttagaatttctattgtttttgtttatatttgttaaacttctcattctgttcagatgttgttttccaaatttcatttttctatccatattttcttgtactttgttgaacttaagaggattagtctaaattatttgtcatttcacaggtctccatttcttctgagtctgttactggagctttcatatccaatgtacaccagaaactttgctaattccacagtaaatgttagaaatgggtttttattgataaaatcagtggcttgctaattagtatgttagaagctggcttgcattggtgaagttgagggtattcgatatacgtatcccggctaaaatacttctaaagaacctctccagctttgcaagaatagagtataggaatctgaggaggcctttaacatggccctcagaaaggacacacagggcttgacctctaaacatataaacatatacagaaacgaggtcagaacatgttttgactgatgccagtagtaacacagcagagcctattgctggtcactgttaccaataccttgtgtaataaacctttcatttaaattgagaggttgtaagccccgaaaaagcaaagggcttacagtacctcttgcctgaacactcctgtgtgtgctttctaagaaatgcttttaaaataatgcctgtggcacaaattgatttgacactggctctctttataagctgctatcaaggttgtggggaatagctaactcttacccactttccctgacaaataggaacccgtagttacctaccaaaagtatgtaaaagctatatccgacccaaaggccagttaggtcaaaacaccttgaaaggagctcagtttaaggaaatcagtggtgtcacagtgccccactagcgtgtaaaagttttcatagtttgactatcggatgtgcttagtttcacagctgagtcttaccttgtgatacttttagagcaaaagtcaaatcaaagatgatttaaaaaaacatttcaaagattcataaagaataaatgcctcctcaggaagtcttctggagttctgcccacttcccttaggtggctctttactgtgcccatctcatagcctgtctccttccacttggtgtattgcagagtaaagctcactgtttacaggggtggtagaaagtgtggtcctttcccagactcctttttcccttcctcttctcatttttcagaagatgtgtttgataataaacgaaacaaaatgactaacacattgagctgagctacataagcagatgtcagtttgacgtgaagagtttaaaagatctatgcattatctggggactcctcccccagacctgaaggatcaggtgctgccttctatgccacctgtgcagacagcaaaagaggaaaaccatacccacgttcagtatgaacaaaggggacatttgaactctgtgtggacccctcattggaagggtgtttcttctcctgctgcatccacaaagagcactccttagccttgccttttgtcagttctctctccaataaggcttgagcagagacaacccagtgcagttcagagagactgaagtctggtgttccaggtctgagtcctagctctagctctctgtgtaactttgggatgtcccaaagtaacttttcacaacttgataggtgttaacttgaattttggatacaggtgactcttagcccatcccctctctgtgcttcagatatgtcatcacttgggccatatgacctctggacacctttcctactttccacaatttcagagcagcagagcagactggagctcctgctgcctctgagcttcagtgaattatcactcgttggagggaagcttcaagcattttgttatctttcaagagcaaacacagtgtctgtcagcaagaatatgtagcagatgctagtgaacagcagtgattagggttgaatgctggatttaaatatggagcttaggctgtgaaggaagcctgaagaacctagagccccatgaagctgccctctgtgagatctgacaccatttcccactgatgagccatgggtctaatgggtagcactgattccacatacagtatgtgagtgcaatacagtgaaagcaaagagaataaaatgatggctaacatgatgttccaaactttaaacaggagaaaaacacacaattccattatgtataagaacccacacagagatcaggagaataacctcattggagaatgaatgacctgtgtggggaatttagggtagagttgagattgaaaaaatgggccgaagtcaggtggccaagggccttaataccttgtatacaagatgtgtagtcaaggaagaccatgccttacttatgcatcaattcccttgggcctacaaagagctgcctagcctgggactgttgtagagaaaagctacagtgttccaatgacacagggactcctccatgtatgtatgagtgcccagctggctctgtaataaatcttatttttatttattaacttttcttgcgcattggcttgatgcatcagttggaagtcagaggccaaacgaagtgaacactgagccaaaggaagttctcagcctttagggaggcagaattcactttaaacacaataaacaaatgaactcacattatacaggagagagtcagaagatcccagtggctggtgtcatcgggccatatttgcccgaagtgcctattccttataggaacccactcccagggttgatgggctacatccttaggaggctttatgcctatgttctcctgaccactggctcctccagggctggccttttttagtctctctgtagaggttcctgtagctggttggatataggctttcacagaagggtcagtgccttgggtctggttacaggactgttaatcttgctttgttaagagtaatgttatttccccatttccaaattctccagggagatggaatgtcaaagatagtatgactgtagcacctaaatcctgggttccagaagcagagaagagaatacactgaagctgtagaaaggcctattagtccatgtcagagattaactggatgcgaggaccatttctgggatggtgtatacagaactggagaactggatagggagtcagaaacaaagagctgaagatgatgcctttgaagtttccaacgtggataactaggtcaacagaatattactcaagaaagtattaacataggattgagatgcagtcagtagtaatggaattgaaatttcaaagtatatacctcatggatctcagggggtgttgagctgatcacctgggctaacactcctatgaccctggggaaaatcaaatgacctggtactgtagccatggtaggggtgtcatcaccttaatccaactgggacagtgctgtttgatattcatctggaacttggtgcagacccacattttgctgggtttcaccacaaccaaggcttttttgattcttttctcttttaacatcagtacatcactgcaaagttaatcctcatataataggagatgaaactaattgcttataaaaacaagatttttacaacactaaaattgttcaagcatatgggcatatttatagttgcaggcagtgtttcagatgcagactgttcttggctgcagtggttgtttacaggcagcatctgttctgattaaatatttgatgattatccccgaatgttttaaagcatagtactgggctctgctgactgtacaacaaactggcatttttgacctatagggcactgggctaggagatacagttctgagggaagtgaaagatagttaacaactgcacaactgaccctttattagttgcaataaagcaatccaaccacccaacccactgtgatggctttcctactatttaaggttggtggtgtcaaagagacaccctcctgtacagtgtgcagtgaatcaacatcatttccacaaaacctccttcctgcacaaaggaattatcatactttgttacgaagtaaaattttcctgtatcagtcacaggagttcaccagttaagatactgttagttgaagacttctggggtgacttaatgaaatagctcagccatctggtttaaaaactggattcttctatccctccacacagctgtccatgcacctgcatcatctcaaggctggtctccctggtggtagaaaggctgacagtaaaaactggagccacatgattccttgcttaggcagtgtttcttcatactctcacatgagagcaggcatgttctttccctaggctcacagtaaacactctgtcaaatctcacaggcccaaagtgcttttggttatccccattccaagccaatctgtggcatggaagatagcattaccctgactgccttagactaatatacctactccacttctggggctgggaataattttgaggtcaaccatccaaactgcatgacagccattccatggaagaggtatggcctaaatctttggggcaacctgaattcatgaaaactctcttagatttatgtaactatttttagaattcacttctgtatcatttaattttactaataaaaataccacctttaccctaaatgtcagccaagcgtaaggctccgttgggacagaaggaactatcaaagctttgtgtttttatacattagcagcatttgacaaagaaataactctgaaggaaggagaataaccaggcagagtctagatgcatggaaaagaagtctttgagaaggcttcgcaggctgaaggaaagggcaggactacttcaggaatacaacgtttaagtaaaagaggttggggtctgttgatcttgaggagagatgaggatggactggaaaataggagtgagatatagtaggagaggaaaggatatggatgatgtatatactgtatgggtaaccatcagtcgaccctaagaagataatagttgttaaatggttagctatttaatgaaagaaacctgaacaagtaataatcttgagttgcaaagtggctggagtcacacaacagactaaatttctggtagaacaaatccagcagcttatgtgaaggttaccatgtctgaagctggatagaaaaggtctaacttccaaccaaagtcacagttcttgagctcggtacacagagacagactgctaacagctgatgtgtcctcagcgagagtgtcctcatttatatcctcgttctcttctgcctcctttttttgttttaaacttttgggaagtctcatcattcaatacagttctaatatatcacaataacagaggcggcccaatttctacaaattgactaattctatccctgaaaagttcatacaataaaactatacaaagcatcattttcaaccatcctatagaaaaatttccctattaaattttaactctaaatcctctatctctgttaataaccttactaaaaacttccccatcactgcctgccagggagatcaaaagaaaccaaatttaagaaacctccaacacctgtacctgactgaaaagcaaacatacagacctttcagtcctgcccctactattcagctcctatcacagagacatggtggatgtccccttgggaagcggatagctcttaggtggaagctaggcctgcaataagcaggccaggaaacatgtctcccaggccccatactcttggcagaagcacttggccaccagacagcatgtcctgtcaacctacagagttcttaaaaacaaacactgggacccagaatagtaccctgtggtcatagtgcccacagttcactaagcaccctcacaggtctttgacagaacactgactgccaggtcacctggtgggcagagaaatggaagattcccaggcccaactagcatctcagggaagaaccacaagcagagcaactttcagagctggtcggccagcgttggcacccagggaagcaagtgctattccatatttggagagaacataaaactcaaggaaacagagaagtcctactcaataccgttctcaactgaaaacaagagaacttgcaaaaaagaaacccagttttctggagtccatggagaaatatgaagccaatgctcggctaacagagcagaaagccttttataaataatgccagtcaaagctccaaaggtcagagctgatgcatgccggattgttctgacaatttcttaggtttctaggcaacaaggagctggtcaaacagctcacctccaaggacatactttataataccaccctggtagacgagagcgaggcagcagtgaaggcaatagtcaagaacaggagagggaggtaaagaacaagatgctcttcagacagtctggacaaagacagtccctacccagctctcaggagttgcctccttaagaaggtggaggcccaggcagctcccagggtgaccctaaagacagactccaaggaaaagggtgtgagggcaatggtgaatataaggaggtcccctttcagtaggcagagcaagtcagttcaggtcttatttttagagtctctgaacagtgaagagaagctttctgtggacagcattccaccaccatgggaggggaaaggtgccatgagagacttctccagtggggcatacaagcattgtgcagtgatccccaagatccagccactggcaggaaatcgaaaggcaagcttcttaaatacatagtatatggagacagaagtgtggagcactcccaaaatgaaaggccaagacccaggaacaacctccacaacctggagtatatacaaatgaacccagccctgctgactcagatccacaccatcctaaagcaggggtttctcatgaatgaaagggctaagtatttggtggagagaatgagaagtctcccactgcccacacattttttcttcagagatttcttttccaagagccccttgaataaaggaagggagggagcactgaatgccccagaaatcagaatgcatggtgcgggaagacgacaggaaatagttctcaaagagatcagaaaataaattggaagcaatttcattaccacaaagaacagacattttttctaaggcacccctcccctttccacccaattgtcattcagcaactactgaatacttaccaatgaaaaacgttacccctgacctcaaggctgctcctgagctctggtagaaaatgctttccttgtctataaaacatggcaagcaaggcaggatttaacagtaagggcacagtagcactgcaagccttcaaatggaaacctggagacaagcagtgagaacaggaagcaaaagcagggcaggtgaagctaggaccagggcatctggaactttccacacaggttggatctccatgccagacaacagttttcaaggaaaaatatctaagaggaacatgactttgggaaactttttggcagtactgcttactgtatactagagagtaaaagaatttggggaacattcaccaatttgcttcttcaggggcttgggtagggaacgtgaacaggaacctggctctaatttctgaacttttttatcagtaaaaacaatccaacaaacgaaagctagtcagtgagagaactgggagggtctgccctccttccctgagtcaagccttctggggggacctcctgacatttaattaagcaaagacaacgcccactgaaggaagctgacctgaaagtgacacgctactgtgaaatgagcatgaagtgggagcttgttacatatatgaaatggccagcgatcctgagcaaagcgcttcagagcttgagacctaagtcttctcatctatatactgagggctggacaagatgatctgtcaagccatttttatccctaatccaccaaaatccaatgctttagtttattgtcacaaaagcaggtatcgaatggctatcctgcagtgcctccaatcaacattcagactttttccctgaggcaatataagataacagttaacatgtttttatcaattaggtggtcatgagataaatatatatgggaagtggtagtttttcacttaaatgcatataataatggtacagctctctttgaatagtatttgtttatttcttaaatatttaagttcctaaagacggtaagaataacccaaggaagtgaaatcaatgtcacaaagcacatggctaaataactgcaggtttgcagtgccatgtgtgagatcagatgacagaagggagaactacctttaggcagaggcttctcatgtcccctggagtggccatgtgctgttctacatgactacttccacttcggttatgtagaagctatttaaagcacacagatgtttgtgatgagaaaaaagccacccttaattgaataatggaaattataagcatgatttgagggtgggggtggaggtgggagtagagatgggtagaaaggagtgcaatggaaacaaaggagccttcataaaattcaagtcacttcttaggataacgtgattgatttactcaccaccttcttaggaacataaagcaaacaagtgggttttccttttactgcttttctgaaatgagctacactcaagaaagcagcacgggggttgtgctgtccctgcacagtggcaggagagtatgaggagcaggtgaatgccacaacagctccatccaagatcatttttcacatgcaggaaccattcttatactaccctttactggtaatttctgtagaaatctggaagtctggttgacaccctcctgtacagtgtgcagtgaatcaacatcatttcccttggactttgcaatcacggtggcattcatacattcattcaacaagtatgtatgtacaggacagtggagtaaagaaaacagatagttcttactctcacgaggcttaaaatttcaggagggaactaggcagtcatgaagtaaacataaaaacacagattgtaataggcactagagaataatgaagactttggggaacacaatttaaattggaaaaatatctcagttccgttaacacatgttgaaggggaatagcattgttttgctggatttggggcctggatgcaagcatgttgggtatgcattgtagggtaatgcatttccttccatttgggcccaagtgtatatttaccacccagttgtgatgagctgggatcctcctgctcaatctcagcttgaagcacttggaggttatctgcctgctgtgggtgattattttggagcaaggtacttcatttgcctcaagaaacagatttgataccactactgtgcccttttggaacagagaagtaggcaagaccccagtgtgaggcagagtgatgggatctttagggacataattgatgatgtaactgatgatgattttggagtttatacatttccaaagtttcaaaatattttcacttggttgatttatctttatggtgatgacactacgtagatatatgcccttcttaaaagttacagtaagaggctgggagcggtggctcacgcctataatcccagcactttggaaggccgaggtgggcagatcatgaggtcaggagattgagaccattctggctaacacggtgaaactccgtctctactaaaaatacaaaaaaattagccgggcgtggtggcgggcgcctgtagtcccagctactcaggaagctgaggcaggagaatggcgtgaacccgggaggcggagcttgcagtgagctgagattccgccactgcactccagccagggcaatgagcgagactccgtctcaaaaaaaaaaaaaaaaaaaaaaaaaaagttacagtgagagttgacattgagaaaagggaggcccagccagggttatgcaaagacacagtagggaggaggatgggaagtttctgagacaccccagtaactgcatgggtccacaaagcacatgtacagtctgctctatgcactgcaggtacagccacgaataagacaaagtctctgccctcatggagcttggtgtctgtctactggagcagacaaaaacaaacctgctttttctcatttgcctctactgtgggttccgtgtataactctcaaatgctagcatttccatgcattccatccttgtccttctctcctctctgccttttccaggagaattttcctagccacaggttcagctatggtctatgtgctggagtcatacatttttatcttctgtgtacgtttttctctagacccatatttttaattgccttaggacagctctccctggatatccctcagacacaactaaaacatcattcaattgtactattaataattttcccttcaaaatccactcctcttctctatagacctaacagcaacactgtccgtccagctctcaaaacctgaaatgtgggagttgtctttgactcttatctttcccatgcatgagcactgaatcaatcagagtccctagcatgtcttggactgtggcctcccttctatcttcagattatctgcctttttagaattatggcaataacttaactgcaaagaccacatgtgcacttgtgctctttcagtttgtaataaatataaaatgaaaagtgattatgtctttgcttaacgtctgcaaaataaaagtccaaagtccttggtatggtctataaggccctcattatctgacctgcctgccttcccaatctcatcttaatcccttacagcctgacactcagacatactagactttccacctaactcactcacatacacctatcctgtatgtcaagattcggcttgaccttcatcacctacctatctgcagtatttttggatctgaattctctgcccacattgtactttcacatacttcttttacccttagtggtttgtacttatgcctggtctaactagattgtctccctgtaacagacttcttgattcaacaaagcagctctgaatcagcctgaaacctatggcacactgcaaaatggcaaacattcaataggtatttgcccagtaaatgttaatgaaaggaaaaaaattcaaacttcagttggaataggattagagacaagttaaaaaaaagtttcccatagaaatcttcctcatcgtaaatatcatcatcctaaatgtcccgagtctttccatgggtctaatttaccaaatcatgaagactctctttctcactgtgtatgtgtagtgggagaggcagagacagaacggtttttttttttttttgcaagtctgctctctggattcgtttttctggggatgaaatctcaggctatgtagcttttctggtccctttttggtaatcaacaaatcatcagtttctctaggtattaaaaagcctacacttttgaaacaccaagaggccaaactctctcttaattgaaaacataattctgcctcttactgaggtctttgggtgggaagcttaaagacagcagtgttggggcaattggtttttcttttcctccctccaccttctttcccattcaagagctgttgctgccctttctggaggggagggaattagaaaaaaagatcctgccttacccagtcactgttaattattactttgagccagggtggggaatgactttctttcctggaatacaccctgctggaaaccacagctgagattctctaagctggcagcttccaacctctcctccctcaacagcttcaacattatatggcactcagctgcagggtgcctggctccaggccggcagtccatttgtgcagggtagttttcaagatggctcatagcccatctctttcagtgaccagcgtggcctatgggaaacatacatcttgactccattattggtaggagtactctttagttaactgccacaggccagttcagacacgtcttaccctgagagccttttcagagtcaggtaccatccctgtgtcccccagcatctgaagatcacaggtgaaggtctccaagtagttcacttaaaaaatacctcagtaggtttaagaacagggaaagctcccaattcattctgtcaggagtatgactctcatccccaaactggacagatatataatatgaaaactacagaccaatattccttatgaatatagatgcaaacattctaaacaaaatactagcaaaccaaatccagcagcatagaacaaggtttatgtagcatggacaactgagattatcccaggaatgcaaagttaattcaatatacaaaagtccattaatacaacatattaataaaggacaaaacaacatgaaaatcttgatacagaagaagcatttaacaaaacccacagcccctcctttttttttttttgattaaaaaacactcagtaaactaggtttaataaaagaatttcctcaacttgaagcaaacccacagctagctaacatcatactcaatggtgaaagactgaatgctttccccttaaaatcaggaacaagaaaaagatgtctgctcttgtcacttctatccaatattgtactggaggtgctagccagcacactaaggcaagaaaatgaaataataggaatctagactggaaaggaagaagtaaaagtatttctgttcacagatgcatgatctcatatgcaaaaaatactaagtgacaaaaaaagaattttagaaaagctacaatatacaaaatcaatatacaaaaattaattttatttctaacaccagtgatgaatacaaaaataaaaattagaaaataatcatataattaaagtggcatcagtaaaatacttagaaaaaaattaaagacgtcaagacttgcacactgaaatctctaaaacatcactgaaataaagacctaagcaaatgcaaagacatcccacagtcatggaacagaaaacctaacaccattaaaatagcagttatttctcaaatttatctaccaattcaactaaatccctatcaagatcccagctgttttgcaggaattgacacaatgatcatataaaaattcatatgcaatgcaagggacccaaaacagacaaaatgattttggggaaaaaaaaaaaaaaaatggaggacttgcacatcccaaatctaaacttactaccaagctacagccatcaagacagtgcggtgctggtataacgacagacatatagggcaatggagtaagactgagaatccagaaagtcttatatttatggtcaactgttctttgacaagggtaccaggaccattcatggggaaataatagtcttttcaacaactggtgcttgggcagatggatatacagataccaatgcacttatgcaaaaaatggatgaaataggaaacttcactacattctactgcatgctcggtcattttcaatcatttaggtggcaacactgacaagataacagaaagatggaggtaataacatgtgaaaggcaaaatggtttgtttttttttttaaaaatgacagtctctatcatgaatttacttacactccaggcaaaggttattagaagaaaaaaagatgtaagaaaattccttaactgaaatgtggaaagagtatcaagaggagaccctaagacactctgtaagaatcccagtgactcctcactgttcaactaagaaatgtaccccattatgctgtgctaccacggaagcattggaggcactttgggggttgatgaagtcttcatggatgaggtactttaaatatctggtcatgaagagtatttgagaaatatgacaagtgaagatgctgggtaggaatgcagcgaggaaagtgtgtaacacaaggccaaattggaaaggtcagtgagggggcaatctgtggaggcactgaatgcagaggatattaaaattagcaagatagtgttctagcacaatgaaaaggattggaagagggagatgagagtcagggagtgaaattaggcagctgctaagaatgtccgggtgagtcagaggatcaggacctgtaagggcagtataaagaaggaaggaatgatgggagaggtttagggggaaagaagtgacaacctgtgacaattgaggaatgagagattttgatgattggggtgaaggttacatttcttaaaaagaaacaaagaatggtcggtcgatagagatgcaagatgaagaattttgtttttaggactactgactatgaggtaacaaagaaatcccagagacagaggtctgagcaaaagatatgggattggggaagaatctttgggagtgactgaggttgactagagggaactgggcaagaacaggtaaggactttaagcagaattggaggagtatccatctaaaatctgggtaaactgggatggaaaaacaagtagccagagaagcaacagcccaagctagggtgttgtcaaggttatagatgttactgattttggcaatgaggagattataaggatccttgaagactgtactttcggtgaaactaccatgcattagtagcctttcacagtgatatctaccccacacctgcatcaaaatcttgcagtgcctattaataaatgccaacccactaacggggaggggagaaaagacttgggactctgtacttgtaaaaccctctaccctccccaggcaattctttacacactaacactgttgaggtaaaaggagacaagtgagggagcaaatggaaggtgtgtttttggattatacagtggctcatggaagggtgggaggggtacagatggcccttagacactggcggaaagtcagagaaaaaaaattacagtaagcaggtaaagggatcaagactacacagggactagtcttgggacgacatttcttcctctgacagtttgtatggaattcttgagaaaaattcctcgaaggggcctgaaatctcagaatggtcatgtttttaatgggaatagggagtgatgctgccccattacaaccatctgttctaacagaatgtctgtaccgaggagggatgagtaacatcggcaagttctgttcgaagcctttttcaagtttctttttgatatgtatctatctatctatcatctccctatacaagcaagcatccccaaaagtagttgtctcaggaaaccagggttaggatgaccagctcatttgctggagctgccaaggtccagaaaagtttggctgcaggctgttttcatgttttatgtatgtttgaaatgttctataataataaaaggttaaaaaagtttacatttatttggaaaaccagttactttagtttatggttcctttttttccctccagagcttcctggagattgaggtctaattcaaagaaaaccaaaatatataatagagtacctgggcaaaaaaagtacttttataacataacatttggggtagaggaagtatccactgtagtcaaaatgtctatgttttgctcttccttattgttcagggacattccattaaatagtaatgaaaaggcagcaaaagtaagaggagtgacaacatgcccggcataattaagcaagctagagcagctattctgtgcaaccgacgattttttttctctaaaattttaagggtaggttcattctgactctgttaaaagtctacttgatgtgaacaactctatatctgataacctatttcaattaccactttaaaacttgtcatatggatacgttattacaattgtagaactttaataaataccataataataaaacttgaga

SEQ ID NO: 2 - DNA, 214759 bp, Mus musculus; identical to SEQ ID NO: 1

SEQ ID NO: 3 - DNA, 20 bp, Artificial
  nucleotide sequence binding target sequence
ctcagttccgatgagagtgg

SEQ ID NO: 4 - DNA, 20 bp, Artificial
  nucleotide sequence binding target sequence
aggaatgtttgactgggaat

SEQ ID NO: 5 - DNA, 20 bp, Artificial
  nucleotide sequence binding target sequence
ctcagttacgatgagagtgg

SEQ ID NO: 6 - DNA, 20 bp, Artificial
  nucleotide sequence binding target sequence
cctcgaaactaccgatctcc

SEQ ID NO: 7 - DNA, 20 bp, Artificial
  nucleotide sequence binding target sequence
taaggtctatgttgtacgac

SEQ ID NO: 8 - DNA, 20 bp, Artificial
  nucleotide sequence binding target sequence
aacactcagccaatgctgtg

SEQ ID NO: 9 - DNA, 20 bp, Artificial
  nucleotide sequence binding target sequence
agggcaagtcagcaacacca

SEQ ID NO: 10 - DNA, 20 bp, Artificial
  nucleotide sequence binding target sequence
aatattttagatggaagcaa

SEQ ID NO: 11 - DNA, 20 bp, Artificial
  nucleotide sequence binding target sequence
aaactggctcagagtaggaa

SEQ ID NO: 12 - DNA, 20 bp, Artificial
  nucleotide sequence binding target sequence
ggggcgacagcctttttgga

SEQ ID NO: 13 - DNA, 20 bp, Artificial
  nucleotide sequence binding target sequence
aaggaggaaagggaaagaag

SEQ ID NO: 14 - DNA, 20 bp, Artificial
  nucleotide sequence binding target sequence
agggcaagtcagcaacacca

SEQ ID NO: 15 - DNA, 20 bp, Artificial
  nucleotide sequence binding target sequence
tttccaggtcgattctgtat

SEQ ID NO: 16 - DNA, 20 bp, Artificial
  nucleotide sequence binding target sequence
aagttcttaaactgacactt

SEQ ID NO: 17 - DNA, 20 bp, Artificial
  nucleotide sequence binding target sequence
tgtcgccctggttgggcctc

SEQ ID NO: 18 - DNA, 20 bp, Artificial
  nucleotide sequence binding target sequence
cccccatagcccgcaggccg

SEQ ID NO: 19 - DNA, 20 bp, Artificial
  nucleotide sequence binding target sequence
gacgcccatagcacctggga

SEQ ID NO: 20 - DNA, 20 bp, Artificial
  nucleotide sequence binding target sequence
atgttccttaccatttaact

SEQ ID NO: 21 - DNA, 20 bp, Artificial
  nucleotide sequence binding target sequence
gtacttggatatcacctaac

SEQ ID NO: 22 - DNA, 20 bp, Artificial
  nucleotide sequence binding target sequence
taaaaattagccaggcatgg

SEQ ID NO: 23 - DNA, 20 bp, Artificial
  nucleotide sequence binding target sequence
ttctgcaactactagatgat

SEQ ID NO: 24 - DNA, 20 bp, Artificial
  nucleotide sequence binding target sequence
tttccaaggaatgtttgact

SEQ ID NO: 25 - DNA, 20 bp, Artificial
  nucleotide sequence binding target sequence
tgttctgaccacttacctga

SEQ ID NO: 26 - DNA, 20 bp, Artificial
  nucleotide sequence binding target sequence
tttcttgatacttggaagaa

SEQ ID NO: 27 - DNA, 20 bp, Artificial
  nucleotide sequence binding target sequence
atgtattaagcgaccaattg

SEQ ID NO: 28 - DNA, 20 bp, Artificial
  nucleotide sequence binding target sequence
aggaatgtttgactgggaat

SEQ ID NO: 29 - DNA, 20 bp, Artificial
  nucleotide sequence binding target sequence
tgtcgccctggttgggcctc

SEQ ID NO: 30 - DNA, 20 bp, Artificial
  nucleotide sequence binding target sequence
aaaggcaacgcgttacacgc

SEQ ID NO: 31 - DNA, 20 bp, Artificial
  nucleotide sequence binding target sequence
aaactggctcagagtaggaa

SEQ ID NO: 32 - DNA, 21 bp, Artificial
  nucleotide sequence binding target sequence
gctgaggcccaaccagggcga

SEQ ID NO: 33 - DNA, 22 bp, Artificial
  nucleotide sequence binding target sequence
gctactctgagccagttttgga

SEQ ID NO: 34 - DNA, 21 bp, Artificial
  nucleotide sequence binding target sequence
gctgaggcccaaccagggcga

SEQ ID NO: 35 - DNA, 20 bp, Artificial
  nucleotide sequence binding target sequence
acttaaaaatcatgctcaat

SEQ ID NO: 36 - DNA, 20 bp, Artificial
  nucleotide sequence binding target sequence
tatattgttctgaagagagg

SEQ ID NO: 37 - DNA, 20 bp, Artificial
  nucleotide sequence binding target sequence
tgctcaataggattacgctg

SEQ ID NO: 38 - DNA, 20 bp, Artificial
  nucleotide sequence binding target sequence
tggtctcctgcactgagctg

SEQ ID NO: 39 - DNA, 20 bp, Artificial
  nucleotide sequence binding target sequence
gaggaaagggaaagaagtca

SEQ ID NO: 40 - DNA, 20 bp, Artificial
  nucleotide sequence binding target sequence
ctctgacgaaatgtcttcca

SEQ ID NO: 41 - DNA, 20 bp, Artificial
  nucleotide sequence binding target sequence
caaattccaaaaggaccgac

SEQ ID NO: 42 - DNA, 20 bp, Artificial
  nucleotide sequence binding target sequence
agaattcggctacctctact

SEQ ID NO: 43 - DNA, 21 bp, Artificial
  nucleotide sequence binding target sequence
tagagccccccaaggagtcaa

SEQ ID NO: 44 - DNA, 20 bp, Artificial
  nucleotide sequence binding target sequence
ttccagaactggctcagagt

SEQ ID NO: 45 - DNA, 21 bp, Artificial
  nucleotide sequence binding target sequence
aagagccccccaaggagtcaa

SEQ ID NO: 46 - DNA, 20 bp, Artificial
  nucleotide sequence binding target sequence
ccatgaagggggaggtcacc

SEQ ID NO: 47 - DNA, 20 bp, Artificial
  nucleotide sequence binding target sequence
agaggagagagacatttttc

SEQ ID NO: 48 - DNA, 20 bp, Artificial
  nucleotide sequence binding target sequence
atccagtcctagacagaatt

SEQ ID NO: 49 - DNA, 20 bp, Artificial
  nucleotide sequence binding target sequence
tgtcgccctggttgggcctc

SEQ ID NO: 50 - DNA, 20 bp, Artificial
  nucleotide sequence binding target sequence
ttgtgggactaggtgcttgt

SEQ ID NO: 51 - DNA, 20 bp, Artificial
  nucleotide sequence binding target sequence
tttgtgaacccatcttgggg

SEQ ID NO: 52 - DNA, 20 bp, Artificial
  nucleotide sequence binding target sequence
agagccccccaaggagtcaa

SEQ ID NO: 53 - DNA, 20 bp, Artificial
  nucleotide sequence binding target sequence
gaggaaagggaaagaagtca

SEQ ID NO: 54 - DNA, 20 bp, Artificial
  nucleotide sequence binding target sequence
gaaaatctcctgagaatcct

SEQ ID NO: 55 - DNA, 20 bp, Artificial
  nucleotide sequence binding target sequence
catttcctctcatgggaaac

SEQ ID NO: 56 - DNA, 20 bp, Artificial
  nucleotide sequence binding target sequence
ttgtgggactaggtgcttgt

SEQ ID NO: 57 - DNA, 20 bp, Artificial
  nucleotide sequence binding target sequence
ttccagaactggctcagagt

SEQ ID NO: 58 - DNA, 20 bp, Artificial
  nucleotide sequence binding target sequence
aggaatgtttgactgggaat

SEQ ID NO: 59 - DNA, 20 bp, Artificial
  nucleotide sequence binding target sequence
ttcttcatgacattgggttg

SEQ ID NO: 60 - DNA, 20 bp, Artificial
  nucleotide sequence binding target sequence
caaattccaaaaggaccgac

SEQ ID NO: 61 - DNA, 20 bp, Artificial
  nucleotide sequence binding target sequence
agaggagagagacatttttc

SEQ ID NO: 62 - DNA, 20 bp, Artificial
  nucleotide sequence binding target sequence
taagaaaggctctgtcattt

SEQ ID NO: 63 - DNA, 20 bp, Artificial
  nucleotide sequence binding target sequence
tttatgaacccatcctgggg

SEQ ID NO: 64 - DNA, 20 bp, Artificial
  nucleotide sequence binding target sequence
tgtcgccctggttgggcctc

SEQ ID NO: 65 - DNA, 20 bp, Artificial
  nucleotide sequence binding target sequence
agagccccccaaggagtcaa

SEQ ID NO: 66 - DNA, 20 bp, Artificial
  nucleotide sequence binding target sequence
tttgtgaacccatcgtgggg

SEQ ID NO: 67 - DNA, 20 bp, Artificial
  nucleotide sequence binding target sequence
aaactggctcagagtaggaa

SEQ ID NO: 68 - DNA, 20 bp, Artificial
  nucleotide sequence binding target sequence
acccagtcctagacagaatt

SEQ ID NO: 69 - DNA, 19 bp, Artificial
  nucleotide sequence binding target sequence
cccagtccaagacagaatt

SEQ ID NO: 70 - DNA, 20 bp, Artificial
  nucleotide sequence binding target sequence
acccagtccaagacagaatt

SEQ ID NO: 71 - DNA, 21 bp, Artificial
  nucleotide sequence binding target sequence
tagagccccccaaggagtcaa

SEQ ID NO: 72 - DNA, 20 bp, Artificial
  nucleotide sequence binding target sequence
caaattccaaaaggacagac

SEQ ID NO: 73 - DNA, 19 bp, Artificial
  nucleotide sequence binding target sequence
ctcatgggaaacagggcaa

SEQ ID NO: 74 - DNA, 19 bp, Artificial
  nucleotide sequence binding target sequence
atggttccattgtgggact

SEQ ID NO: 75 - DNA, 20 bp, Artificial
  nucleotide sequence binding target sequence
taagaaaggctctgtcattt

SEQ ID NO: 76 - DNA, 20 bp, Artificial
  nucleotide sequence binding target sequence
ctctgacgaaatgtcttcca

SEQ ID NO: 77 - DNA, 20 bp, Artificial
  nucleotide sequence binding target sequence
gctgcccaagttctatggat

SEQ ID NO: 78 - DNA, 21 bp, Artificial
  nucleotide sequence binding target sequence
gacccagtccaagacagaatt

SEQ ID NO: 79 - DNA, 20 bp, Artificial
  nucleotide sequence binding target sequence
tcacttttcttcatgacatt

SEQ ID NO: 80 - DNA, 20 bp, Artificial
  nucleotide sequence binding target sequence
tttgtgaacccatcctgggg

SEQ ID NO: 81 - DNA, 20 bp, Artificial
  nucleotide sequence binding target sequence
aaactggctcagagtaggaa

SEQ ID NO: 82 - DNA, 20 bp, Artificial
  nucleotide sequence binding target sequence
taagaaaggctctgtcactt

SEQ ID NO: 83 - DNA, 20 bp, Artificial
  nucleotide sequence binding target sequence
tttttgaacccatcctgggg

SEQ ID NO: 84 - DNA, 20 bp, Artificial
  nucleotide sequence binding target sequence
gagaacccatgtctatacat

SEQ ID NO: 85 - DNA, 20 bp, Artificial
  nucleotide sequence binding target sequence
tttgtgaacccatcttgggg

SEQ ID NO: 86 - DNA, 20 bp, Artificial
  nucleotide sequence binding target sequence
aacctaatccaaccacttac

SEQ ID NO: 87 - DNA, 21 bp, Artificial
  nucleotide sequence binding target sequence
tagagccccccaaggagtcaa

SEQ ID NO: 88 - DNA, 20 bp, Artificial
  nucleotide sequence binding target sequence
cttaccttcctttccaaatc

SEQ ID NO: 89 - DNA, 20 bp, Artificial
  nucleotide sequence binding target sequence
gtgaccctgtcaccctggtt

SEQ ID NO: 90 - DNA, 20 bp, Artificial
  nucleotide sequence binding target sequence
ttggaagaatgtcttcccta

(19) United States
(12) Patent Application Publication
(10) Pub. No.: US 2021/0054370 A1
(43) Pub. Date: Feb. 25, 2021

(54) METHODS AND COMPOSITIONS FOR TREATING ANGELMAN SYNDROME

(71) Applicant: The University of North Carolina at Chapel Hill, Chapel Hill, NC (US)

(72) Inventors: Mark J. Zylka, Chapel Hill, NC (US); Justin Matthew Wolter, Pittsboro, NC (US); Giulia Fragola, Durham, NC (US); Jeremy Mark Simon, Carrboro, NC (US); Hangqian Mao, Durham, NC (US)

(21) Appl. No.: 16/976,386

(22) PCT Filed: Feb. 27, 2019
(86) PCT No.: PCT/US19/19789

§ 371 Date: Aug. 27, 2020

Related U.S. Application Data
(60) Provisional application No. 62/635,815, filed on Feb. 27, 2018.

(51) Int. Cl. C12N 15/11; C12N 9/22; A61K 38/46; A61K 31/7088; A61P 25/00; C12N 7/00

(52) U.S. Cl. CPC C12N 15/11; C12N 9/22; A61K 38/465; A61K 31/7088; C12N 2750/14143; C12N 7/00; C12N 2310/20; C12N 2800/80; A61P 25/00

(57) ABSTRACT

This invention relates to methods and compositions for treating Angelman syndrome, including administering to a subject an effective amount of a clustered regularly interspersed short palindromic repeat (CRISPR)-associated endonuclease and one or more than one guide RNA molecules having complementarity to a target nucleotide sequence in UBE3A-ATS in cells of the subject.

### STATEMENT OF PRIORITY

This application claims the benefit, under 35 U.S.C. § 119(e), of U.S. Provisional Application Ser. No. 62/635,815, filed Feb. 27, 2018, the entire contents of which are incorporated by reference herein.

### STATEMENT OF GOVERNMENT SUPPORT

This invention was made with government support under Grant Number ES024088 awarded by the National Institutes of Health. The government has certain rights in the invention.

### FIELD OF THE INVENTION

This invention relates to methods and compositions for treating Angelman syndrome.

### STATEMENT REGARDING ELECTRONIC FILING OF A SEQUENCE LISTING

A Sequence Listing in ASCII text format, submitted under 37 C.F.R. § 1.821, entitled 5470-839WO_ST25.txt, 577,227 bytes in size, generated on Feb. 27, 2019 and filed via EFS-Web, is provided in lieu of a paper copy. This Sequence Listing is hereby incorporated herein by reference into the specification for its disclosure.

### BACKGROUND OF THE INVENTION

Angelman syndrome (AS) is a severe neurodevelopmental disorder caused by deletion or mutation of the maternal allele of UBE3A. UBE3A is biallelically expressed in nearly all cells of the body except in mature neurons, where the paternal allele is silenced by an extremely long non-coding RNA called UBE3A-ATS. In light of this biology, the most direct way to treat neural and behavioral dysfunctions associated with AS is to unsilence the intact paternal UBE3A allele. CRISPR/Cas9 technology can be used to permanently modify specific regions of the mammalian genome, such as when active Cas9 is used. CRISPR/Cas9 technology can also be used to repress transcription at specific regions of the mammalian genome, such as when dead Cas9 is used alone or fused to a repressor domain.

The present invention overcomes previous shortcomings in the art by providing methods and compositions for treating neurodevelopment disorders such as Angelman Syndrome.

### SUMMARY OF THE INVENTION

In one aspect, the present invention provides a method of unsilencing paternal UBE3A in a human subject in need thereof, comprising administering to the subject an effective amount of a clustered regularly interspersed short palindromic repeat (CRISPR)-associated endonuclease (which is catalytically active or inactive/dead) and one or more than one guide RNA (gRNA) molecule having complementarity to a target nucleotide sequence in UBE3A-ATS in cells of the subject.

In a further aspect, the present invention provides a method of treating Angelman Syndrome (AS) in a subject in need thereof, comprising administering to the subject an effective amount of a clustered regularly interspersed short palindromic repeat (CRISPR)-associated endonuclease (which is catalytically active or inactive/dead) and one or more than one guide RNA molecule having complementarity to a target nucleotide sequence in UBE3A-ATS in cells of the subject.

### BRIEF DESCRIPTION OF THE DRAWINGS

FIG. 1. Molecular pathways to unsilence the intact paternal UBE3A allele.

FIGS. 2A-B. (2A) Ube3a-ATS blocks paternal Ube3a in neurons via a transcriptional interference/collision mechanism. (2B) Boundary element truncates Ube3a-ATS in non-neuronal cells. X=methylated/imprinted region.

FIGS. 3A-D. (3A) Location of gRNAs relative to genes and regulatory elements in a gRNA library. (3B) Gene annotations. (3C) Ribozero RNA-seq from cortical neurons. (3D) Regulatory element marks.

FIG. 4. SpCas9 and jw33 gRNA upregulates paternal Ube3a while it also downregulates Snord115 and Ube3a-ATS without affecting other genes in the vicinity. (Panel A) RT-qPCR of total RNA from wild-type mouse neurons transduced with lentivirus carrying SpCas9 and a gRNA targeting mouse Snord115 or human SNORD115 (negative control, normalized to dashed line). (Panel B) Ube3a^{WT/YTP} neurons transduced with the same viruses as in Panel A. RT-qPCR using primers that selectively interrogate expression of the maternal allele (WT Ube3a) or paternal allele (Ube3a: YFP). Paternal (black) and maternal (gray) allele.

FIG. 5. Active SpCas9, dead (d)SpCas9, dSpCas9 fused to KRAB repressor domain, active SaCas9, and dSaCas9 can all unsilence paternal Ube3a in cultured mouse neurons. Cas9 plasmid and a gRNA were transfected along with tdTomato into Ube3a^{m+/patYFP} neuron cultures. Scr.=scrambled gRNA; jw33 gRNA; Sajw33 gRNA. Percent co-localization between YFP and tdTomato was quantified using high content imaging. N=4. *P<0.0005.

FIG. 6. Single nucleotide polymorphisms (SNPs) in UBE3A-ATS show strong allelic bias in human neurons. Paternal (black) and maternal (gray) allele.

FIG. 7. SNPs in UBE3A show strong allelic bias in human neurons. Single nucleotide polymorphisms identified in the reading frame of UBE3A using strand specific RNA-seq in primary human neural progenitor cells (phNPC) and phNPC-derived neurons after 8 weeks neuronal differentiation. Paternal (black) and maternal (gray) allele.

FIG. 8. (Panel A) Data showing lentiviral delivery of SpCas9 and hsa jw33 unsilences paternal UBE3A in human neurons differentiated from phNPCs. The scrambled gRNA negative control did not unsilence paternal UBE3A. TaqMan genotyping probes specific to an A/T SNP in exon 5 of UBE3A (Chr15:25,371,697; rs530054948) were used to quantify expression differences between each allele. Cas9−: neurons that were not transduced with Cas9. Cas9+: neurons that were transduced with Cas9. (Panel B). Expression of genes near the guide RNA target site following lentiviral delivery of SpCas9 and hsa jw33 in phNPC-derived neurons. Dashed line marks expression values relative to the scrambled gRNA (negative) control.

FIG. 9. In utero electroporation of SaCas9 and Sajw33 gRNA unsilences paternal Ube3a in mouse brain. E15.5 patUbe3a:YFP embryos in utero electroporated with AAV-hSyn1::SaCas9-Sajw33 and pCAGG-mCherry plasmids. At P30, mouse brains were colabeled for mCherry (Panel A) and paternal UBE3A-YFP (Panel B).

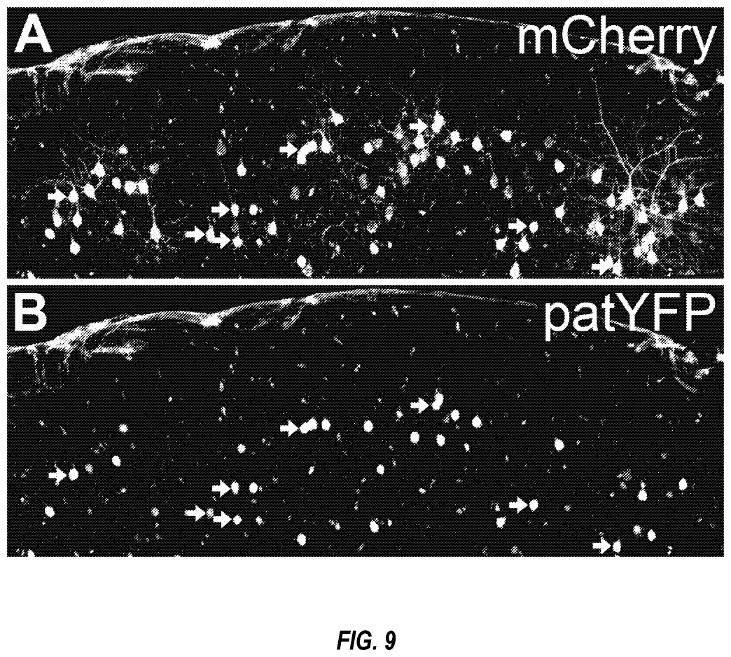

FIG. 10. AAV2 gene therapy vector containing SaCas9 and Sajw33 gRNA unsilences paternal Ube3a in mouse brain. (Panel A) E16.5 patUbe3a:YFP embryos injected intraventricularly with AAV2-hSyn1-SaCas9-Sajw33. At P30, mouse brains were dissected and stained with antibodies to YFP. (Panels B-C) Zoom in shows increased expression of patUBE3A:YFP in neurons. Note uniform maximal expression levels and predominant nuclear localization, indicative of proper expression from the paternal Ube3a promoter and proper isoform usage.

FIG. 11. AAV9 gene therapy vector containing SaCas9 and Sajw33 gRNA unsilences paternal Ube3a in mouse brain when injected intraventricularly on embryonic day 15.5. (Panel A) E15.5 patUbe3a:YFP embryos injected in utero with AAV9-hSyn1-SaCas9-Sajw33. At P30, mouse brains were dissected and stained with antibodies to YFP. (Panel B) Zoom in shows increased expression of patUBE3A:YFP in hippocampal neurons. (Panels C-D) Zoom in shows increased expression of patUBE3A:YFP in cerebral cortex neurons. Note the uniform maximal expression levels and predominant nuclear localization, which are indicative of proper expression from the paternal UBE3A promoter and proper isoform usage. This vector achieves UBE3A unsilencing across the extent of the brain.

FIG. 12. AAV9 gene therapy vector containing SaCas9 and Sajw33 gRNA unsilences paternal Ube3a in mouse brain when injected intraventricularly on postnatal day 1. (Panel A) Postnatal day 1 (P1) patUbe3a:YFP mice injected with AAV9-hSyn1-SaCas9-Sajw33. At P30, mouse brains were dissected and stained with antibodies to YFP. (Panel B) Zoom in shows increased expression of patUBE3A:YFP in hippocampal neurons. (Panels C-D) Zoom in shows increased expression of patUBE3A:YFP in cerebral cortex neurons.

FIG. 13. AAV9 gene therapy vector containing SaCas9 and Sajw33 gRNA unsilences paternal Ube3a in Angelman syndrome model mouse (Ube3a^{m-/p+}) when injected intraventricularly on postnatal day 1. (Panel A) Postnatal day 1 (P1) Ube3a^{m-/p+} mouse was treated with AAV9-hSyn1-SaCas9-Sajw33 gRNA through intraventricular injection. At P30, treated mice were perfused and brains were dissected and stained for UBE3A protein. (Panel B) Zoom in shows increased expression of patUBE3A in hippocampal neurons. (Panels C-D) Zoom in shows increased expression of patUBE3A in cerebral cortex neurons.

FIG. 14. AAV9 gene therapy vector containing SaCas9 and Sajw33 gRNA rescues hindlimb clasping phenotype in Angelman syndrome model mice (Ube3a^{m-/p+}). (Panel A) Hindlimb clasping phenotype scored on scale from 0 (no phenotype), 1 (mild), 2 (severe). (Panel B) WT and Ube3a^{m-/p+} Angelman syndrome model mice injected with AAV9:SaCas9+Sajw33 (or scrambled gRNA) at P1, hindlimb clasping scored at P30. N=6-10/group. n.s.=nonsignificant. *P<0.05.

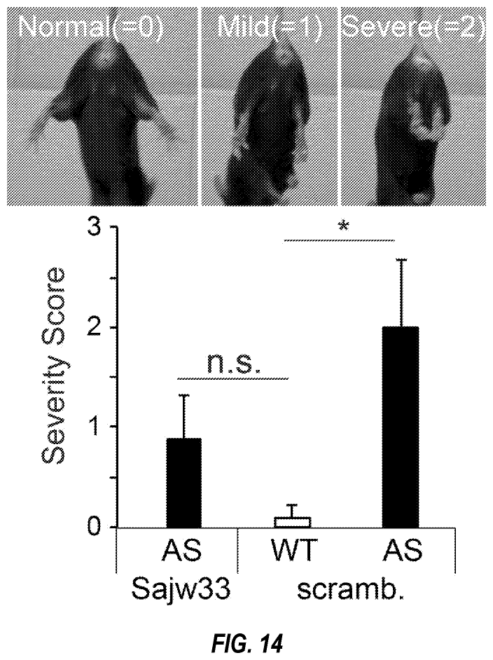

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

The present invention is based on the unexpected discovery that the paternal UBE3A gene can be unsilenced using protocols employing clustered regularly interspersed short palindromic repeat (CRISPR) technology. Thus, in one aspect, the present invention provides a method of unsilencing paternal UBE3A in a human subject in need thereof, comprising administering to the subject an effective amount of a CRISPR-associated endonuclease and one or more than one guide RNA molecule having complementarity to a target nucleotide sequence in UBE3A-ATS in cells of the subject.

In an additional aspect, the present invention provides a method of treating Angelman Syndrome (AS) in a subject in need thereof, comprising administering to the subject an effective amount of a clustered regularly interspersed short palindromic repeat (CRISPR)-associated endonuclease and one or more than one guide RNA molecule having complementarity to a target nucleotide sequence in UBE3A-ATS in cells of the subject.

In bacteria the CRISPR/Cas loci encode RNA-guided adaptive immune systems against mobile genetic elements (viruses, transposable elements and conjugative plasmids). CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonuclease, e.g., Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 isolated from Streptococcus pyogenes (SpCas9) recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA, also referred to as “gRNA”) via a synthetic stem loop (AGAAAU) to mimic the natural crRNA/tracrRNA duplex. Such sgRNA, like shRNA, can be synthesized or in vitro transcribed for direct RNA transfection, or expressed from a U6 or H1-promoter, although cleavage efficiencies of the artificial sgRNA are lower than those for systems with the crRNA and tracrRNA expressed separately.

In a further embodiment, the present invention provides a composition comprising a CRISPR-associated endonuclease and one or more guide RNA molecules having complementarity to a target nucleotide sequence in UBE3A-ATS. In some embodiments, the CRISPR-associated endonuclease is present in the composition as a nucleic acid molecule that encodes the CRISPR-associated nuclease. In some embodiments, the nucleic acid molecule that encodes the CRISPR-associated endonuclease and the one or more than one guide RNA molecules are present in a single nucleic acid construct. In some embodiments, the nucleic acid molecule that encodes the CRISPR-associated endonuclease and the nucleic acid molecule that encodes the one or more than one guide RNA molecule are present on two or more (e.g., 2, 3, 4, 5, 6, 7, 8, 9, 10, etc.) separate nucleic acid constructs.

In some embodiments, the CRISPR-Cas nuclease is catalytically active. In some embodiments, the CRISPR-associated endonuclease is Cas9 or a variant thereof, a human-optimized Cas9, including SpCas9, SaCas9, St1Cas9, BlatCas9, NmCas9, FnCas9, CjCas9, xCas9, Cas9 base editors, C2C2 RNA guided, and/or RNA targeting nucleases (e.g., such as targeting UBE3A-ATS). In some embodiments, the CRISPR-Cas nuclease is a Cpf1 nuclease and/or variant thereof. Exemplary Cpf1 variants include, but are not limited to, AsCpf1 and/or LbCpf1. Exemplary CRISPR-Cas nucleases and Cpf1 nucleases include, but are not limited to the CRISPR-Cas nucleases as described in Nakade et al., Bioengineered 8(3):265-273 (2017).

In some embodiments, the CRISPR-Cas nuclease is a CasX nuclease and/or variant thereof. Exemplary CasX variants include, but are not limited to DpbCasX and/or PlmCasX (Lui et al. Nature 566 (7743):218-223 (2019)). In some embodiments, the CRISPR-Cas nuclease is a Cas12 nuclease and/or variant thereof. Exemplary Cas12 variants include, but are not limited to Cas12b, Cas12c, Cas12g (Yan et al. Science 363 (6422):88-91 (2019)).

In some embodiments, the CRISPR-associated endonuclease is catalytically inactive and/or dead. Exemplary CRISPR-associated endonucleases that are catalytically inactive and/or dead include, but are not limited to, dead SaCas9, dead SpCas9 and dead CasX.

In some embodiments, the CRISPR-associated endonuclease is fused to a repressor domain. In some embodiments, the repressor gene is a KRAB repressor. (Urrutia. “KRAB-containing zinc-finger repressor proteins” Genome Biology 4(10):231 (2003)). Examples of such CRISPR-associated endonucleases include, but are not limited to, dead SpCas9-KRAB and/or dead SaCas9-KRAB (e.g., combined with guide RNAs (gRNAs) targeting a nucleotide sequence in UBE3A-ATS (e.g., Snord115).

Thus, in some embodiments, the present invention provides a method of unsilencing paternal UBE3A in a human subject in need thereof, comprising administering to the subject an effective amount of a dead CRISPR-associated endonuclease (e.g., dead SpCas9 and/or dead SaCas9) and one or more than one guide RNA molecule having complementarity to a target nucleotide sequence (e.g., Snord115) in UBE3A-ATS in cells of the subject. In some embodiments, the dead CRISPR-associated endonuclease is fused to a repressor domain (e.g., dead SpCas9-KRAB and/or dead SaCas9-KRAB)

In an additional aspect, the present invention provides a method of treating Angelman Syndrome (AS) in a subject in need thereof, comprising administering to the subject an effective amount of a dead CRISPR-associated endonuclease (e.g., dead SpCas9 and/or dead SaCas9) and/or a dead CRISPR-associated endonuclease fused to a repressor domain (e.g., dead SpCas9-KRAB and/or dead SaCas9-KRAB) and one or more than one guide RNA molecule having complementarity to a target nucleotide sequence (e.g., Snord115) in UBE3A-ATS in cells of the subject.

In some embodiment, the present invention provides a nucleic acid molecule encoding a CRISPR-associated endonuclease, wherein the CRISPR-associated endonuclease is a CRISPR-Cas nuclease. In some embodiments, the present invention provides a nucleic acid molecule encoding a CRISPR-associated endonuclease (e.g., a CRISPR-Cas nuclease), and a nucleic acid molecule encoding one or more than one guide RNA that is complementary to a target sequence in UBE3A-ATS in cells of a subject.

The present invention provides one or more than one guide RNA (gRNA) molecule comprising a sequence that is complementary to a target nucleotide sequence in UBE3A-ATS. The gRNA molecule(s) can be complementary to a coding and/or a non-coding sequence. In some embodiments, the gRNA molecule(s) can comprise about 1 to about 30 nucleotides, about 5 to about 25 nucleotides, or about 10 to about 20 nucleotides (including about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides). In some embodiments, the gRNA molecule(s) or a portion thereof can base-pair with a target nucleotide sequence in UBE3A-ATS. For example, in some embodiments, the number of nucleotides base-pairing with the target nucleotide sequence can be from about 1 to about 10, or from about 1 to about 20 nucleotides (including 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides).

In a further embodiment, the invention provides a composition comprising a nuclease that has been engineered to bind a target nucleotide sequence in UBE3A-ATS as described herein. In some embodiments, such a nuclease binds directly to the target nucleotide sequence of this invention in the absence of one or more guide RNA molecules. Examples of such nucleases include, but are not limited to, TAL nucleases (Hockemeyer et al. Nat. Biotechnol. 29(8):731-734 (2012); Wood et al. Science 333(6040):1-8) (2011)), zinc finger nucleases, and any other nuclease now known or later identified. In some embodiments, the target nucleotide sequence in UBE3A-ATS is the same target sequence targeted by the one or more guide RNA molecules of this invention. TAL and zinc finger nucleases can be engineered using methods known in the art by fusing a series of TAL or zinc finger DNA recognition domains together that have sequence specificity for the target sequence.

In some embodiments of this invention, methods are provided of unsilencing paternal UBE3A in a human subject in need thereof, comprising administering to the subject an effective amount of a Transcription activator-like effector (TALE) DNA-binding domain, a Transcription activator-like effector nuclease (TALEN), a zinc finger DNA-binding domain, and/or a zinc finger nuclease that binds to a target nucleotide sequence in UBE3A-ATS in cells of the subject.

In a further aspect, the present invention provides a method of treating Angelman Syndrome (AS) in a subject in need thereof, comprising administering to the subject an effective amount of a Transcription activator-like effector (TALE) DNA-binding domain, a Transcription activator-like effector nuclease (TALEN), a zinc finger DNA-binding domain, and/or a zinc finger nuclease that binds a target nucleotide sequence in UBE3A-ATS in cells of the subject.

In some embodiments, the target nucleotide sequence in UBE3A-ATS includes, but is not limited to, any nucleotide sequence that is able to reduce expression and/or transcript levels of UBE3A-ATS. In some embodiments, the target nucleotide sequence in UBE3A-ATS includes, but is not limited to, any nucleotide sequence that is able to treat Angelman Syndrome, as identified herein or later identified. Such target nucleotide sequences may or may not be conserved across species. In embodiments of the invention wherein the target nucleotide sequence is not conserved across species, the corresponding transcript of such a target sequence is conserved across species (e.g., see Tables 1-4). In some embodiments, the target nucleotide sequence in UBE3A-ATS is in one or more Snord115 (a.k.a. HBII-52 in human, MBII-52 in mouse), Snord115HG, SNHG14, SNRPN, Snord64, IPW, Snord116, and/or Snord116HG genes. In some embodiments, the target nucleotide sequence in UBE3A-ATS is one or more Snord115 genes. In some embodiments, the target nucleotide sequence in UBE3A-ATS is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 Snord 115 genes. Snord 115 genes may be differentiated by adding a numeric suffix, e.g., Snord115-1, Snord155-2, etc. In some embodiments, the target nucleotide sequence in UBE3A-ATS is one or more Snord116 (a.k.a. HBII-85 in human, MBII-85 in mouse) genes.

In some embodiments, the target nucleotide sequence is the entire nucleotide sequence of human UBE3A-ATS (SEQ ID NO:1) or any contiguous region of at least about ten nucleotides (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, etc., including any number of nucleotides up to the total number of nucleotides in SEQ ID NO:1 not expressly set forth herein) of the nucleotide sequence of SEQ ID NO:1. This human sequence (SEQ ID NO:1) encompasses the 3′end of the IPW transcript, through all the snord115 repeats, and ends just before the annotated 3′UTR of UBE3A.

The gRNA molecule(s) of the invention can comprise a nucleotide sequence that is complementary to any target nucleotide sequence in UBE3A-ATS, wherein the target nucleotide sequence can be SNORD116, SNORD115 and/or IPW or any portion thereof. In some embodiments, the target nucleotide sequence is the entire nucleotide sequence of mouse Ube3a-ATS (SEQ ID NO:2) or any contiguous region of at least about ten nucleotides (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, etc., including any number of nucleotides up to the total number of nucleotides in SEQ ID NO:2 not expressly set forth herein) of the nucleotide sequence of SEQ ID NO:2. This mouse sequence (SEQ ID NO:2) encompasses the 3′end of the IPW transcript, through all the snord115 repeats, and ends just before the annotated 3′UTR of ube3a.

The gRNA molecule(s) of the invention can comprise a nucleotide sequence that is complementary to any target nucleotide sequence in Ube3a-ATS, wherein the target nucleotide sequence can be Snord116, Snord115 and/or IPW or any portion thereof.

In some embodiments, the location of the target nucleotide sequence and/or gene is provided in the form of an Hg transcript annotation (Table 8). In some embodiments, the gRNA molecule(s) can be selected from the gRNA sequences in Tables 1-5. For example, in some embodiments, the gRNA molecule of this invention can comprise the nucleotide sequence of any of SEQ ID NOs:3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 72, 74, 75, 76, 77, 78, 79, 80, 81, 82, 83, 84, 85, 86, 87, 88, 89 or 90.

In some embodiments, the nucleic acid molecule of this invention can be present in a vector, which can be a non-viral vector or a viral vector. Non-limiting examples of a viral vector of this invention include, but are not limited to, a retroviral vector, a lentiviral vector, an adenoviral vector, an adeno-associated viral vector, an alphavirus vector, a vaccinia viral vector, a herpesviral vector, etc., as are known in the art or later identified.

In some embodiments, the nucleic acid molecule of this invention can be present in an adeno-associated viral (AAV) vector, which can be an AAV2 vector and/or an AAV 9 vector or a viral vector of any other AAV serotype. In some embodiments, the nucleic acid molecule can be an AVV9 vector comprising a nucleotide sequence encoding a Cas9 endonuclease selected from SpCas9, SaCas9, NmCas9, and CjCas9 and can further encode one or more than one gRNA molecule.

In some embodiments, the vector of this invention can be a non-viral vector (e.g., a plasmid, a liposome or any other nucleic acid delivery vehicle now known or later identified).

In some embodiments, a nucleic acid construct of this invention is a vector that can comprise, for example, a promoter and intron located upstream of a nucleotide sequence encoding Cas9 and a separate U6 promoter located upstream of a nucleotide sequence encoding the gRNA(s). In some embodiments, the promoter upstream of the nucleotide sequence encoding Cas9 can be a cell-type specific promoter such as, but not limited to, synapsin (i.e., to drive expression in neurons) and/or a ubiquitous promoter (e.g., cytomegalovirus (CMV) and/or a chicken beta-acting (CBA) hybrid (CBh) promoter).

In some embodiments, the CRISPR-associated endonuclease and the one or more than one guide RNA can be attached to or linked to a nanoparticle or microparticle comprising a Cas9 ribonucleoprotein complex.

Furthermore, the vector of this invention can comprise a vector genome that has been optimized relative to a wild type vector genome, e.g., to enhance the activity of viral cis elements required for replication, packaging and/or delivery, etc., as would be well known in the art. Such an optimized vector can comprise an optimized transcription cassette, optimized terminal repeats, etc., as would be well known in the art.

In some embodiments, the complementarity of the one or more than one guide RNA (gRNA) molecule to a target nucleotide sequence in UBE3A-ATS can vary, but typically is at least about 75%, 80%, 85%, 90%, 91%, 92%, 93%, 94%, 95%, 96%, 97%, 98%, 99%, or 100%. In some embodiments, single base pair mismatches may be tolerated although the targeting efficiency may be reduced.

In some embodiments, a CRISPR-associated endonuclease can have a nucleotide sequence identical to the wild type sequence. For example, in some embodiments, the nucleotide sequence of the CRISPR-Cas nuclease (e.g., Cas9) can have a nucleotide sequence identical to the wild type Streptococcus pyogenes sequence (Deltcheva et al. Nature 471(7340):602-607 (2011); Mali et al. Science 339(6121):823-826 (2013)); Cong et al. Science 339(6121):819-823 (2013)). In some embodiments, the CRISPR-associated endonuclease can be a nucleotide sequence from other species, including for example other species such as Streptococcus thermophilus; Pseudomonas aeruginosa, Escherichia coli, or other sequenced bacteria genomes and archaea, or other prokaryotic microorganisms. Examples include, but are not limited to, Staphylococcus aureus (SaCas9) (Ran et al. Nature 520(7456):186-191 (2015); Friedland et al. Genome Biology 16:257 (2105)); Neisseria meningitides (NmCas9) (Hou et al. PNAS 110(39):15644-15649 (2103)); Campylobacter jejuni (CjCas9) (Kim et al. Nature Comm 8:1-12 (2017)); evolved SpCas9 (xCas9) (Hu et al. Nature 556(7699):57-63 (2108)); Acidaminococcus sp. (AsCpfl) (Kleinstiver et al. Nat. Biotechnol. 34(8):869-874 (2016)); Lachnospiracea bacterium (LbCpfl) (Kleinstiver et al. Nat. Biotechnol. 34(8):869-874 (2106)); Cas12 (Yan et al. Science 363(6422):88-91 (2019)); Deltaproteobacteria (DpbCasX); and Planctomycetes (PlmCasX) (Lui et al. Nature 566 (743):218-223 (2019)).

In some embodiments, a CRISPR-associated endonuclease can include CRISPRi (e.g., dead SpCas9, dead SpCas9-KRAB (Lui et al. Science 355(6320):1-19 (2107), Mandegar et al. Cell Stem 18(4):541-553 (2106)); Cas9 base editors (Korner et al. Nature 533(7603):420-424 (2106)); and/or C2C2 RNA guided RNA targeting (such as targeting UBE3A-ATS (Abudayyeh et al. Science 353(6299):1-23 (2016), Gilbert et al. Cell 159(3):647-661 (2014)).

Alternatively, in some embodiments, the wild type CRISPR-associated endonuclease can be modified. For example, in some embodiments the wild type Cas9 sequence (e.g., Streptococcus pyogenes Cas9 sequence) can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors identified under GenBank accession numbers KM099231.1 GI:669193757; KM099232.1 GI:669193761; KM099233.1 GI:669193765; and/or LP885305.1.

In some embodiments, a CRISPR-associated endonuclease sequence can be provided in a commercially available vector. For example, in some embodiments, the Cas9 nuclease sequence can be contained within a commercially available vector such as but not limited to PX330, PX260, PX600, or PX601 from Addgene (Cambridge, Mass.). In some embodiments, the Cas9 endonuclease can have an amino acid sequence that is a variant or a fragment of any of the Cas9 endonuclease sequence of GenBank accession numbers KM099231.1 GI:669193757; KM099232.1 GI:669193761; or KM099233.1 GI:669193765 or the Cas9 amino acid sequence of PX330, PX260. PX600, or PX601 (Addgene, Cambridge, Mass.). In some embodiments, the nucleotide sequence of the CRISPR-associated endonuclease can be modified to encode biologically active variants and these variants can have or can include, for example, an amino acid sequence that differs from a wild type by comprising one or more mutations (e.g., an addition, insertion, deletion and/or or substitution, or any combination of such mutations). For example, in some embodiments, a Cas9 nuclease sequence (e.g., Cas9) can be modified to encode a biologically active variant of Cas (e.g., Cas9), and such a variant can have or can include an amino acid sequence that differs from a wild type Cas (e.g., Cas9) due to the presence of one or more mutations (e.g., an addition, deletion, insertion, or substitution or a combination of such mutations). One or more of the mutations can be conservative amino acid substitution or a nonconservative amino acid substitution, in any combination. For example, a biologically active variant of a CRISPR-associated polypeptide such as a Cas polypeptide (e.g., Cas9) can have an amino acid sequence with at least about 50% sequence identity (e.g., at least about 50%, 55%, 60%, 65%, 70%, 75%, 80%, 85%, 90%, 95%, 97%, 98%, or 99% sequence identity) to a wild type CRISPR-associated polypeptide such as Cas (e.g., Cas9) polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

Naturally occurring amino acid residues include those naturally encoded by the genetic code as well as non-standard amino acids (e.g., amino acids having the D-configuration instead of the L-configuration). The polypeptides of this invention can include amino acid residues that are modified versions of standard residues (e.g., pyrrolysine can be used in place of lysine and selenocysteine can be used in place of cysteine).

In some embodiments, the amino acid residues in the CRISPR-associated polypeptide (e.g., Cas9) amino acid sequence can include non-naturally occurring amino acid residues. Non-naturally occurring amino acid residues are those that have not been found in nature, but that conform to the basic formula of an amino acid and can be incorporated into an amino acid sequence. These include D-alloisoleucine (2R,3S)-2-amino-3-methylpentanoic acid and L-cyclopentyl glycine (S)-2-amino-2-cyclopentyl acetic acid.

In some embodiments, the Cas (e.g., Cas9) nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks.

In some embodiments, the Cas (e.g., Cas9) nuclease can be rendered catalytically inactive with mutations in the conserved domains of both RuvC and HNH domains, which are responsible for nuclease activity. For example, mutations of D10A and H840A in the RuvC and HNH domains, respectively, result in a catalytically inactive, i.e., dead, Cas (e.g., dead Cas9). In some embodiments, fusing a repressor domain to the dead Cas (e.g., dead Cas9) allows transcription to be even further repressed by inducing heterochromatinization. For example, the Krüppel associated box (KRAB) domain can be fused to dead Cas (e.g., dead Cas9) to repress transcription of a target gene.

The bonds between the amino acid residues of the polypeptides of this invention can be conventional peptide bonds or another covalent bond (such as an ester or ether bond), and the polypeptides can be modified by amidation, phosphorylation and/or glycosylation. A modification can affect the polypeptide backbone and/or one or more side chains. Chemical modifications can be naturally occurring modifications made in vivo following translation of an mRNA encoding the polypeptide (e.g., glycosylation in a bacterial host) or synthetic modifications made in vitro. A biologically active variant of a CRISPR-associated endonuclease can include one or more structural modifications resulting from any combination of naturally occurring (i.e., made naturally in vivo) and/or synthetic modifications (i.e., naturally occurring or non-naturally occurring modifications made in vitro). Examples of modifications include, but are not limited to, amidation (e.g., replacement of the free carboxyl group at the C-terminus by an amino group); biotinylation (e.g., acylation of lysine or other reactive amino acid residues with a biotin molecule); glycosylation (e.g., addition of a glycosyl group to either asparagine, hydroxylysine, serine or threonine residues to generate a glycoprotein or glycopeptide); acetylation (e.g., the addition of an acetyl group, typically at the N-terminus of a polypeptide); alkylation (e.g., the addition of an alkyl group); isoprenylation (e.g., the addition of an isoprenoid group); lipoylation (e.g. attachment of a lipoate moiety); and phosphorylation (e.g., addition of a phosphate group to serine, tyrosine, threonine or histidine).

A biologically active variant of a CRISPR-associated endonuclease polypeptide will retain sufficient biological activity to be useful in the present methods. The biologically active variants will retain sufficient activity to function in targeted DNA cleavage. The biological activity can be assessed in ways known to one of ordinary skill in the art and includes, without limitation, in vitro cleavage assays or functional assays.

The present invention also provides compositions (e.g., pharmaceutical compositions) of the CRISPR-associated endonuclease, the nuclease (e.g., TALE DNA binding domain, TALEN, zinc finger DNA binding domain and/or zinc finger nuclease), the guide RNA molecule(s), vector, nucleic acid construct, nucleic acid molecule, nanoparticle or microparticle, etc., of this invention in a pharmaceutically acceptable carrier and/or a suitable diluent known in the art. Such compositions can comprise buffers such as neutral buffered saline, phosphate buffered saline, sterile saline and the like; carbohydrates such as glucose, mannose, sucrose or dextrans, mannitol; proteins; polypeptides or amino acids such as glycine; antioxidants; chelating agents such as EDTA and/or glutathione; adjuvants (e.g., aluminum hydroxide) and/or preservatives, singly or in any combination.

In some embodiments, the composition of this invention can comprise, consist essentially of or consist of a CRISPR-Cas endonuclease and one or more gRNA molecules comprising a nucleotide sequence of any of SEQ ID NOs:3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 72, 74, 75, 76, 77, 78, 79, 80, 81, 82, 83, 84, 85, 86, 87, 88, 89 or 90, in a pharmaceutically acceptable carrier.

In some embodiments, the composition of this invention can comprise, consist essentially of or consist of a nucleic acid molecule encoding a CRISPR-Cas endonuclease and a nucleic acid molecule encoding one or more gRNA molecules comprising a nucleotide sequence of any of SEQ ID NOs:3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 72, 74, 75, 76, 77, 78, 79, 80, 81, 82, 83, 84, 85, 86, 87, 88, 89 or 90, in a pharmaceutically acceptable carrier.

In some embodiments, the composition of this invention can comprise, consist essentially of or consist of a nucleic acid molecule encoding a TALE DNA binding domain that recognizes one or more nucleotide sequence of any of SEQ ID NOs:3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 72, 74, 75, 76, 77, 78, 79, 80, 81, 82, 83, 84, 85, 86, 87, 88, 89 or 90, in a pharmaceutically acceptable carrier.

In some embodiments, the composition of this invention can comprise, consist essentially of or consist of a nucleic acid molecule encoding a zinc finger DNA binding domain that recognizes one or more nucleotide sequence of any of SEQ ID NOs:3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 72, 74, 75, 76, 77, 78, 79, 80, 81, 82, 83, 84, 85, 86, 87, 88, 89 or 90, in a pharmaceutically acceptable carrier.

Administration of the composition of this invention to a subject of this invention can be accomplished by any of several different routes. In specific embodiments, the composition(s) can be administered intravenously, intrathecally, intramuscularly, subcutaneously, intraperitoneally, intradermally, intranasally, intracranially, sublingually, intravaginally, intrarectally, and/or orally. In some embodiments, the composition of the invention can be administered via intracerebroventricular (i.e., intraventricular) injection or delivery. In some embodiments, the composition and/or pharmaceutical composition can be administered as a single dose or in more than one (e.g., 2, 3, 4, 5, 6, 7, 8, 9, 10, etc.) dose. Dosages of the composition of this invention to be administered to a subject depend on the mode of administration, the particular nucleic acid to be delivered, and the like, and can be determined in a routine manner by one of skill in the art. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, or 10¹⁵ virus genomes (vg)/kg, optionally about 10⁸ to about 10¹⁴ vg/kg.

Non-limiting examples of delivery or administration of the nucleic acid molecules of this invention include transduction, transfection, electroporation, liposomes and/or any other methods known in the art or later identified, in any combination. In some embodiments, the nucleic acid molecule of this invention can be administered or delivered as a naked nucleic acid molecule.

In some embodiments, the vector of this invention can be introduced into a subject via several different routes including, but not limited to, intravenous, intrathecal, and/or intracerebroventricular administration.

In some embodiments, the CRISPR-associated endonuclease and the one or more guide RNAs can be introduced into the subject as a nucleic acid molecule, which can be present, e.g., in a nucleic acid construct, as naked DNA, as a plasmid and/or as a viral vector. In some embodiments, the nucleic acid molecule of the invention can be in an adeno-associated virus (AAV) vector comprising a nucleotide sequence encoding the nuclease of the invention. In some embodiments, the AAV vector can comprise one or more engineered capsid proteins. Exemplary capsid proteins include, but are not limited to, chimeric capsid proteins that are not typically found in nature, and are engineered to enhance tissue and cell delivery and/or to enhance transduction, etc., as are known in the art. In particular embodiments, the nucleic acid molecule is in an AAV9 vector (that optionally comprises engineered capsid proteins) comprising a nucleotide sequence encoding a Cas9 nuclease, which can be e.g., SpCas9, SaCas9, NmCas9, or CjCas9, and/or a nucleotide sequence encoding one or more gRNAs.

In some embodiments, the present invention provides a method of unsilencing paternal UBE3A in a human subject (e.g., a subject in need thereof), comprising administering to the subject an effective amount of a nuclease that associates with Snord115 and/or Snord116 and one or more than one guide RNA molecule having complementarity to a target nucleotide sequence in UBE3A-ATS in cells of the subject. In some embodiments, the human subject can be a fetus, an infant, a juvenile or an adult. In some embodiments, the nuclease that associates with Snord115 and/or Snord116 and the one or more than one guide RNA molecule having complementarity to a target nucleotide sequence in UBE3A-ATS in cells of the subject can be administered prenatally (i.e., first, second, and/or third trimester) and/or postnatally (i.e., about 0 to about 30 days after birth) to the infant.

In some embodiments, the present invention provides a method of unsilencing paternal UBE3A in a human subject for at least 3, 6, 9, 12, or 15 months or at least 1.5, 2, 2.5, 3, 3.5, 4, 4.5, 5, 6, 7, 8, 9, 10, 11, 12, 13, 14, or at least 15 years.

In some embodiments, the methods of the present invention can unsilence paternal UBE3A in a human subject in need thereof by at least about 50%, 60%, 70%, 80%, 90%, or 100%.

### Definitions

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising.”

As used herein, the terms “guide RNA” or “gRNA” refer to the polynucleotide sequence comprising the guide sequence, the tracer sequence and the tracer mate sequence. The term “guide sequence” refers to the about 20 bp sequence within the guide RNA that specifies the target site and may be used interchangeably with the terms “guide” or “spacer.” The term “tracer mate sequence” may also be used interchangeably with the term “direct repeat(s).”

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which it is naturally associated in nature and as found in nature.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide,” “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. In some embodiments, the subject is a human. In some embodiments, the subject is a newborn, wherein the newborn is an infant that is no more than about 28 days old.

Tissues, cells and their progeny of a biological entity obtained in vivo or cultured in vitro are also encompassed.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or altered nucleotide bases that permit correct readthrough by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA) and the term “deoxyribonucleic acid” (DNA) is inclusive of cDNA and genomic DNA and DNA-RNA hybrids.

The terms “nucleic acid segment,” “nucleotide sequence,” or more generally “segment” will be understood by those in the art as a functional term that includes both genomic sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art. Thus, all or a portion of the nucleic acids of the present codons may be synthesized using codons preferred by a selected host. Species-preferred codons may be determined, for example, from the codons used most frequently in the proteins expressed in a particular host species. Other modifications of the nucleotide sequences may result in mutants having slightly altered activity.

A “vector” refers to a nucleic acid molecule used as a vehicle to carry foreign genetic material into another cell, where it can be replicated and/or expressed. A cloning vector containing foreign nucleic acid is termed a recombinant vector. Examples of vectors are plasmids, viral vectors, cosmids, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs) are for the expression of the transgene in the target cell, and generally have a promoter sequence that drives expression of the transgene. Insertion of a vector into the target cell is referred to transformation or transfection for bacterial and eukaryotic cells, although insertion of a viral vector is often called transduction.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types, as is well known in the art.

The terms “promoter element,” “promoter,” or “promoter sequence” as used herein, refer to a DNA sequence that is located at the 5′ end (i.e., precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. Promoters may be constitutive or regulatable.

The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

“Sequence identity” refers to a measure of relatedness between two or more nucleic acids or proteins, and is typically given as a percentage with reference to the total comparison length. The identity calculation takes into account those nucleotide or amino acid residues that are identical and in the same relative positions in their respective larger sequences.

Calculations of identity may be performed by algorithms contained within computer programs such as “GAP” (Genetics Computer Group, Madison, Wis.) and “ALIGN” (DNAStar, Madison, Wis.) using default parameters. In certain embodiments, sequence “identity” refers to the number of exactly matching residues (expressed as a percentage) in a sequence alignment between two sequences of the alignment. In certain embodiments, percentage identity of an alignment may be calculated using the number of identical positions divided by the greater of the shortest sequence or the number of equivalent positions excluding overhangs wherein internal gaps are counted as an equivalent position. For example the polypeptides GGGGGG and GGGGT have a sequence identity of 4 out of 5 or 80%. For example, the polypeptides GGGPPP and GGGAPPP have a sequence identity of 6 out of 7 or 85%. In certain embodiments, for any contemplated percentage sequence identity, it is also contemplated that the sequence may have the same percentage of sequence similarity.

Percent “similarity” is used to quantify the extent of similarity, e.g., hydrophobicity, hydrogen bonding potential, electrostatic charge, of amino acids between two sequences of the alignment. This method is similar to determining the identity except that certain amino acids do not have to be identical to have a match. In certain embodiments, sequence similarity may be calculated with well-known computer programs using default parameters. Typically, amino acids are classified as matches if they are among a group with similar properties, e.g., according to the following amino acid groups: Aromatic—F Y W;

As used herein, “effective amount” refers to an amount of a population or composition or formulation of this invention that is sufficient to produce a desired effect, which can be a therapeutic effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, delay of the onset of the disorder, disease or illness, and/or change in any of the clinical parameters of a disorder, disease or illness, etc., as would be well known in the art.

By “pharmaceutically acceptable” is meant a material that is not biologically or otherwise undesirable, i.e., the material may be administered to a subject along with the selected particles, and/or populations thereof, without causing substantial deleterious biological effects or interacting in a deleterious manner with any of the other components of the composition in which it is contained. The pharmaceutically acceptable carrier is suitable for administration or delivery to humans and other subjects of this invention. The carrier would naturally be selected to minimize any degradation of the active ingredient and to minimize any adverse side effects in the subject, as would be well known to one of skill in the art (see, e.g., Remington's Pharmaceutical Science; latest edition). Pharmaceutical formulations, such as vaccines or other immunogenic compositions of the present invention can comprise an immunogenic amount of the alphavirus particles of this invention, in combination with a pharmaceutically acceptable carrier. Exemplary pharmaceutically acceptable carriers include, but are not limited to, sterile pyrogen-free water and sterile pyrogen-free physiological saline solution.

The present subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### EXAMPLES

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

### Example 1: CRISPR/Cas9-Based Gene Therapy for Angelman Syndrome

Angelman syndrome (AS) is a severe neurodevelopmental disorder for which no effective treatment or cure currently exists. AS is characterized by developmental delays, severe intellectual disabilities, lack of speech, debilitating seizures, and problems with movement and balance. AS is classified as an autism spectrum disorder because of phenotypic overlap. With a prevalence of 1:15,000 and a need for constant care across a full lifespan, the family burden and health care costs are immense. There is a significant biomedical need to develop therapeutics that treat some or all symptoms associated with this pediatric-onset genetic disorder.

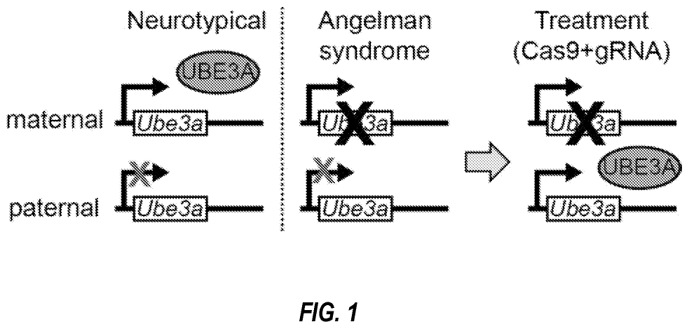

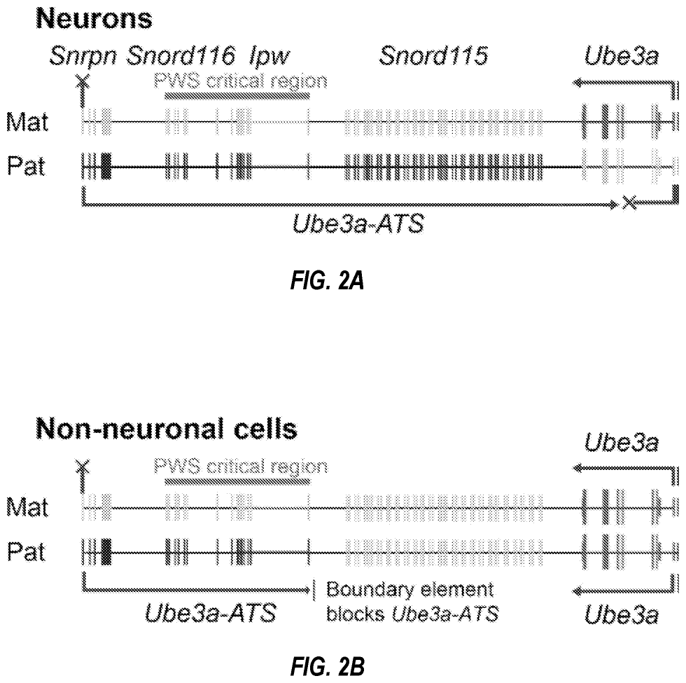

In most cases, AS is caused by deletion or mutation of the maternally-inherited UBE3A allele. UBE3A is expressed biallelically in nearly all cells of the body except mature neurons (FIGS. 2A-2B). In neurons, UBE3A is expressed only from the maternally-inherited allele. This biology explains why loss of the maternal allele causes AS and impairs brain function. The paternal allele is silenced by UBE3A-ATS, an extremely long antisense transcript that interferes with the paternal UBE3A transcript in cis. In light of this biology, the most direct way to treat neural and behavioral dysfunctions associated with AS is to unsilence the intact paternal UBE3A allele (FIG. 1). The present invention is based on the scientific premise that disruption or truncation of UBE3A-ATS so that it does not interfere with paternal UBE3A, will unsilence paternal UBE3A and treat behavioral phenotypes associated with AS.

Previously, one of the inventors performed a >2,300 drug screen and found that topoisomerase inhibitors, including topotecan, potently unsilenced the paternal Ube3a allele in mouse and human neurons by reducing Ube3a-ATS expression. Topotecan increased paternal UBE3A to near normal levels in cultured neurons, in the brain (intracerebroventricular or i.c.v. route), and in the spinal cord. However, topoisomerase inhibitors have side effects in humans and, as the inventors found, downregulate other long genes, including synaptic genes linked to autism.

In subsequent studies, Ube3a-ATS was truncated by knocking-in a transcription stop cassette. This germ line modification unsilenced paternal Ube3a in neurons and rescued behavioral deficits in AS model mice. Antisense oligonucleotides (ASOs) directed to a similar region of Ube3a-ATS unsilenced paternal Ube3a, although these ASOs were not as effective as topotecan. When delivered i.c.v. to adult AS mice (2-4 mo. old), these ASOs modestly rescued one behavioral phenotype (% freezing in contextual fear assay), and partially normalized body weight. However, other behavioral phenotypes were not rescued, consistent with the need to restore Ube3a in younger animals for more complete phenotype rescue (Table 6). Moreover, the effects of ASOs were transient (16 weeks), necessitating repeated invasive injections throughout life, which is not practical or desirable, especially in children.

In the studies described herein, the inventors used AAV9 to deliver Cas9 and gRNAs to the brain in an AS model. On- and off-target effects were examined. These experiments provide proof-of-concept that delivery of Cas9 and gRNAs that target Ube3a-ATS can unsilence Ube3a for an extended period of time (e.g., at least six months) and rescue phenotypes in an AS model. In some embodiments, non-viral approaches to deliver Cas9 protein or RNA may also be used, with the gRNAs of this invention.

AS mosaics indicate some behavioral recovery can be achieved even if UBE3A is restored in as few as 10% of all neurons. While rare, imprinting deficits can inactivate maternal UBE3A and cause AS. Imprinting deficits are typically mosaic, meaning some neurons express “normal” maternal UBE3A while other neurons lack maternal UBE3A expression. AS symptoms are classified as “exceptionally mild” when as few as 10% of all cells contain normal levels of UBE3A. Mild phenotypes include near normal speech, near normal motor performance, and absence of seizures. The inventors can unsilence paternal Ube3a in >50% of all cortical neurons by delivering Cas9 and a gRNA to the brain with AAV9, so they already surpassed the 10% threshold required to move an otherwise severe phenotype to exceptionally mild. Improvements of this magnitude would significantly improve the lives of individuals with AS and their caregivers.

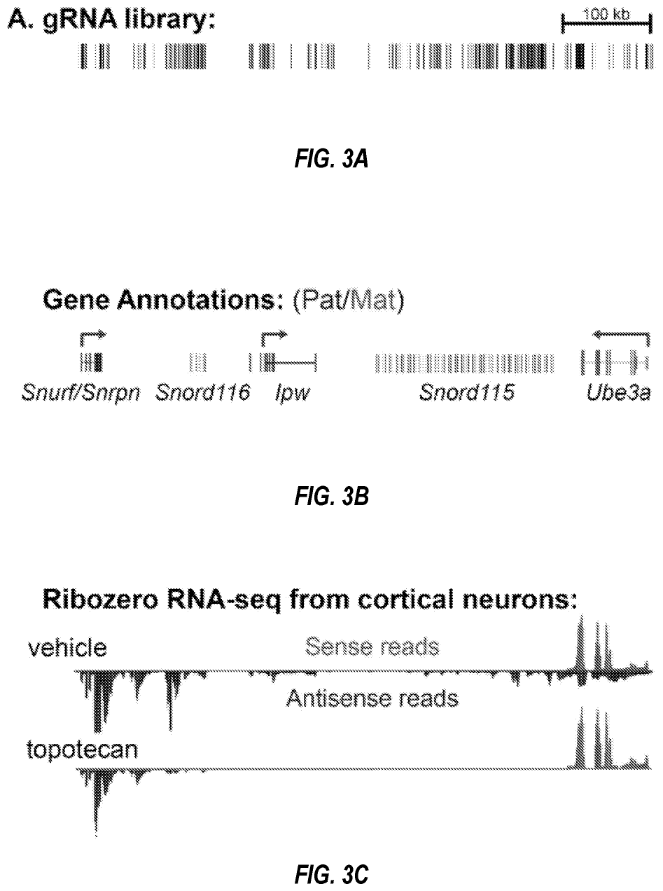

In the present invention, regions in Ube3a-ATS have been identified that can be targeted by Cas9 to unsilence paternal Ube3a in neurons. In these studies, 288 SpCas9-compatible gRNAs were designed that target Ube3a-ATS and nearby genes (FIGS. 3A-3B; using the MIT CRISPR Design Tool to identify gRNAs with a low probability of off target effects. gRNAs that target potential regulatory regions were also designed. It was hypothesized that regions in Ube3a-ATS that are sensitive to topotecan treatment might also be sensitive to disruption by Cas9. To identify such regions, the inventors treated cortical neuron cultures with vehicle or topotecan and then used Ribo-zero RNA-seq to interrogate nascent (non-polyA selected) transcripts at extremely deep coverage (>280 million mapped reads/condition; 3 replicates/condition) (FIG. 3C). The inventors also targeted putative regulatory elements (FIG. 3D), including CTCF binding sites, DNase hypersensitive sites, chromatin epigenetic marks, polyadenylation sites, predicted sites of RNA secondary structure, and transcription factor ChIP-seq binding sites (identified from ENCODE and other publically available data sets). All gRNAs were cloned into the pLenti CRISPR v2 plasmid, which can be used for transient transfection or lentiviral delivery. The inventors used a PCR-based approach to verify that each plasmid contained the correct gRNA.

The inventors cultured neurons from Ube3a yellow fluorescent protein (YFP) knock-in reporter mice, using a high-content 384-well format assay. The inventors transiently co-transfected Ube3a^{m+/patYFP} primary cortical neurons with CamKIIα-tdTomato (to identify transfected neurons; the CamKIIα promoter is neuron-selective), SpCas9, and each gRNA from their library. Wells were imaged in their entirety with a high content imager (GE IN Cell Analyzer 2200, 9 frames per well). The percentage of CamKIIα-tdTomato+ transfected neurons that were YFP+ (unsilenced UBE3A-YFP) was quantified using Cellprofiler. The inventors ranked all gRNAs relative to topotecan, the benchmark positive control, and relative to other positive and negative controls. Several gRNAs that unsilenced paternal Ube3a as effectively as topotecan were identified. Several “hits” were located in or near Snord115 and Snord116 genes (also known as H/MBII-52 and H/MBII-85, respectively), two clusters of C/D box snoRNAs that are processed from introns of Ube3a-ATS.

### Example 2: Identification of First Candidate Therapeutic gRNAs for AS

Deletion of SNORD116 genes causes Prader-Willi Syndrome (PWS). The PWS critical region overlaps and is restricted to SNORD116 genes (Bieth et al. Eur J Hum Genet 23:252-255 (2015); deSmith et al. Hum Mol Genet 18:3257-3265 (2009)). Thus, gRNAs that delete all SNORD116 genes or that downregulate the entire Ube3a-ATS transcript (which will eliminate all SNORD116 genes) are not likely to be of therapeutic utility. In contrast, gRNAs that target regions outside (3′) of the PWS critical region are likely to be efficacious at treating AS with few to no side effects (gRNAs in this region are defined as “candidate therapeutic gRNAs”). By targeting these regions, the inventors can simultaneously overcome major limitations associated with other therapeutic approaches while also achieving the following:

1. Long-lasting unsilencing of paternal Ube3a with a single treatment. AAV drives expression in the primate brain and behavioral recovery for at least 15 years. AAV-mediated delivery of dead Cas9 and a Snord115 gRNA will block transcription for a very long time, covering most if not all critical periods of brain development, especially if administered to newborns. AAV drives long-lasting gene expression following a single injection, and contrasts with transient effects of ASOs, small molecules, and protein-based repressors.

2. Built-in redundancy to restrict changes in paternal Ube3a expression to neurons. Cas9 expression can be restricted by using the neuron-specific promoter, hSyn1. In the event that Cas9 is expressed in non-neuronal cells, the underlying biology provides an additional layer of restriction: In nonneuronal cells, there is a boundary element that truncates Ube3a-ATS within the PWS critical region (FIGS. 2A-2B). In neurons, Ube3a-ATS extends beyond this boundary element and silences paternal Ube3a via a transcriptional collision mechanism (FIGS. 2A-2B). By focusing on gRNAs that target Ube3a-ATS downstream of this boundary element, one can restrict changes in paternal Ube3a expression to neurons.

3. Limit side effects by harnessing the extreme redundancy of SNORD115 genes. While SNORD115 genes regulate alternative splicing of several genes in the brain, they also are highly redundant. There are 48 SNORD115 genes in humans and 110 Snord115 genes in mice. The sequence and genomic organization of Snord115 genes is conserved between species. Snord115 genes are located 3′ of the PWS critical region, so their deletion will not cause PWS. Mice with a large deletion encompassing Snord115 and other nearby genes are viable, indicating that all Snord115 genes can be deleted without affecting viability.

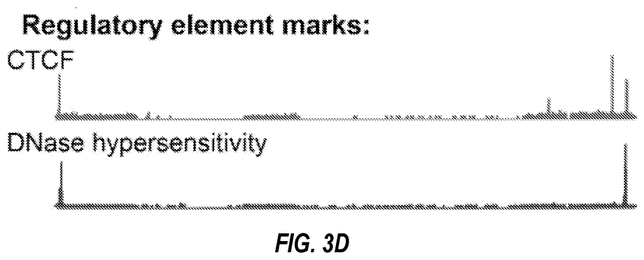

As a key innovation, the most effective Snord115 gRNA (jw33; transfected with active SpCas9, Tables 1-5) had an on-target effect, as evidenced by downregulation of Snord115 and the 3′ Ube3a-ATS region (FIG. 4). jw33 did not alter the levels of upstream genes (Snrpn, Snord116; FIG. 4). This approach has the unique potential to unsilence paternal Ube3a without compromising other Snord115 functions, and is highly unlikely to compromise Snrpn and Snord116 functions. These studies demonstrate the therapeutic potential of using CRISPR/Cas9 to target this highly redundant cluster of Snord115 genes for the treatment of AS.

Other effective gRNAs target Snord116 genes (Tables 1-5), reproducing results with an ASO targeted to Snord116. While Snord116 genes are highly redundant, their complete deletion is linked to PWS pathogenesis, making them a risky target for the treatment of AS.

4. Restore Ube3a to normal levels. UBE3A levels must be tightly maintained within a narrow range for normal brain development, as evidenced by the fact that loss of maternal UBE3A causes AS while duplication of UBE3A increases autism risk. Thus, traditional gene replacement therapies are not ideal, as they often drive gene expression of a single Ube3a isoform at abnormally high levels, with variability between cells due to vector copy number. In contrast, in the present invention expression of paternal Ube3a is driven from the endogenous promoter (which is identical to the maternal promoter). As a result, protein levels and isoform distribution will be equivalent to the maternal copy, providing an optimal treatment for the vast majority of all AS individuals who are missing maternal Ube3a.

### Example 3: Preliminary Studies to Assess Allele-Specific Expression of UBE3A and UBE3A-ATS in Human Neurons

Snord115 genes are conserved between mice and humans, making it possible to translate the studies described in the present invention to human neurons. Allele-specific expression of human UBE3A can be quantified using single nucleotide polymorphisms (SNPs) or repeat length polymorphisms that differ between the maternal and paternal chromosome (i.e., are heterozygous). 107 primary human neuronal progenitor cell (phNPC) lines from presumed neurotypical fetal human brains were evaluated. These lines can be differentiated into neurons with higher fidelity than embryonic stem cells or induced pluripotent stem cells (IPSCs). All phNPC lines were genotyped using single nucleotide polymorphism (SNP) chips, and stranded RNA-seq data were collected from most of the phNPC lines before and after differentiation into neurons.

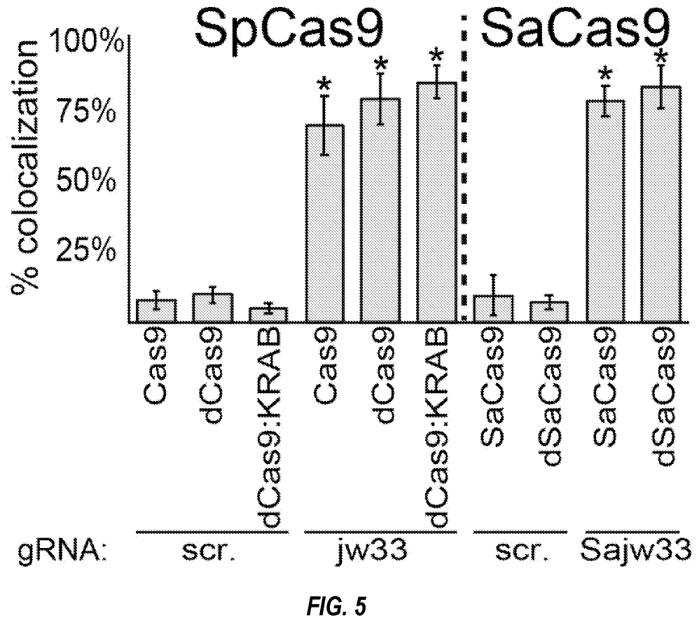

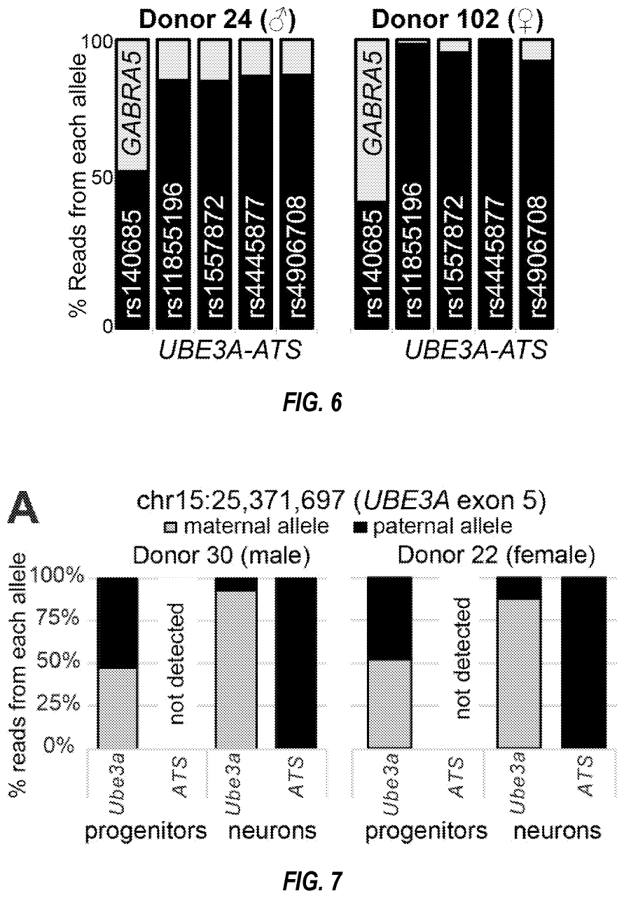

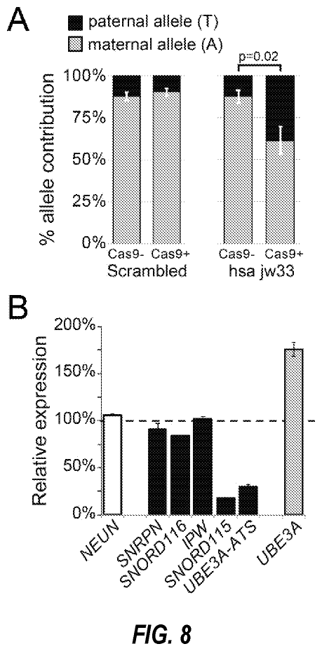

By analyzing these RNA-seq data, SNPs were identified in UBE3A-ATS, in regions that overlap introns of UBE3A (FIG. 6), and seven SNPs were identified in exons of UBE3A (FIG. 7, Table 7), that are heterozygous in some of the donor cell lines. These SNPs can be used to detect biallelic expression of UBE3A in progenitors and monoallelic (presumably maternal) expression of UBE3A in mature neurons (FIG. 7). We can also quantify monoallelic (presumably paternal) expression of the UBE3A ATS in mature neurons (this region is not expressed in progenitors, as expected, FIG. 7). We next treated phNPC-derived neurons with lentiviruses containing active SpCas9 and a human gRNA that is similar to jw33 (“hsa jw33”, e.g., Snord115-6). Importantly, this treatment unsilenced paternal UBE3A in human neurons (FIG. 8).

As an additional approach, a repeat length polymorphism in intron 9 of human UBE3A can be used to distinguish maternal from paternal expression in IPSCs (using allele-specific RT-PCR with sense and antisense pre-mRNA transcripts). In preliminary studies, it was confirmed that several of the donor lines are polymorphic in intron 9 of human UBE3A, allowing for the use a repeat length polymorphism in intron 9 and pre-mRNA RT-PCR to quantify allelic expression of UBE3A.

### Example 4: Quantifying Allele Specific Expression in Differentiated Human Neurons

Primary human neural progenitor cells (phNPCs) are expanded and plated in 6 well plates at a density of 4×10⁵ cells per well. 48 hours after plating, proliferation media is replaced with differentiation media containing human recombinant NT3 and BDNF. For 8 weeks, cells undergo 50% media change every 2-3 days. At week 4, cells are infected with a lentivirus cocktail containing pLC2-SpCas9:mCherry-Snord115 gRNA (or negative control) and pLenti CamkIIa:eGFP to mark neurons. At 8 weeks post differentiation, cells are collected and sorted for mCherry+/eGFP+/DAPI+ events. RNA is extracted using Trizol and DNaseI treated. cDNA is synthesized using a combination of random hexamers and polydT primers. Allele specific expression is assayed by TaqMan genotyping probes using a SNP identified from RNAseq (Table 7). The expected allelic expression pattern following differentiation in specific donor lines is shown in FIG. 7. The relative change in SNP expression in response to targeting SNORD115 with a gRNA is shown in FIG. 8.

To quantify the difference in expression between the two alleles of each SNP in the UBE3A mRNA, we applied the ΔΔCt method qPCR experiments using allele specific TaqMan probes. Specifically, we compared expression levels of the presumed paternal/maternal alleles between phNPC differentiated neurons treated with a) SpCas9 scrambled gRNA, and b) SpCas9 various SNORD115 targeting gRNAs. Preliminary experiments using this approach demonstrate feasibility and efficacy of human SNORD115 targeting gRNAs to unsilence the presumed paternal allele of UBE3A (Table 5).

### Example 5: Active and Dead Cas9 are Effective at Unsilencing Paternal Ube3a

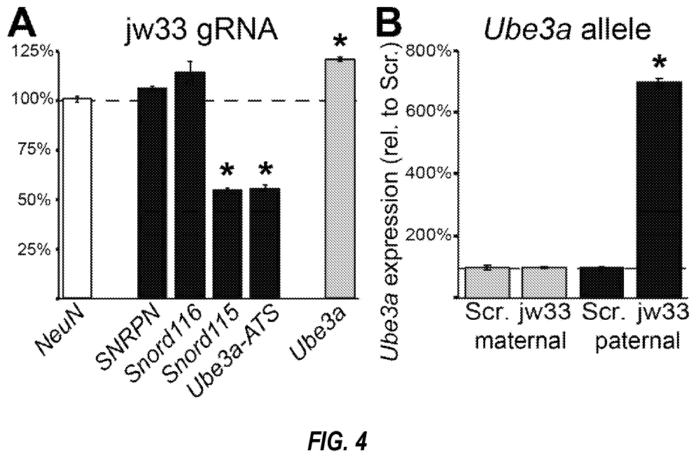

Active Cas9, dead Cas9 (not fused to anything), and dead Cas9 fused to a KRAB repressor domain were equally effective at unsilencing paternal Ube3a (FIG. 5). These data indicate that Cas9 can unsilence paternal Ube3a by binding to Snord115 genes and blocking transcription. This discovery makes it possible to use dead Cas9 to treat AS and minimize side effects. Dead Cas9 does not contain a chromatin modifying domain, nor does it cut the genome, so there is little-to-no risk of mutagenesis, cancer, or p53 activation/double strand breaks. The inventors discovered that dead Cas9 can be used to bypass the main weaknesses of CRISPR as a therapeutic. Moreover, dead Cas9 can be combined with anti-CRISPRs or other technologies to deactivate dCas9 should reversibility be desirable.

### Example 6: Prenatal Intraventricular Injection of AAV2-Hsyn1-SaCas9-Sajw33 or AAV9-Hsyn1-SaCas9-Sajw33

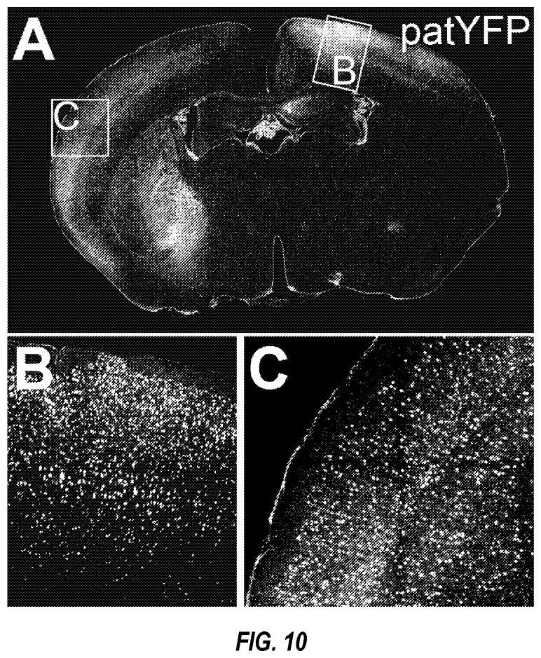

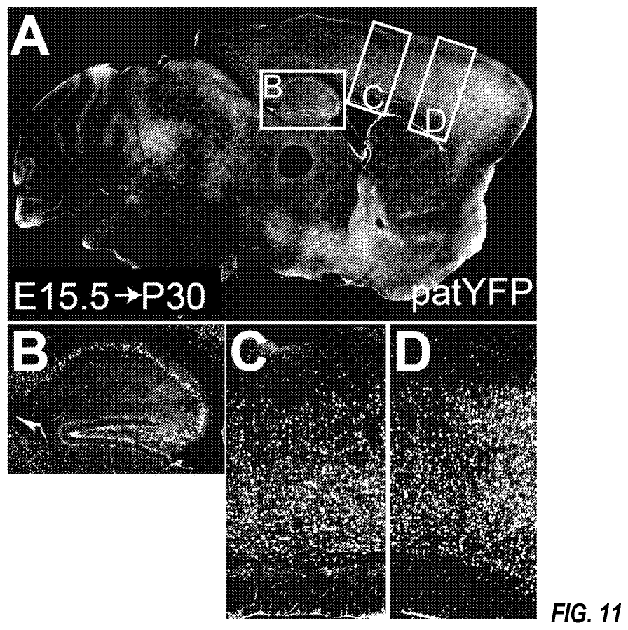

Virus stock at 2.4×10¹³ virus molecules/ml was mixed with 10% Fast green prepared in sterile PBS for visualization of the injection site. Timed mating is set up using either C57BL/6 male with Ube3a^{mat+/pat-} female or Ube3a^{m+/patUbe3a} male with C57BL/6 female mice. At E15.5, pregnant females were anesthetized with 2% isoflurane throughout the procedure. Embryos in the uterus were injected with virus solution at 1 μl/hemisphere in the ventricle. Approximately 2.2×10¹⁰ virus molecules were injected to each side of the brain. Injected embryos were repositioned into the abdominal cavity. Operated females were treated with 5 mg/Kg Ketamine daily for three days. Pups were allowed to give birth naturally and kept with a foster mom till weaning. At P30, Ube3a^{mat+/patYFP} or Ube3a^{mat-/pat+} pups were perfused with 4% PFA in PBS. Mouse brains were dissected and postfixed in 4% PFA via overnight incubation at 4° C. Brains were sectioned to 100 μm slices using a Leica Vibratome. Brain slices were then stained with rabbit anti-GFP antibody (Ube3a^{mat+/patYFP}) or mouse anti-UBE3A antibody (Ube3a^{mat-/pat+}) and imaged through a Zeiss 780 Confocal microscope at 10× magnification (FIGS. 10, 11).

### Example 7: Postnatal Intraventricular Injection of AAV9-Hsyn1-SaCas9-Sajw33

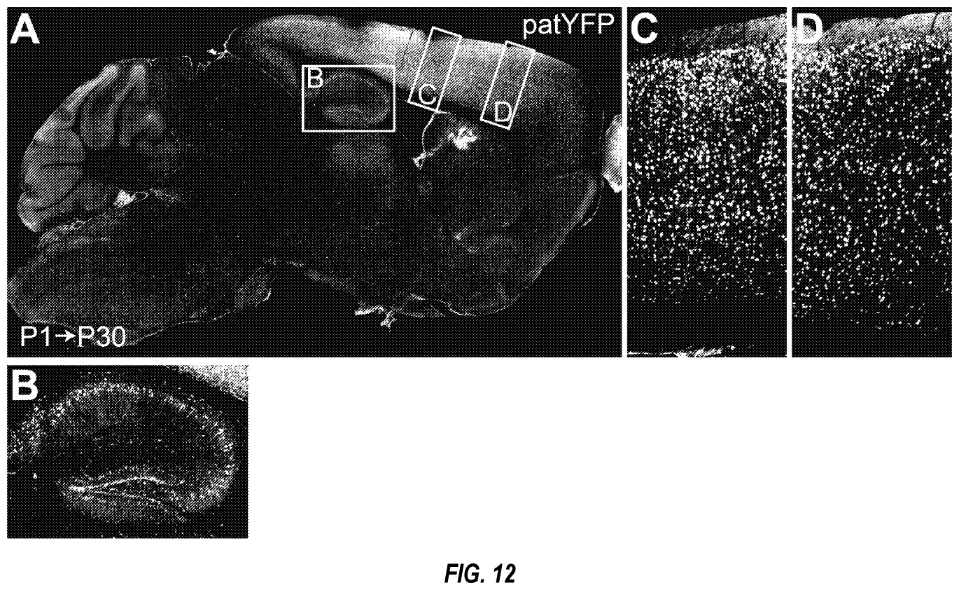

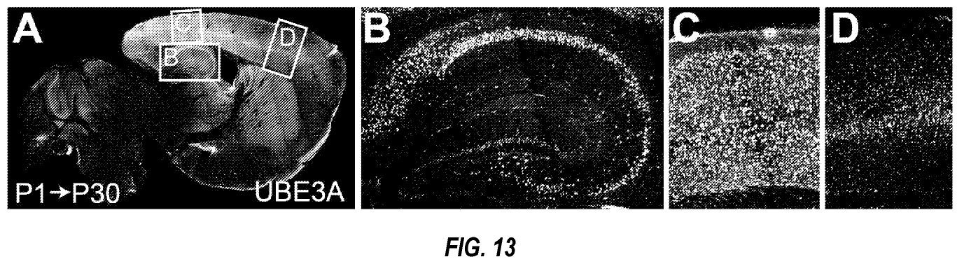

Virus stock at 2.4×10¹³ virus molecules/mL was mixed with 10% Fast green prepared in sterile PBS for visualization of the injection site. P1 neonatal Ube3a^{mat+/patYFP} or Ube3a^{mat-/pat+} pups were immobilized via cryo-anesthesia for 3.5 minutes. Virus was injected into both sides of the ventricle with 32G Hamilton needle attached to a 25 μl injection syringe. 1 μl of virus solution, approximately 2.2×10¹⁰ virus molecules were injected to each side of the brain. Injected pups were allowed to recover on a heated mat at 37° C. and returned to the home cage. Virus injected Ube3a^{mat-/patYFP} or Ube3a^{mat-/pat+} pups were perfused with 4% PFA in PBS at P30, brains were dissected and postfixed in 4% PFA via overnight incubation at 4° C. Brains were sectioned to 100 μm slices using a Leica Vibratome. Brain slices were then stained with rabbit anti-GFP antibody (Ube3a^{mat+/patYFP}) or mouse anti-UBE3A antibody (Ube3a^{mat-/pat+}) and imaged through a Zeiss 780 Confocal microscope at 10× magnification (FIGS. 12, 13).

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

Lengthy table referenced here
US20210054370A1-20210225-T00001
Please refer to the end of the specification for access instructions.

Lengthy table referenced here
US20210054370A1-20210225-T00002
Please refer to the end of the specification for access instructions.

Lengthy table referenced here
US20210054370A1-20210225-T00003
Please refer to the end of the specification for access instructions.

Lengthy table referenced here
US20210054370A1-20210225-T00004
Please refer to the end of the specification for access instructions.

Lengthy table referenced here
US20210054370A1-20210225-T00005
Please refer to the end of the specification for access instructions.

Lengthy table referenced here
US20210054370A1-20210225-T00006
Please refer to the end of the specification for access instructions.

Lengthy table referenced here
US20210054370A1-20210225-T00007
Please refer to the end of the specification for access instructions.

Lengthy table referenced here
US20210054370A1-20210225-T00008
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20210054370A1). An electronic copy of the table will also be available from the USPTO upon request and payment of the fee set forth in 37 CFR 1.19(b)(3).

1. A method of unsilencing paternal UBE3A in a human subject in need thereof, comprising administering to the subject:
a) an effective amount of a clustered regularly interspersed short palindromic repeat (CRISPR)-associated endonuclease and one or more than one guide RNA molecule having complementarity to a target nucleotide sequence in UBE3A ATS (SEQ ID NO:1) in cells of the subject;
b) an effective amount of a dead SpCas9-KRAB fusion and/or a dead SaCas9-KRAB fusion and one or more than one guide RNA molecule having complementarity to a target nucleotide sequence in Snord115 in UBE3A-ATS in cells of the subject and/or
c) an effective amount of a dead SpCas9 and/or a dead SaCas9 and one or more than one guide RNA molecule having complementarity to a target nucleotide sequence in Snord115 in UBE3A-ATS in cells of the subject.

2. A method of treating Angelman syndrome in a subject in need thereof, comprising administering to the subject:
a) an effective amount of a clustered regularly interspersed short palindromic repeat (CRISPR)-associated endonuclease and one or more than one guide RNA molecule having complementarity to a target nucleotide sequence in UBE3A-ATS (SEQ ID NO:1) in cells of the subject;
b) an effective amount of a dead SpCas9-KRAB fusion and/or a dead SaCas9-KRAB fusion and one or more than one guide RNA molecule having complementarity to a target nucleotide sequence in Snord115 in UBE3A-ATS in cells of the subject and/or
c) an effective amount of a dead SpCas9 and/or a dead SaCas9 and one or more than one guide RNA molecule having complementarity to a target nucleotide sequence in Snord115 in UBE3A-ATS in cells of the subject.

3. The method of claim 1, wherein the CRISPR-associated endonuclease is Cas9, CasX, Cas12, or a variant thereof.

4. The method of claim 3, wherein the CRISPR-associated endonuclease is human-optimized Cas9, CasX, Cas12, or a variant thereof.

5. The method of claim 4, wherein the Cas9, CasX, Cas12, or a variant thereof, is dead and/or catalytically inactive.

6. The method of claim 5, wherein the dead Cas9, CasX, Cas12, or a variant thereof, is fused to a transcriptional repressor domain.

7. The method of claim 1, wherein the target nucleotide sequence in UBE3A-ATS is in one or more SNORD115 (HBII-52 in human, MBII-52 in mouse) and/or SNORD115HG genes.

8. The method of claim 1, wherein the target nucleotide sequence in UBE3A-ATS is in one or more SNHG14 genes.

9. The method of claim 1, wherein the target nucleotide sequence in UBE3A-ATS is in one or more SNORD109B genes.

10. The method of claim 1, wherein the target nucleotide sequence in UBE3A-ATS is in one or more SNORD116 and/or SNORD116HG genes.

11. The method of claim 1, wherein the guide RNA comprises the nucleotide sequence of any of SEQ ID NOs:3-90.

12. The method of claim 1, wherein the CRISPR-associated endonuclease and the one or more guide RNAs are introduced into the subject as one or more nucleic acid molecules.

13. The method of claim 12, wherein the nucleic acid molecules are present in a vector.

14. The method of claim 13, wherein the vector is a viral vector.

15. The method of claim 14, wherein the viral vector is an adeno-associated virus (AAV) vector.

16-19. (canceled)

20. A composition comprising:
a) a CRISPR-associated endonuclease and one or more guide RNA molecules having complementarity to a target nucleotide sequence in UBE3A-ATS; and/or
b) a nucleic acid molecule encoding a CRISPR-associated endonuclease and a nucleic acid molecule encoding one or more than one guide RNA having complementarity to a target nucleotide sequence in UBE3A-ATS,
in a pharmaceutically acceptable carrier.

21. (canceled)

22. The composition of claim 20, wherein the nucleic acid molecule encoding the CRISPR-associated endonuclease and the nucleic acid molecule encoding the one or more than one guide RNA molecule are present on a single nucleic acid construct or on two or more separate nucleic acid constructs.

23. (canceled)

24. The composition of claim 22, wherein the single nucleic acid construct is in a viral vector and/or the two or more separate nucleic acid constructs are each present in a viral vector.

25. (canceled)

26. The viral vector of claim 24, wherein the viral vector is an AAV vector.

27. The viral vector of claim 26, wherein the AAV vector is from serotype AAV9.
